(19) United States
(12) Patent Application Publication
(10) Pub. No.: US 2025/0340598 A1
(43) Pub. Date: Nov. 6, 2025

(54) COMPOSITIONS COMPRISING V2 OPT HIV ENVELOPES

(71) Applicants: Duke University, Durham, NC (US); Triad National Security, LLC,, Los Alamos, NM (US); The Trustees of The University of Pennsylvania, Philadelphia, PA (US)

(72) Inventors: Barton F. HAYNES, Durham, NC (US); Kevin O. SAUNDERS, Durham, NC (US); Bette T. KORBER, Los Alamos, NM (US); Kshitij G. WAGH, Los Alamos, NM (US); Beatrice HAHN, Philadelphia, PA (US); George M. SHAW, Philadelphia, PA (US); Wenge DING, Philadelphia, PA (US); Ronnie M. Russell, Philadelphia, PA (US); Drew Weissman, Philadelphia, PA (US); Mohamad-Gabriel ALAMEH, Philadelphia, PA (US)

(21) Appl. No.: 18/700,679

(22) PCT Filed: Oct. 12, 2022
(86) PCT No.: PCT/US2022/046491

§ 371 Date: Apr. 11, 2024

Related U.S. Application Data
(60) Provisional application No. 63/254,867, filed on Oct. 12, 2021.
(60) Provisional application No. 63/338,547, filed on May 5, 2022.

(51) Int. Cl. C07K 14/005; A61K 39/00; A61K 39/21; A61P 37/04

(52) U.S. Cl. CPC C07K 14/005; A61K 39/21; A61P 37/04; A61K 2039/53; A61K 2039/55555; C12N 2740/16122; C12N 2740/16134

(57) ABSTRACT

In certain aspects the invention provides HIV-1 immunogens, including HIV-1 envelopes with optimized V2 loop for antibody induction.

This invention was made with government support under Center for HIV/AIDS Vaccine Immunology-Immunogen Design grant UM1-A1100645 and UM1-A1144371 from the NIH, NIAID, Division of AIDS. The government has certain rights in the invention.

The United States government has rights in this invention pursuant to Contract No. 89233218CNA000001 between the United States Department of Energy and Triad National Security, LLC for the operation of Los Alamos National Laboratory.

This application claims the benefit and priority of U.S. Application Ser. Nos. 63/254,867 filed Oct. 12, 2021 and 63/338,547 filed May 5, 2022 the contents each of which are incorporated by reference in its entirety.

### TECHNICAL FIELD

The present invention relates in general, to a composition suitable for use in inducing anti-HIV-1 antibodies, and, in particular, to immunogenic compositions comprising envelope proteins and nucleic acids to induce cross-reactive neutralizing antibodies and increase their breadth of coverage. The invention also relates to methods of inducing such broadly neutralizing anti-HIV-1 antibodies using such compositions.

### BACKGROUND

The development of a safe and effective HIV-1 vaccine is one of the highest priorities of the scientific community working on the HIV-1 epidemic. While anti-retroviral treatment (ART) has dramatically prolonged the lives of HIV-1 infected patients, ART is not routinely available in developing countries.

### SUMMARY OF THE INVENTION

In certain embodiments, the invention provides compositions and method for induction of immune response, for example cross-reactive (broadly) neutralizing Ab (bNAb) induction.

In certain aspects, the invention provides a CH505, CAP256SU, CAP256wk34.80, CAM13, Q23, or T250 envelope immunogens comprising optimized V2 loop, for example but not limited to initiate VIV2, and/or CD4 binding site and/or Fusion Peptide unmutated common ancestor (UCA) broadly neutralizing antibody (bnAbs) precursors. In certain aspects the invention provides CH505 T/F envelopes comprising optimized V2 loop. In certain aspects the invention provides CAP256SU envelopes comprising optimized V2 loop. In certain aspects the invention provides CAP256wk34.80 envelopes comprising optimized V2 loop. In certain aspects the invention provides CAM13 envelopes comprising optimized V2 loop. In certain aspects the invention provides Q23 envelopes comprising optimized V2 loop. In certain aspects the invention provides T250 envelopes comprising optimized V2 loop.

In certain embodiments, the compositions contemplate nucleic acid, as DNA and/or RNA, or proteins immunogens either alone or in any combination. In certain embodiments, the methods contemplate genetic, as DNA and/or RNA, immunization either alone or in combination with envelope protein(s).

In certain embodiments the nucleic acid encoding an envelope is operably linked to a promoter inserted in an expression vector. In certain aspects the compositions comprise a suitable carrier. In certain aspects the compositions comprise a suitable adjuvant.

In certain embodiments the induced immune response includes induction of antibodies, including but not limited to autologous and/or cross-reactive (broadly) neutralizing antibodies against HIV-1 envelope. Various assays that analyze whether an immunogenic composition induces an immune response, and the type of antibodies induced are known in the art and are also described herein.

In certain aspects the invention provides a nucleic acid sequence encoding any of the polypeptides of the invention, wherein the nucleic acid is operably linked to a promoter. In certain aspects the invention provides a nucleic acid consisting essentially of a nucleic acid sequence encoding any of the polypeptides of the invention, wherein the nucleic acid is operably linked to a promoter. In certain aspects the invention provides an expression vector comprising any of the nucleic acid sequences of the invention, wherein the nucleic acid is operably linked to a promoter. In certain aspects the invention provides an expression vector comprising a nucleic acid sequence encoding any of the polypeptides of the invention, wherein the nucleic acid is operably linked to a promoter. In certain aspects the invention provides an expression vector consisting essentially a nucleic acid sequence encoding any of the polypeptides of the invention, wherein the nucleic acid is operably linked to a promoter. In certain embodiments, the nucleic acids are codon optimized for expression in a mammalian cell, in vivo or in vitro. In certain aspects the invention provides nucleic acids comprising any one of the nucleic acid sequences of invention. In certain aspects the invention provides nucleic acids consisting essentially of any one of the nucleic acid sequences of invention. In certain aspects the invention provides nucleic acids consisting of any one of the nucleic acid sequences of invention. In certain embodiments the nucleic acid of the invention, is operably linked to a promoter and is inserted in an expression vector. In certain aspects the invention provides an immunogenic composition comprising the expression vector.

In certain aspects the invention provides a composition comprising at least one of the nucleic acid sequences of the invention. In certain aspects the invention provides a composition comprising any one of the nucleic acid sequences of invention. In certain aspects the invention provides a composition comprising at least one nucleic acid sequence encoding any one of the polypeptides of the invention.

In certain aspects the invention provides a composition comprising at least one nucleic acid encoding an HIV-1 envelope of the invention.

In certain embodiments, the compositions and methods employ an HIV-1 envelope as polypeptide instead of a nucleic acid sequence encoding the HIV-1 envelope. In certain embodiments, the compositions and methods employ an HIV-1 envelope as polypeptide, a nucleic acid sequence encoding the HIV-1 envelope, or a combination thereof. In certain embodiments, the polypeptides are recombinantly produced.

The envelope used in the compositions and methods of the invention can be a gp160, gp150, gp145, gp140, gp120, gp41, or N-terminal deletion variants thereof as described herein, cleavage resistant variants thereof as described herein, or codon optimized sequences thereof. In certain embodiments the composition comprises envelopes as trimers. In certain embodiments, envelope proteins are multimerized, for example trimers are attached to a particle such that multiple copies of the trimer are attached and the multimerized envelope is prepared and formulated for immunization in a human. In certain embodiments, the compositions comprise envelopes, including but not limited to trimers as particulate, high-density array on liposomes or other particles, for example but not limited to nanoparticles. In some embodiments, the trimers are in a well ordered, near native like or closed conformation. In some embodiments the trimer compositions comprise a homogenous mix of native like trimers. In some embodiments the trimer compositions comprise at least 65%, 70%, 75%, 80%, 85%, 90%, 95% native like trimers.

The polypeptide contemplated by the invention can be a polypeptide comprising any one of the polypeptides described herein. The polypeptide contemplated by the invention can be a polypeptide consisting essentially of any one of the polypeptides described herein. The polypeptide contemplated by the invention can be a polypeptide consisting of any one of the polypeptides described herein. In certain embodiments, the polypeptide is recombinantly produced. In certain embodiments, the polypeptides and nucleic acids of the invention are suitable for use as an immunogen, for example to be administered in a human subject.

In certain embodiments the envelope is any of the forms of HIV-1 envelope. In certain embodiments the envelope is a gp120, gp140, gp145 (i.e. with a transmembrane), gp150 envelope. In certain embodiments, gp140 is designed to form a stable trimer. In certain embodiments envelope protomers form a trimer which is not a SOSIP timer. In certain embodiment the trimer is a SOSIP based trimer wherein each protomer comprises additional modifications. In certain embodiments, envelope trimers are recombinantly produced. In certain embodiments, envelope trimers are purified from cellular recombinant fractions by antibody binding and reconstituted in lipid comprising formulations. See for example WO2015/127108 titled “Trimeric HIV-1 envelopes and uses thereof” which content is herein incorporated by reference in its entirety. In certain embodiments the envelopes of the invention are engineered and comprise non-naturally occurring modifications.

In certain embodiments, the envelope is in a liposome. In certain embodiments the envelope comprises a transmembrane domain with a cytoplasmic tail embedded in a liposome. In certain embodiments, the nucleic acid comprises a nucleic acid sequence which encodes a gp120, gp140, gp145, gp150, gp160.

In certain embodiments, where the nucleic acids are operably linked to a promoter and inserted in a vector, the vectors are any suitable vector. Non-limiting examples include, VSV, replicating rAdenovirus type 4, MVA, Chimp adenovirus vectors, pox vectors, and the like. In certain embodiments, the nucleic acids are administered in NanoTaxi block polymer nanospheres. In certain embodiments, the composition and methods comprise an adjuvant. Non-limiting examples include, AS01 B, AS01 E, gla/5E, alum, Poly I poly C (poly IC), polyIC/long chain (LC) TLR agonists, TLR7/8 and 9 agonists, or a combination of TLR7/8 and TLR9 agonists (see Moody et al. (2014) J. Virol. March 2014 vol. 88 no. 6 3329-3339), or any other adjuvant. Non-limiting examples of TLR7/8 agonist include TLR7/8 ligands, Gardiquimod, Imiquimod and R848 (resiquimod). A non-limiting embodiment of a combination of TLR7/8 and TLR9 agonist comprises R848 and oCpG in STS (see Moody et al. (2014) J. Virol. March 2014 vol. 88 no. 6 3329-3339).

In non-limiting embodiments, the adjuvant is an LNP. See e.g., without limitation Shirai et al. “Lipid Nanoparticle Acts as a Potential Adjuvant for Influenza Split Vaccine without Inducing Inflammatory Responses” Vaccines 2020, 8, 433; doi: 10.3390/vaccines8030433, published 3 Aug. 2020. In non-limiting embodiments, LNPs used as adjuvants for proteins or mRNA compositions are composed of an ionizable lipid, cholesterol, lipid conjugated with polyethylene glycol, and a helper lipid. Non-limiting embodiment include LNPs without polyethylene glycol.

In certain aspects the invention provides a cell comprising a nucleic acid encoding any one of the envelopes of the invention suitable for recombinant expression. In certain aspects, the invention provides a clonally derived population of cells encoding any one of the envelopes of the invention suitable for recombinant expression. In certain aspects, the invention provides a stable pool of cells encoding any one of the envelopes of the invention suitable for recombinant expression.

In certain aspects, the invention provides a recombinant HIV-1 envelope polypeptide listed in Table 1, 2, 3, and/or 4. In certain embodiments, the polypeptide is a non-naturally occurring protomer designed to form an envelope trimer. The invention also provides nucleic acids encoding these recombinant polypeptides. Non-limiting examples of amino acids and nucleic acids of such protomers are shown in FIGS. 3A-5E, 12F, 13, 14, 16, 17, and 18F.

In certain aspects the invention provides a recombinant trimer comprising three identical protomers of an envelope from Table 1, 2, 3 and/or 4. In certain aspects the invention provides an immunogenic composition comprising the recombinant trimer and a carrier, wherein the trimer comprises three identical protomers of an HIV-1 envelope listed in Table 1, 2, 3 and/or 4. In certain aspects the invention provides an immunogenic composition comprising a nucleic acid encoding these recombinant HIV-1 envelope and a carrier.

In certain aspects the invention provides nucleic acids encoding HIV-1 envelopes for immunization wherein the nucleic acid encodes a gp120 envelope, gp120D8 envelope, a gp140 envelope (gp140C, gp140CF, gp140CFI) as soluble or stabilized protomer of a SOSIP trimer, a gp145 envelope, a gp150 envelope, or a transmembrane bound envelope.

In certain aspects the invention provides a selection of HIV-1 envelopes for immunization wherein the HIV-1 envelope is a gp120 envelope or a gp120D8 variant. In certain embodiments a composition for immunization comprises protomers that form stabilized SOSIP trimers.

In certain embodiments, the compositions for use in immunization further comprise an adjuvant.

In certain embodiments, wherein the compositions comprise a nucleic acid, the nucleic acid is operably linked to a promoter, and could be inserted in an expression vector. In certain embodiments, the nucleic acid is a mRNA. In certain embodiments, the nucleic acid is encapsulated in a lipid nanoparticle.

In one aspect the invention provides a composition for a prime boost immunization regimen comprising one or more envelopes from Table 1, 2, 3 and/or 4, wherein the polypeptide is a non-naturally occurring protomer designed to form an envelope trimer, wherein the envelope is a prime or boost immunogen. In one aspect the invention provides a composition for a prime boost immunization regimen comprising one or more envelopes of the invention.

In certain aspects the invention provides methods of inducing an immune response in a subject comprising administering a composition comprising a polypeptide and/or any suitable form of a nucleic acid(s) encoding an HIV-1 envelope(s) in an amount sufficient to induce an immune response.

In certain embodiments, the nucleic acid encodes a gp120 envelope, gp120D8 envelope, a gp140 envelope (gp140C, gp140CF, gp140CFI) as soluble or stabilized protomer of a SOSIP trimer, a gp145 envelope, a gp150 envelope, or a transmembrane bound envelope. In certain embodiments, the polypeptide is gp120 envelope, gp120D8 envelope, a gp140 envelope (gp140C, gp140CF, gp140CFI) as soluble or stabilized protomer of a SOSIP trimer, a gp145 envelope, a gp150 envelope, or a transmembrane bound envelope.

In certain embodiments, the methods comprise administering an adjuvant. In certain embodiments, the methods comprise administering an agent which modulates host immune tolerance. In certain embodiments, the administered polypeptide is multimerized in a liposome or nanoparticle. In certain embodiments, the methods comprise administering one or more additional HIV-1 immunogens to induce a T cell response. Non-limiting examples include gag, nef, pol, etc.

In certain aspects, the invention provides a recombinant HIV-1 Env ectodomain trimer, comprising three gpl20-gp41 protomers comprising a gpl20 polypeptide and a gp41 ectodomain, wherein each protomer is the same and each protomer comprises portions from envelope BG505 HIV-1 strain and gp120 polypeptide portions from a CH505 HIV-1 strain and stabilizing mutations A316W and E64K. In certain embodiments, the trimer is stabilized in a prefusion mature closed conformation, and wherein the trimer does not comprise non-natural disulfide bond between cysteine substitutions at positions 201 and 433 of the HXB2 reference sequence. Non-limited examples of envelopes contemplated as trimers are listed in Table 1. In some embodiments, the amino acid sequence of one monomer comprised in the trimer is shown in FIG. 3-5, 12F, 13, 14, 16, 17, and 18F. In some embodiments, the trimer is immunogenic. In some embodiments the trimer binds to any one of the antibodies PGT145, PGT151, CH103UCA, CH103, VRC01, PGT128, or any combination thereof. In some embodiments the trimer does not bind to antibody 19B and/or 17B.

In certain aspects, the invention provides a pharmaceutical composition comprising any one of the recombinant trimers of the invention. In certain embodiments the compositions comprising trimers are immunogenic. The percent trimer in such immunogenic compositions could vary. In some embodiments the composition comprises 70%, 71%, 72%, 73%, 74%, 75%, 76%, 77%, 78%, 79%, 80%, 81%, 82%, 83%, 84%, 85%, 86%, 87%, 89%, 90%, 91%, 92%, 93%, 94%, 95%, 96%, 97%, 98%, or 99% stabilized trimer.

In certain embodiments, the envelope comprises ferritin. In certain embodiments, the inventive designs comprise modifications, including without limitation linkers between the envelope and ferritin designed to optimize ferritin nanoparticle assembly.

In certain aspects, the invention provides a composition comprising any one of the inventive envelopes or nucleic acid sequences encoding the same. In certain embodiments, the nucleic acid is mRNA. In certain embodiments, the mRNA is comprised in a lipid nano-particle (LNP).

In certain aspects, the invention provides compositions comprising a nanoparticle which comprises any one of the envelopes of the invention.

In certain embodiments, the nanoparticle is ferritin self assembling nanoparticle.

In certain aspects, the invention provides a method of inducing an immune response in a subject comprising administering an immunogenic composition comprising any one of the stabilized envelopes of the invention. In certain embodiments, the composition is administered as a prime and/or a boost. In certain embodiments, the composition comprises nanoparticles. In certain embodiments, methods of the invention further comprise administering an adjuvant.

In certain aspects, the invention provides a composition comprising a plurality of nanoparticles comprising a plurality of the envelopes/trimers of the invention. In non-limiting embodiments, the envelopes/trimers of the invention are multimeric when comprised in a nanoparticle. The nanoparticle size is suitable for delivery. In non-liming embodiments the nanoparticles are ferritin based nanoparticles.

In certain aspects, the invention provides nucleic acids comprising sequences encoding polypeptides or proteins of the invention. In certain embodiments, the nucleic acids are DNAs. In certain embodiments, the nucleic acids are mRNAs. In certain aspects, the invention provides expression vectors comprising the nucleic acids of the invention.

In certain aspects, the invention provides a pharmaceutical composition comprising mRNAs encoding the inventive envelopes. In certain embodiments, these are optionally formulated in lipid nanoparticles (LNPs). In certain embodiments, the mRNAs are modified. Modifications include without limitations modified ribonucleotides, poly-A tail, 5′cap.

In certain aspects the invention provides nucleic acids encoding the inventive polypeptide or protein designs. In non-limiting embodiments, the nucleic acids are mRNA, modified or unmodified, suitable for use any use, e.g but not limited to use as pharmaceutical compositions. In certain embodiments, the nucleic acids are formulated in lipid, such as but not limited to LNPs.

In non-limiting embodiments, the invention provides compositions comprising an envelope selected from FIGS. 4C-4D, 7, 8, 9, 10, 11, 12, 13, 14, 16, 17, 18, 19 or any combination thereof. Non-limiting embodiments of combinations include CAP256SU_UCA_OPT_3.0_K170R (also referred to as CAP256SU_OPT_4.0), CAP256SU_UCA_OPT_2.0, CAM13RRK_K130H, CH505_UCA_OPT3_D167N, or any combination thereof. See FIGS. 8-12. Non-limiting embodiments of combinations includes HIV_CAP256SU_OPT_4.0, CAM13RRRK, CAP256wk34.80_V2_UCA_OPT_4.0, CAP256wk34.80 V2UCAOPT_RRK, CAP256wk34.80_V2UCAOPT_R171K, CAP256wk34.80 PCT64UCA_OPT and A.Q23_17CHIM.SOSIPV5.2.8/293F (HV1301552) or any combination thereof (FIGS. 14-16). In non-limiting embodiments, the composition comprises CAP256wk34.80_V2_UCA_OPT_4.0. In non-limiting embodiments, the composition comprises HIV_CAP256SU_OPT4.0, CAP256wk34.80_V2UCAOPT_R17IK, CAM13RRRK, and Q23.17 (natural Env). In non-limiting embodiments, the composition comprises CAP256wk34.80_V2UCAOPT_RRK, CAP256wk34.80_V2UCAOPT_R17IK, CAM13RRRK, and Q23.17 (natural Env). In non-limiting embodiments, the composition comprises HIV_CAP256SU_OPT4.0, CAP256wk34.80_V2UCAOPT_R17IK, CAM13RRK, and Q23.17 (natural Env). In non-limiting embodiments, the composition comprises CAP256wk34.80_V2UCAOPT_RRK, CAP256wk34.80_V2UCAOPT_R17IK, CAM13RRK, and Q23.17 (natural Env). In non-limiting embodiments, the composition comprises CAP256wk34.80_V2UCAOPT_RRK. In non-limiting embodiments, the composition comprises CAM13RRK. In non-limiting embodiments, the invention provides compositions comprising nucleic acids encoding one or more envelope selected from FIGS. 4C-4D, 7, 8, 9, 10, 11, 12, 13, 14, 16, 17, 18, 19, or any combination thereof. Provided are also methods of using these envelopes and/or nucleic acids, and/or compositions comprising administering an amount sufficient to induce immune responses in a subject.

In certain aspects, the invention provides a recombinant HIV-1 envelope polypeptide according to Table 2, FIGS. 4C-D, FIG. 12F, FIG. 13, or Table 3, FIG. 14, FIG. 15, FIG. 16, FIG. 17, or FIG. 18F, or Table 4 or an envelope polypeptide encoded by a nucleic acid according to FIG. 19. In certain aspects, the invention provides a recombinant HIV-1 envelope polypeptide CAP256SU_UCA_OPT_3.0_K170R (also referred to as CAP256SU_OPT_4.0) or HIV_CAP256.wk34.c80_V2UCA_OPT_4.0 R171K. In certain embodiments, the polypeptide is a non-naturally occurring protomer. In some embodiments, the polypeptide is designed to form an envelope trimer. In certain embodiments, the envelope is based on CH505 T/F envelope and comprises optimized sequence for binding to V2 antibodies, including without limitation V2 UCAs. In certain embodiments the envelope is based on CAP256. In certain embodiments the envelope is based on HIV_CAP256SU (based on the HIV sequence). In certain embodiments the envelope is based on CAP256 SU (based on the SHIV.CAP256SU sequence). SHIV.CAP256SU differs in HXB2 position 375 and has a SIVmac cytoplasmic tail from HXB2 position 721 to the terminus. In certain embodiments the envelope is based on CAP256 SU_375S (the same as CAP256 SU sequence with a serine at HXB2 position 375). As used herein, an envelope based on CAP256 includes envelopes based at least on these three variants of CAP256SU. In certain embodiments, the envelope is based on CAP256wk34.80. In certain embodiments the envelope is based on CAM13. In certain embodiments, the envelope is based on Q23.17. In certain embodiment, the envelope comprises mutations H130D, D167N, K169R, Q170R and Q171K, or a combination thereof. In certain embodiments, the V1 hypervariable loop at wildtype Env HXB2 positions 132-152 is replaced with the sequence STYNNTHNISK. In certain embodiments, the V2 hypervariable loop at wildtype Env HXB2 positions 185-190 is replaced with the sequence NKNGRQ. In certain embodiments the V1 hypervariable loop at wildtype Env HXB2 positions 132-152 is replaced with the sequence STYNNTHNISK and the V2 hypervariable loop at wildtype Env HXB2 positions 185-190 is replaced with the sequence NKNGRQ. In certain embodiments, the envelope comprises glycan knock-in mutations as described in Wagh et al. Cell Reports 25 (4): 893-908 (2018) (pubmed.ncbi.nlm.nih.gov/30355496/), the content of which is hereby incorporated by reference. In certain embodiments the envelope polypeptide is designed to multimerize. In some embodiments the envelope sequence comprises a self-assembling protein. In certain embodiments, the self-assembling protein is a ferritin. In other embodiments, the self assembling protein is added via a sortase A reaction.

Is some embodiments, the envelope is based on CAM13RRK, CAM13RRRK, HIV_CAP256SU_UCA_OPT_4.0, CAP256SU_UCA_OPT_4.0 375S, CAP256SU_UCA_OPT_4.0_Y375S_D167N, CAP256_wk34.80_V2UCA_OPT, CAP256 wk34.80 PCT64UCA_OPT, CAP256_wk34.80_V2UCA_OPT_R17IK, CAP256 wk34.80 V2UCA_OPT_RRK, CAP256_wk34.80_V2UCA_OPT_RRK_D167N, Q23.17 (natural_wildtype), Q23.17_V2UCAOPT, Q23.17_V2UCAOPT_GLY, Q23.17_V2UCAOPT_ALT, Q23.17_V2UCAOPT_GLY_ALT, Q23.17_V2UCAOPT_GLY_ALT_R170Q, CH505_V2UCAOPT2_N332, CH505_V2UCAOPT_v3.0. See Table 2.

In certain embodiments, the optimized V2 loop modifications described herein can be incorporated into an envelope from Table 1 or Table 3.

In some embodiments, the invention provides a nucleic acid of FIG. 19 or 17 or encoding a recombinant HIV-1 envelope polypeptide according to Table 2, FIGS. 4C-D, FIG. 12F, FIG. 13, or Table 3, FIG. 14, FIG. 15, FIG. 16, FIG. 17, or FIG. 18F, or Table 4 or an envelope polypeptide encoded by a nucleic acid according to FIG. 19. In non-limiting embodiments, the nucleic acid is an mRNA. In some embodiments, the mRNA comprises the nucleic acids according to FIG. 19, wherein thymine (T) will be uridine (U). In some embodiments, the mRNA comprises the nucleic acids according to FIG. 19, wherein thymine (T) will be 1-methyl-psuedouridine. In some embodiments, the mRNA is modified. In some embodiments, the modification is a modified nucleotide such as 5-methyl-cytidine and/or 6-methyl-adenosine and/or modified uridine. In some embodiments, the mRNA comprises the nucleic acids according to FIG. 19, wherein the poly A tail is about 85 to about 200 nucleotides long. In some embodiments, the mRNA comprises the nucleic acids according to FIG. 19, wherein the poly A tail is about 85 to about 110 nucleotides long. In some embodiments, the mRNA comprises the nucleic acids according to FIG. 19, wherein the poly A tail is about 90 to about 110 nucleotides long. In some embodiments, the mRNA comprises the nucleic acids according to FIG. 19, wherein thymine (T) will be uridine (U) and wherein the sequence comprises the nucleotides up to the poly A tail, wherein the mRNA comprises a poly A tail about 85 to about 200 nucleotides long. In some embodiments, the mRNA comprises the nucleic acids according to FIG. 19, wherein thymine (T) will be uridine (U) and wherein the sequence comprises the nucleotides up to the poly A tail, wherein the mRNA comprises a poly A tail about 85 to about 110 nucleotides long. In some embodiments, the mRNA comprises the nucleic acids according to FIG. 19, wherein thymine (T) will be uridine (U) and wherein the sequence comprises the nucleotides up to the poly A tail, wherein the mRNA comprises a poly A tail about 90 to about 110 nucleotides long. In some embodiments, the mRNA comprises the nucleic acids according to FIG. 19, wherein thymine (T) will be 1-methyl-psuedouridine and wherein the sequence comprises the nucleotides up to the poly A tail, wherein the mRNA comprises a poly A tail about 85 to about 200 nucleotides long. In some embodiments, the mRNA comprises the nucleic acids according to FIG. 19, wherein thymine (T) will be 1-methyl-psuedouridine and wherein the sequence comprises the nucleotides up to the poly A tail, wherein the mRNA comprises a poly A tail about 85 to about 110 nucleotides long. In some embodiments, the mRNA comprises the nucleic acids according to FIG. 19, wherein thymine (T) will be 1-methyl-psuedouridine and wherein the sequence comprises the nucleotides up to the poly A tail, wherein the mRNA comprises a poly A tail about 90 to about 110 nucleotides long. In non-limiting embodiments, the mRNA is administered as an LNP.

In some aspects, the invention provides a recombinant trimer comprising three identical protomers of an envelope from Table 2, FIGS. 4C-D, FIG. 12F, FIG. 13, or Table 3, FIG. 14, FIG. 15, FIG. 16, FIG. 17, or FIG. 18F, or Table 4 or encoded by a nucleic acid according to FIG. 19. In some embodiments, the invention provides an immunogenic composition comprising the recombinant trimer and a carrier, wherein the trimer comprises three identical protomers of an HIV-1 envelope listed in Table 2, FIG. 4C, FIG. 12F, FIG. 13, or Table 3, FIG. 14, FIG. 15, FIG. 16, FIG. 17, or FIG. 18F, or Table 4 or encoded by a nucleic acid according to FIG. 19.

In some embodiments, the invention provides an immunogenic composition comprising a nucleic acid encoding the recombinant HIV-1 envelope and a carrier. In some embodiments, the compositions comprise at least two different immunogens targeting different V2 UCAs. In non-limiting embodiments, the immunogens are from Table 1, Table 2 Table 3 and/or Table 4. Non-limiting embodiment of a combination includes CAP256SU_UCA_OPT_3.0_K170R (also referred to as CAP256SU_OPT_4.0), CAP256SU_UCA_OPT_2.0, CAM13RRK_K130H, CH505_UCA_OPT3_D167N, or any combination thereof. See FIGS. 8-12. Non-limiting embodiment of a combination includes CAP256SU_OPT_4.0, CAM13RRK, CAP256wk34.80_V2_UCA_OPT, CAP256wk34.80_PCT64UCA_OPT or any combination thereof (FIGS. 14-16). In non-limiting embodiments, the composition comprises CAP256wk34.80_V2 UCA_OPT_4.0. In non-limiting embodiments, the composition comprises HIV_CAP256SU_OPT4.0, CAP256wk34.80_V2UCAOPT_R17IK, CAM13RRRK, and Q23.17 (natural Env). In non-limiting embodiments, the composition comprises CAP256wk34.80_V2UCAOPT_RRK, CAP256wk34.80_V2UCAOPT_R17IK, CAM13RRRK, and Q23.17 (natural Env). In non-limiting embodiments, the composition comprises HIV_CAP256SU_OPT4.0, CAP256wk34.80_V2UCAOPT_R17IK, CAM13RRK, and Q23.17 (natural Env). In non-limiting embodiments, the composition comprises CAP256wk34.80_V2UCAOPT_RRK, CAP256wk34.80_V2UCAOPT_R17IK, CAM13RRK, and Q23.17 (natural Env). In non-limiting embodiments, the composition comprises CAP256wk34.80_V2UCAOPT_RRK. In non-limiting embodiments, the composition comprises CAM13RRK.

In some embodiments, the envelopes are or are designed as trimers, and/or nanoparticles.

In some embodiments the immunogenic composition further comprises an adjuvant.

In some embodiments, the nucleic acid encoding one or more envelope selected from FIGS. 4C-4D, 7, 8, 9, 10, 11, 12, 13, 14, 16, 17, 18F, or 19 or any combination thereof is operably linked to a promoter. In some embodiments, the nucleic acid is inserted in an expression vector.

In some aspects, the invention provides a method of inducing an immune response in a subject comprising administering a composition comprising any suitable form of a nucleic acid(s) encoding one or more envelope selected from FIGS. 4C-4D, 7, 8, 9, 10, 11, 12, 13, 14, 16, 17, 18F, or 19 or any combination thereof or an envelope selected from FIGS. 4C-4D, 7, 8, 9, 10, 11, 12, 13, 14, 16, 17, or 18F, or any combination thereof in an amount sufficient to induce an immune response.

In some embodiments, the composition administered comprises a nucleic acid encoding a gp120 envelope, gp120D8 envelope, a gp140 envelope (gp140C, gp140CF, gp140CFI) as soluble or stabilized protomer of a SOSIP trimer, a gp145 envelope, a gp150 envelope, a transmembrane bound envelope, a gp160 envelope or an envelope designed to multimerize.

In some embodiments, the composition administered comprises a polypeptide, wherein the polypeptide is gp120 envelope, gp120D8 envelope, a gp140 envelope (gp140C, gp140CF, gp140CFI) as soluble or stabilized protomer of a SOSIP trimer, a gp145 envelope, a gp150 envelope, a transmembrane bound envelope, or an envelope designed to multimerize.

In some embodiments, the composition administered further comprises an adjuvant.

In some embodiments, the method further comprises administering an agent which modulates host immune tolerance. In some embodiment, the polypeptide administered is multimerized in a liposome or nanoparticle.

In some embodiments, the method further comprising administering one or more additional HIV-1 immunogens to induce a T cell response.

In some aspects, the invention provides a composition comprises a nanoparticle and a carrier, wherein the nanoparticle comprises an envelope, wherein the envelope is selected from FIGS. 4C-4D, 7, 8, 9, 10, 11, 12, 13, 14, 16, 17, 18F, or 19, or any combination thereof. In some embodiments, the compositions comprises two, three, four or more different immunogens. In some embodiments the immunogens target different V2 UCAs. In non-limiting embodiments the different immunogens are selected from the various V2 OPT designs described herein.

In some embodiments, the nanoparticle of the composition is ferritin self-assembling nanoparticle.

In some aspects, the invention provides a composition comprising a nanoparticle and a carrier, wherein the nanoparticle comprises (a) a nucleic acid according to FIG. 17 or 19 or encoding the recombinant HIV-1 envelope polypeptide from Table 2, FIGS. 4C-D, FIG. 12F, FIG. 13, or Table 3, FIG. 14, FIG. 15, FIG. 16, FIG. 17, or FIG. 18F, or Table 4 or (b) a recombinant trimer comprising three identical protomers of an envelope from Table 2, FIG. 4C, FIG. 12F, FIG. 13, or Table 3, FIG. 14, FIG. 15, FIG. 16, FIG. 17, or FIG. 18F, or Table 4 or encoded by a nucleic acid according to FIG. 19.

In some embodiments, the nanoparticle of the composition is a ferritin self-assembling nanoparticle.

In some embodiments, the nanoparticle of the composition comprises multimers of trimers.

In some embodiments, the nanoparticle of the composition comprises 1-8 trimers.

In some aspects, the invention provides a method of inducing an immune response in a subject comprising administering an immunogenic composition comprising any one of the recombinant envelopes or compositions described herein. In some embodiments the methods comprise administering two, three, four or more different immunogens. In some embodiments, the different immunogens target different V2 UCAs. In non-limiting embodiments the different immunogens are selected from the V2 OPT designs described herein-Tables 1, 2, 3, and/or 4, FIGS. 4C-4D, 7, 8, 9, 10, 11, 12, 13, 14, 16, 17, or 18F.

In some embodiments, the composition is administered as a prime.

In some embodiments, the composition is administered as a boost.

In some aspects, the invention provides a nucleic acid encoding any of the recombinant envelopes described herein. In some embodiments, the invention provides a composition comprising the nucleic acid and a carrier. In some embodiments, the nucleic acid is an mRNA. In some embodiments, the mRNA is encapsulated in a lipid nanoparticle (LNP).

In some embodiments, the invention provides a method of inducing an immune response in a subject comprising administering an immunogenic composition comprising the nucleic acid encoding any of the recombinant envelopes described herein. In some embodiments, the immunogenic composition further comprises a carrier.

In certain aspects, the invention provides an immunogenic composition or composition, wherein the composition comprises at least two different HIV-1 envelope polypeptides or nucleic acids encoding a recombinant HIV-1 envelope polypeptide, or a combination thereof.

In certain aspects, the invention provides an immunogenic composition comprising a first immunogen and a second immunogen, wherein the first immunogen is a recombinant HIV-1 envelope polypeptide from Table 2, FIG. 4C, FIG. 12F, FIG. 13, or Table 3, FIG. 14, FIG. 15, FIG. 16, FIG. 17, or FIG. 18F, or Table 4 or encoded by a nucleic acid according to FIG. 19 or a nucleic acid encoding said recombinant HIV-1 envelope polypeptide, and wherein the second immunogen is a different recombinant HIV-1 envelope polypeptide from Table 2, FIG. 4C, FIG. 12F, FIG. 13, or Table 3, FIG. 14, FIG. 15, FIG. 16, FIG. 17, or FIG. 18F, or Table 4 or encoded by a nucleic acid according to FIG. 19 or a nucleic acid encoding said different recombinant HIV-1 envelope polypeptide. In certain aspects, the invention provides a method of inducing an immune response in a subject comprising administering the immunogenic composition in an amount sufficient to induce an immune response. In certain embodiments, the method further comprising administering an agent which modulates host immune tolerance.

In certain embodiments, at least one of the first immunogen and the second immunogen is a recombinant HIV-1 envelope polypeptide. In certain embodiments, at least one of the first immunogen and the second immunogen is a recombinant trimer comprising three identical protomers of the recombinant HIV-1 envelope polypeptide. In certain embodiments, the first immunogen and the second immunogen are a recombinant HIV-1 envelope polypeptide. In certain embodiments, at least one of the first immunogen and the second immunogen is a nucleic acid. In certain embodiments, the first immunogen and the second immunogen are a nucleic acid. In certain embodiments, the nucleic acid is an mRNA. In certain embodiments, the mRNA is encapsulated in an LNP. In certain embodiments, the immunogenic composition further comprises one or more additional immunogens, wherein the one or more additional immunogens is different to the first and second immunogens.

In certain aspects, the invention provides an immunogenic composition comprising HIV-1 envelopes HIV_CAP256SU_OPT4.0, CAP256wk34.80_V2UCAOPT_R17IK, CAM13RRRK, and Q23.17. In certain aspects, the invention provides a method of inducing an immune response in a subject comprising administering the immunogenic composition in an amount sufficient to induce an immune response. In certain embodiments, the method further comprising administering an agent which modulates host immune tolerance.

In certain embodiments, the HIV-1 envelopes are in the form of a recombinant HIV-1 envelope polypeptides or nucleic acid, or a combination thereof. In certain embodiments, one or more of the HIV-1 envelopes is a recombinant trimer comprising three identical protomers of the recombinant HIV-1 envelope polypeptide. In certain embodiments, the nucleic acid is an mRNA. In certain embodiments, the composition comprises a carrier. In certain embodiments, the composition further comprises an adjuvant.

### BRIEF DESCRIPTION OF THE DRAWINGS

The patent or application file contains at least one drawing executed in color. To conform to the requirements for PCT patent applications, many of the figures presented herein are black and white representations of images originally created in color.

FIG. 1 shows CH505 Mature Optimized Design. Shown are CH505 amino acid substitutions that are statistically associated with for V2 apex mature bNAb sensitivity. The letters represent single amino acids, and the height of the letter in the sequence LOGO indicates its frequency in the population. The numbers underneath the LOGO are HXB2 reference strain positions in the viral sequence. O stands for an N embedded in a N-linked glycosylation site. Blue are amino acids that are associated with sensitivity, red are amino acids associated with resistance, black are amino acids that were not associated with either sensitivity or resistance. The V2 SET OPT chimeric SOSIP (last row) carries all the design mutations from the full length CH505 TF V2 SET OPT except at 31, 33 and 588, 644. For the former, the SOSIP construct has the favorable mutations.

For 588, we suggest mutating to K (quite common aa, signature p-value=0.0005-0.026 depending on the V2 bnab, odd's ratio (OR)=2-5). For 644, we suggest mutating to R (most common sensitive aa, p=0.0006−0.007, OR=2.7−7.9).

To minimize the number of constructs, we propose adding these to UCA_OPT1 SOSIP constructs (note: our UCA_OPT1 carried all the sensitive signatures for mature bNAbs also in addition to most UCAs/intermediates). Gp41 mutations could also be added in some embodiments.

FIG. 2 shows additional signature amino acids associated with V2 bNAb unmutated common ancestor or early intermediate antibodies from early stages of V2 apex bNAb maturation. See FIG. 1 for details. UCA_OPT1 SOSIP construct just has one sub-optimal aa at PG9 germline reverted Ab signature sites as compared to the full length UCA_OPT1—it has an M-535 instead of I-535. We suggest using I-535 (fairly common aa, signature p=0.01, OR=3.3). Data not shown for other V2 UCAs/intermediates (CH04, PCT64) but the SOSIP UCA_OPT1 construct carries all the favorable mutations for their signature sites as well. Gp41 mutations could also be added in some embodiments.

FIGS. 3A-3C show non-limiting embodiments of amino acid sequences. These are continuous sequences where dashes represents gaps if these sequences were aligned.

FIGS. 4A and 4B show non-limiting embodiments of amino acid and nucleic acid sequences. In FIG. 4B, VDAT=cloning site and Kozak sequence. Underlined=signal peptide that is cleaved from mature protein. FIG. 4C shows a non-limiting embodiment of a gp160 envelope amino acid sequence for CH505.V2UCAOPT.ver2. FIG. 4D shows a non-limiting embodiment of a nucleic acid sequence encoding the envelope in FIG. 4C.

FIGS. 5A, 5B, 5C and SD show non-limiting embodiment of sortase designs and nucleic acid and protein sequences. FIG. 5E shows non-limiting embodiments of ferritin designs. The linker between the envelope sequence and the ferritin protein sequence could be any suitable linker. The ferritin protein could be any suitable ferritin. See e.g. without limitation U.S. Pat. No. 10,961,283, incorporated herein by reference. The envelopes in these designs are CH505 T/F or CH505 M5. A skilled artisan can readily incorporate the V2 optimization into these envelopes.

FIG. 6A shows neutralization data for optimized designs of the invention. FIG. 6B summarized the neutralization data of FIG. 6A and shows IC50 ((μg/ml) titers). In FIG. 6B K170R should be Q170R. The neutralization data is from a standard assay in the field: see for e.g. Barbian et al. PMID: 25900654 or Montefiori et al. PMID: 18432938. In this assay Envs being tested are inserted in a standard HIV backbone with a luciferase reporter, the viruses are then expressed in 293T cells and then tested for ability to infect TZM-bl cells in presence of varying concentrations of antibodies measured by luciferase based luminosity. The neutralization results show the drop in infectivity of each pseudotyped Env in the presence of different concentrations of the UCAs. These data show that UCA OPT2 N332 (ver 1) showed reduced infectivity in presence of high concentrations of only 2 UCAs (CH01 and PCT64), while UCA_OPT2 N332 ver2 shows substantially reduced infectivity at high concentrations of CH01, PCT64, PG9 and PG16 UCAs. These results show that version 2 can bind to the 4 V2 apex UCAs, thus suggesting that it could trigger such rare V2 apex precursors when used as an immunogen.

FIGS. 7A-7I depict the second round design strategy. FIG. 7A depicts the detection of the R170 signature which is a polar contact with Tyr111. FIG. 7B depicts PG16 RUA and PG9 RUA sensitivity for CAM13K (i.e. CAM13+Q171K), CAM13K+K169R, CAM13K +K169R+K170R and CAM13K+K169R+K170Q. FIG. 7C depicts A161 interactions. FIG. 7D depicts the PCT64 LMCA signature determined using CH505 UCA_OPT+H130D was tested to determine. FIGS. 7E-7G depict sensitivity of CH505 TF, CH505 UCA_OPT_2+N332, CH505 UCA_OPT_2+H130D, CH505 UCA_OPT_2+Q170R, or CH505 UCA_OPT_2 +H130D+K169R+Q170R to VRC26 UCA, CHOIRUA3, PG9 RUA, PG16 RUA, PCT64LMCA, and RM5695 UCA. FIG. 7H depicts the sensitivity of candidates to five UCA lineages. FIG. 7I depicts sensitivity of CH50ST; CH505 OPT2 N332; CH505 OPT2 N332 Q170R; or CH505 OPT2 N332 H130D, K169R, Q170R to VRC26 UCA, CH01 RUA3, PG9 RUA, PG16RUA, PCT64 LMCA, or 5695 rhesus UCA.

FIGS. 8A-8B depict identified V2 apex UCA neutralization constructs. FIG. 8A shows the leading constructs that together as a cocktail are sensitive to all V2 apex UCAs. FIG. 8B depicts other neutralization constructs.

FIGS. 9A-9S depict initial determination of attractive V2 apex bNAbs targets for immunogen design. FIG. 9A depicts the viral membrane structure. FIG. 9B depicts the V2 apex bNAB from SHIV CH505 infected RM. FIG. 9C shows schematic of signature based approach of immunogen design. See also Bricault et al. Cell Host Microbe 2019 25 (1) 59-72. FIG. 9D depicts phylogenetic and/or contact sites, robustness across bNAbs and datasets, and were used for designing CH505 SET OPT. FIG. 9E depicts neutralization data for 208 global viruses against CH04 & CAP256 UCAs, and heavy and/or light chain germline reverted PG9. FIG. 9F shows analyses for CAP256 IA4. For CAP256 IA4 weak signatures found due to low statistical power (3 out of 208 viruses neutralized). Only resistant signatures outside the epitope. Change to neutral at most sites would involve mutation to rare amino acid and/or removing glycans that could introduce vulnerable gaps in the glycan shield. Only two mutations introduce at 736 & 842. Designed UCA optimized constructs without (UCA OPT1) and with (UCA_OPT2) these weak signatures. FIG. 9G shows Hypervariable Loop Characteristic. Hypervariable loops cannot be aligned due to extreme length & sequence variation. Tested for associations with net charge, length & number of glycans. Found two significant hypervariable loop associations with sensitivity to V2 apex bNAbs: Positively charged V2 loops; V2 apex bNAbs have long anionic CDRH3. Smaller hypervariable V1 & V2 combined: possible steric hindrance due to the dynamic loops. FIG. 9H shows Hypervariable V1 & V2 substitutions: Optimizing for Positive Charge and optimizing for smaller length based on M-group Hypervariable length distribution. FIG. 9I depicts M-group bypervariable length distribution. FIG. 9J depicts mature signature and germline signature sensitivity to neutralization by mature V2 bNAbs. It shows that mature signature introduction increases sensitivity to neutralization by mature V2 bNAbs. Shown are results for CH505 TF and CH505 V2 SET envelopes as gp160 constructs in a pseudovirus neutralization assay. The assay is a standard TZM-BI cell neutralization assay as describer in Sarzotti-Kelsoe et al. J Immunol Methods. 2014 July; 409:131-46. doi: 10.1016/j.jim.2013.11.022. Epub 2013 Dec. 1. Antibody is shown in each panel. It further shows that germline signatures further increase sensitivity to neutralization by mature V2 bNAbs. Shown are results for CH505 TF, CH505 V2 SET, and CH505 UCA_OPT1 envelopes as gp160 constructs in a pseudovirus neutralization assay. Antibody is shown in each panel. The thick arrow shows CH505 UCA OPT1 curve, which in panels A and E overlaps with CH505 V2 SET curve. FIG. 9K shows that UCA signatures increase neutralization sensitivity of CH505 envelopes by unmutated common ancestor (UCA) or reverted common ancestor (RUA) antibodies. Shown are results for CH505 TF, CH505 V2 SET, and CH505 UCA_OPT1 envelopes as gp160 constructs in a pseudovirus neutralization assay. Antibody is shown in each panel. UCA signatures increased the sensitivity of CH505 to neutralization by both CH01 and the PCT64 V2 bNAb UCAs. V2 SET OPT also gains CH01 UCA sensitivity, likely due to H-130. UCA_OPT2 that had CAP256 VRC26 UCA signatures did not confer sensitivity to this UCA. FIG. 9L depicts V2 UCA neutralization. FIG. 9M show sensitivity to neutralization by mature V2 apex bnAbs. Respective antibodies are listed in each panel. N332 represents a predicted V2 apex bNab resistance signature, but is critical for V3 bNabs (CH505 Env has N334). Moving the N334 glycan to N332 did not reduce its sensitivity to mature V2 bNabs, and rendered it highly sensitive to PGT121. The legend listed in FIG. 9M is applicable to all panels in this figure. FIG. 9N shows summary of expression and binding data for various optimized designs expressed as SOSIP designs. Various non-limiting embodiments of SOSIP designs are shown in FIGS. 3 and 4. FIG. 9O shows SET OPT & UCA_OPT constructs expressed as chimeric CH505-BG505 SOSIPs. Different constructs tested with varying quality & expression. Expression of UCA_OPT1 with NxST 332 and gp41 mutations resulted in highest level of trimer formation (88% versus 12% monomer) as shown. It further shows antibody binding consistent with neutralization results. Binding data consistent with neutralization results. FIG. 9P depicts three classes of sites in the CH505 TF considered for mutation to increase sensitivity. FIG. 9Q depicts the mutations present in the CH505 V2 initial design (CH505 TF V2 SET OPT). FIG. 9R depicts the additional mutations present in the CH505 TF UCA_OPT1. FIG. 9S shows a summary of the neutralization data. The table shows that introduction of V2 apex mature signatures in CH505 TF improved sensitivity to mature bNAbs, and gained sensitivity to CH01 UCA-SET OPT column. Introduction of UCA signatures further improved sensitivity to mature bNAbs, to CH01 UCA and gained sensitivity to PCT64 LMCA—UCA OPT column. In this figure the UCA_OPT label shows UCA_OPT2+N332 —the slope of the curve where the curve for CH505 UCA_OPT2+N332 is bending for the PCT64LMCA, whereas it is not for PG9RUA. This indicates that when measured the neutralization up to 250 μg/ml, 50% neutralization could be reached at 105 ug/ml. First column lists the antibody. “WT” refers to CH505 TF sequences without optimization signatures.

FIGS. 10A-10D depict results of second round of designs. FIG. 10A depicts longitudinal Env evolution data demonstrating escape predominantly at particular amino acid. FIG. 10B depicts D167N association with escape from early (13 month) PCT64 lineage Abs. FIG. 10C depicts M4C054's sensitivity to PCT64-LMCA with glycan deletions at 130 and 133. FIG. 10D depicts CH505.V2UCAOPT.v3.D167N design and neutralization testing.

FIGS. 11A-11F depict CAM13RRK V2 UCA development. FIG. 11A depicts CAM13 mutated at R-169, R-170 and K-171 (‘CAM13RRK’) is sensitive to CH01, PG9 and PG16 UCAs. FIG. 11B depicts signatures for CAM13RRK. FIG. 11C depicts design construct CAM13RRK delV1 reducing the hypervariable V1 loop length. FIG. 11 D depicts modifications of the natural loops to introduce deletions and positive charges. FIG. 11E depicts CAM13RRK glycan holes. FIG. 11F depicts results from neutralization testing.

FIGS. 12A-12H depict CAP256SU based Env designs. FIG. 12A depicts month 35 Abs (35B, 35D, 35G, 350 and 35S; no 35M since on a different branch) signature sites. FIG. 12B depicts several other identified signatures. FIG. 12C depicts a sorted list of the 208 global virus panel based on most charge per unit hypervariable V1 or hypervariable V2 length. FIG. 12D depicts the M-group distributions of V1, V2 and V1+V2 length and charge with CAP256SU WT (each in blue, medians in red and constructs in purple). FIG. 12E depicts CAP256SU design including 10 mutations. FIG. 12F depicts sequences of SHIV_CAP256SU, CAP256SU_UCA_OPT, CAP256SU_UCA_OPT_2.0, CAP256SU_UCA_OPT_3.0, and UCA_OPT_3.0_K170R. FIG. 12G depicts neutralization of VR26UCA or VRC26.25, CH01 or CH01 RUA, PG9 or PG9999 RUA, PG16 or PG16 RUA, PCT64 LMCA or PCT64, or Rh-1A or RhA-I neutralization by CAP256SU_V2UCAOPTv3.0K170R_UCA or CAP256SU_V2UCAOPTv3.0K170R_maturebNAb. FIG. 12H depicts CAP256SU constructs and glycan shield filling.

FIG. 13 shows non-limiting embodiments of amino acid sequences listed in Table 2. These sequences comprise a signal peptide. A skilled artisan understands that any form of a recombinantly expressed protein based on these designs does not include a signal peptide which removed during cell processing.

FIG. 14 shows non-limiting embodiments of optimized immunogens-sortase designs.

FIGS. 15A to 15J show rationale and design for a cocktail of V2 apex bNAb germline targeting Envelopes comprising optimized CAP256_wk34.80 based envelopes. FIG. 15A depicts a predicted CAP256UCAOPT_v3 structure. FIG. 15B depicts an improved hypervariable V1 loop. FIG. 15C depicts an improved hypervariable V2 loop. FIG. 15D depicts the glycan holes of CAP256wk34.80. FIG. 15E depicts possible PCT64UCA escape mutations. FIG. 15F depicts the predicted structure of PCT64 UCA interacting with a positively charged region (light chain) of the hypervariable V2 loop. FIG. 15G depicts variation in PCT64 Envs. FIG. 15H depicts a summary of the designs. FIG. 15I depicts neutralization testing experimental data for V2 apex UCA neutralization. FIG. 15J depicts construct designs CAP256SU_UCA_OPT_4.0_D167N and CAP256SU_wk34.80_V2UCA_OPT_R17IK.

FIG. 16 shows amino acid sequences of non-limiting embodiments of optimized envelopes.

FIG. 17 shows amino acid sequences and nucleic acid sequences encoding amino acid sequences of non-limiting embodiments of optimized envelopes. HV1303230 to HV1303254 are gp150 and gp160 mRNA constructs designed for HIV_CAP256SU_UCA_OPT_v4.0.

FIGS. 18A-18F depict examples and sequences for development of improved constructs and mRNAs FIG. 18A depicts the CAM13RRK+K168R (CAM13RRRK) construct reactivity tests. FIG. 18B depicts the CAP256wk34.80_V2_UCA_OPT_R171K reactivity to several UCAs. FIG. 18C depicts HIV-1 CAP256SU with all CAP256SU_UCA_OPT_4.0 backbone mutations introduced reactivity test. FIG. 18D depicts the CAP256_wk34.80_V2UCA_OPT_R17IK construct reactivity tests. FIG. 18E depicts the SOSIP mutations of strategy 1 for HIV_CAP256SU_UCA_OPT_4.0 mRNA designs. FIG. 18F depicts the alignment of sequences for HIV_CAP256SU_UCA_OPT_4.0; mRNAI_CAP256SU_UCA_OPT_4.0; and mRNA2_CAP256SU_UCA_OPT_4.0; is depicted in FIG. 18F. Dots indicate deletions and dashes indicate identities.

FIG. 19 discloses exemplary mRNA sequences encoding an immunogen.

### DETAILED DESCRIPTION OF THE INVENTION

The development of a safe, highly efficacious prophylactic HIV-1 vaccine is of paramount importance for the control and prevention of HIV-1 infection. A major goal of HIV-1 vaccine development is the induction of broadly neutralizing antibodies (bnAbs) (Immunol. Rev. 254:225-244, 2013). BnAbs are protective in rhesus macaques against SHIV challenge, but as yet, are not induced by current vaccines.

For the past 25 years, the HIV vaccine development field has used single or prime boost heterologous Envs as immunogens, but to date has not found a regimen to induce high levels of bnAbs.

Recently, a new paradigm for design of strategies for induction of broadly neutralizing antibodies was introduced, that of B cell lineage immunogen design (Nature Biotech. 30:423, 2012) in which the induction of bnAb lineages is recreated. It was recently demonstrated the power of mapping the co-evolution of bnAbs and founder virus for elucidating the Env evolution pathways that lead to bnAb induction (Nature 496:469, 2013).

### Sequences/Clones

Described herein are nucleic and amino acids sequences of HIV-1 envelopes. The sequences for use as immunogens are in any suitable form. In certain embodiments, the described HIV-1 envelope sequences are gp160s. In certain embodiments, the described HIV-1 envelope sequences are gp120s. Other sequences, for example but not limited to stable SOSIP trimer designs, gp145s, gp140s, both cleaved and uncleaved, gp140 Envs with the deletion of the cleavage (C) site, fusion (F) and immunodominant (I) region in gp41—named as gp140ACFI (gp140CFI), gp140 Envs with the deletion of only the cleavage (C) site and fusion (F) domain—named as gp140ACF (gp140CF), gp140 Envs with the deletion of only the cleavage (C)—named gp140AC (gp140C) (See e.g. Liao et al. Virology 2006, 353, 268-282), gp150s, gp41s, which are readily derived from the nucleic acid and amino acid gp160 sequences. In certain embodiments the nucleic acid sequences are codon optimized for optimal expression in a host cell, for example a mammalian cell, a rBCG cell or any other suitable expression system.

An HIV-1 envelope has various structurally defined fragments/forms: gp160; gp140 —including cleaved gp140 and uncleaved gp140 (gp140C), gp140CF, or gp140CFI; gp120 and gp41. A skilled artisan appreciates that these fragments/forms are defined not necessarily by their crystal structure, but by their design and bounds within the full length of the gp160 envelope. While the specific consecutive amino acid sequences of envelopes from different strains are different, the bounds and design of these forms are well known and characterized in the art.

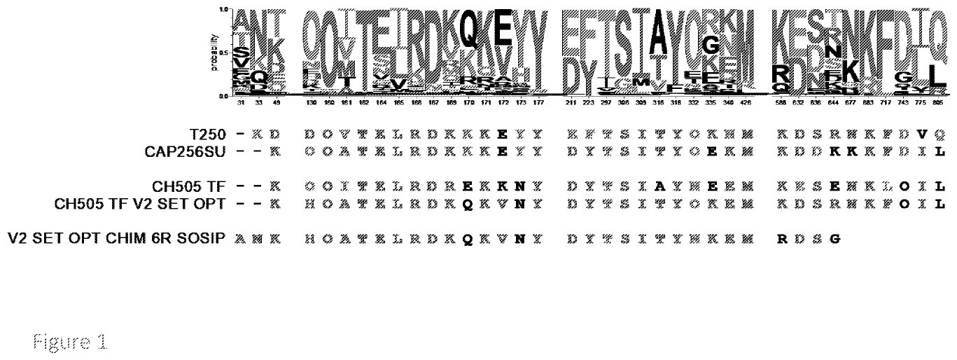

For example, it is well known in the art that during its transport to the cell surface, the gp160 polypeptide is processed and proteolytically cleaved to gp120 and gp41 proteins. Cleavages of gp160 to gp120 and gp41 occurs at a conserved cleavage site “REKR.” See Chakrabarti et al. Journal of Virology vol. 76, pp. 5357-5368 (2002) see for example FIG. 1, and Second paragraph in the Introduction on p. 5357; Binley et al. Journal of Virology vol. 76, pp. 2606-2616 (2002) for example at Abstract; Gao et al. Journal of Virology vol. 79, pp. 1154-1163 (2005); Liao et al. Virology vol. 353 (2): 268-282 (2006).

The role of the furin cleavage site was well understood both in terms of improving cleave efficiency, see Binley et al. supra, and eliminating cleavage, see Bosch and Pawlita, Virology 64 (5): 2337-2344 (1990); Guo et al. Virology 174:217-224 (1990); McCune et al. Cell 53:55-67 (1988); Liao et al. J Virol. Apr; 87 (8): 4185-201 (2013).

Likewise, the design of gp140 envelope forms is also well known in the art, along with the various specific changes which give rise to the gp140C (uncleaved envelope), gp140CF and gp140CFI forms. Envelope gp140 forms are designed by introducing a stop codon within the gp41 sequence. See Chakrabarti et al. at FIG. 1.

Envelope gp140C refers to a gp140 HIV-1 envelope design with a functional deletion of the cleavage (C) site, so that the gp140 envelope is not cleaved at the furin cleavage site. The specification describes cleaved and uncleaved forms, and various furin cleavage site modifications that prevent envelope cleavage are known in the art. In some embodiments of the gp140C form, two of the R residues in and near the furin cleavage site are changed to E, e.g., RRVVEREKR is changed to ERVVEREKE, and is one example of an uncleaved gp140 form. Another example is the gp140C form which has the REKR site changed to SEKS. See supra for references.

Envelope gp140CF refers to a gp140 HIV-1 envelope design with a deletion of the cleavage (C) site and fusion (F) region. Envelope gp140CFI refers to a gp140 HIV-1 envelope design with a deletion of the cleavage (C) site, fusion (F) and immunodominant (I) region in gp41. See Chakrabarti et al. Journal of Virology vol. 76, pp. 5357-5368 (2002) see for example FIG. 1, and Second paragraph in the Introduction on p. 5357; Binley et al. Journal of Virology vol. 76, pp. 2606-2616 (2002) for example at Abstract; Gao et al. Journal of Virology vol. 79, pp. 1154-1163 (2005); Liao et al. Virology vol. 353 (2): 268-282 (2006).

In certain embodiments, the envelope design in accordance with the present invention involves deletion of residues (e.g., 5-11, 5, 6, 7, 8, 9, 10, or 11 amino acids) at the N-terminus. For delta N-terminal design, amino acid residues ranging from 4 residues or even fewer to 14 residues or even more are deleted. These residues are between the maturation (signal peptide, usually ending with CX, X can be any amino acid) and “VPVXXXX . . . “. In case of CH505 T/F Env as an example, 8 amino acids (italicized and underlined in the below sequence) were deleted: MRVMGIQRNYPQWWIWSMLGFWMLMICNGMWVTVYYGVPVWKEAKTTLFCASDA KAYEKEVHNVWATHACVPTDPNPQE . . . (rest of envelope sequence is indicated as” . . . ”). In other embodiments, the delta N-design described for CH505 T/F envelope can be used to make delta N-designs of other CH505 envelopes. In certain embodiments, the invention relates generally to an immunogen, gp160, gp120 or gp140, without an N-terminal Herpes Simplex gD tag substituted for amino acids of the N-terminus of gp120, with an HIV leader sequence (or other leader sequence), and without the original about 4 to about 25, for example 4, 5, 6, 7, 8, 9, 10, 11, 12, 13, 14, 15, 16, 17, 18, 19, 20, 21, 22, 23, 24, 25 amino acids of the N-terminus of the envelope (e.g. gp120). See U.S. Pat. No. 10,040,826, e.g. at pages 10-12, the contents of which is hereby incorporated by reference in its entirety.

The general strategy of deletion of N-terminal amino acids of envelopes results in proteins, for example gpl20s, expressed in mammalian cells that are primarily monomeric, as opposed to dimeric, and, therefore, solves the production and scalability problem of commercial gp120 Env vaccine production. In other embodiments, the amino acid deletions at the N-terminus result in increased immunogenicity of the envelopes.

In certain aspects, the invention provides composition and methods which CH505 Envs, as gp120s, gp140s cleaved and uncleaved, gp145s, gp150s and gp160s, stabilized and/or multimerized trimers, as proteins, DNAs, RNAs, or any combination thereof, administered as primes and boosts to elicit immune response. CH505 Envs as proteins would be co-administered with nucleic acid vectors containing Envs to amplify antibody induction. In certain embodiments, the compositions and methods include any immunogenic HIV-1 sequences to give the best coverage for T cell help and cytotoxic T cell induction. In certain embodiments, the compositions and methods include mosaic and/or consensus HIV-1 genes to give the best coverage for T cell help and cytotoxic T cell induction. In certain embodiments, the compositions and methods include mosaic group M and/or consensus genes to give the best coverage for T cell help and cytotoxic T cell induction. In some embodiments, the mosaic genes are any suitable gene from the HIV-1 genome. In some embodiments, the mosaic genes are Env genes, Gag genes, Pol genes, Nef genes, or any combination thereof. See e.g. U.S. Pat. No. 7,951,377. In some embodiments the mosaic genes are bivalent mosaics. In some embodiments the mosaic genes are trivalent. In some embodiments, the mosaic genes are administered in a suitable vector with each immunization with Env gene inserts in a suitable vector and/or as a protein. In some embodiments, the mosaic genes, for example as bivalent mosaic Gag group M consensus genes, are administered in a suitable vector, for example but not limited to HSV2, would be administered with each immunization with Env gene inserts in a suitable vector, for example but not limited to HSV-2.

### Nucleic Acid Sequences

In certain aspects the invention provides compositions and methods of Env genetic immunization either alone or with Eny proteins to recreate the swarms of evolved viruses that have led to bnAb induction. Nucleotide-based vaccines offer a flexible vector format to immunize against virtually any protein antigen. Currently, two types of genetic vaccination are available for testing—DNAs and mRNAs.

In certain aspects the invention contemplates using immunogenic compositions wherein immunogens are delivered as DNA. See Graham B S, Enama M E, Nason M C, Gordon I J, Peel S A, et al. (2013) DNA Vaccine Delivered by a Needle-Free Injection Device Improves Potency of Priming for Antibody and CD8+ T-Cell Responses after rAd5 Boost in a Randomized Clinical Trial. PLoS ONE 8 (4): c59340, page 9. Various technologies for delivery of nucleic acids, as DNA and/or RNA, so as to elicit immune response, both T-cell and humoral responses, are known in the art and are under developments. In certain embodiments, DNA can be delivered as naked DNA. In certain embodiments, DNA is formulated for delivery by a gene gun. In certain embodiments, DNA is administered by electroporation, or by a needle-free injection technologies, for example but not limited to Biojector® device. In certain embodiments, the DNA is inserted in vectors. The DNA is delivered using a suitable vector for expression in mammalian cells. In certain embodiments the nucleic acids encoding the envelopes are optimized for expression. In certain embodiments DNA is optimized, e.g. codon optimized, for expression. In certain embodiments the nucleic acids are optimized for expression in vectors and/or in mammalian cells. In non-limiting embodiments these are bacterially derived vectors, adenovirus based vectors, rAdenovirus (e.g. Barouch DH, et al. Nature Med. 16:319-23, 2010), recombinant mycobacteria (e.g. rBCG or M smegmatis) (Yu, J S et al. Clinical Vaccine Immunol. 14:886-093, 2007; ibid 13:1204-11, 2006), and recombinant vaccinia type of vectors (Santra S. Nature Med. 16:324-8, 2010), for example but not limited to ALVAC, replicating (Kibler K V et al., PLoS One 6: e25674, 2011 Nov. 9.) and non-replicating (Perreau M et al. J. virology 85:9854-62, 2011) NYVAC, modified vaccinia Ankara (MVA)), adeno-associated virus, Venezuelan equine encephalitis (VEE) replicons, Herpes Simplex Virus vectors, and other suitable vectors.

In certain aspects the invention contemplates using immunogenic compositions wherein immunogens are delivered as DNA or RNA in suitable formulations. Various technologies which contemplate using DNA or RNA or may use complexes of nucleic acid molecules and other entities to be used in immunization. In certain embodiments, DNA or RNA is administered as nanoparticles consisting of low dose antigen-encoding DNA formulated with a block copolymer (amphiphilic block copolymer 704). See Cany et al., Journal of Hepatology 2011 vol. 54 j 115-121; Arnaoty et al., Chapter 17 in Yves Bigot (ed.), Mobile Genetic Elements: Protocols and Genomic Applications, Methods in Molecular Biology, vol. 859, pp293-305 (2012); Arnaoty et al. (2013) Mol Genet Genomics. 2013 August; 288 (7-8): 347-63. Nanocarrier technologies called Nanotaxi® for immunogenic macromolecules (DNA, RNA, Protein) delivery are under development. See for example technologies developed by incellart.

In certain aspects, the invention provides nucleic acids comprising sequences encoding envelopes of the invention. In certain embodiments, the nucleic acids are DNAs. In certain embodiments, the nucleic acids are mRNAs. In certain aspects, the invention provides expression vectors comprising the nucleic acids of the invention.

In certain aspects, the invention provides a pharmaceutical composition comprising mRNAs encoding the inventive antibodies. In certain embodiments, these are optionally formulated in lipid nanoparticles (LNPs). In certain embodiments, the mRNAs are modified. Modifications include without limitations modified ribonucleotides, poly-A tail, 5′cap.

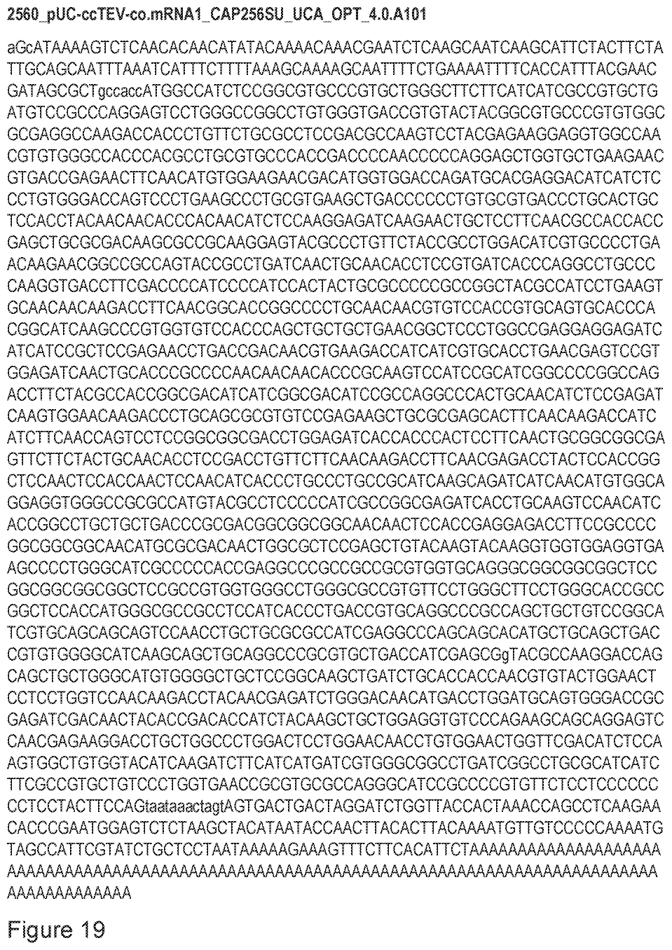

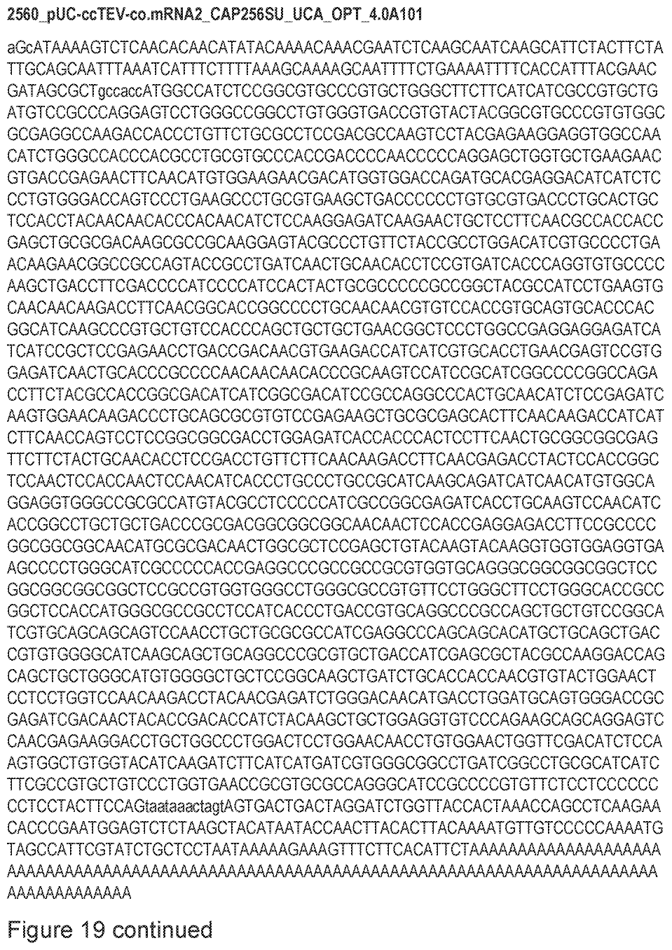

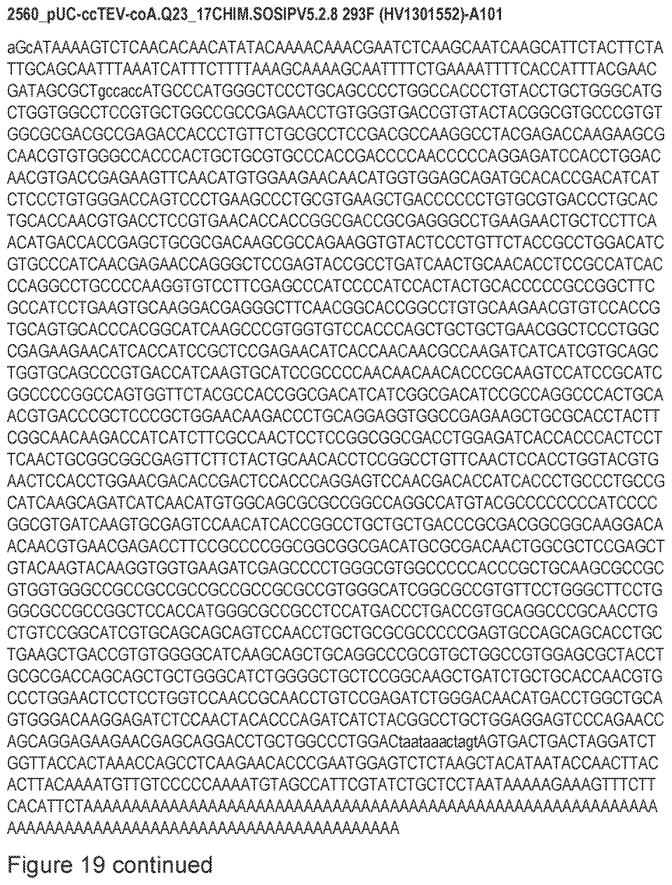

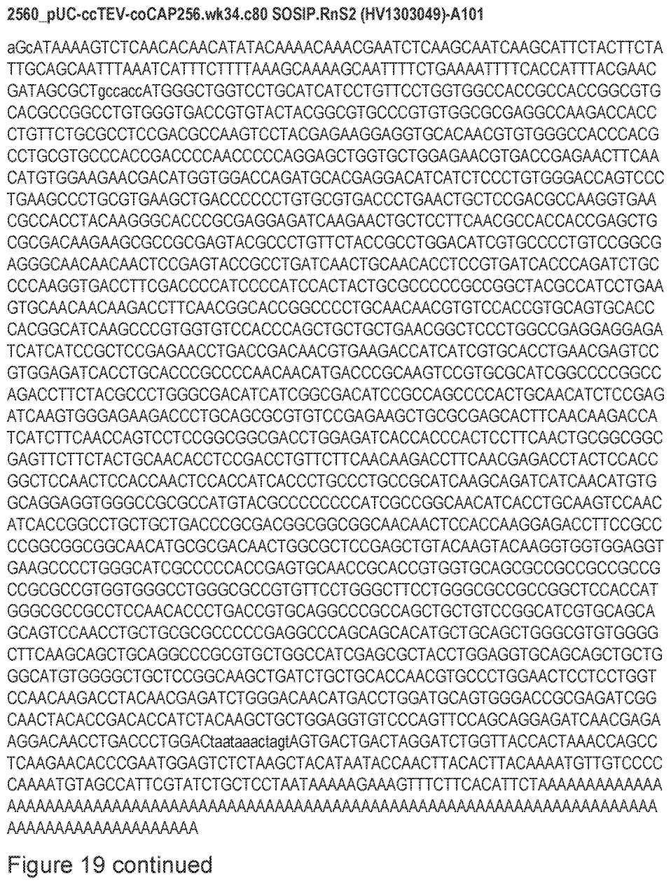

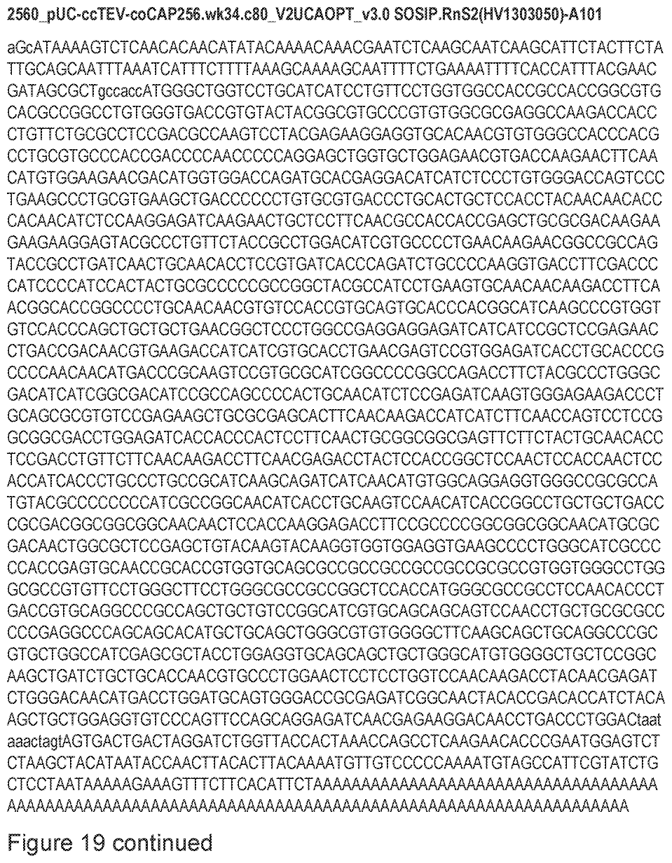

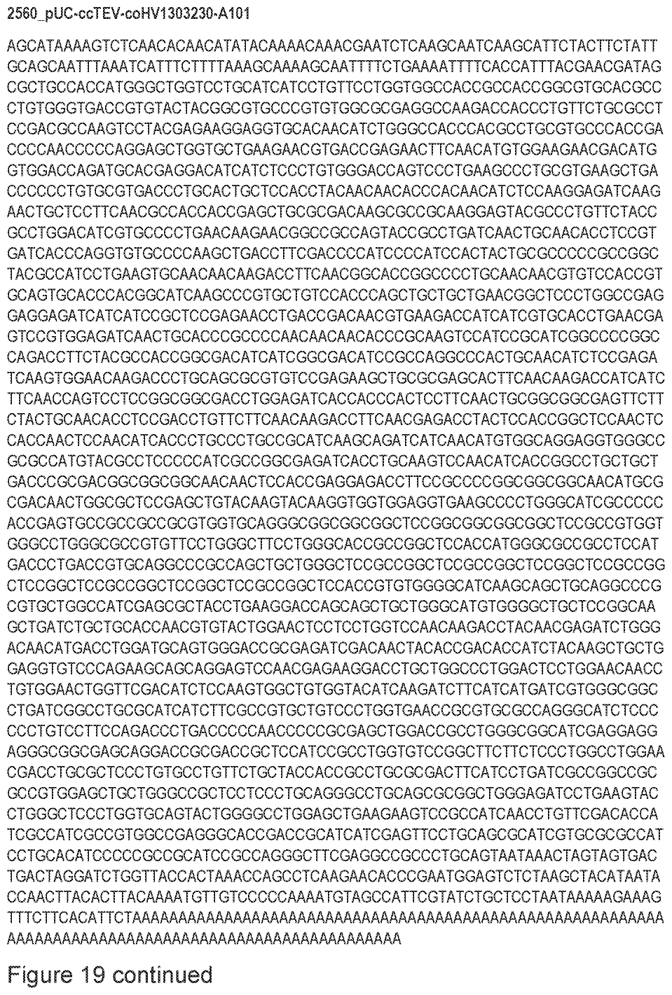

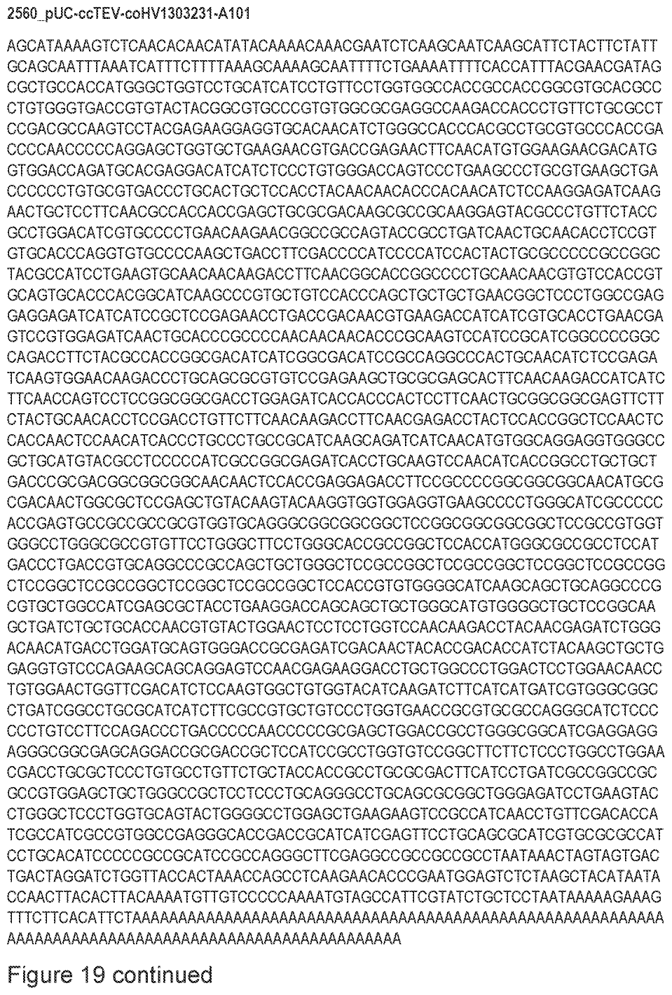

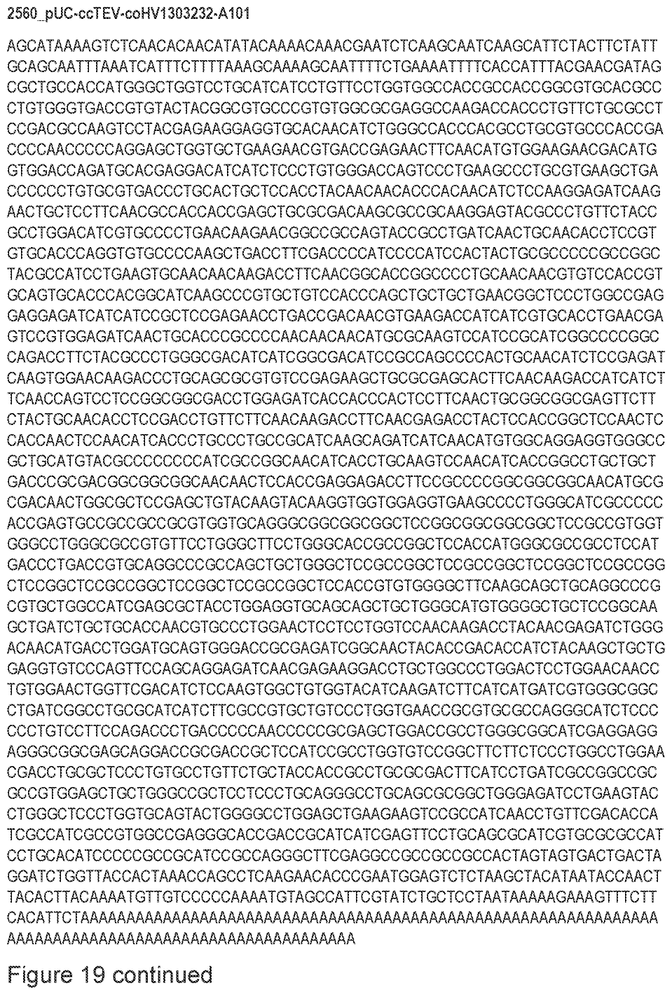

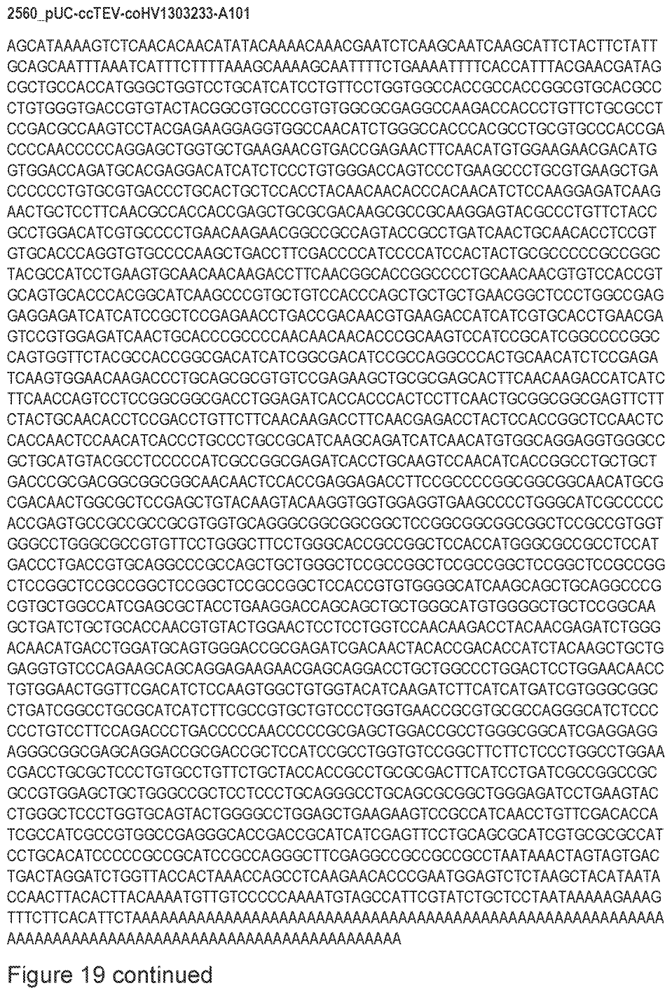

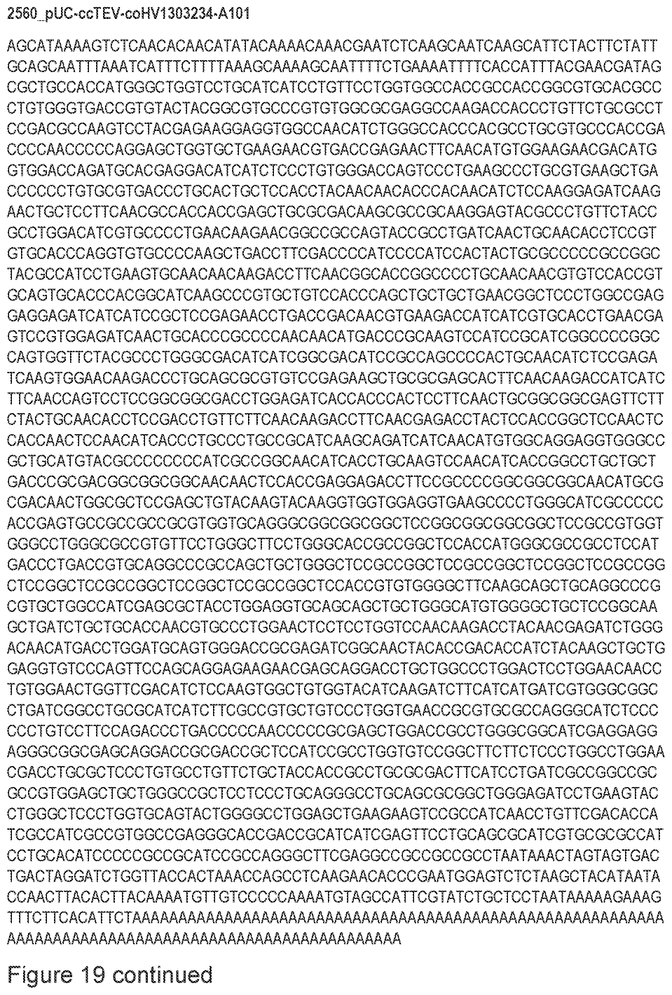

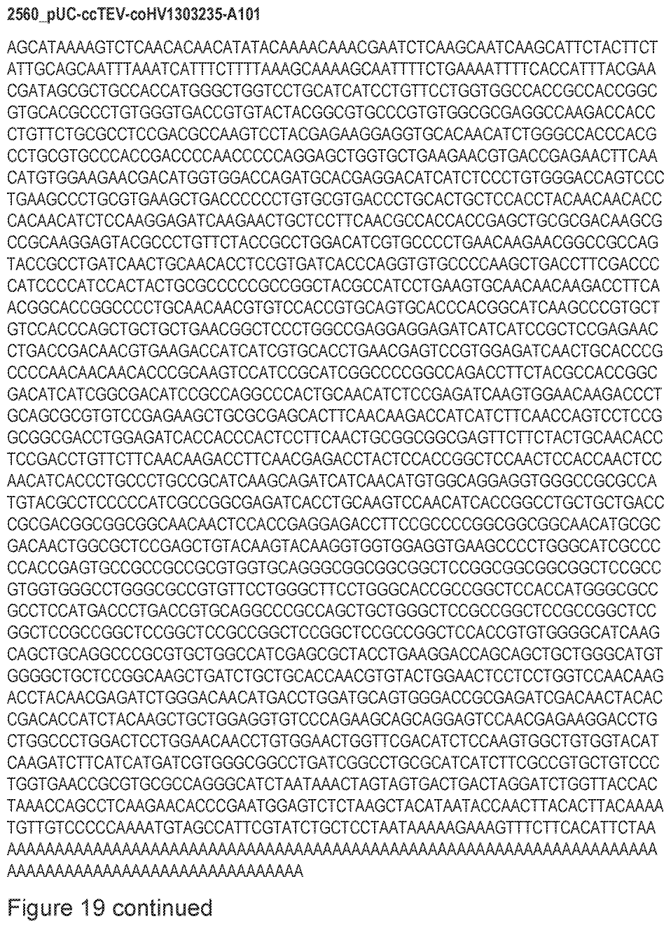

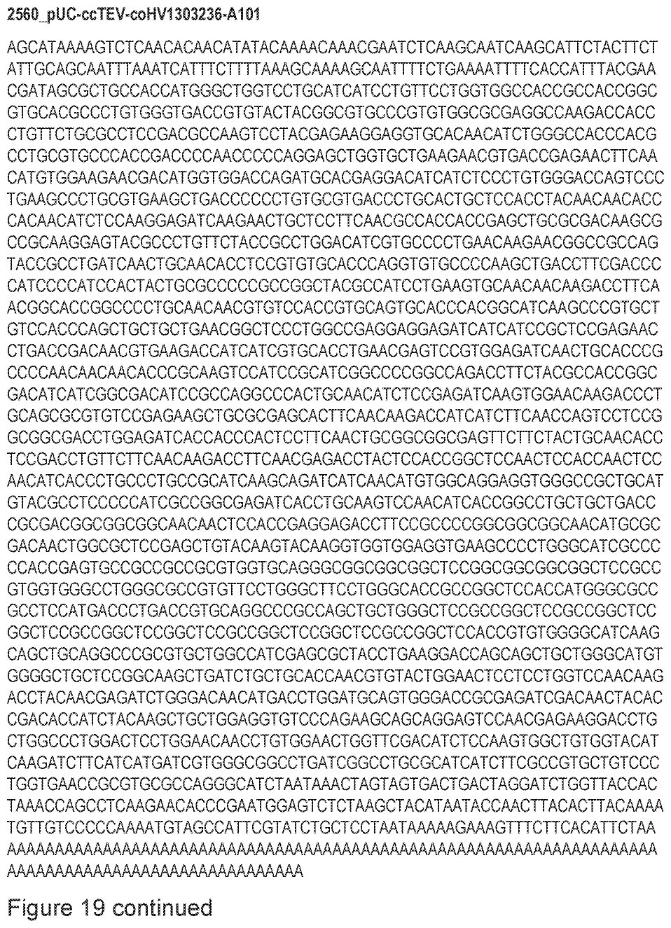

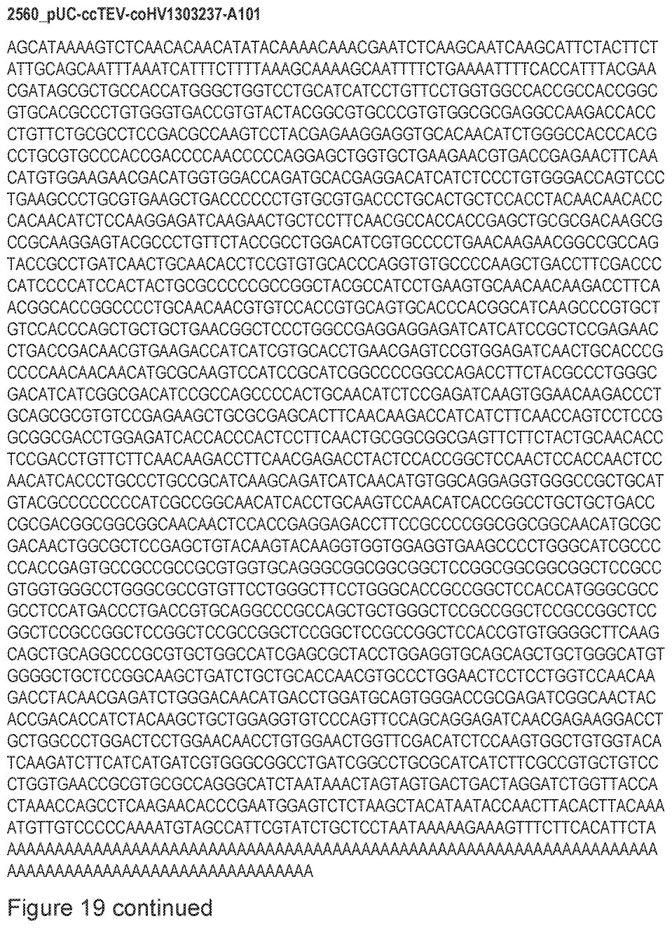

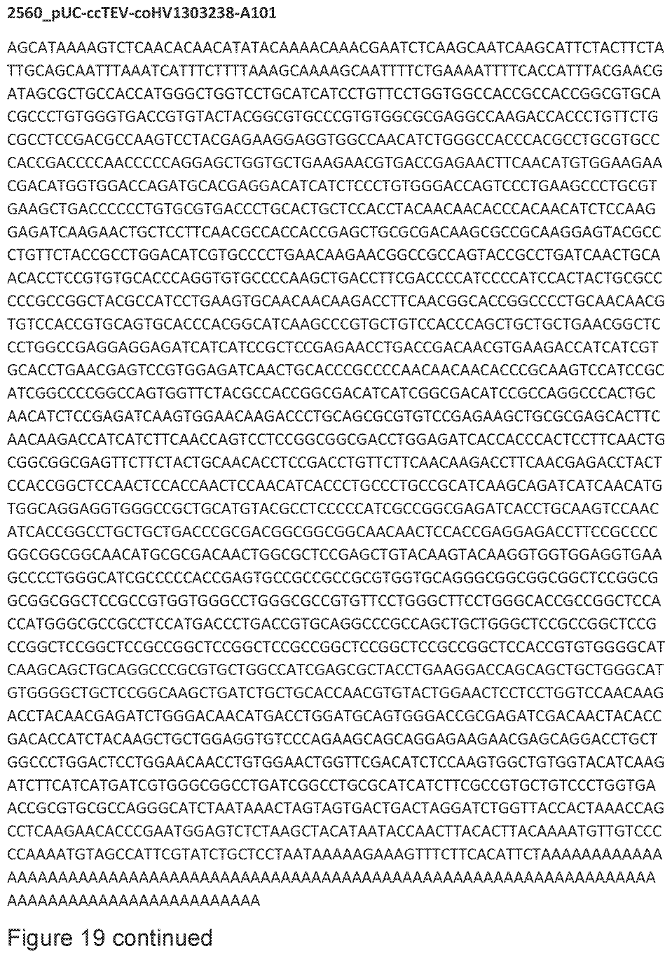

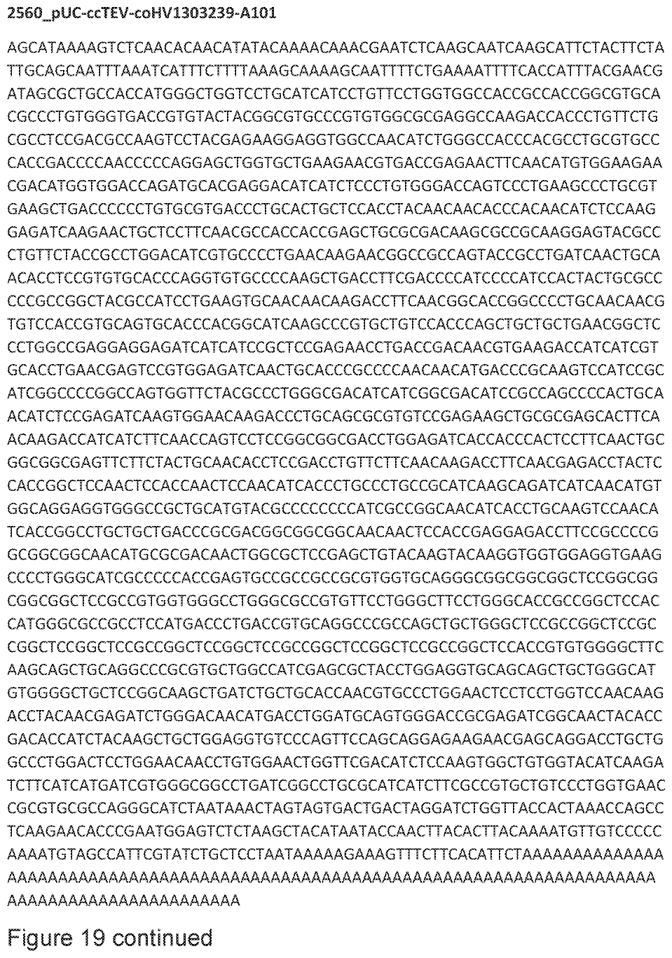

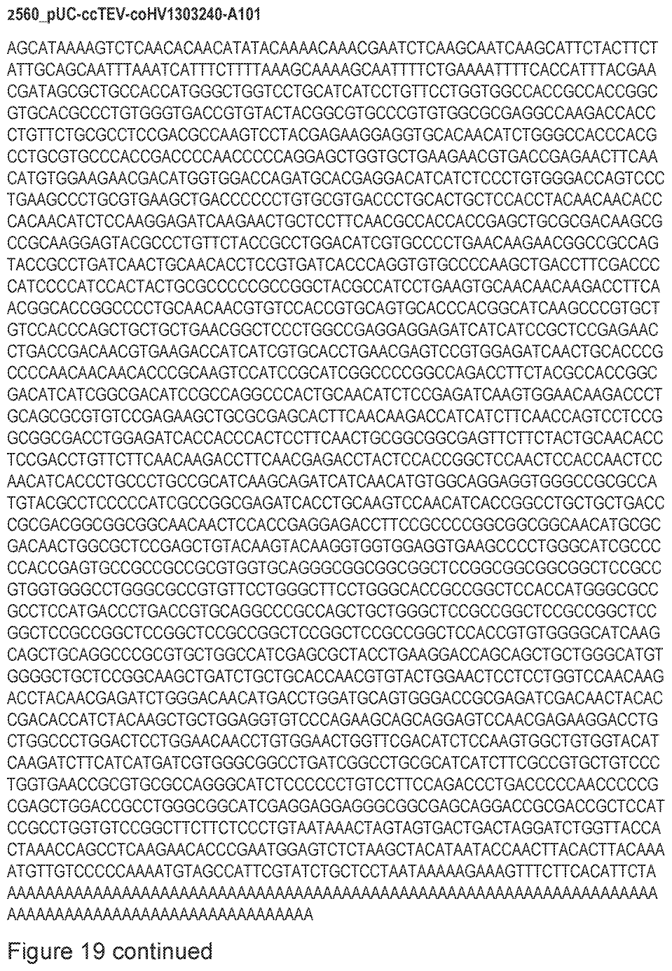

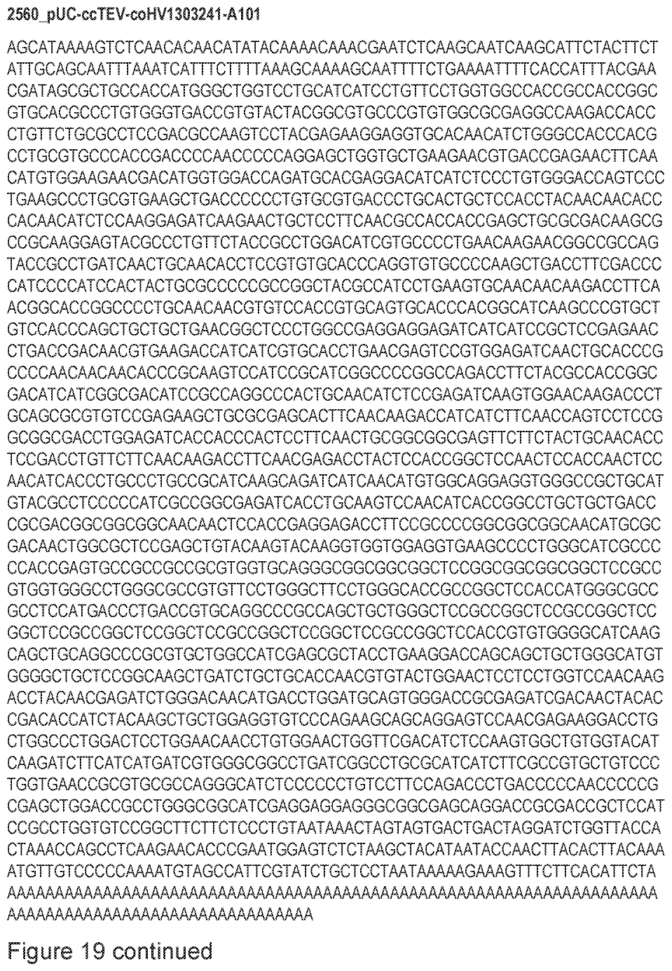

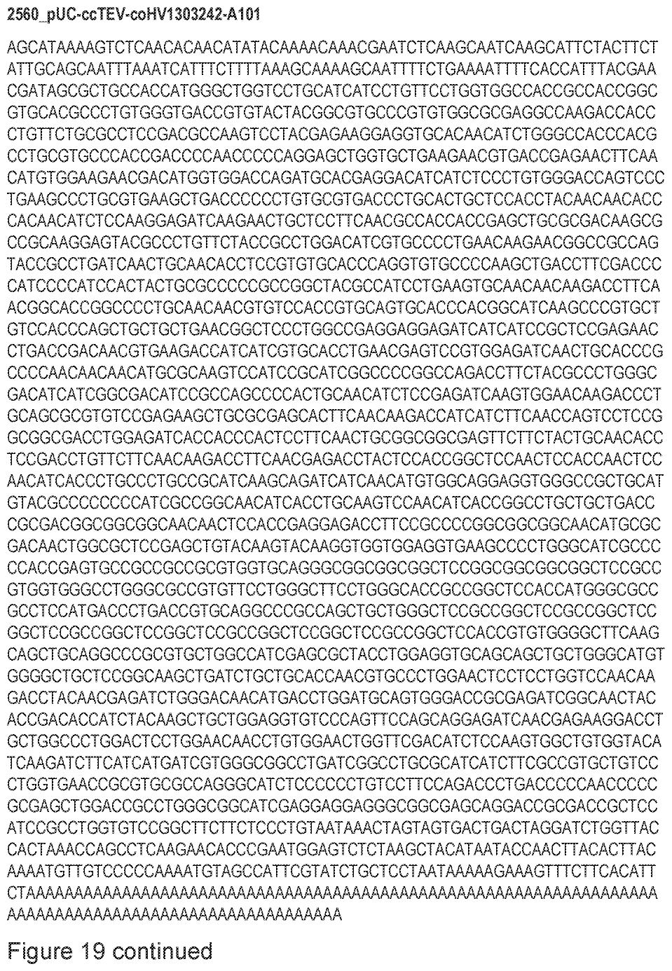

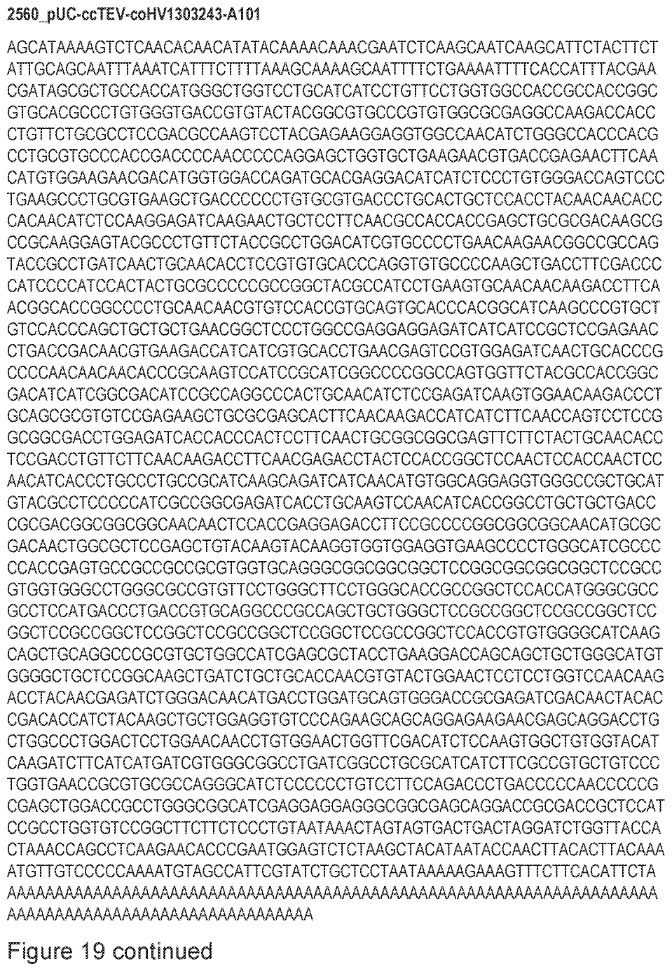

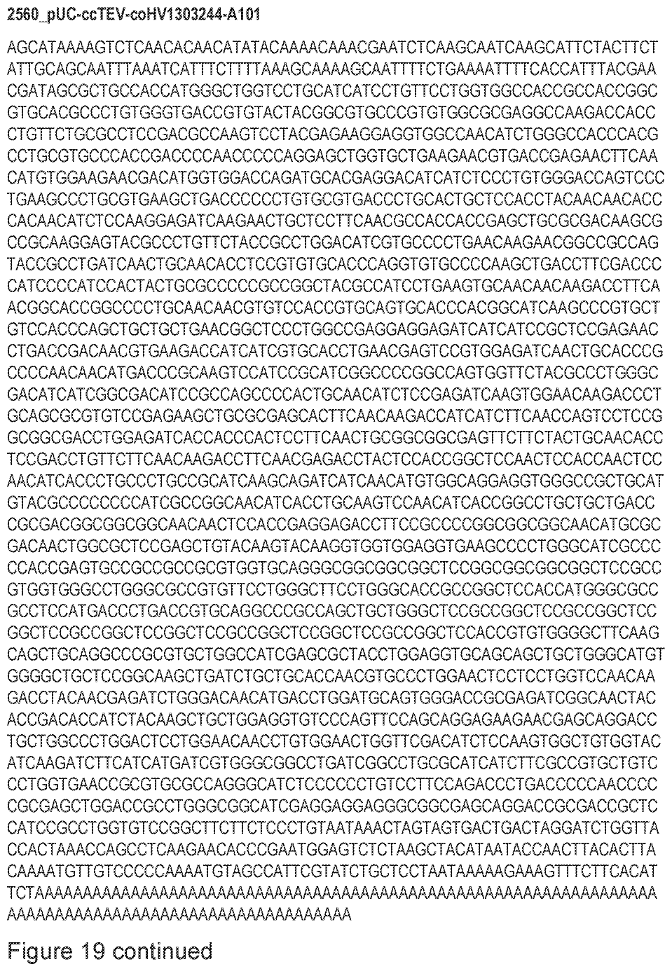

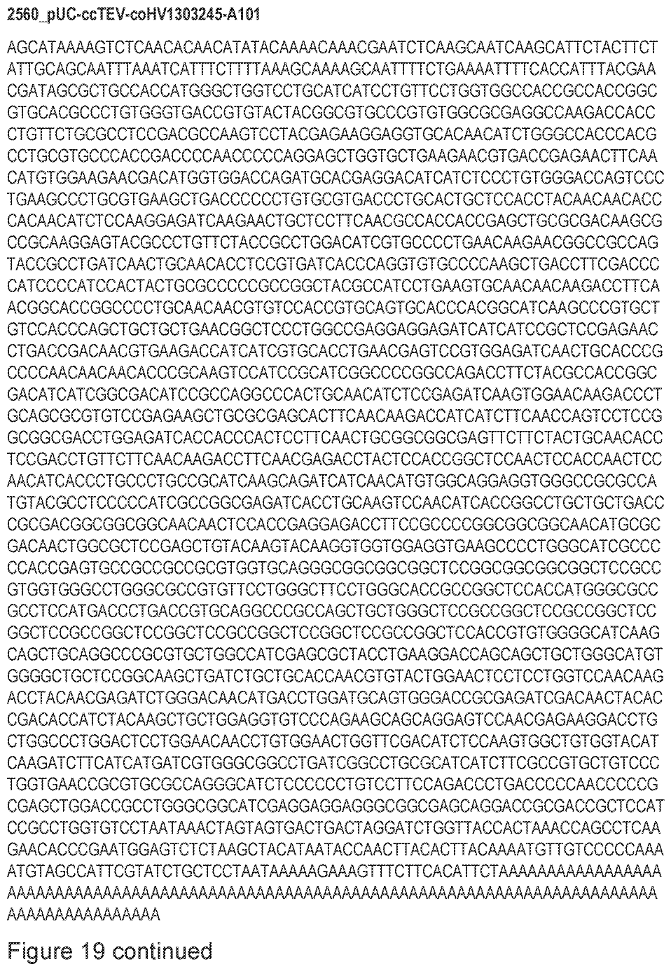

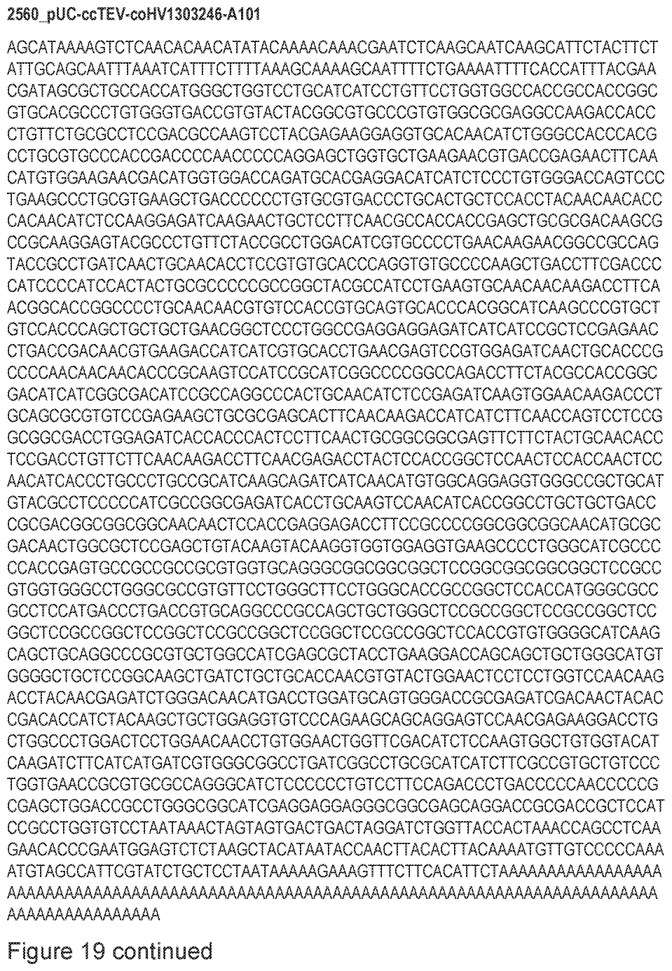

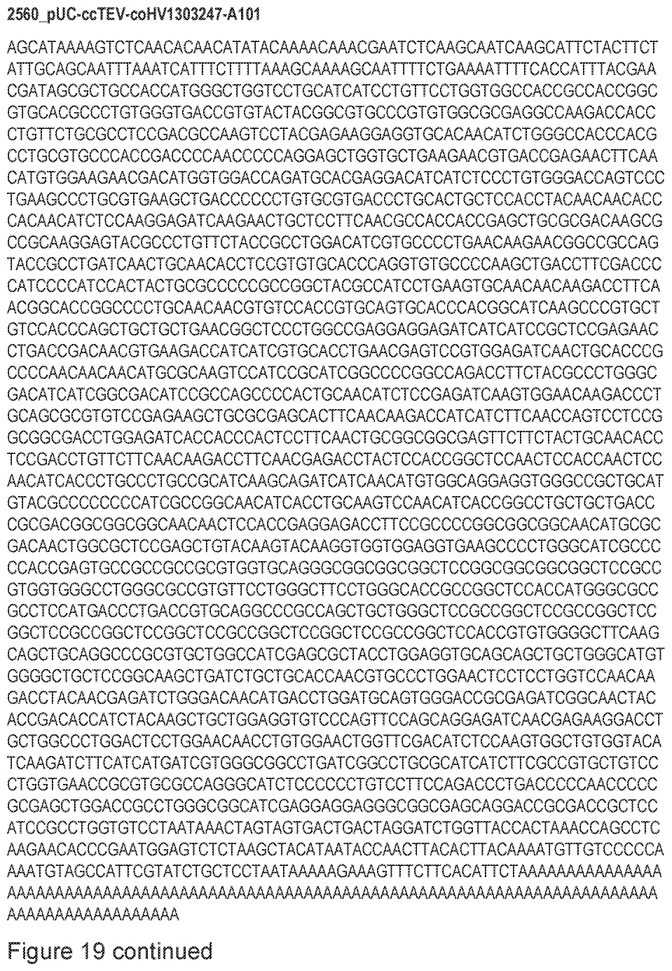

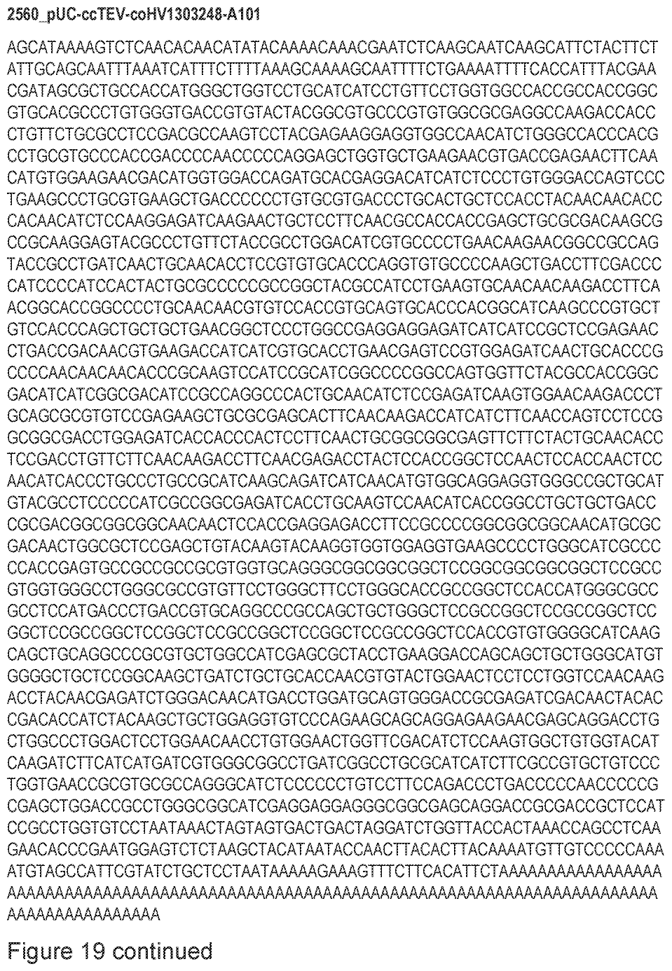

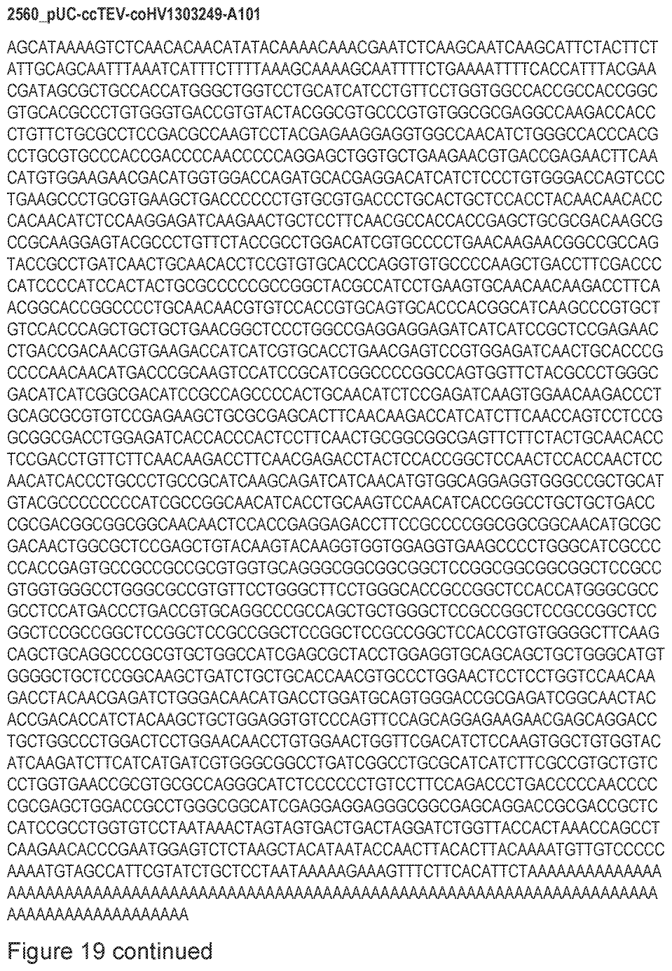

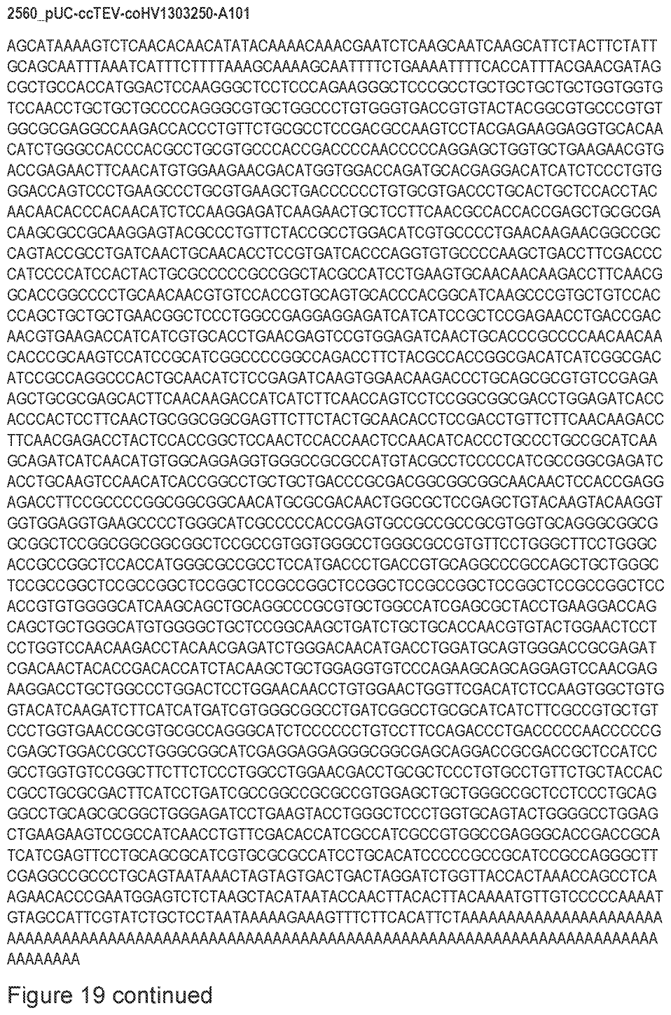

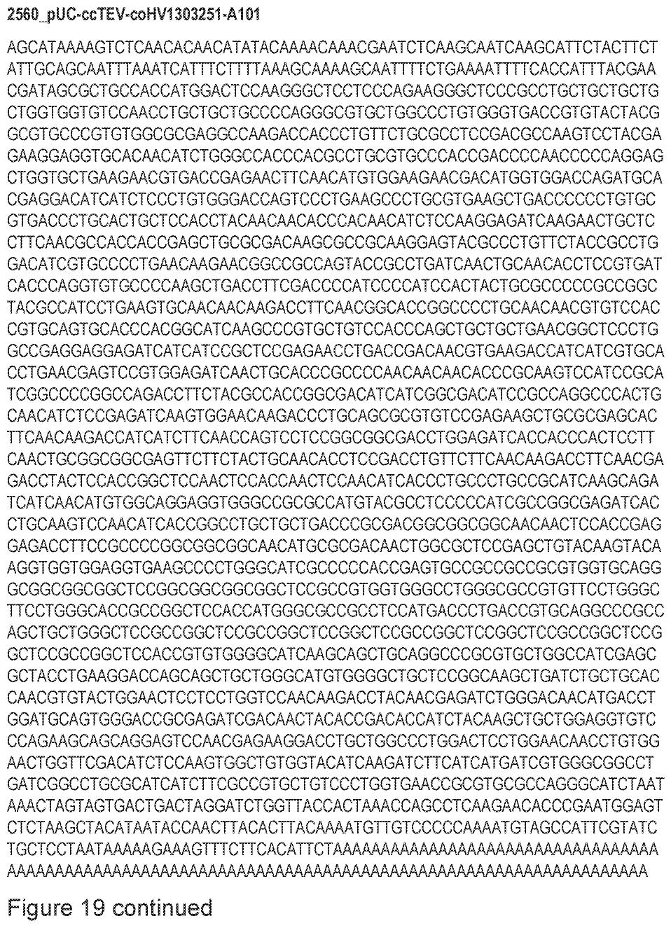

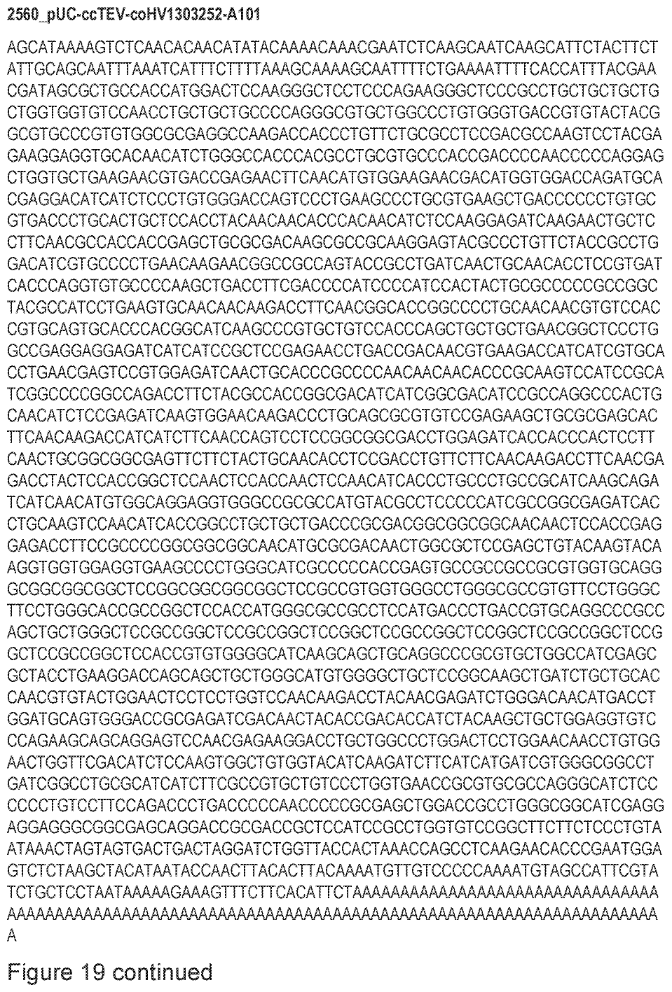

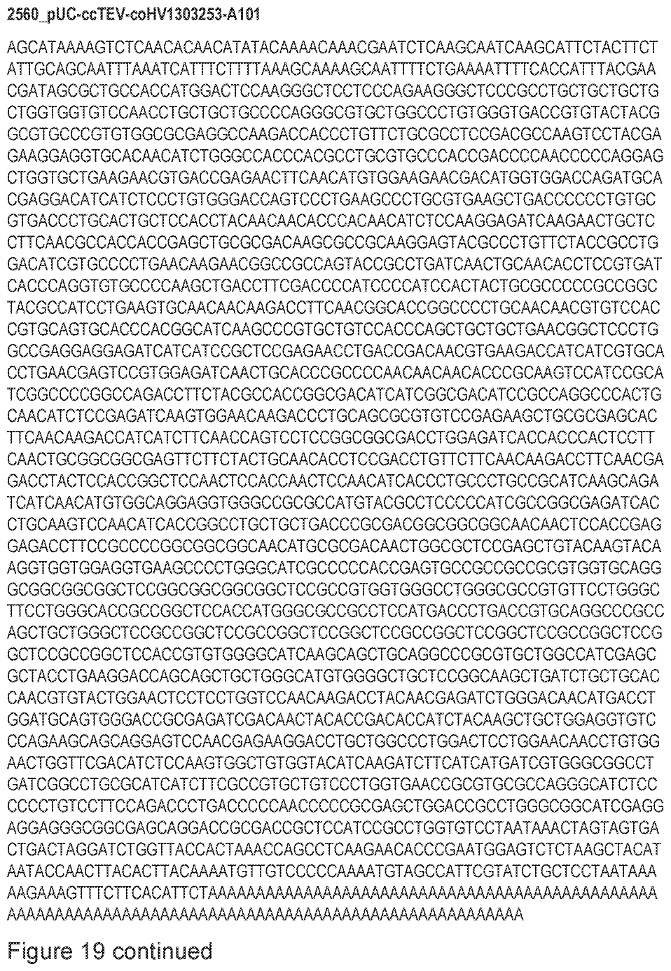

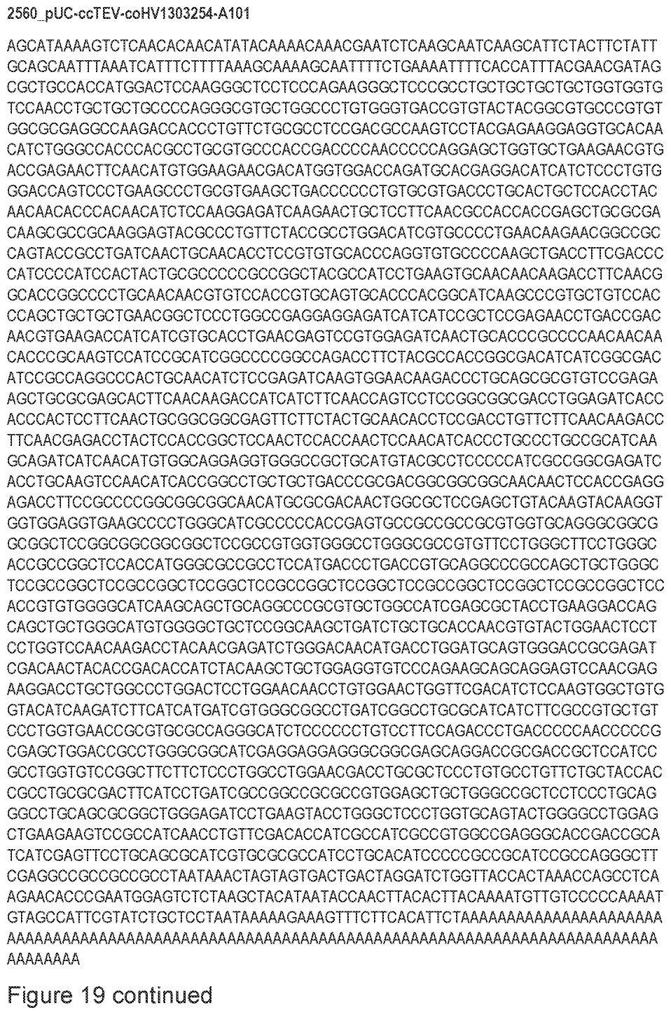

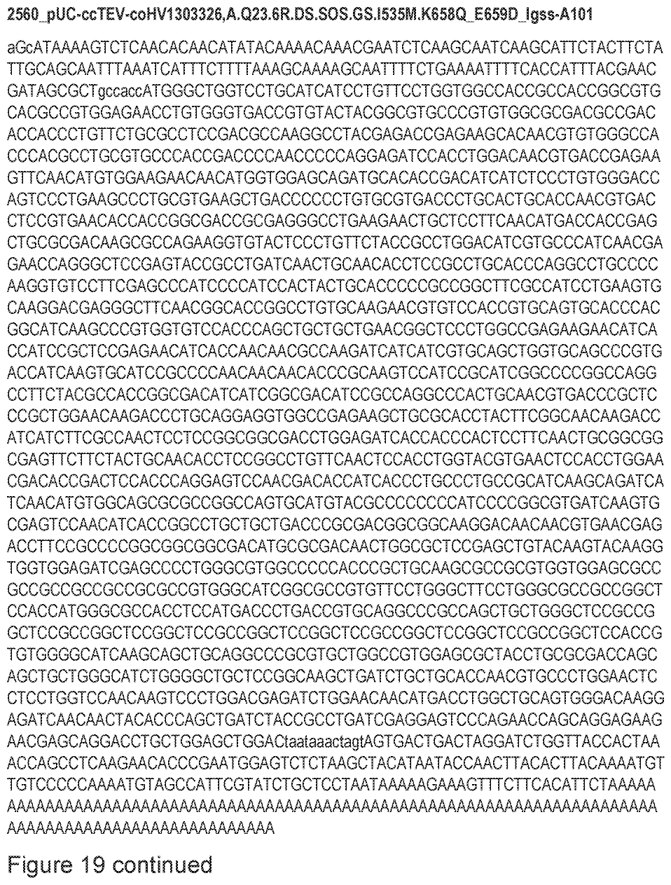

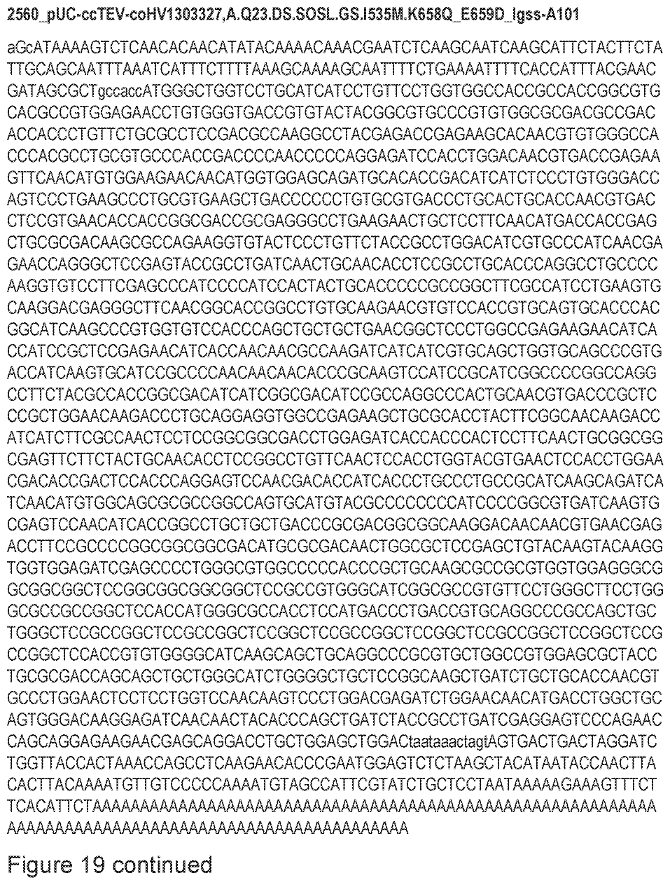

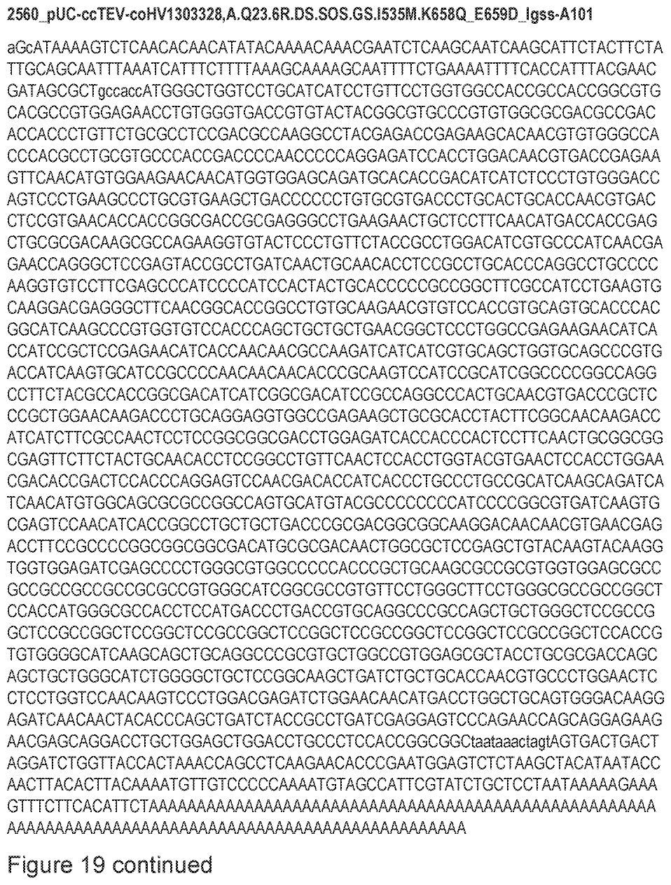

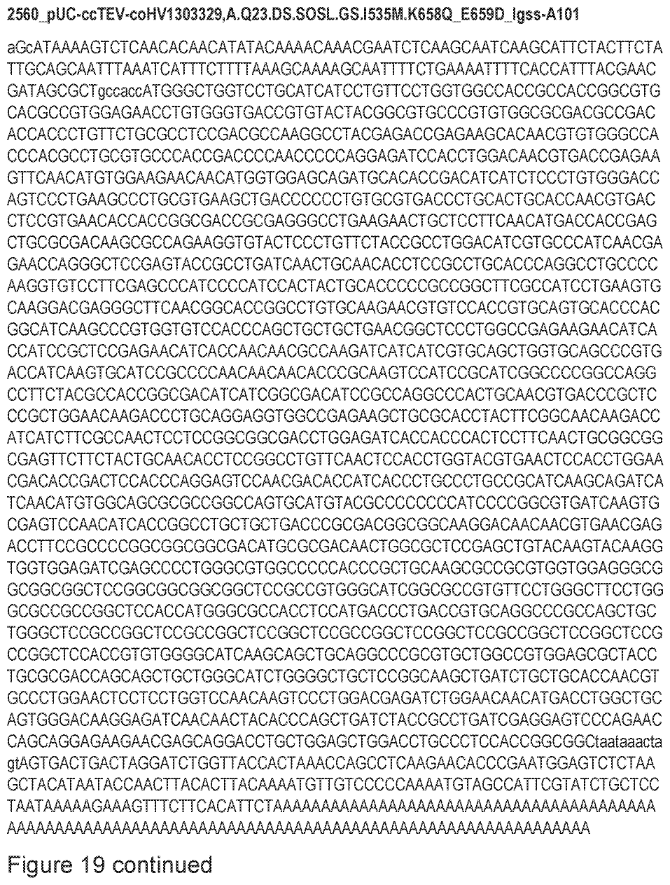

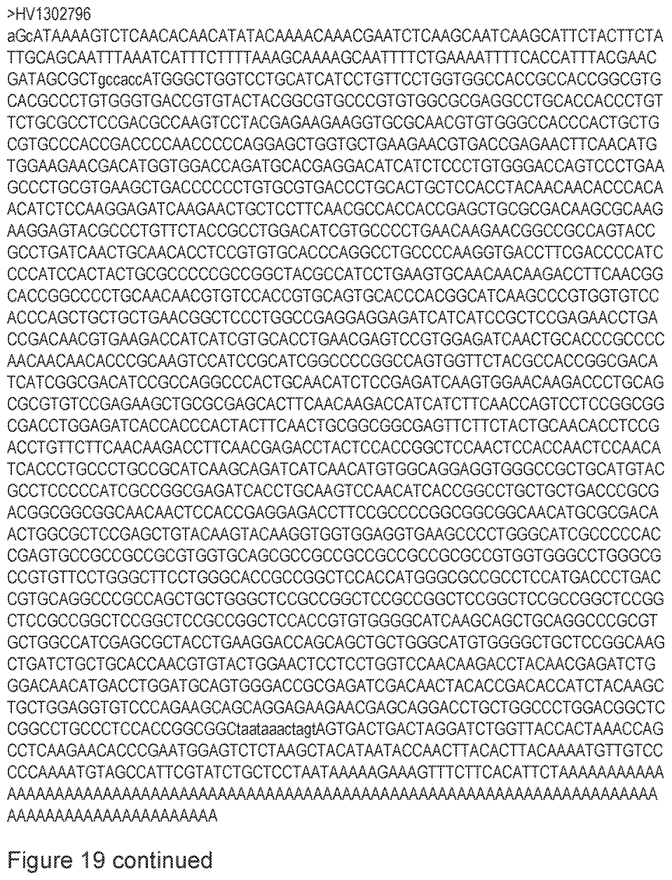

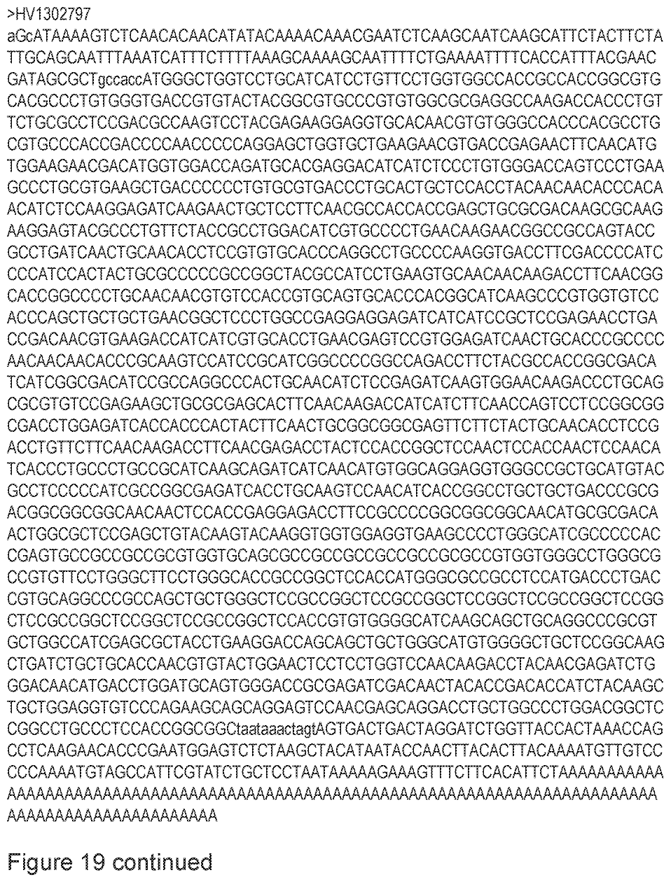

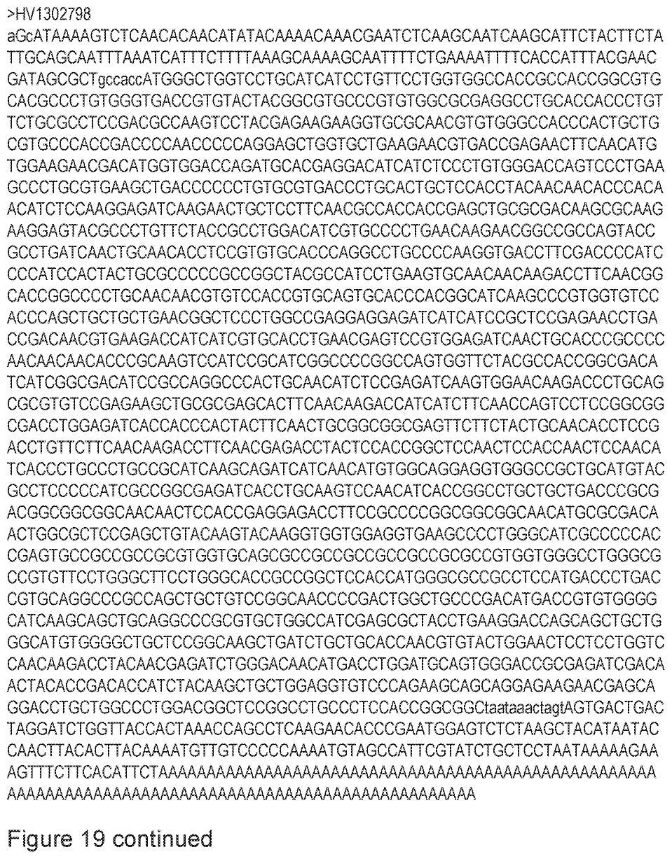

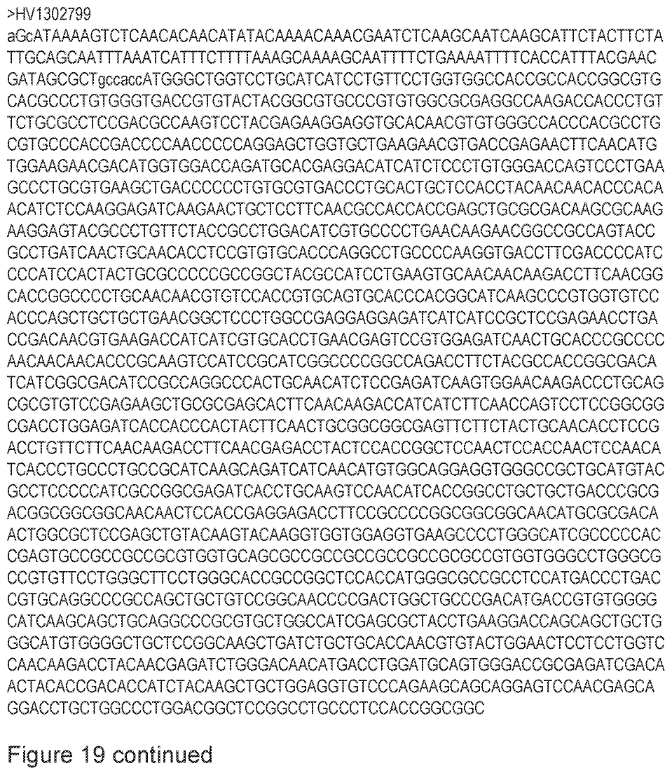

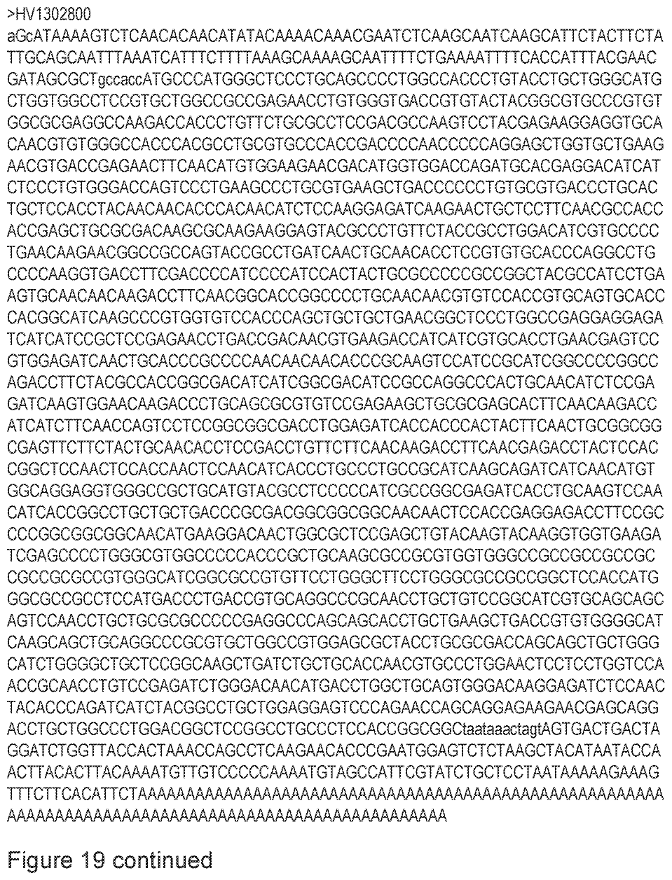

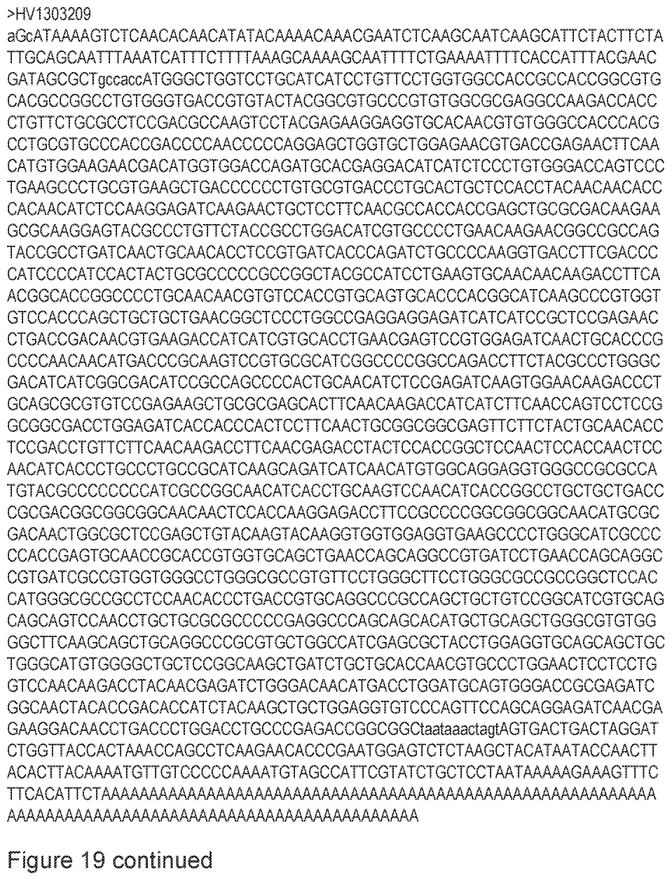

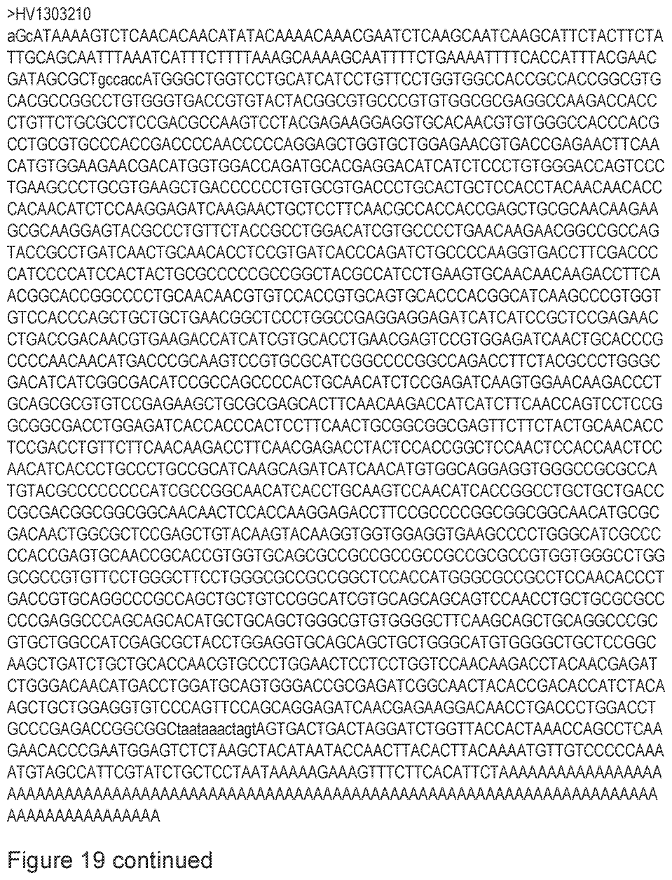

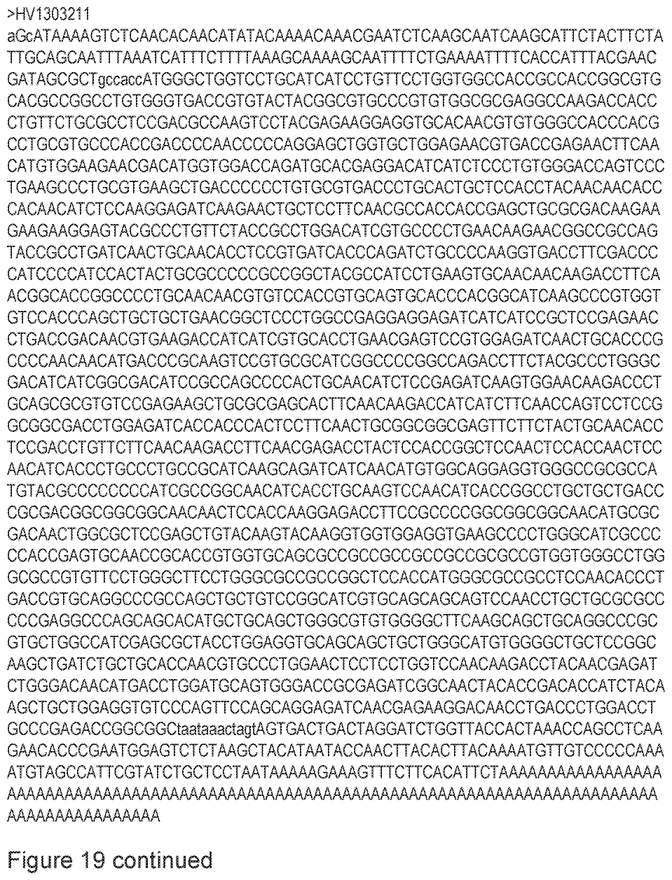

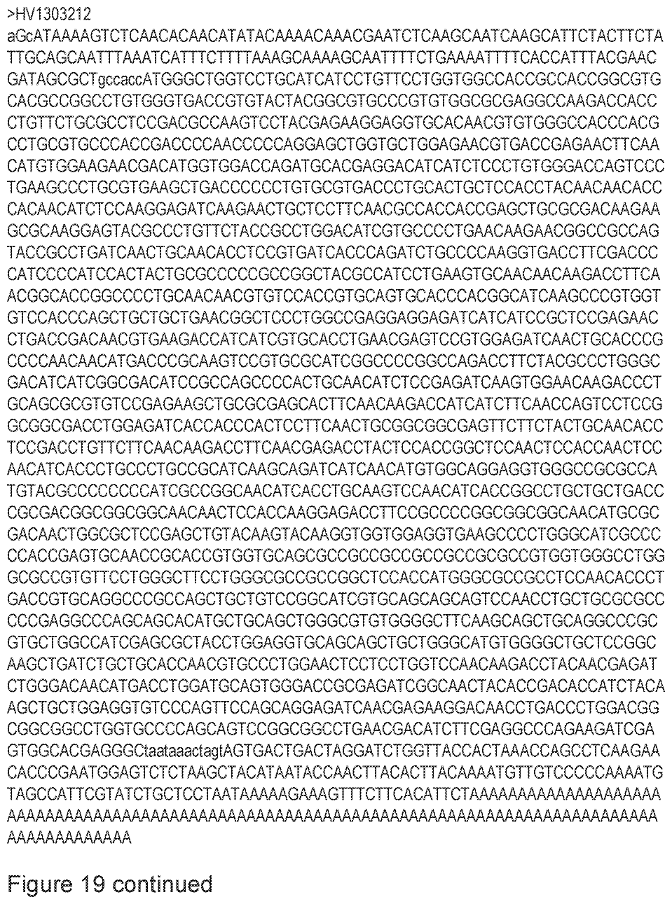

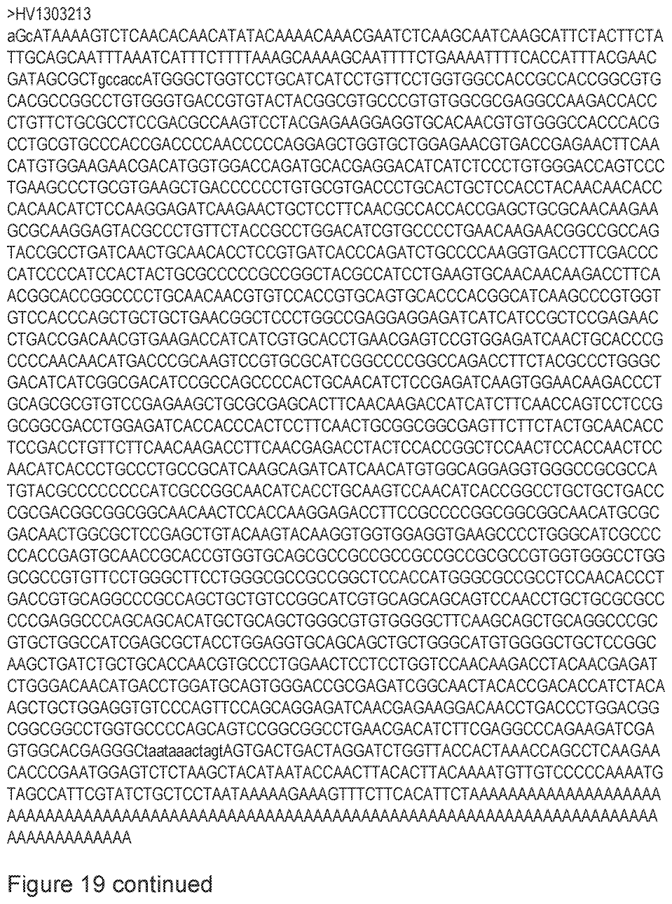

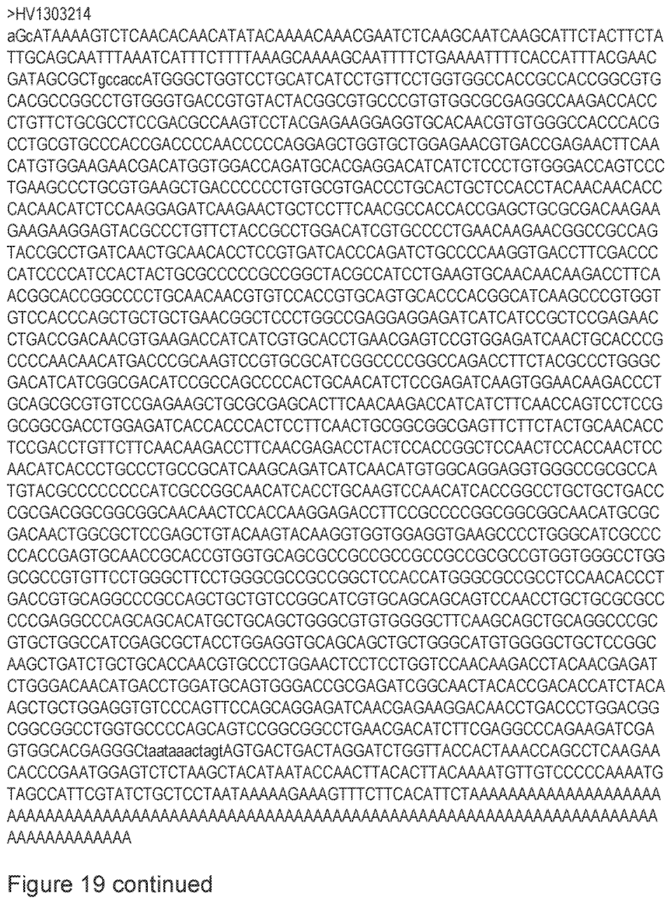

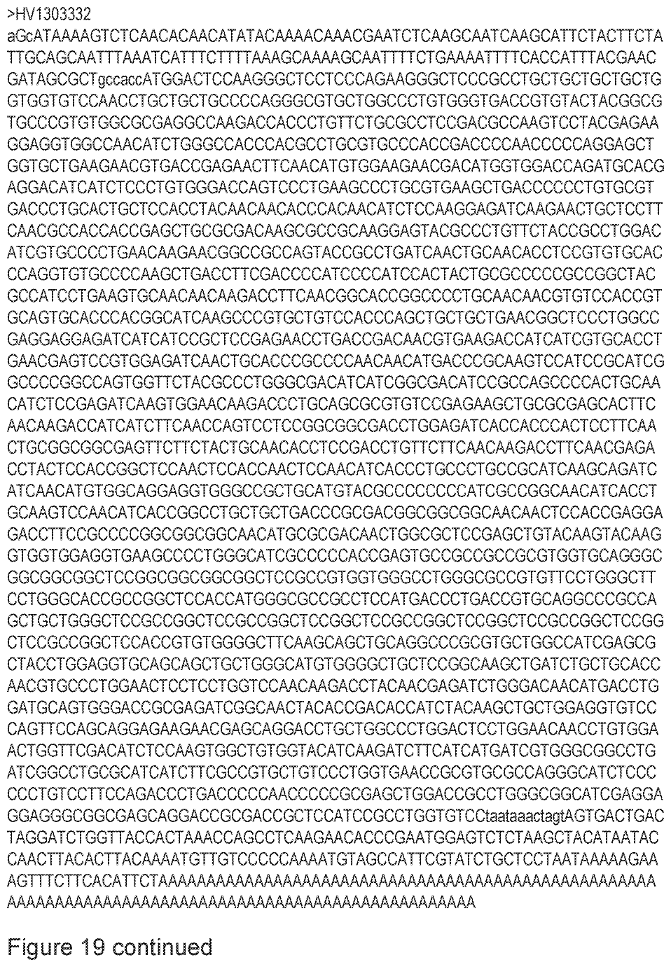

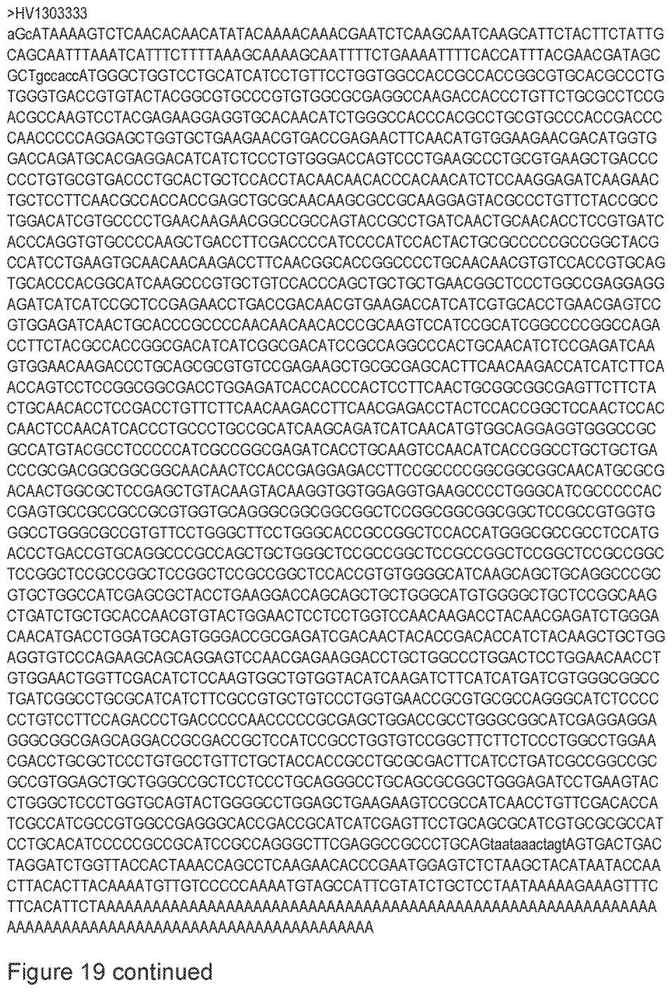

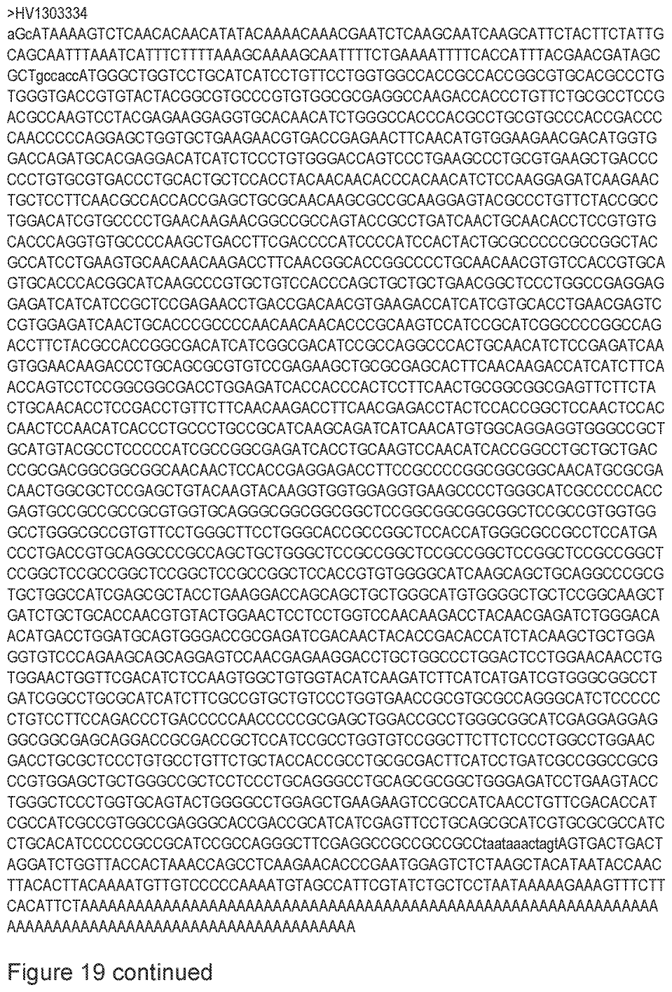

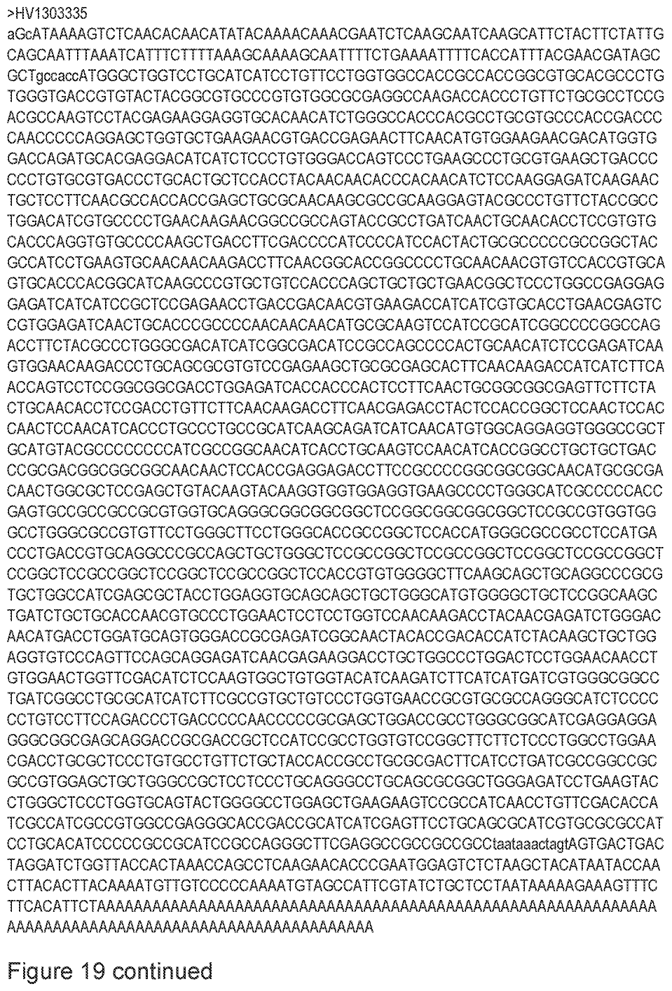

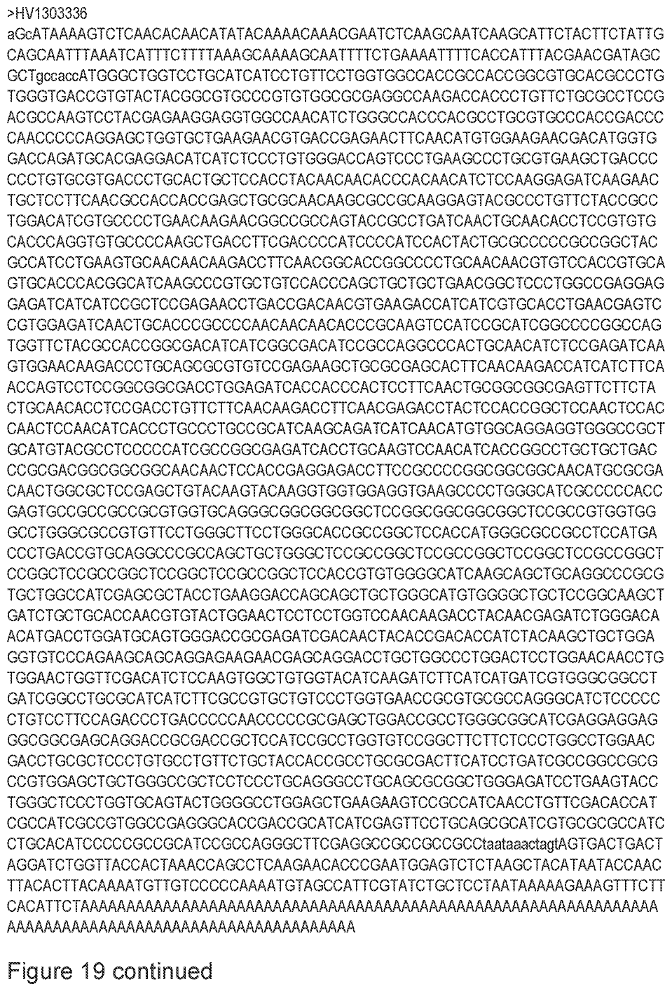

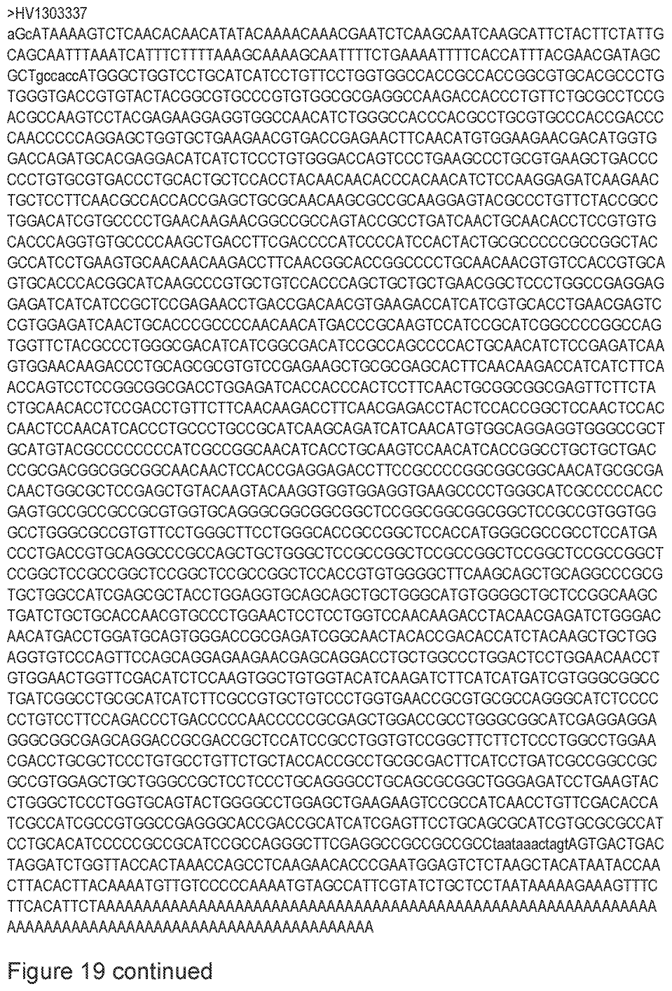

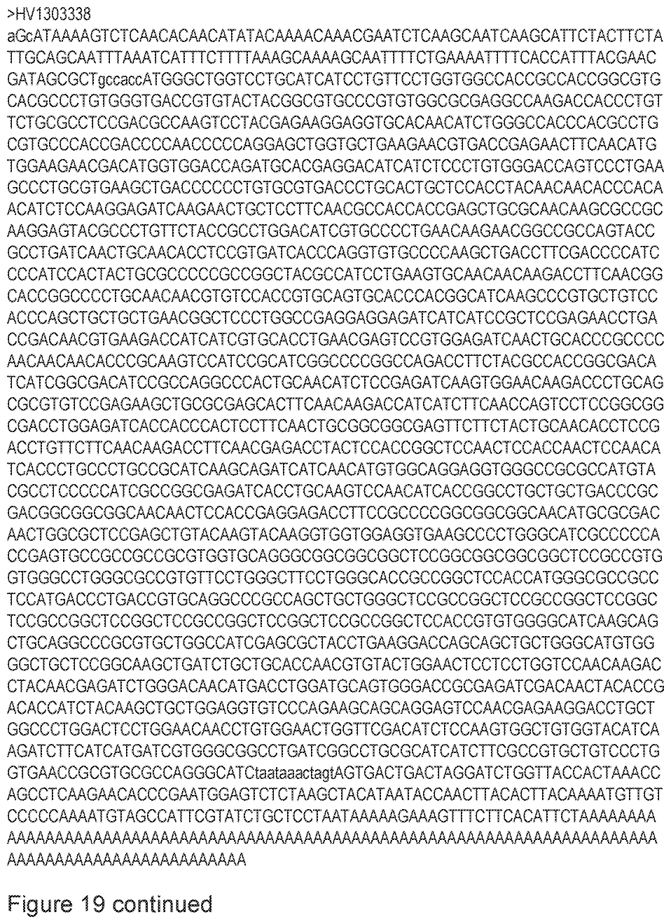

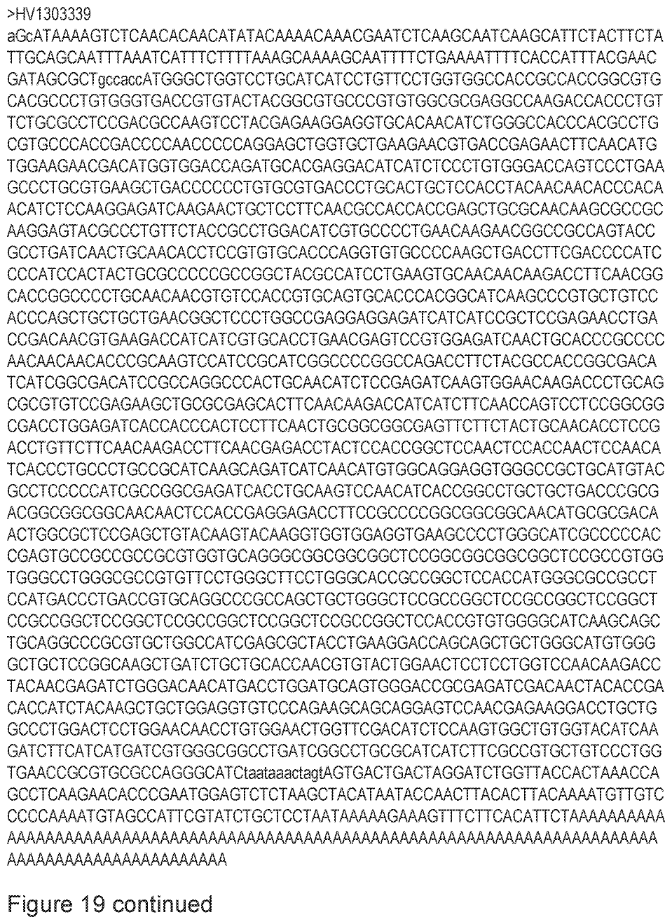

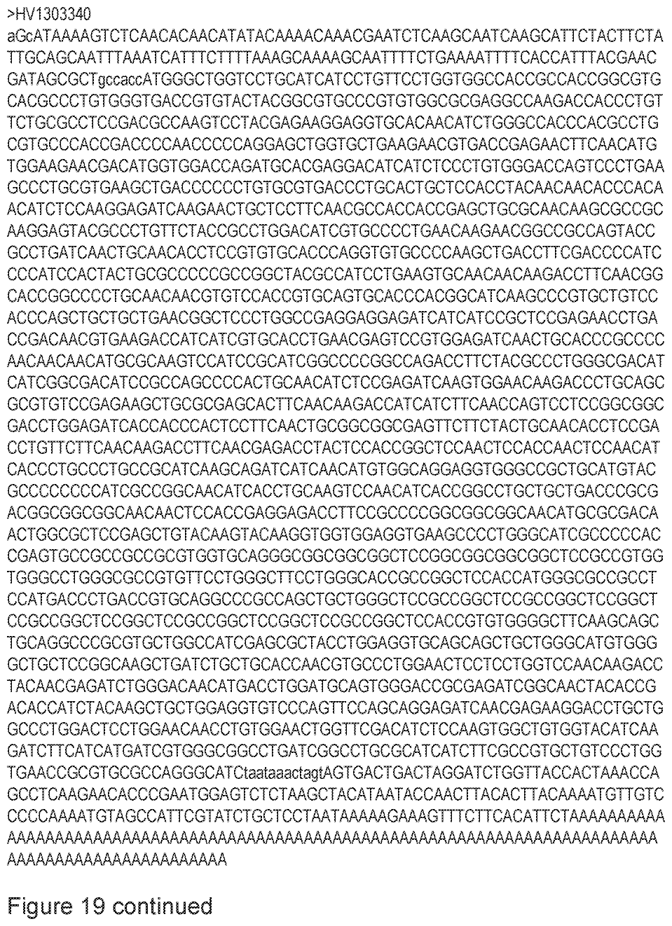

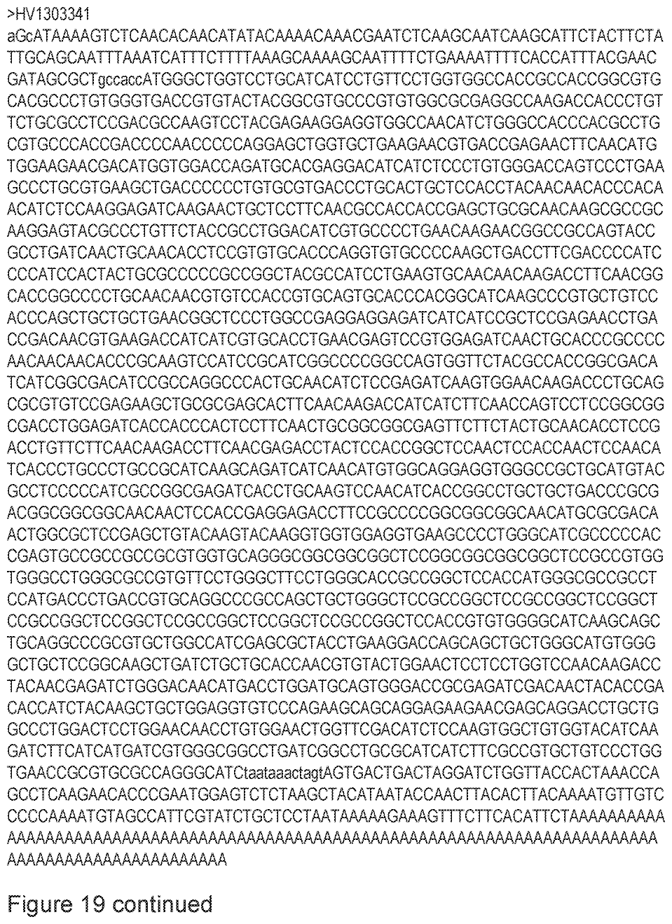

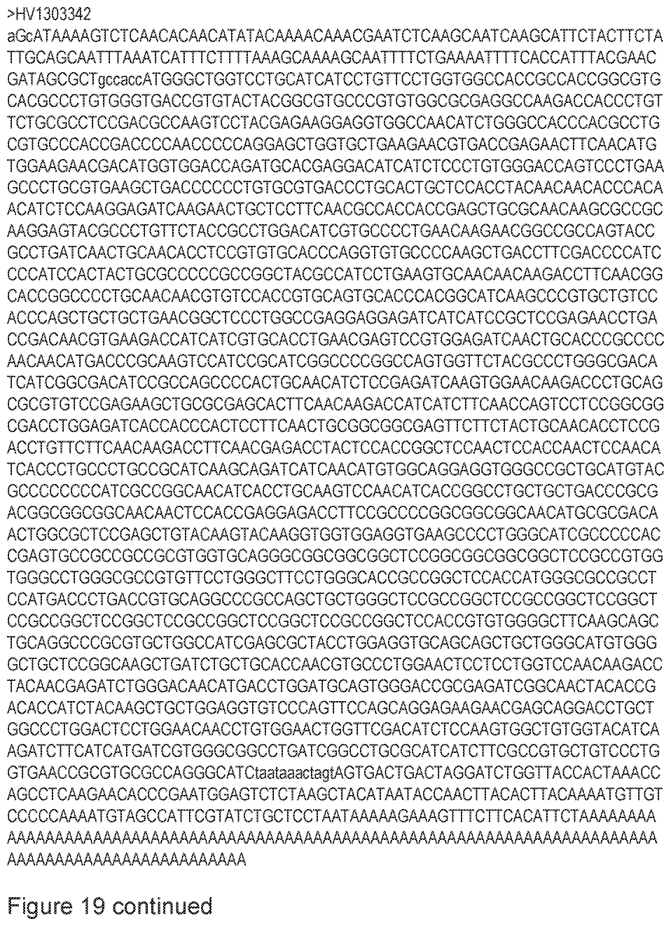

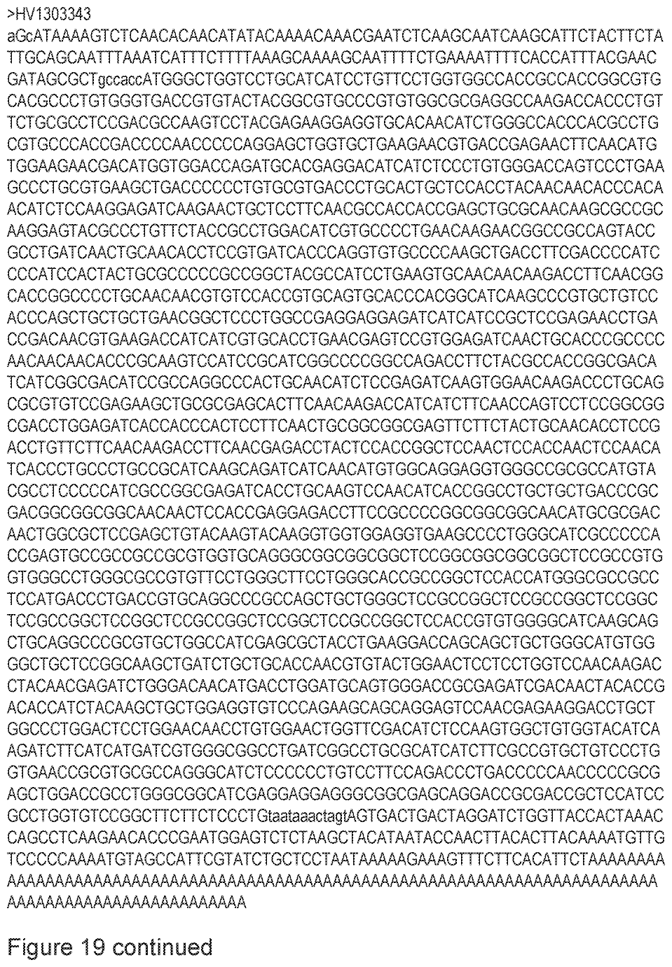

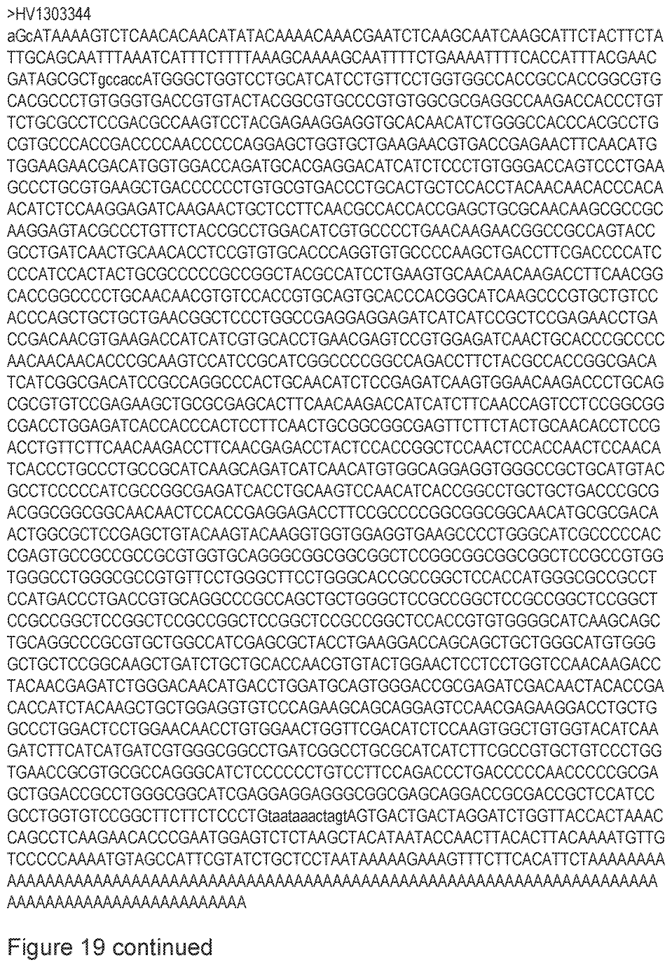

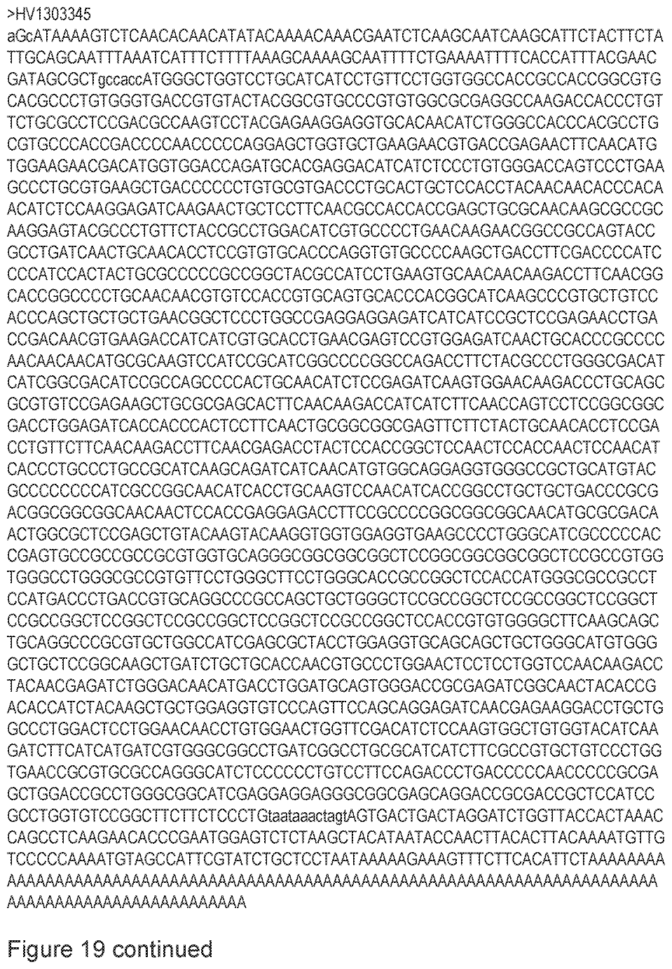

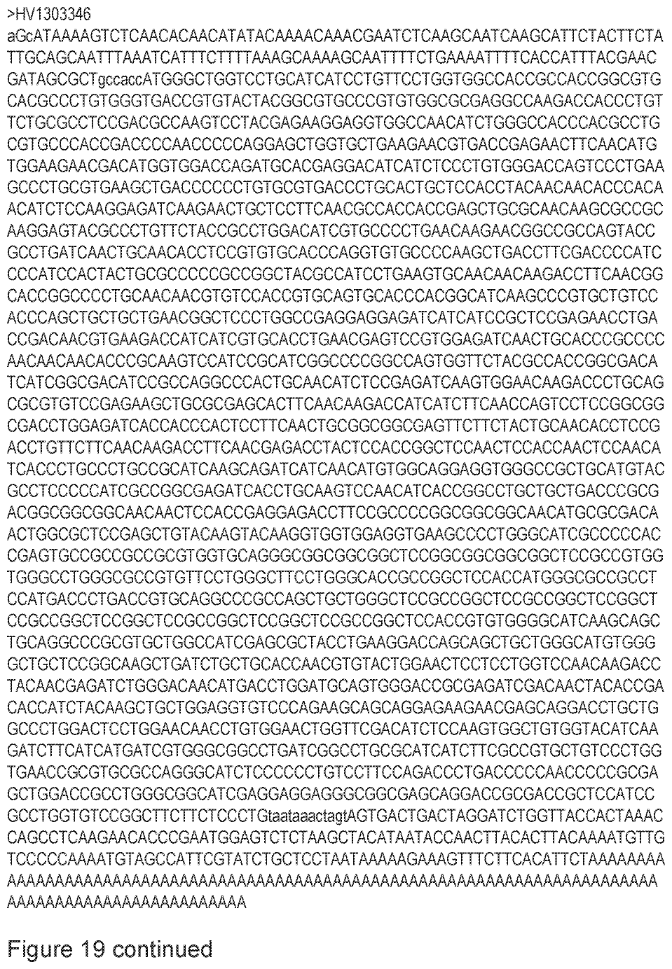

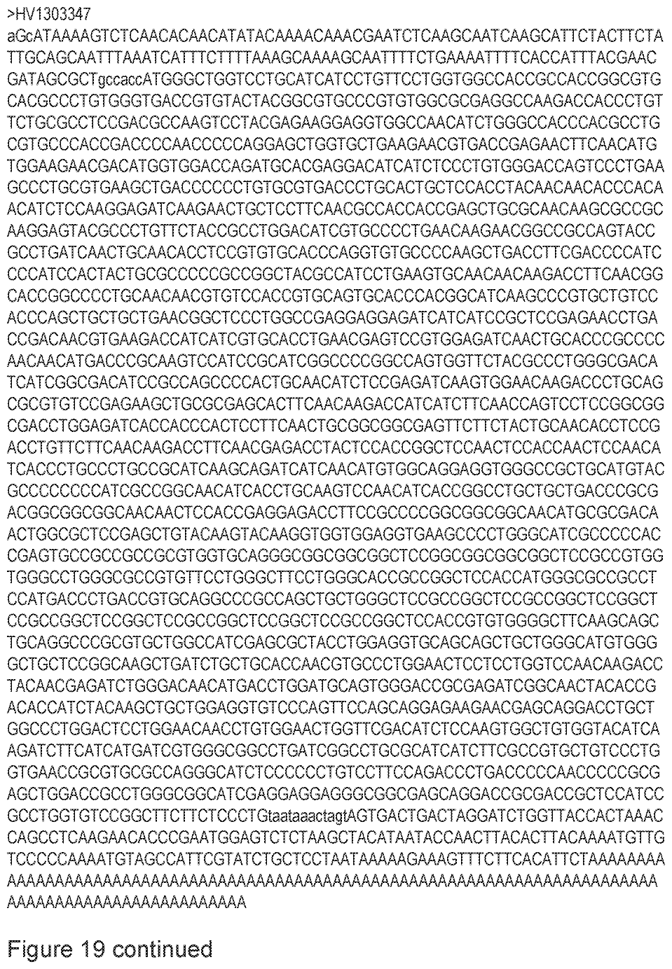

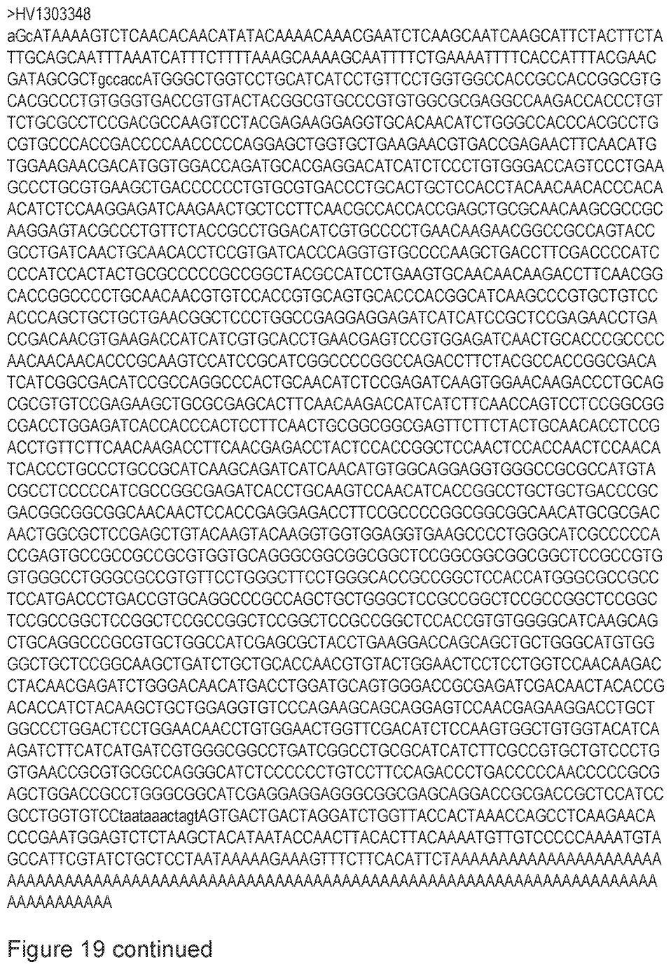

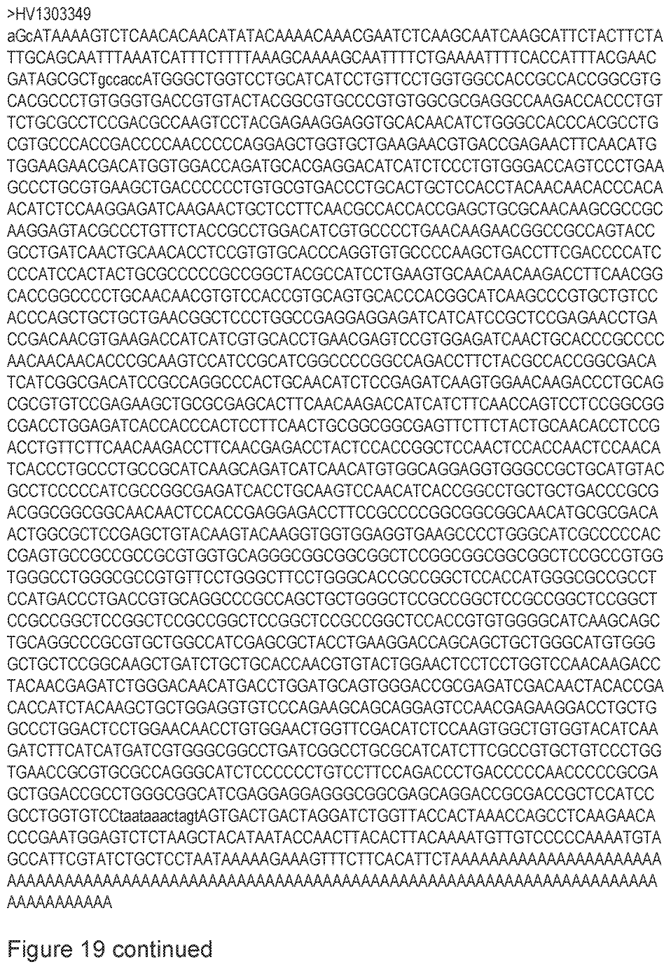

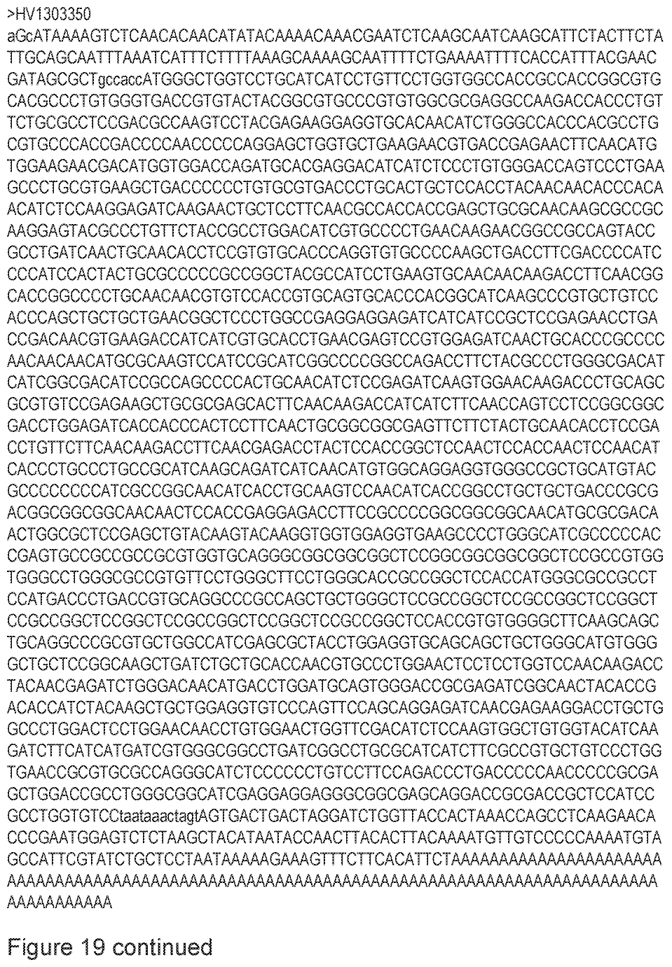

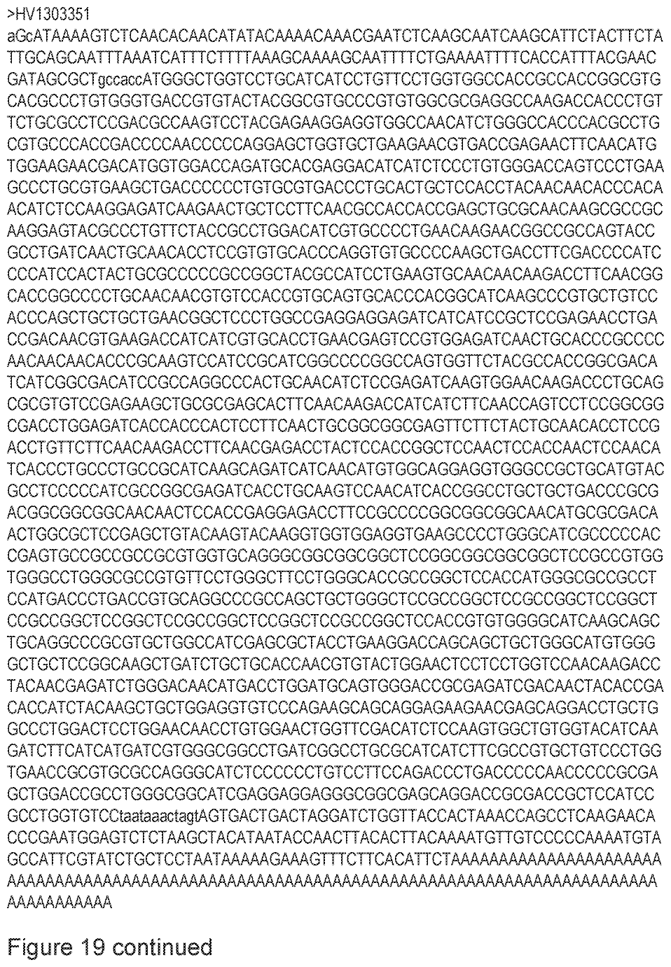

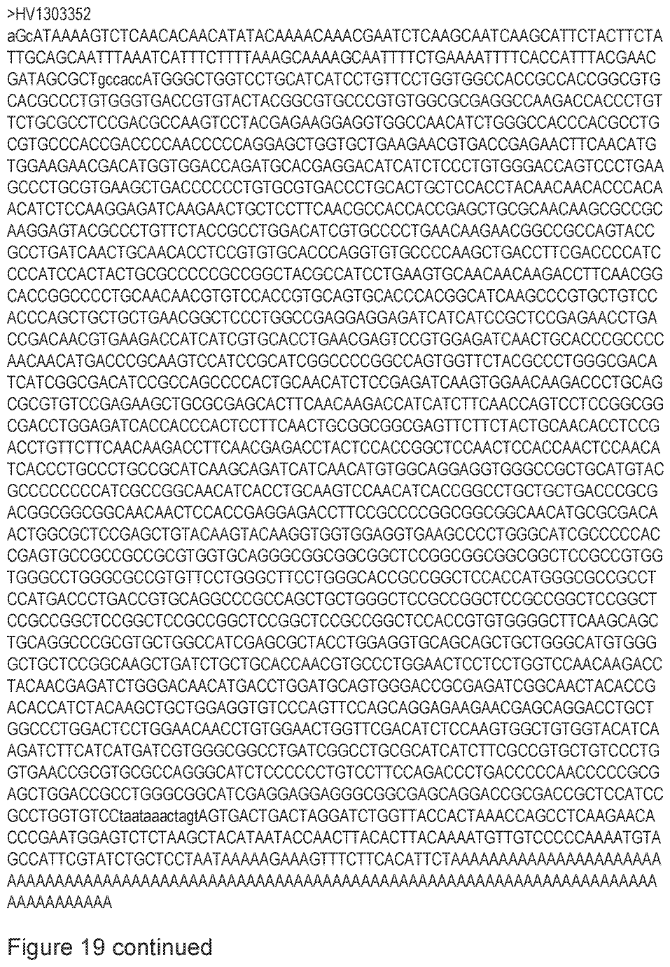

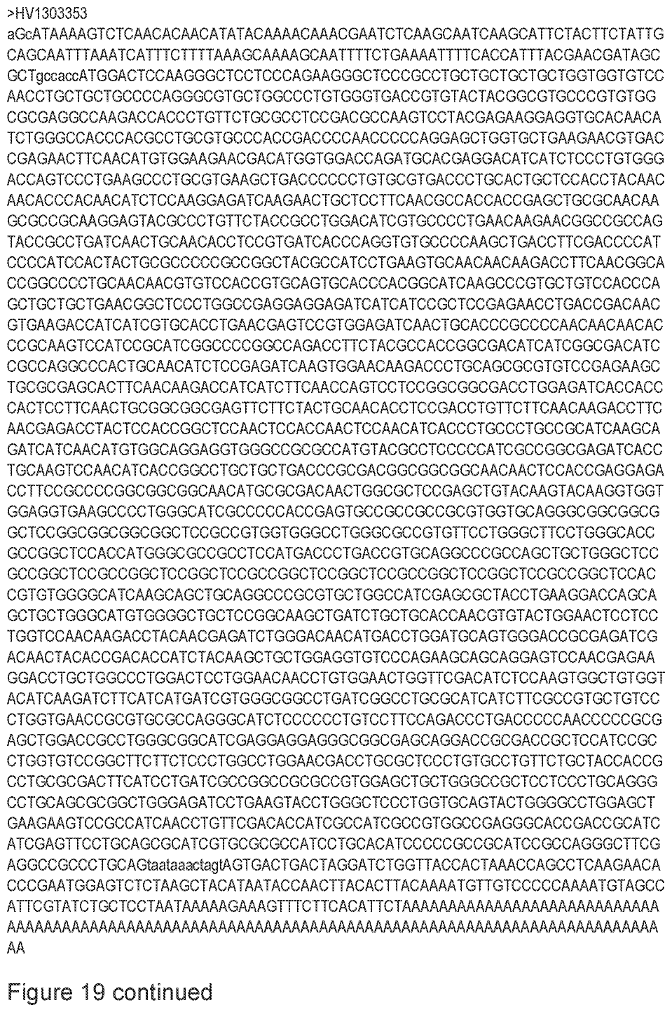

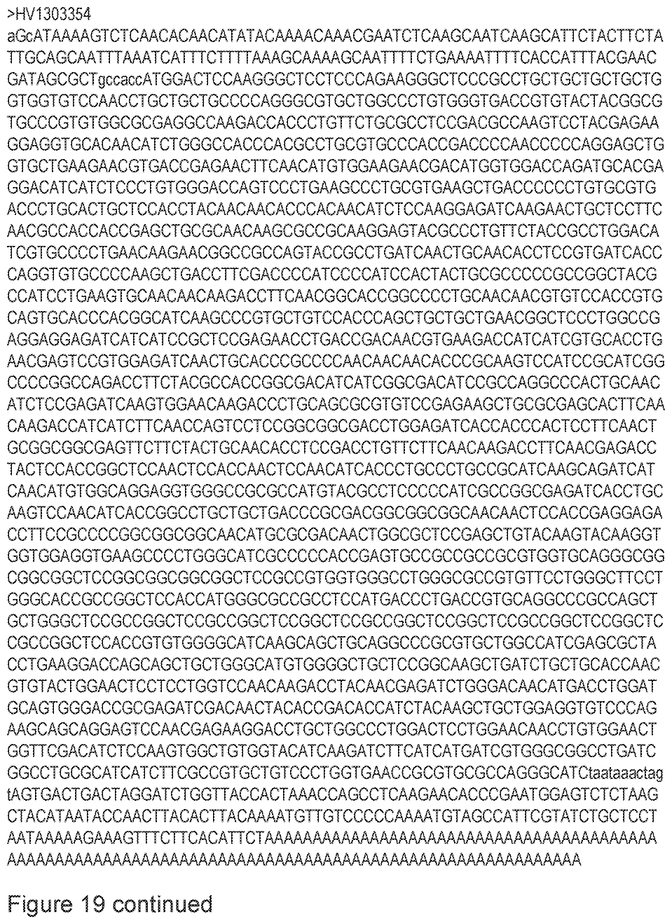

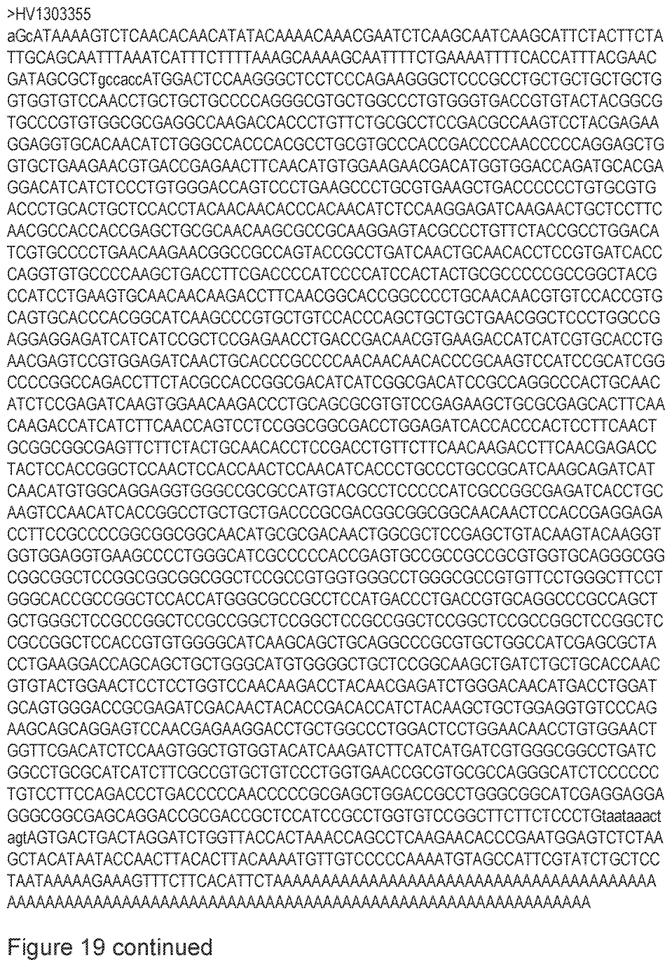

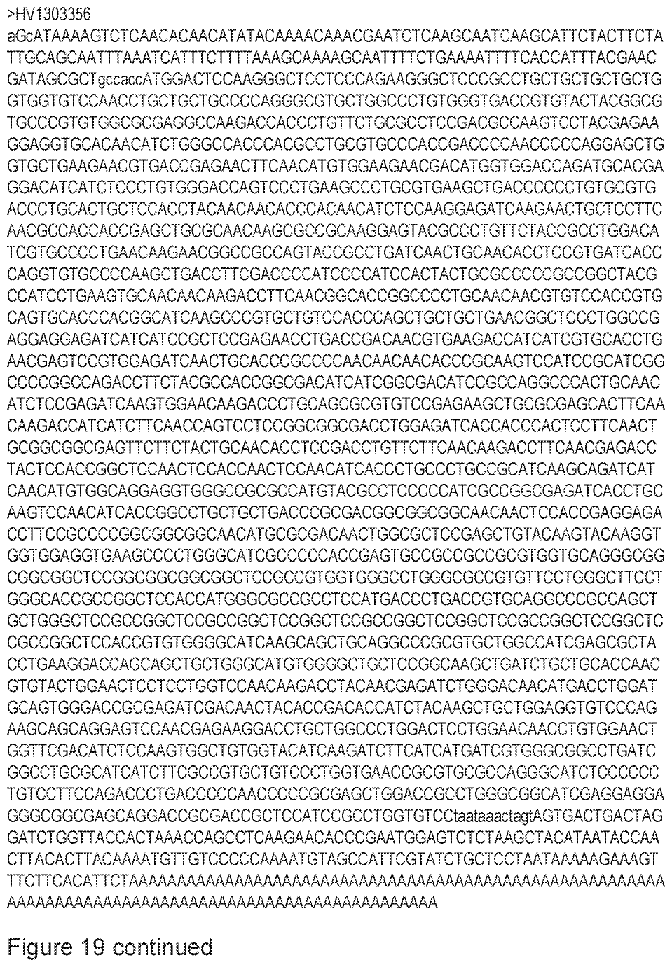

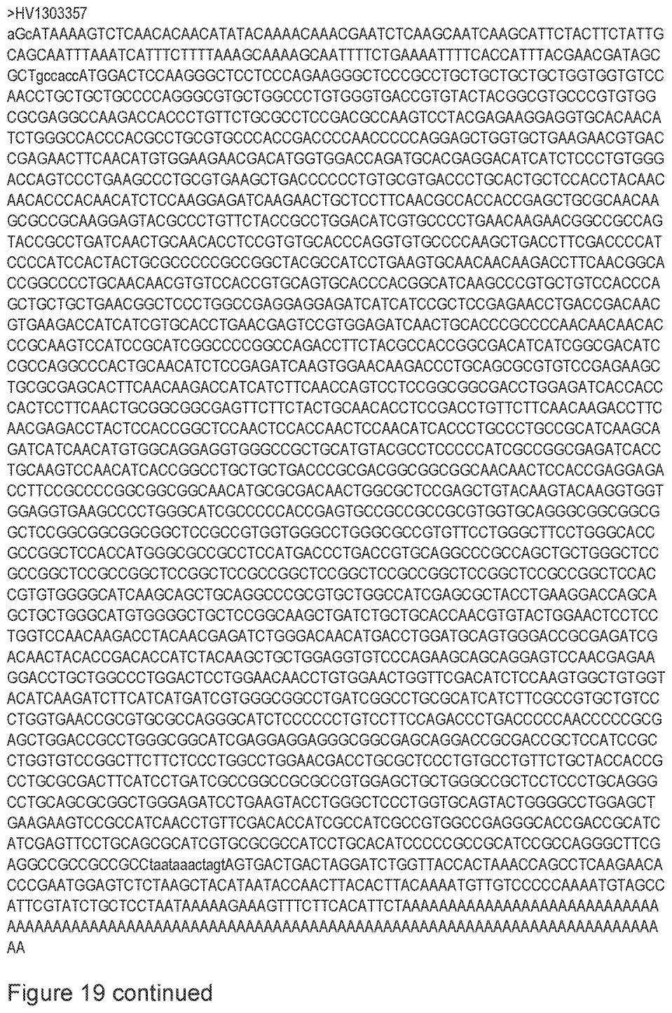

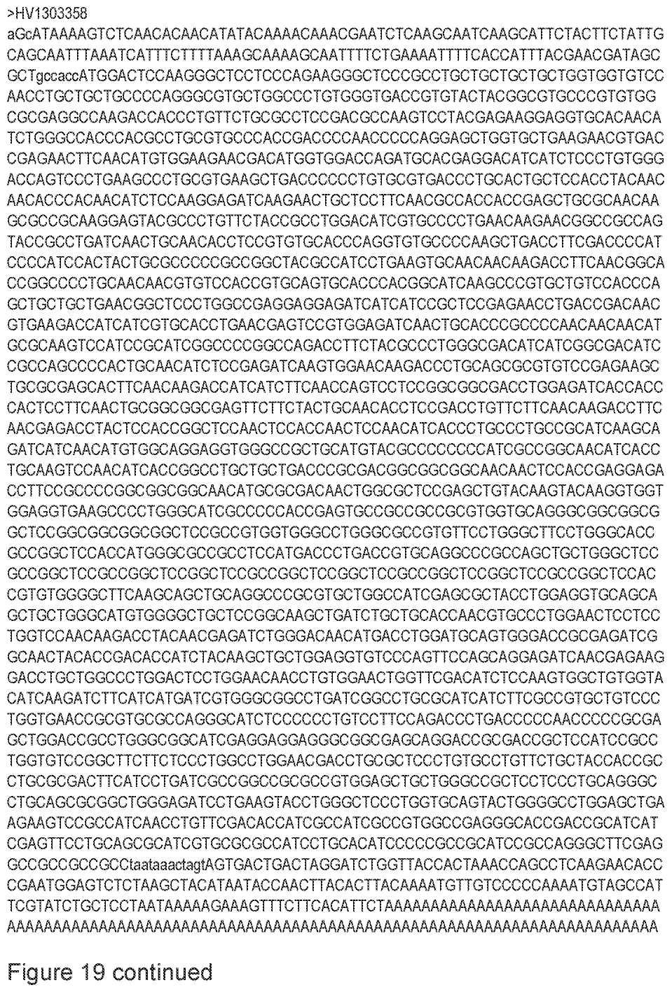

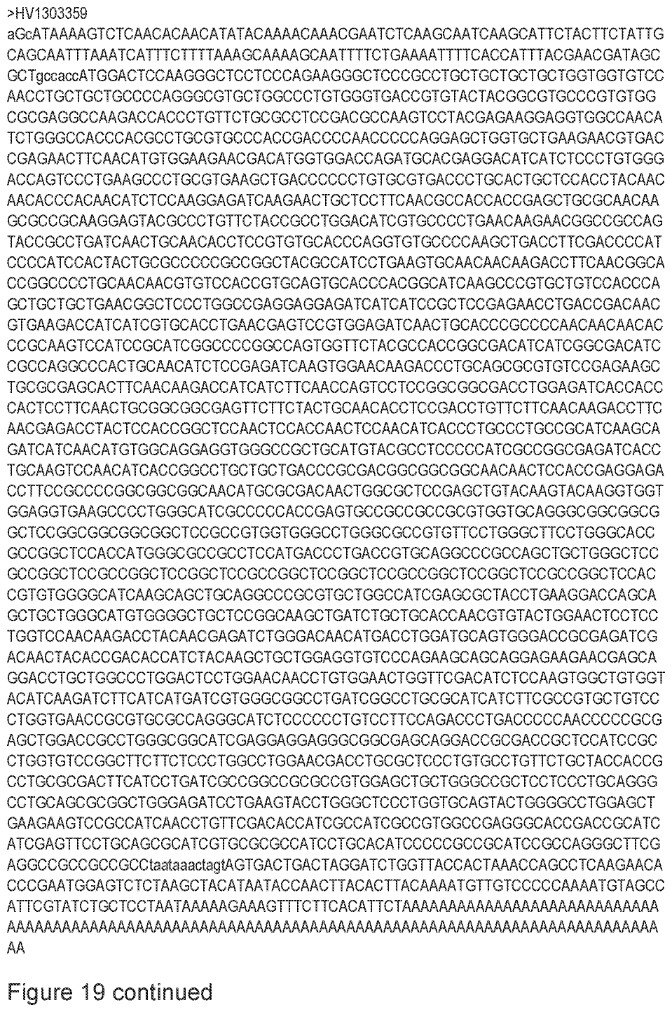

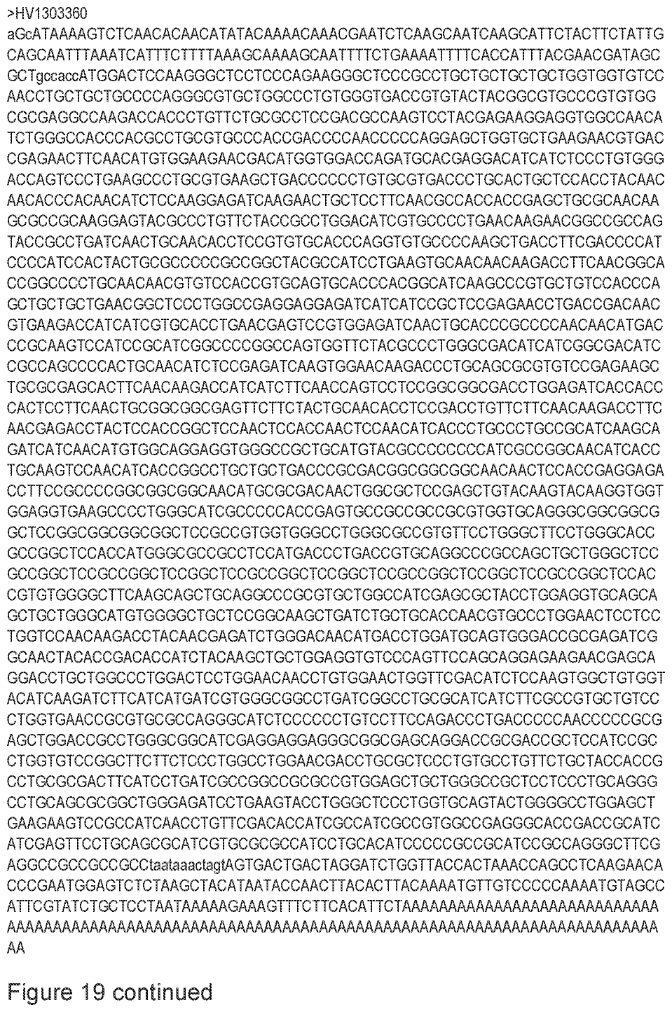

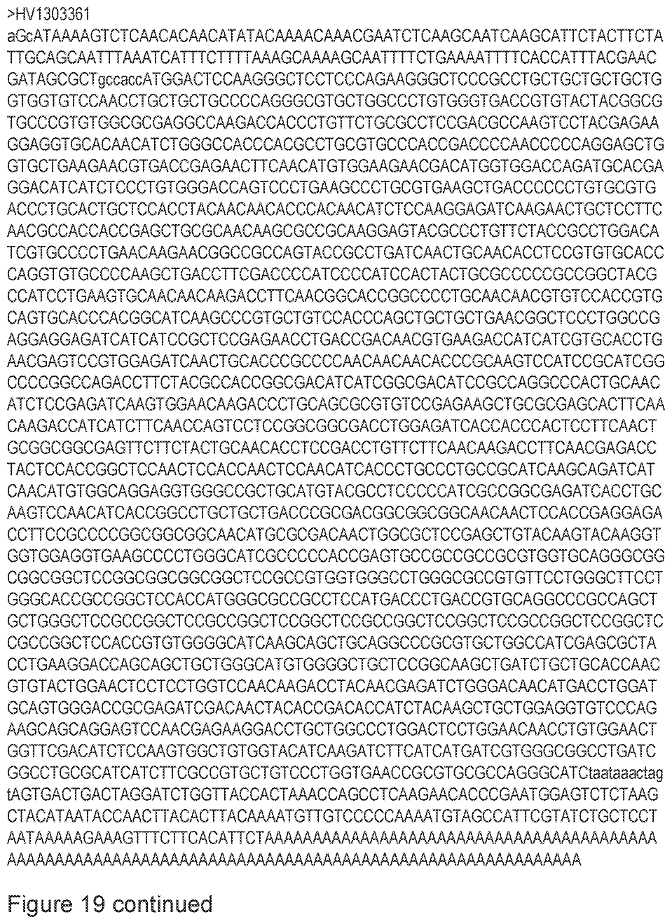

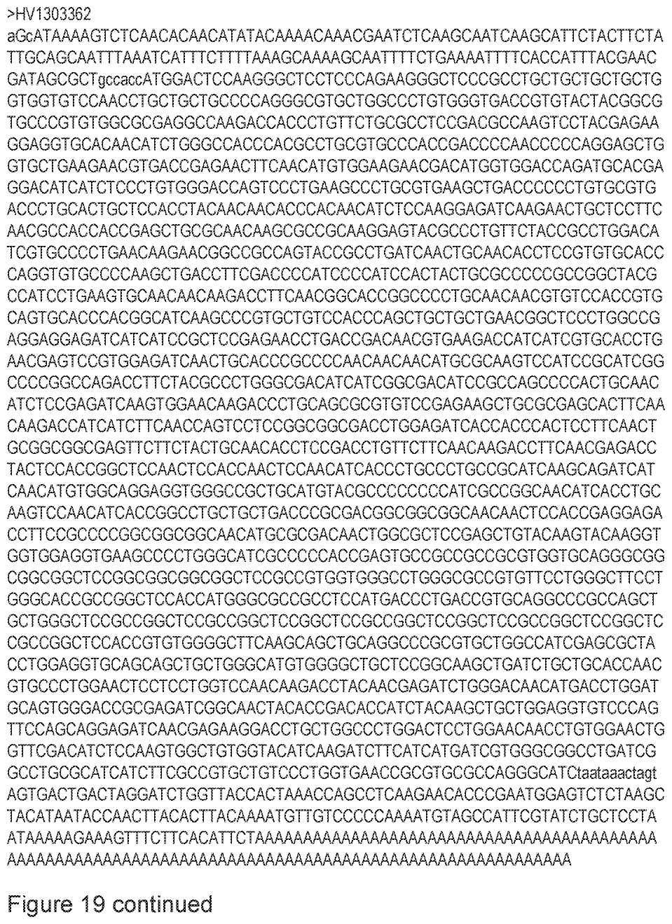

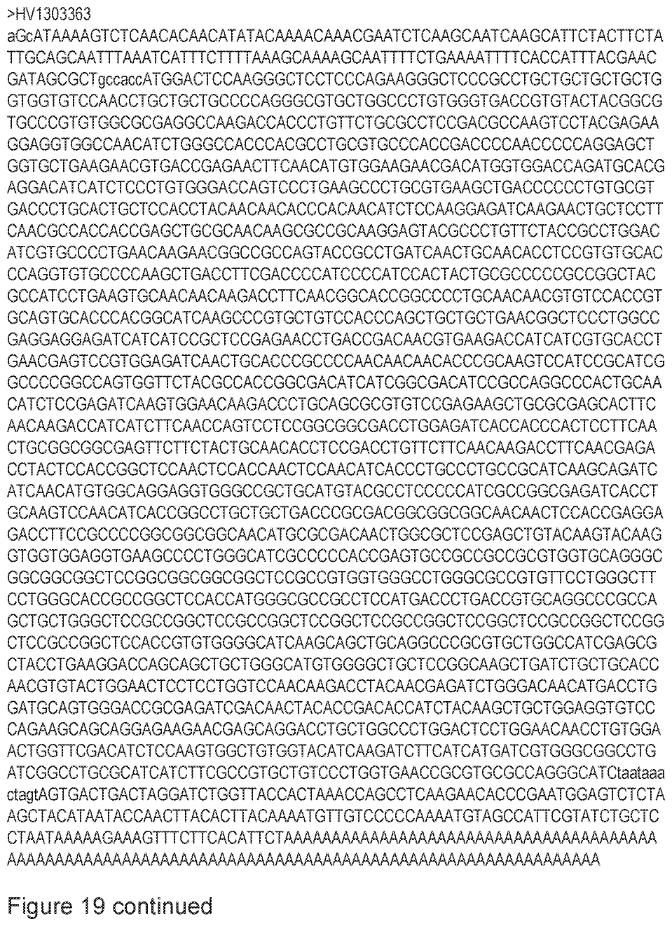

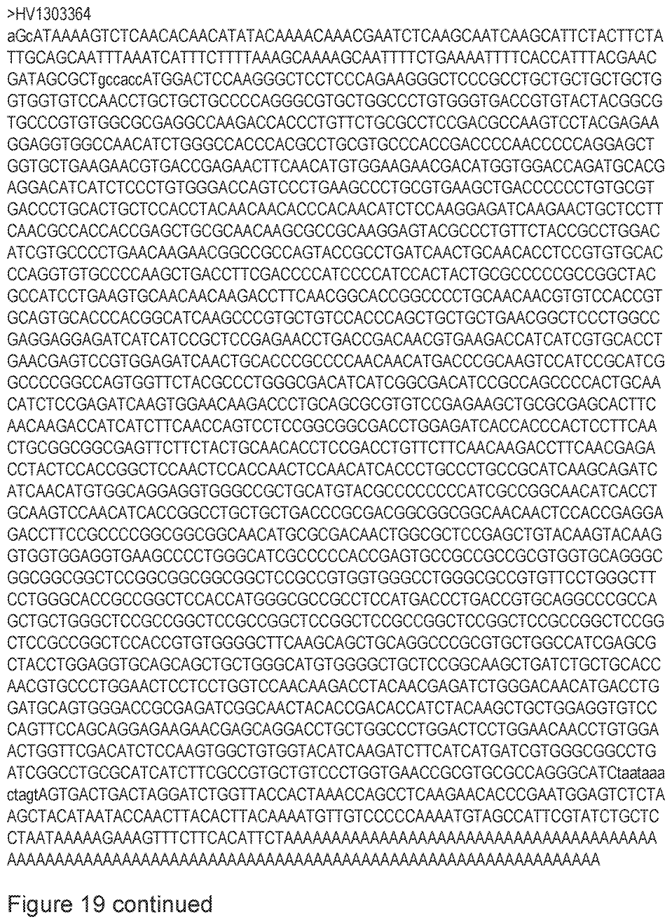

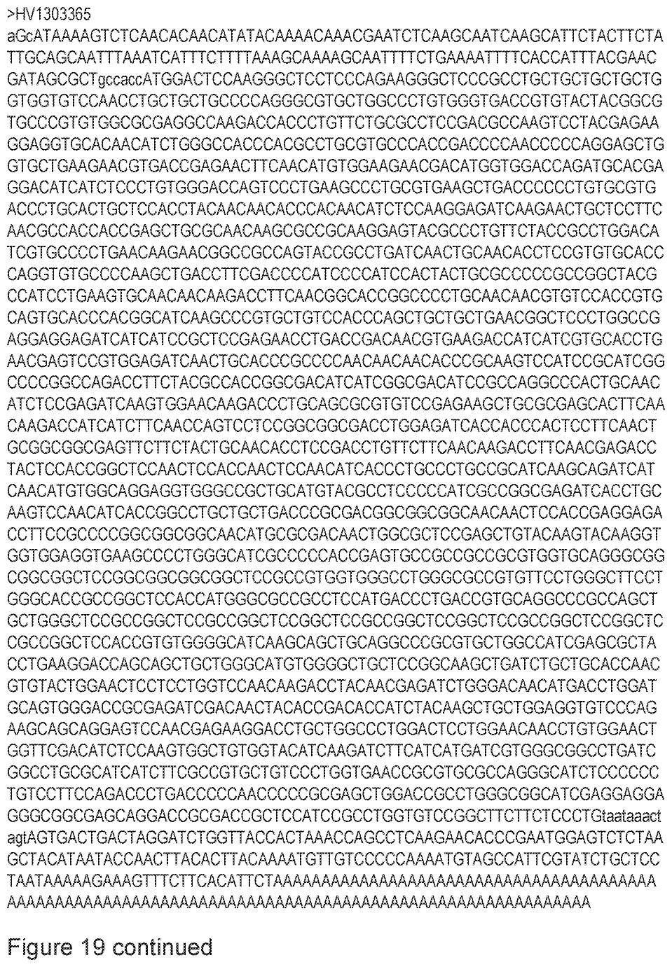

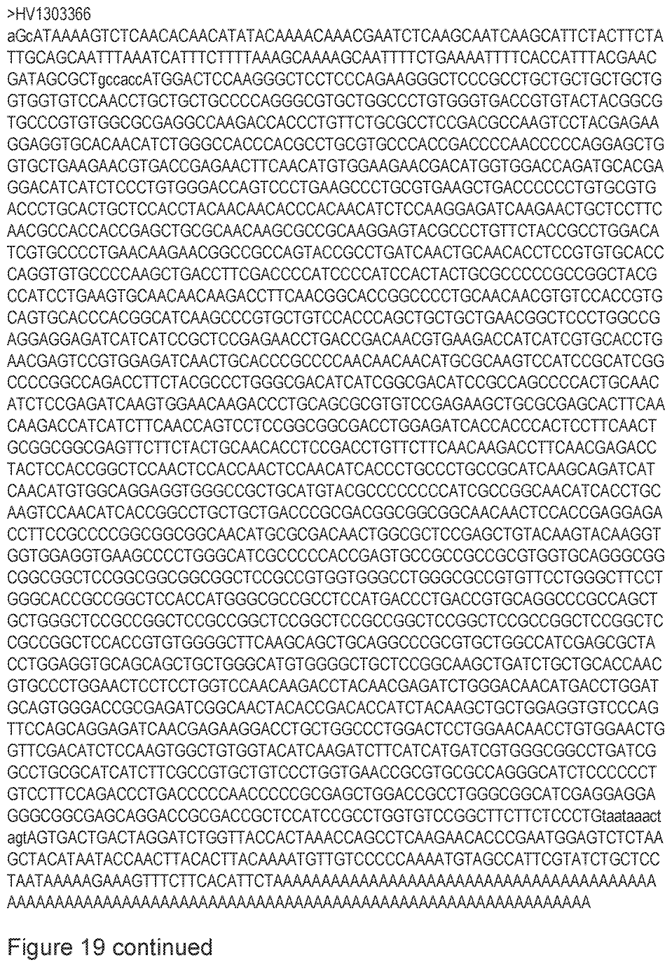

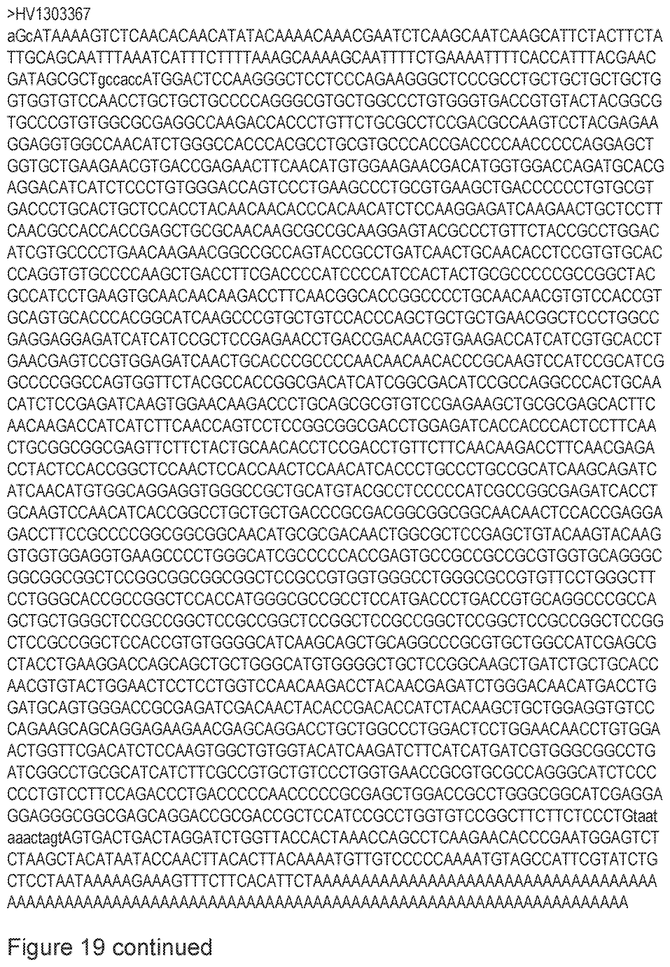

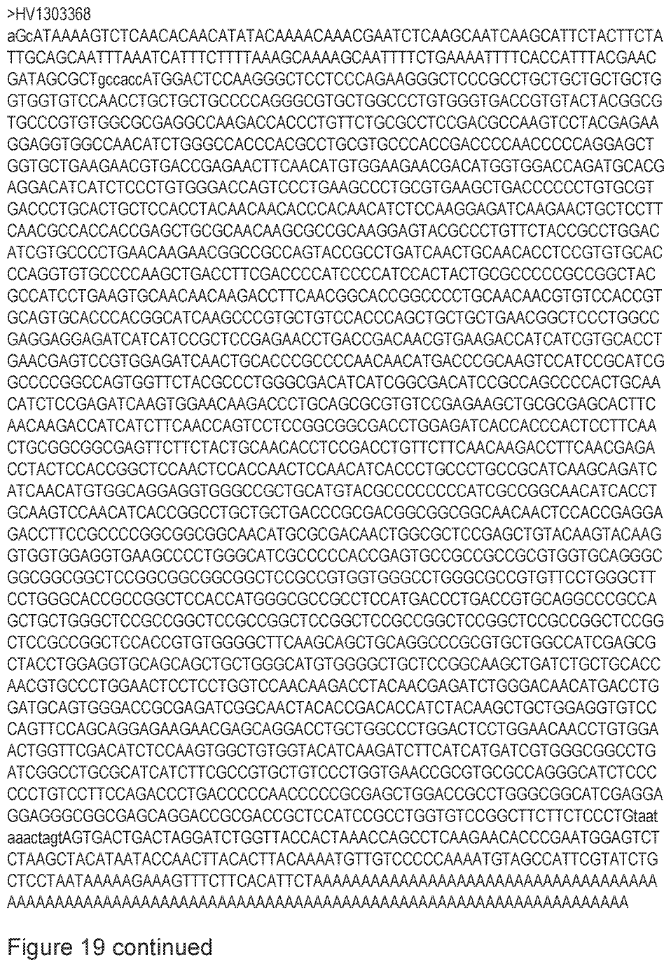

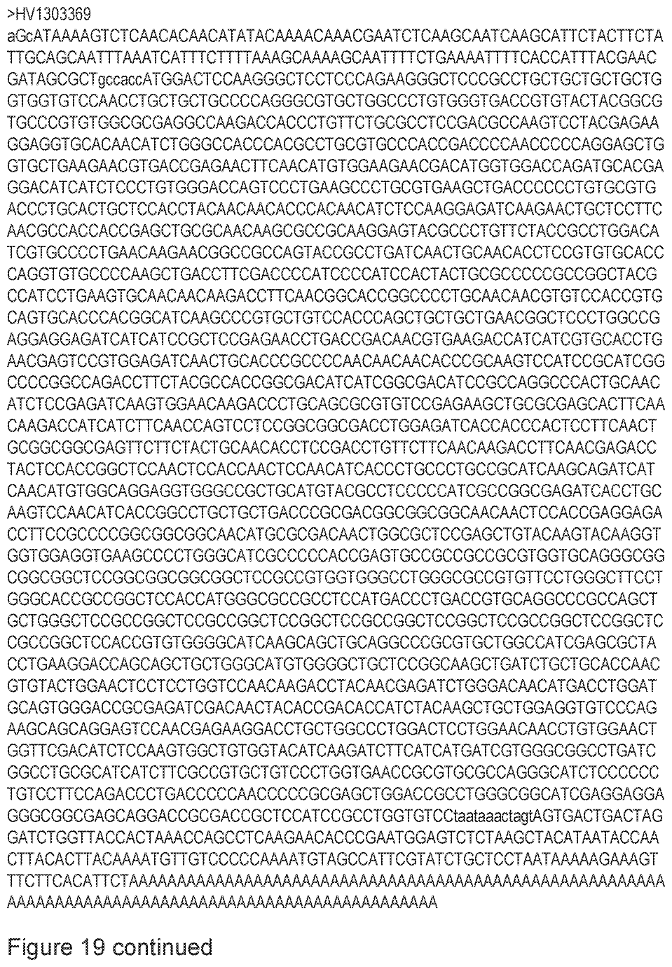

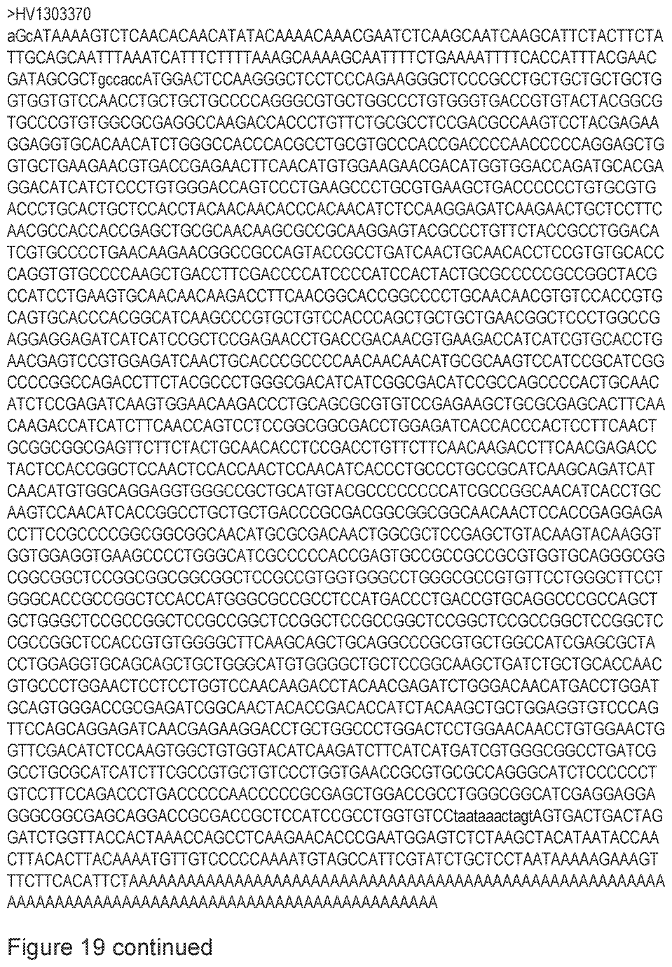

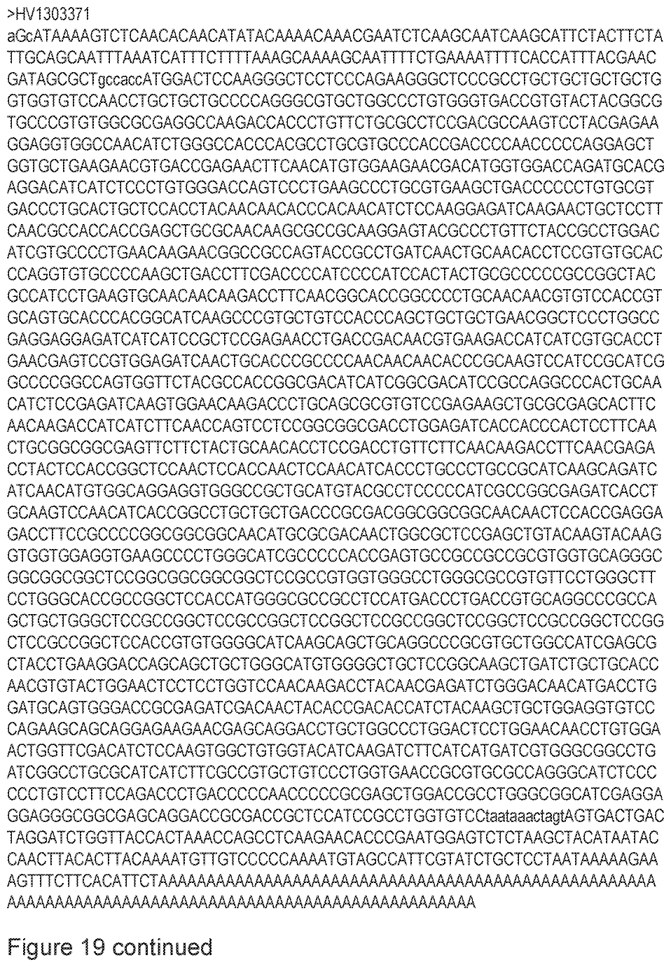

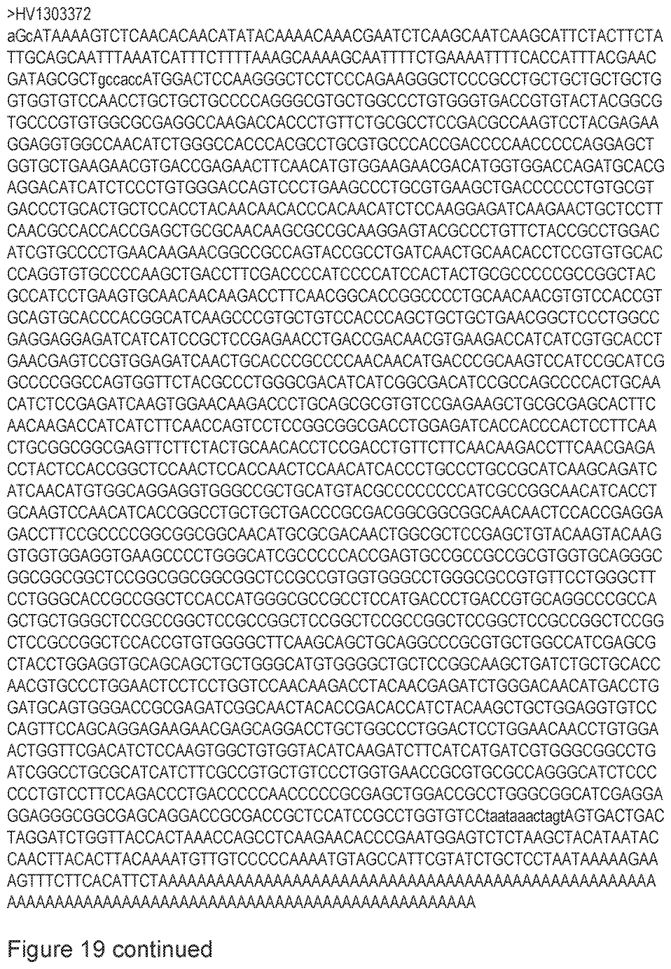

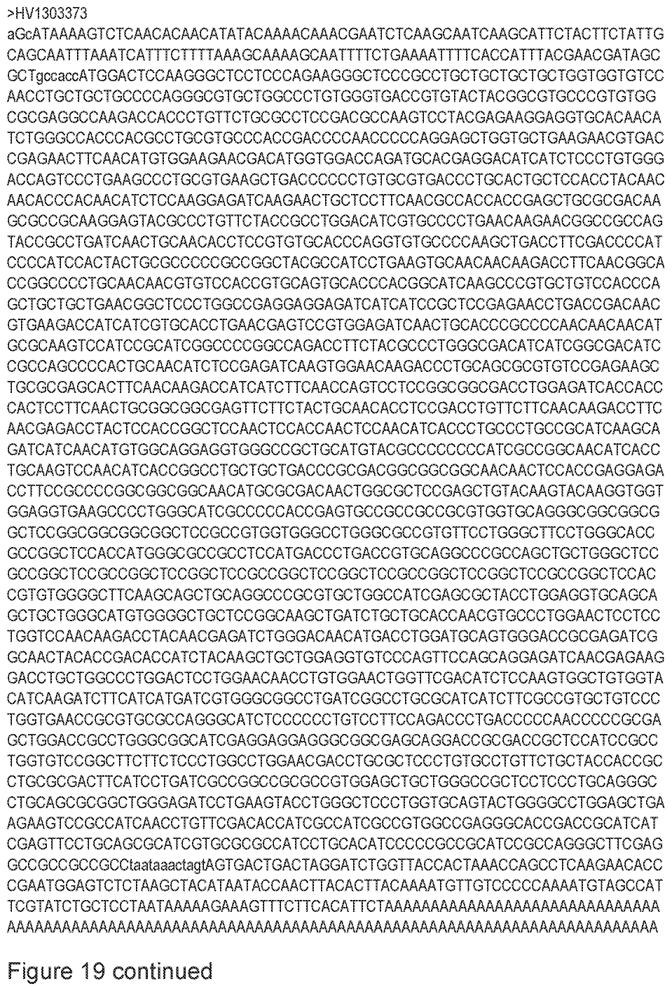

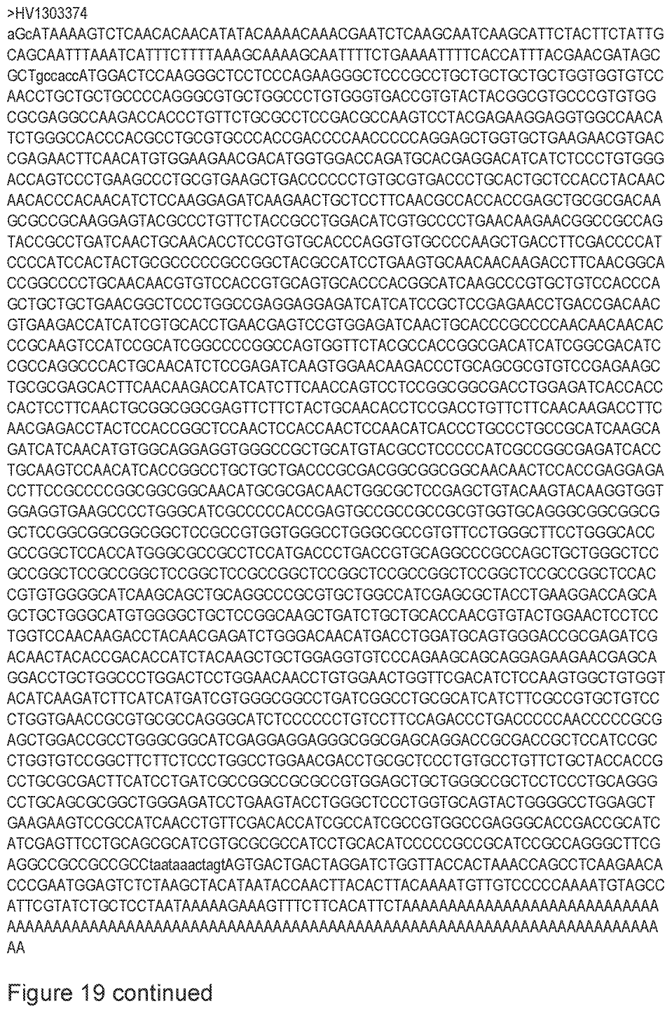

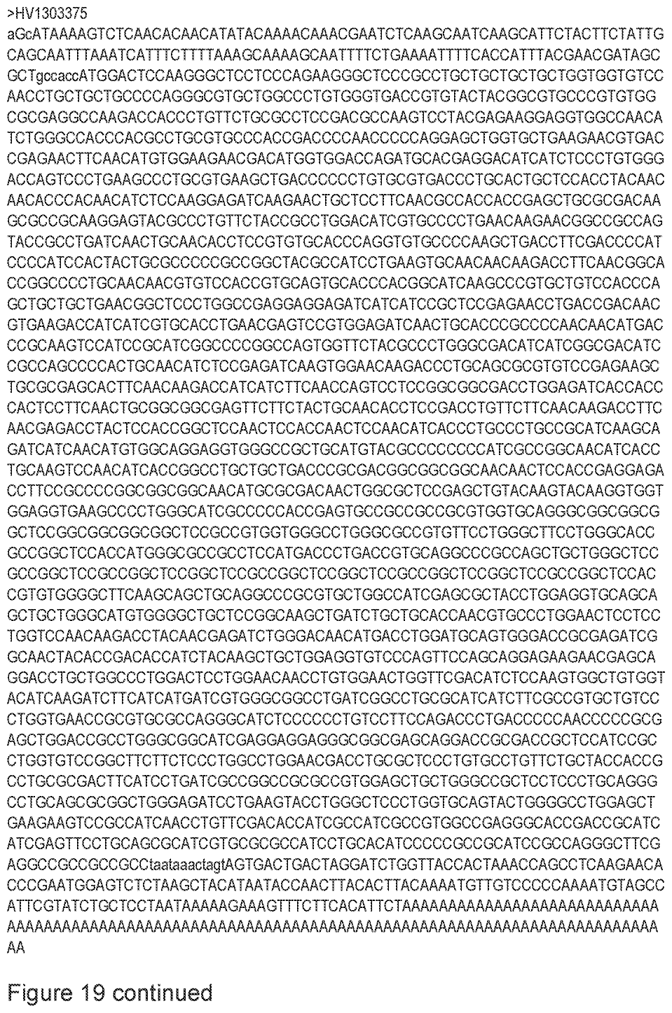

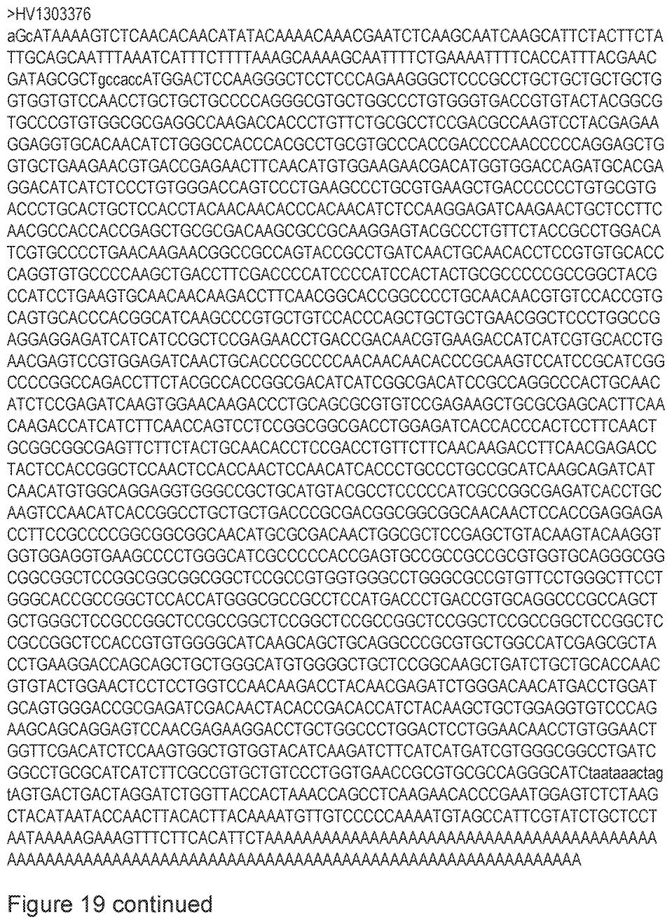

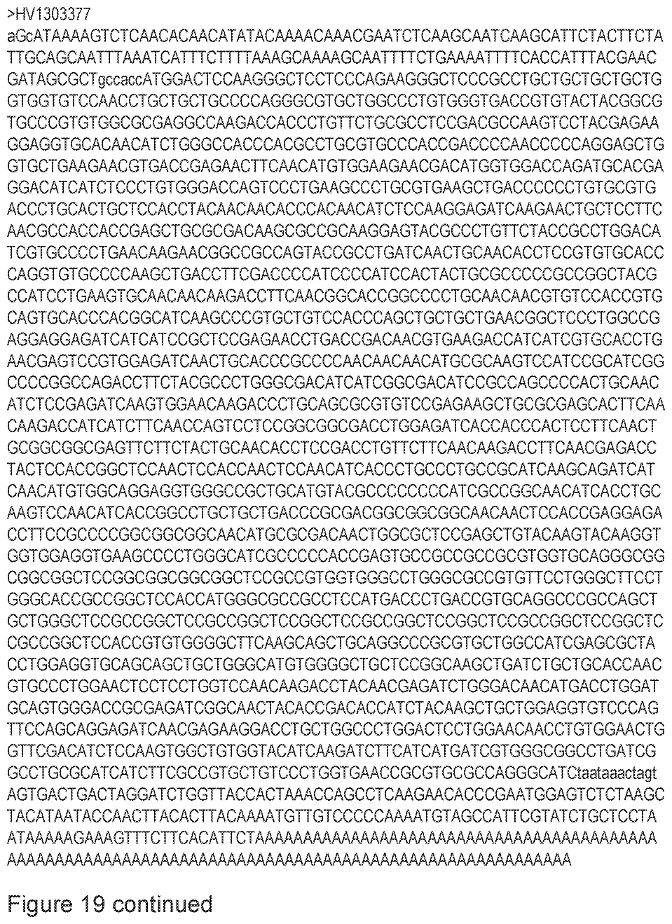

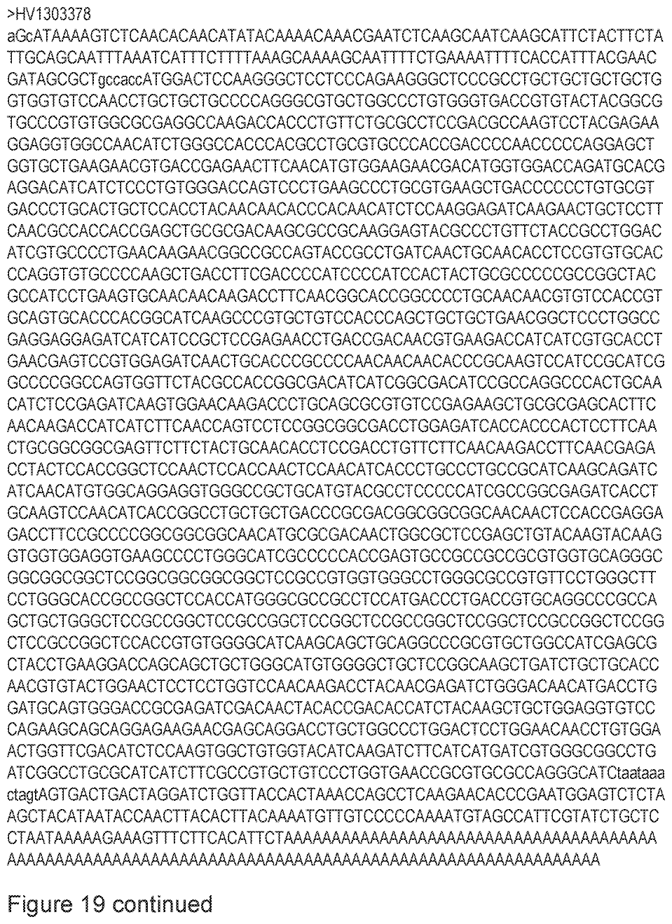

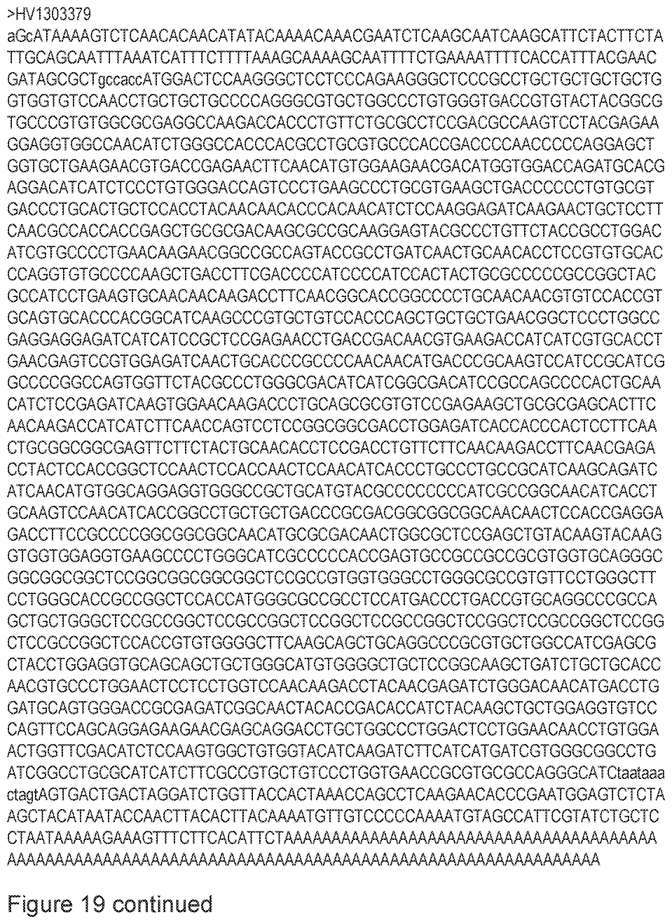

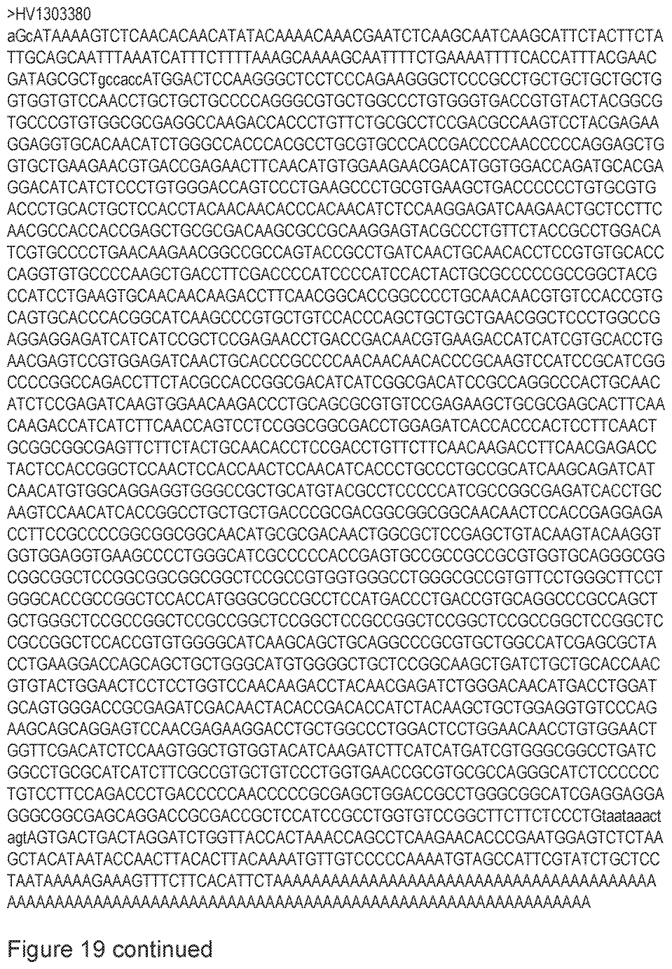

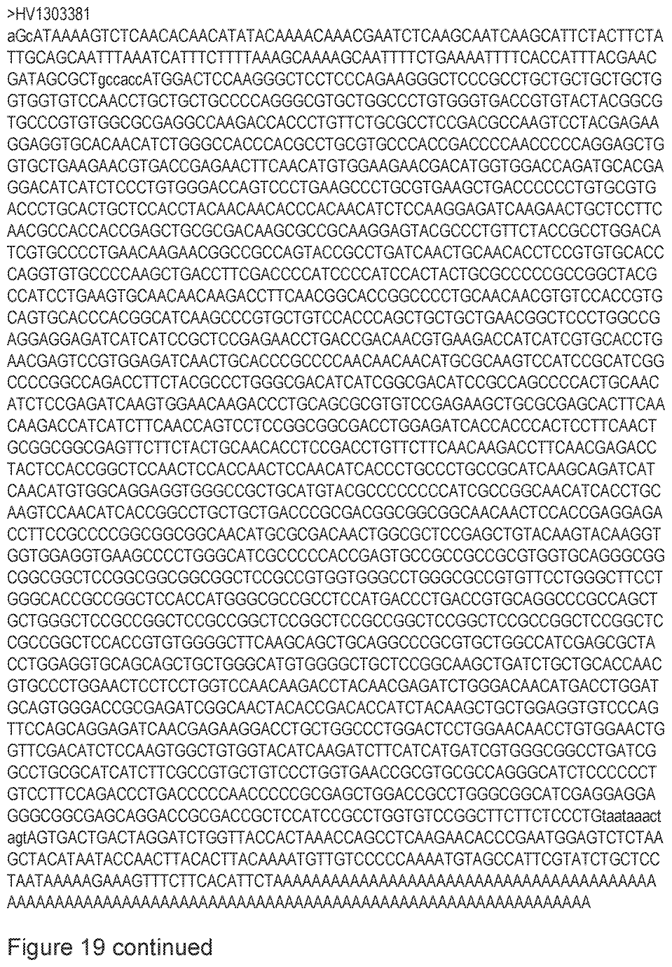

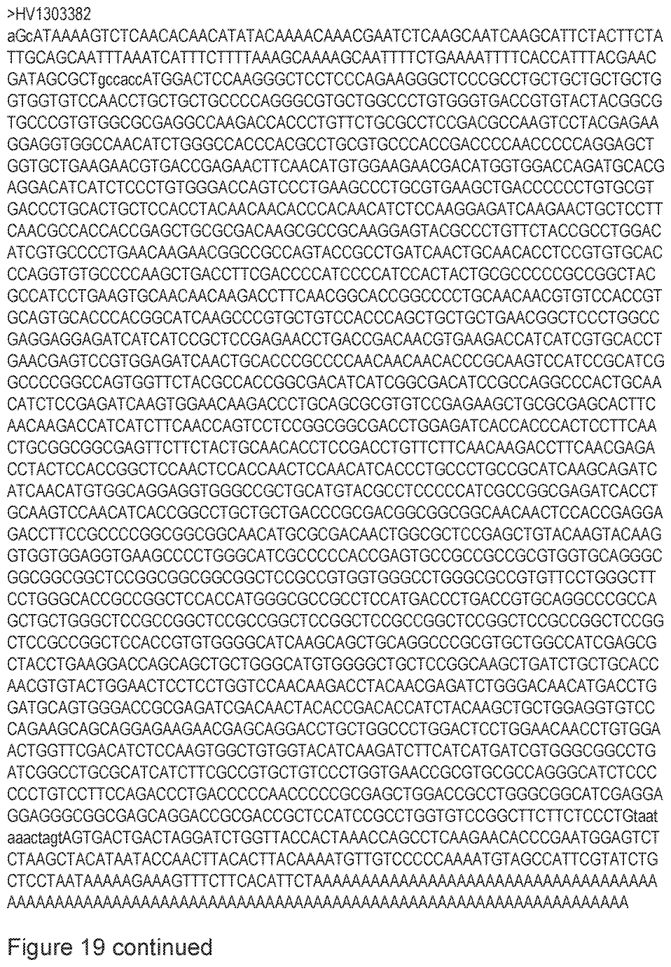

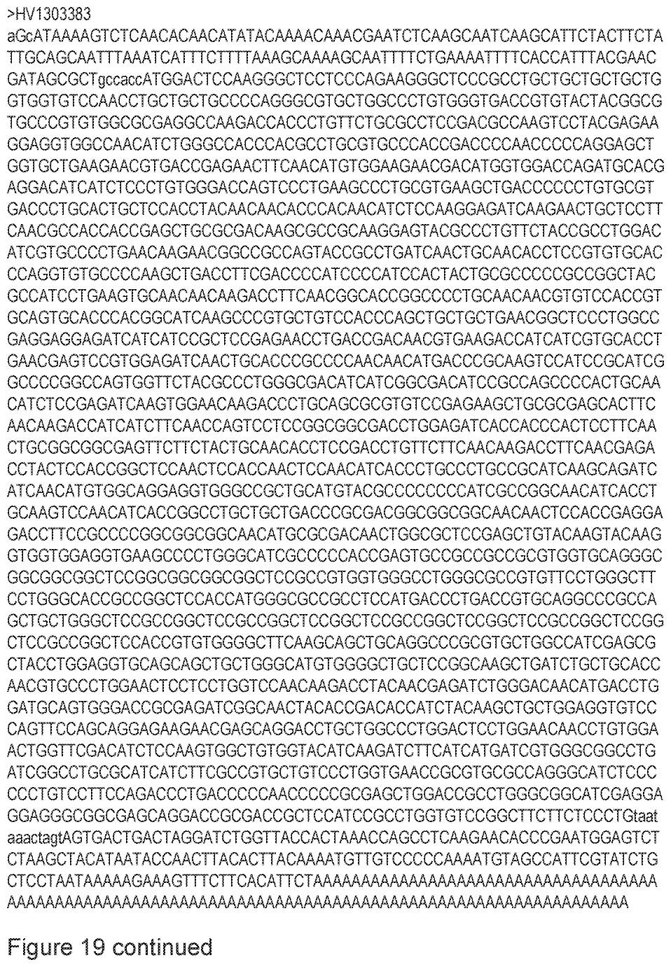

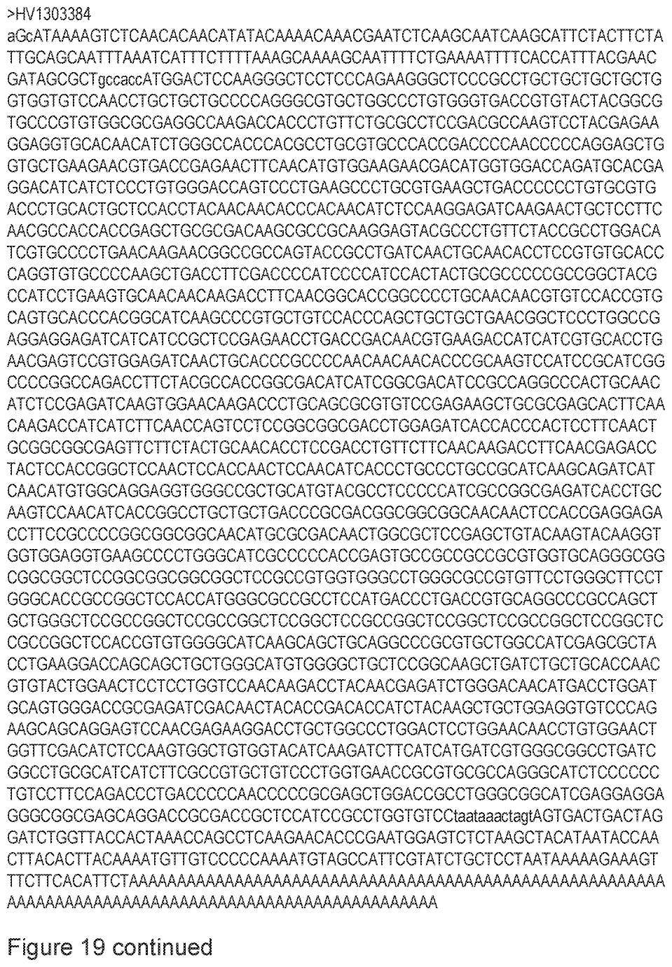

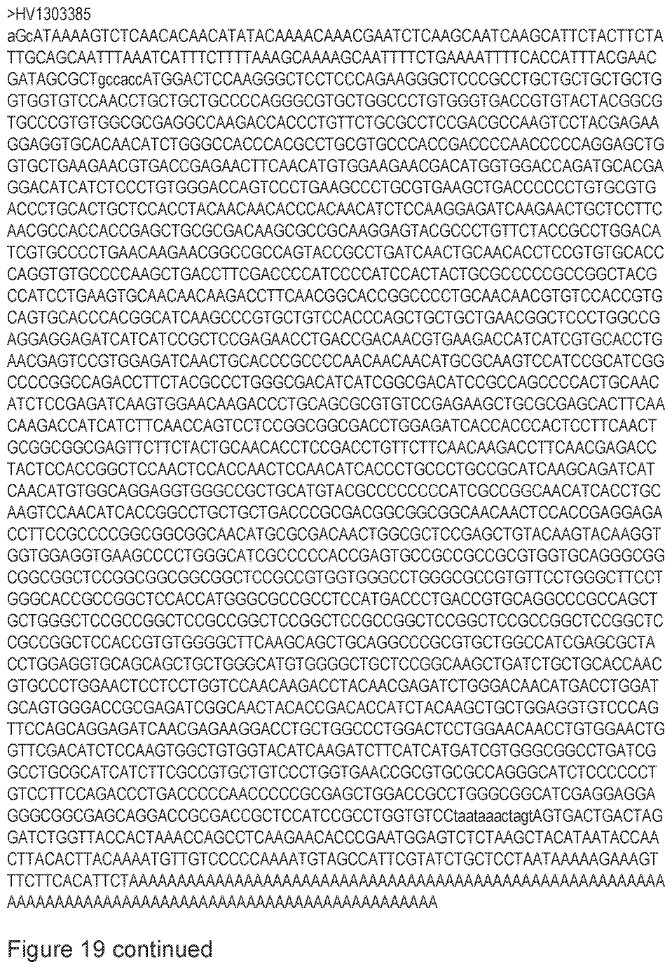

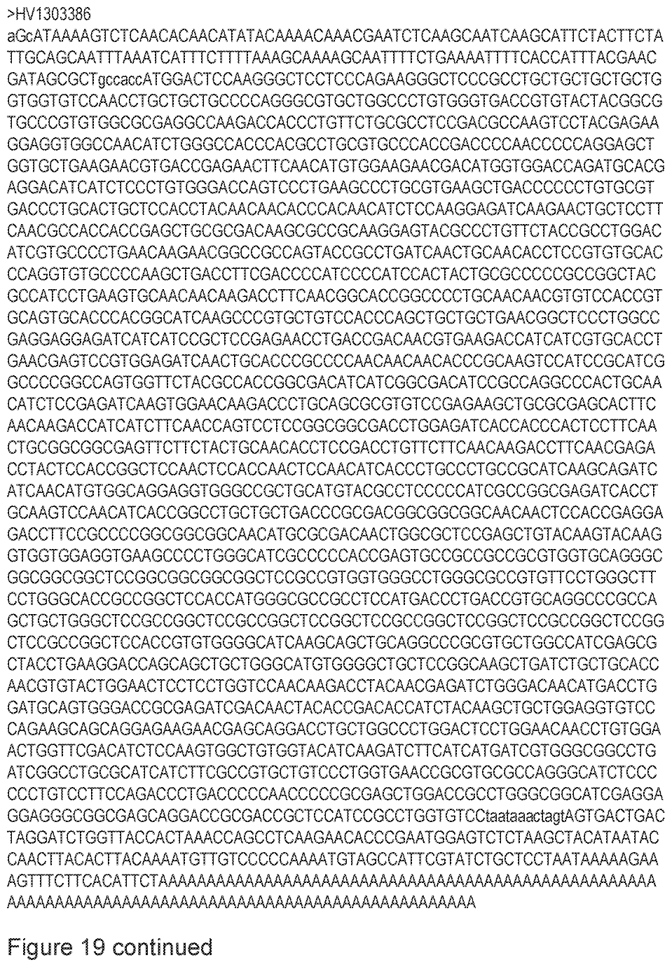

Nucleic acid sequences provided herein, e.g. see FIG. 19, are provided as DNA sequences. However, it should be understood that such sequences also represent RNA sequences, for example, mRNA. For example, RNA polymerase can be used to make RNA sequences from DNA sequences. In RNA sequences, thymine will be uridine. In some embodiments, uridine will be 1-methyl-pseudouridine. In some embodiments, nucleic acids of the invention, including RNA sequences or mRNAs, can further comprise any type of modified nucleotides, including, but not limited to 5-methyl-cytidine, 6-methyl-adenosine, or modified uridine.

Nucleic acid sequences provided herein, e.g. see FIG. 19, are provided with a poly A tail length of 101 nucleotides. However, it should be understood that mRNA sequences can comprise different lengths of poly A tail. For example, in some embodiments the poly A tail is about 85 to about 200 nucleotides long. For example, in some embodiments the poly A tail is 85 to 200 nucleotides long. In some embodiments the poly A tail is about 85 to about 110 nucleotides long. In some embodiments the poly A tail is 85 to 110 nucleotides long. In some embodiments the poly A tail is about 90 to about 110 nucleotides long. In some embodiments the poly A tail is 90 to 110 nucleotides long.

In certain aspects the invention provides nucleic acids encoding the inventive envelopes. In non-limiting embodiments, the nucleic acids are mRNA, modified or unmodified, suitable for any use, e.g. but not limited to use as pharmaceutical compositions. In certain embodiments, the nucleic acids are formulated in lipid, such as but not limited to LNPs.

In some embodiments the antibodies are administered as nucleic acids, including but not limited to mRNAs which could be modified and/or unmodified. See US Pub 20180028645A1, US Pub 20090286852, US Pub 20130111615, US Pub 20130197068, US Pub 20130261172, US Pub 20150038558, US Pub 20160032316, US Pub 20170043037, US Pub 20170327842, U.S. Pat. Nos. 10,006,007, 9,371,511, 9,012,219, US Pub 20180265848, US Pub 20170327842, US Pub 20180344838A1 at least at paragraphs [0260]-[0281], US Pub 20190153425 for non-limiting embodiments of chemical modifications, wherein each content is incorporated by reference in its entirety.

mRNAs delivered in LNP formulations have advantages over non-LNPs formulations. See US Pub 20180028645A1, US Pub 20190274968, US Pub 20180303925, wherein each content is incorporated by reference in its entirety.

In certain embodiments the nucleic acid encoding an envelope is operably linked to a promoter inserted an expression vector. In certain aspects the compositions comprise a suitable carrier. In certain aspects the compositions comprise a suitable adjuvant.

In certain aspects the invention provides an expression vector comprising any of the nucleic acid sequences of the invention, wherein the nucleic acid is operably linked to a promoter. In certain aspects the invention provides an expression vector comprising a nucleic acid sequence encoding any of the polypeptides of the invention, wherein the nucleic acid is operably linked to a promoter. In certain embodiments, the nucleic acids are codon optimized for expression in a mammalian cell, in vivo or in vitro. In certain aspects the invention provides nucleic acids comprising any one of the nucleic acid sequences of invention. In certain aspects the invention provides nucleic acids consisting essentially of any one of the nucleic acid sequences of invention. In certain aspects the invention provides nucleic acids consisting of any one of the nucleic acid sequences of invention. In certain embodiments the nucleic acid of the invention, is operably linked to a promoter and is inserted in an expression vector. In certain aspects the invention provides an immunogenic composition comprising the expression vector.

In certain aspects the invention provides a composition comprising at least one of the nucleic acid sequences of the invention. In certain aspects the invention provides a composition comprising any one of the nucleic acid sequences of invention. In certain aspects the invention provides a composition comprising at least one nucleic acid sequence encoding any one of the polypeptides of the invention.

In one embodiment, the nucleic acid is an RNA molecule. In one embodiment, the RNA molecule is transcribed from a DNA sequence described herein. In some embodiments, the RNA molecule is encoded by one of the inventive sequences. In another embodiment, the nucleotide sequence comprises an RNA sequence transcribed by a DNA sequence encoding the polypeptide sequence of the sequences of the invention, or a variant thereof or a fragment thereof. Accordingly, in one embodiment, the invention provides an RNA molecule encoding one or more of inventive antibodies. The RNA may be plus-stranded. Accordingly, in some embodiments, the RNA molecule can be translated by cells without needing any intervening replication steps such as reverse transcription.

In some embodiments, a RNA molecule of the invention may have a 5′ cap (e.g. but not limited to a 7-methylguanosine, 7mG(5′)ppp(5′)NImpNp, CleanCap® (e.g., the AG, GG, AU, 3′OMe AG, or 3′OMe GG CleanCap®), or ARCA). This cap can enhance in vivo translation of the RNA. The 5′ nucleotide of an RNA molecule useful with the invention may have a 5′ triphosphate group. In a capped RNA this may be linked to a 7-methylguanosine via a 5′-to-5′ bridge. A RNA molecule may have a 3′ poly-A tail. It may also include a poly-A polymerase recognition sequence (e.g. AAUAAA) near its 3′ end. In some embodiments, a RNA molecule useful with the invention may be single-stranded. In some embodiments, a RNA molecule useful with the invention may comprise synthetic RNA.

The recombinant nucleic acid sequence can be an optimized nucleic acid sequence. Such optimization can increase or alter the immunogenicity of the envelope. Optimization can also improve transcription and/or translation. Optimization can include one or more of the following: low GC content leader sequence to increase transcription; mRNA stability and codon optimization; addition of a Kozak sequence (e.g., GCC ACC) for increased translation; addition of an immunoglobulin (lg) leader sequence encoding a signal peptide; and eliminating to the extent possible cis-acting sequence motifs (i.e., internal TATA boxes).

Methods for in vitro transfection of mRNA and detection of envelope expression are known in the art.

Methods for expression and immunogenicity determination of nucleic acid encoded envelopes are known in the art.

In certain aspects the invention contemplates using immunogenic compositions wherein immunogens are delivered as recombinant proteins. Various methods for production and purification of recombinant proteins, including trimers such as but not limited to SOSIP based trimers, suitable for use in immunization are known in the art. In certain embodiments recombinant proteins are produced in CHO cells.

The immunogenic envelopes can also be administered as a protein boost in combination with a variety of nucleic acid envelope primes (e.g., HIV-1 Envs delivered as DNA expressed in viral or bacterial vectors)

Dosing of proteins and nucleic acids can be readily determined by a skilled artisan. A single dose of nucleic acid can range from a few nanograms (ng) to a few micrograms (μg) or milligram of a single immunogenic nucleic acid. Recombinant protein dose can range from a few ug micrograms to a few hundred micrograms, or milligrams of a single immunogenic polypeptide.

Administration: The compositions can be formulated with appropriate carriers using known techniques to yield compositions suitable for various routes of administration. In certain embodiments the compositions are delivered via intramuscular (IM), via subcutaneous, via intravenous, via nasal, via mucosal routes, or any other suitable route of immunization.

The compositions can be formulated with appropriate carriers and adjuvants using techniques to yield compositions suitable for immunization. The compositions can include an adjuvant, such as, for example but not limited to, alum, 3M052, poly IC, MF-59 or other squalene-based adjuvant, ASOIB, or other liposomal based adjuvant suitable for protein or nucleic acid immunization. In certain embodiments, the adjuvant is GSK AS01E adjuvant containing MPL and QS21. This adjuvant has been shown by GSK to be as potent as the similar adjuvant ASOIB but to be less reactogenic using HBsAg as vaccine antigen [Leroux-Roels et al., IABS Conference, April 2013]. In certain embodiments, TLR agonists are used as adjuvants. In other embodiment, adjuvants which break immune tolerance are included in the immunogenic compositions.

In certain embodiments, the compositions and methods comprise any suitable agent or immune modulation which could modulate mechanisms of host immune tolerance and release of the induced antibodies. In non-limiting embodiments modulation includes PD-1 blockade; T regulatory cell depletion; CD40L hyperstimulation; soluble antigen administration, wherein the soluble antigen is designed such that the soluble agent eliminates B cells targeting dominant epitopes, or a combination thereof. In certain embodiments, an immunomodulatory agent is administered in at time and in an amount sufficient for transient modulation of the subject's immune response so as to induce an immune response which comprises broad neutralizing antibodies against HIV-1 envelope. Non-limiting examples of such agents is any one of the agents described herein: e.g. chloroquine (CQ), PTPIB Inhibitor-CAS 765317-72-4-Calbiochem or MSI 1436 clodronate or any other bisphosphonate; a Foxol inhibitor, e.g. 344355 | Foxol Inhibitor, AS1842856-Calbiochem; Gleevac, anti-CD25 antibody, anti-CCR4 Ab, an agent which binds to a B cell receptor for a dominant HIV-1 envelope epitope, or any combination thereof. In non-limiting embodiments, the modulation includes administering an anti-CTLA4 antibody. Non-limiting examples are ipilimumab and tremelimumab. In certain embodiments, the methods comprise administering a second immunomodulatory agent, wherein the second and first immunomodulatory agents are different

There are various host mechanisms that control bnAbs. For example, highly somatically mutated antibodies become autoreactive and/or less fit (Immunity 8:751, 1998; PloS Comp. Biol. 6 e1000800, 2010; J. Thoret. Biol. 164:37, 1993); Polyreactive/autorcactive naïve B cell receptors (unmutated common ancestors of clonal lineages) can lead to deletion of Ab precursors (Nature 373:252, 1995; PNAS 107:181, 2010; J. Immunol. 187:3785, 2011); Abs with long HCDR3 can be limited by tolerance deletion (JI 162:6060, 1999; JCI 108:879, 2001). BnAb knock-in mouse models are providing insights into the various mechanisms of tolerance control of MPER BnAb induction (deletion, anergy, receptor editing). Other variations of tolerance control likely will be operative in limiting BnAbs with long HCDR3s, high levels of somatic hypermutations.

For a summary of CH505 sequences and designs see U.S. Pat. No. 10,968,255, e.g. but not limited to Table 1, FIGS. 22-24, and U.S. Pat. No. 10,004,800 (FIG. 17).

It is readily understood that the envelope glycoproteins referenced in various examples and figures comprise a signal/leader sequence. It is well known in the art that HIV-I envelope glycoprotein is a secretory protein with a signal or leader peptide sequence that is removed during processing and recombinant expression (without removal of the signal peptide, the protein is not secreted). See for example Li et al. Control of expression, glycosylation, and secretion of HIV-1 gp120 by homologous and heterologous signal sequences. Virology 204 (1): 266-78 (1994) (“Li et al. 1994″), at first paragraph, and Li et al. Effects of inefficient cleavage of the signal sequence of HIV-1 gp120 on its association with calnexin, folding, and intracellular transport. PNAS 93:9606-9611 (1996) (“Li et al. 1996”), at 9609, Any suitable signal sequence could be used. In some embodiments the leader sequence is the endogenous leader sequence. Most of the gp120 and gp160 amino acid sequences include the endogenous leader sequence. In other non-limiting examples, the leader sequence is human Tissue Plasminogen Activator (TPA) sequence, human CD5 leader sequence (e.g. MPMGSLQPLATLYLLGMLVASVLA). Most of the chimeric designs include CD5 leader sequence. A skilled artisan appreciates that when used as immunogens, and for example when recombinantly produced, the amino acid sequences of these proteins do not comprise the leader peptide sequences.

### HIV-1 Envelope Trimers and Other Envelope Designs

This example shows that stabilized HIV-1 Env trimer immunogens show enhanced antigenicity for broadly neutralizing antibodies and are not recognized by non-neutralizing antibodies. The example also describes additional envelope modifications and designs. In some embodiments these envelopes, including but not limited to trimers are further multimerized, and/or used as particulate, high-density array in liposomes or other particles, for example but not limited to nanoparticles. Any one of the envelopes of the invention could be designed and expressed as described herein.

A stabilized chimeric SOSIP designs were used to generate CH505 trimers. This design was applicable to diverse viruses from multiple clades.

Elicitation of neutralizing antibodies is one goal for antibody-based vaccines. Neutralizing antibodies target the native trimeric HIV-1 Env on the surface virions. The trimeric HIV-1 envelope protein consists of three protomers each containing a gp120 and gp41 heterodimer. Recent immunogen design efforts have generated soluble near-native mimics of the Env trimer that bind to neutralizing antibodies but not non-neutralizing antibodies. The recapitulation of the native trimer could be a key component of vaccine induction of neutralizing antibodies. Neutralizing Abs target the native trimeric HIV-1 Env on the surface of viruses (Poignard et al. J Virol. 2003 January; 77 (1): 353-65; Parren et al. J Virol. 1998 December; 72 (12): 10270-4.; Yang et al. J Virol. 2006 November; 80 (22): 11404-8.). The HIV-1 Env protein consists of three protomers of gp120 and gp41 heterodimers that are noncovalently linked together (Center et al. J Virol. 2002 August; 76 (15): 7863-7.). Soluble near-native trimers preferentially bind neutralizing antibodies as opposed to non-neutralizing antibodies (Sanders et al. PLoS Pathog. 2013 September; 9 (9): e1003618).

Sequential Env vaccination has elicited broad neutralization in the plasma of one macaque. The overall goal of our project is to increase the frequency of vaccine induction of bnabs in the plasma of primates with Env vaccination. We hypothesized that vaccination with immunogens that target bnAb B cell lineage and mimic native trimers will increase the frequency of broadly neutralizing plasma antibodies. One goal is increasing the frequency of vaccine induction of bnAb in the plasma of primates by Env vaccination. It is expected that vaccination with immunogens that target bnAb B cell lineages and mimic the native trimers on virions will increase the frequency of broadly neutralizing plasma antibodies.

Previous work has shown that CH505 derived soluble trimers are hard to produce. From a study published by Julien et al in 2015 (Proc Natl Acad Sci USA. 2015 Sep. 22; 112 (38): 11947-11952.) it was shown that while CH505 produced comparable amounts of protein by transient transfection, only 5% of the CH505 protein formed trimer which 5 times lower than the gold standard viral strain BG505. Provided here are non-limiting embodiments of well-folded trimers for Env immunizations.

Near-native soluble trimers using the 6R.SOSIP.664 design are capable of generating autologous tier 2 neutralizing plasma antibodies in the plasma (Sanders et al. 2015), which provides a starting point for designing immunogens to elicit broadly neutralizing antibodies. While these trimers are preferentially antigenic for neutralizing antibodies, they still possess the ability to expose the V3 loop, which generally results in strain-specific binding and neutralizing antibodies after vaccination. Using the unliganded structure the BG505.6R.SOSIP.664 has been stabilized by adding cysteines at position 201 and 433 to constrain the conformational flexibility such that the V3 loop is maintained unexposed (Kwon et al. Nat Struct Mol Biol. 2015 July; 22 (7): 522-531.).

Provided are engineered trimeric immunogens derived from multiple viruses from CH505. We generated chimeric 6R.SOSIP.664, chimeric disulfide stabilized (DS) 6R.SOSIP.664 (Kwon et al Nat Struct Mol Biol. 2015 July; 22 (7): 522-531.), chimeric 6R.SOSIP.664v4.1 (DeTaeye et al. Cell. 2015 Dec. 17; 163 (7): 1702-15. doi: 10.1016/j.cell.2015.11.056), and chimeric 6R.SOSIP.664v4.2 (DeTaeye et al. Cell. 2015 Dec. 17; 163 (7): 1702-15. doi: 10.1016/j.cell.2015.11.056). The 6R.SOSIP.664 is the basis for all of these designs and is made as a chimera of C.CH0505 and A.BG505. The gp120 of C.CH505 was fused with the BG505 inner domain gp120 sequence within the alpha helix 5 (α5) to result in the chimeric protein. The chimeric gp120 is disulfide linked to the A.BG505 gp41 as outlined by Sanders et al. (PLoS Pathog. 2013 September; 9 (9): e1003618). These immunogens were designed as chimeric proteins that possess the BG505 gp41 connected to the CH505 gp120, since the BG505 strain is particularly adept at forming well-folded, closed trimers. This envelope design retains the CH505 CD4 binding site that is targeted by the CH103 and CH235 broadly neutralizing antibody lineages that were isolated from CH505.

Based on the various designs, any other suitable envelope, for example but not limited to CH505 envelopes as described in U.S. Pat. No. 10,004,800, incorporated herein by reference, can be designed. Other suitable envelopes include, but are not limited to, CAP256SU, CAP256wk34.80, CAM13, Q23, an T250 envelopes.

Recombinant envelopes as trimers could be produced and purified by any suitable method. For a non-limiting example of purification methods see Ringe R P, Yasmeen A, Ozorowski G, Go E P, Pritchard L K, Guttman M, Ketas T A, Cottrell C A, Wilson I A, Sanders R W, Cupo A, Crispin M, Lee K K, Desaire H, Ward A B, Klasse P J, Moore J P. 2015. Influences on the design and purification of soluble, recombinant native-like HIV-1 envelope glycoprotein trimers. J Virol 89:12189-12210. doi: 10.1128/JVI.01768-15.

### Multimeric Envelopes

Presentation of antigens as particulates reduces the B cell receptor affinity necessary for signal transduction and expansion (See Baptista et al. EMBO J. 2000 Feb. 15; 19 (4): 513-520). Displaying multiple copies of the antigen on a particle provides an avidity effect that can overcome the low affinity between the antigen and B cell receptor. The initial B cell receptor specific for pathogens can be low affinity, which precludes vaccines from being able to stimulate and expand B cells of interest. In particular, very few naïve B cells from which HIV-1 broadly neutralizing antibodies arise can bind to soluble HIV-1 Envelope. Provided are envelopes, including but not limited to trimers as particulate, high-density array on liposomes or other particles, for example but not limited to nanoparticles. See e.g. He et al. Nature Communications 7, Article number: 12041 (2016), doi: 10.1038/ncomms12041; Bamrungsap et al. Nanomedicine, 2012, 7 (8), 1253-1271.

To improve the interaction between the naïve B cell receptor and immunogens, envelope designed can be created to wherein the envelope is presented on particles, e.g. but not limited to nanoparticle. In some embodiments, the HIV-1 Envelope trimer could be fused to ferritin. Ferritin protein self assembles into a small nanoparticle with three fold axis of symmetry. At these axes the envelope protein is fused. Therefore, the assembly of the three-fold axis also clusters three HIV-1 envelope protomers together to form an envelope trimer. Each ferritin particle has 8 axes which equates to 8 trimers being displayed per particle. See e.g. Sliepen et al. Retrovirology201512: 82, DOI: 10.1186/s12977-015-0210-4.

Any suitable ferritin sequence could be used. In non-limiting embodiments, ferritin sequences are disclosed in U.S. Pat. No. 10,961,283, incorporated herein by reference.

Ferritin nanoparticle linkers: The ability to form HIV-1 envelope ferritin nanoparticles relies self-assembly of 24 ferritin subunits into a single ferritin nanoparticle. The addition of a ferritin subunit to the C-terminus of HIV-1 envelope may interfere with the ability of the ferritin subunit to fold properly and or associate with other ferritin subunits. When expressed alone ferritin readily forms 24-subunit nanoparticles, however appending it to envelope only yields nanoparticles for certain envelopes. Since the ferritin nanoparticle forms in the absence of envelope, the envelope could be sterically bindering the association of ferritin subunits. Thus, we designed ferritin with elongated glycine-serine linkers to further distance the envelope from the ferritin subunit. To make sure that the glycine linker is attached to ferritin at the correct position, we created constructs that attach at second amino acid position or the fifth amino acid position. The first four n-terminal amino acids of natural Helicobacter pylori ferritin are not needed for nanoparticle formation but may be critical for proper folding and oligomerization when appended to envelope. Thus, we designed constructs with and without the Leucine, serine, and lysine amino acids following the glycine-serine linker. The goal will be to find a linker length that is suitable for formation of envelope nanoparticles when ferritin is appended to most envelopes. Any suitable linker between the envelope and ferritin could be uses, so long as the fusion protein is expressed and the trimer is formed.

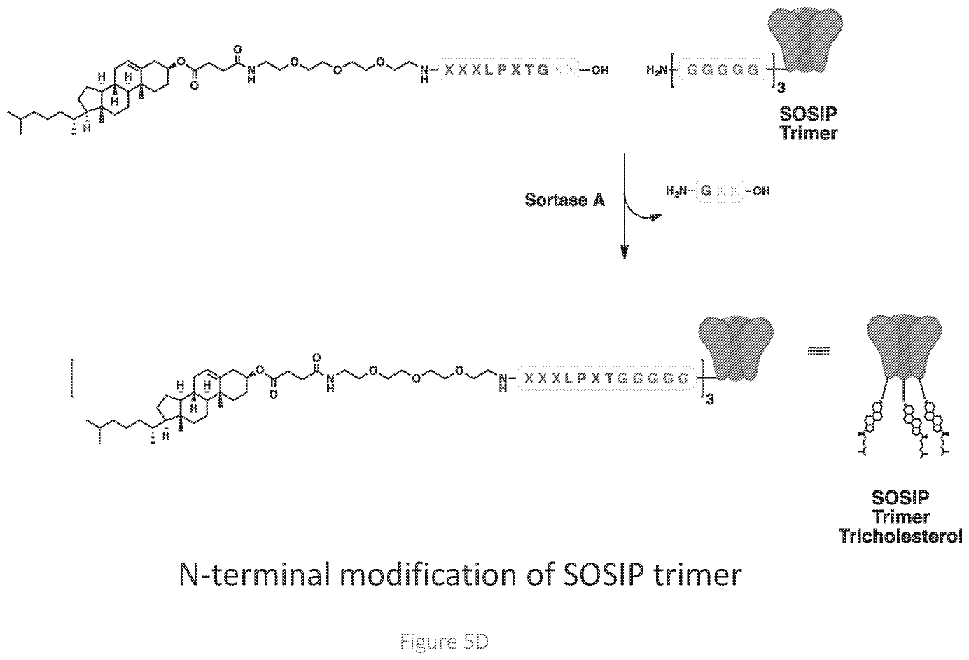

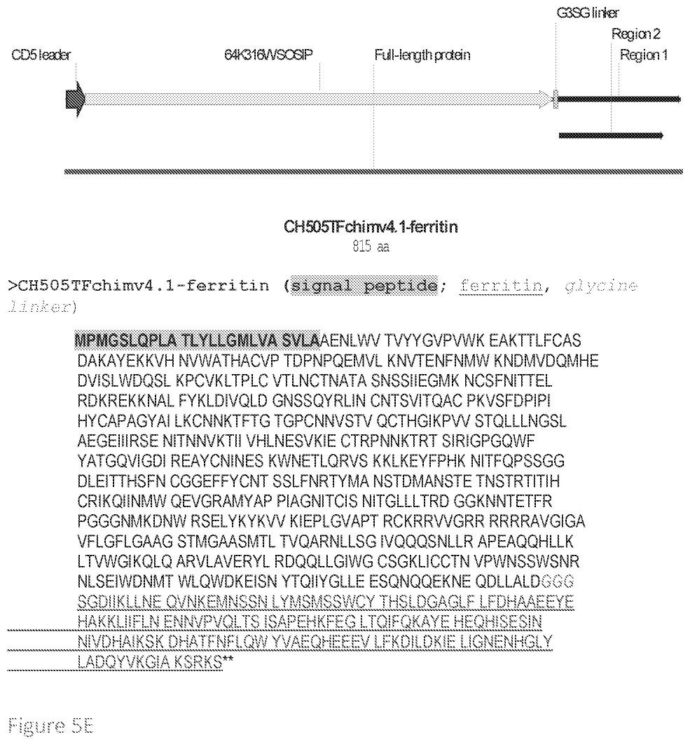

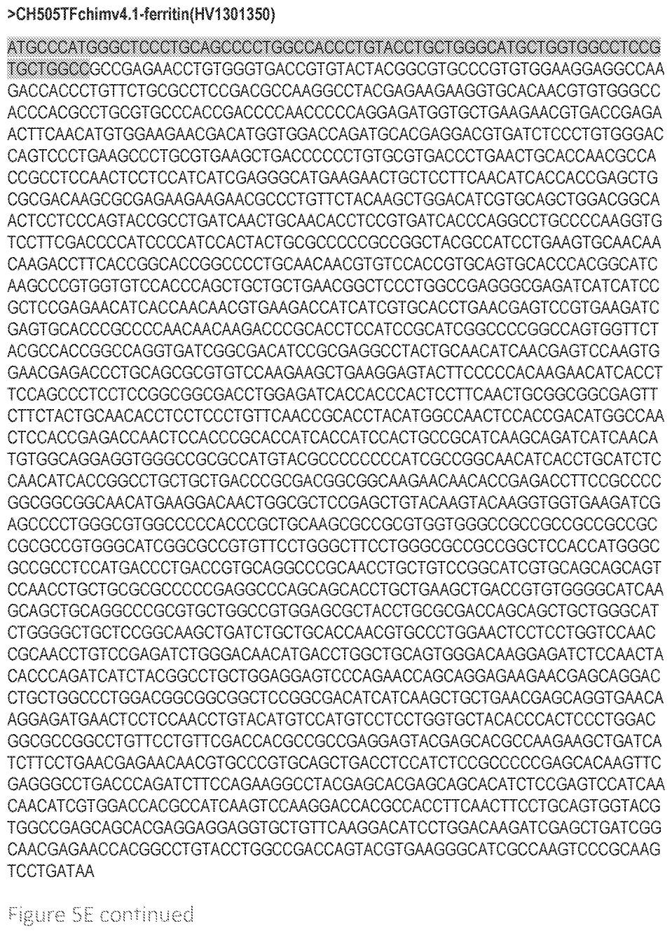

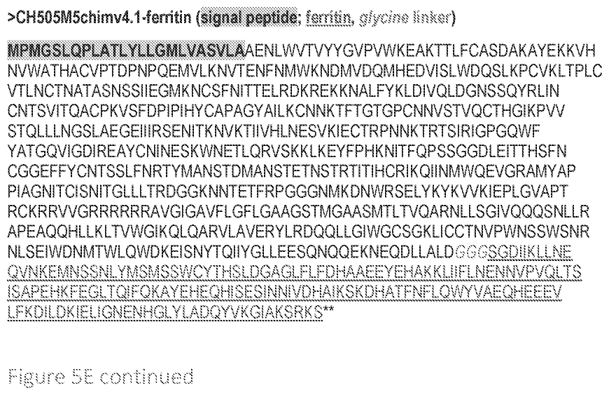

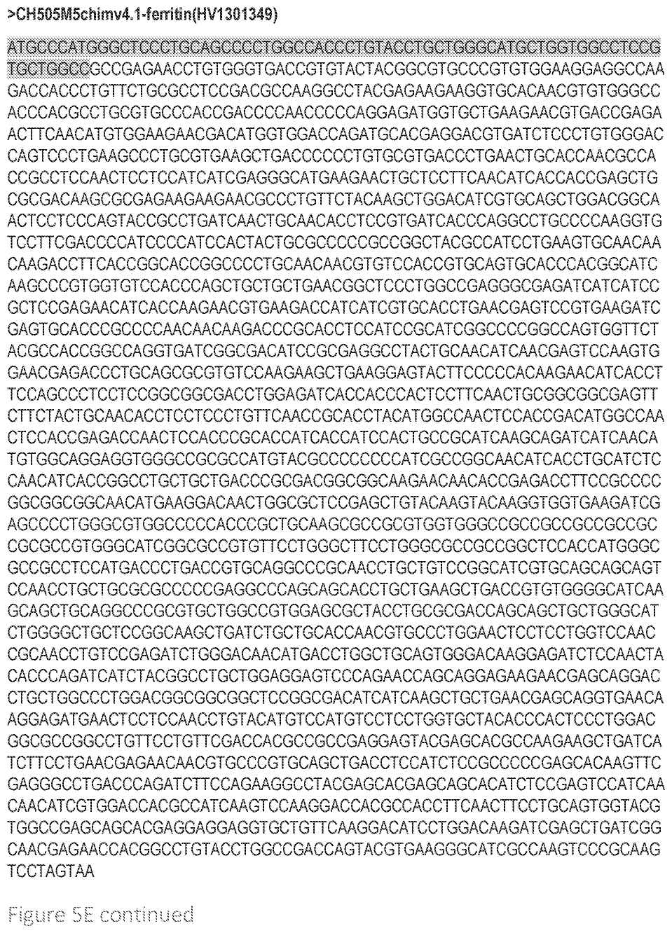

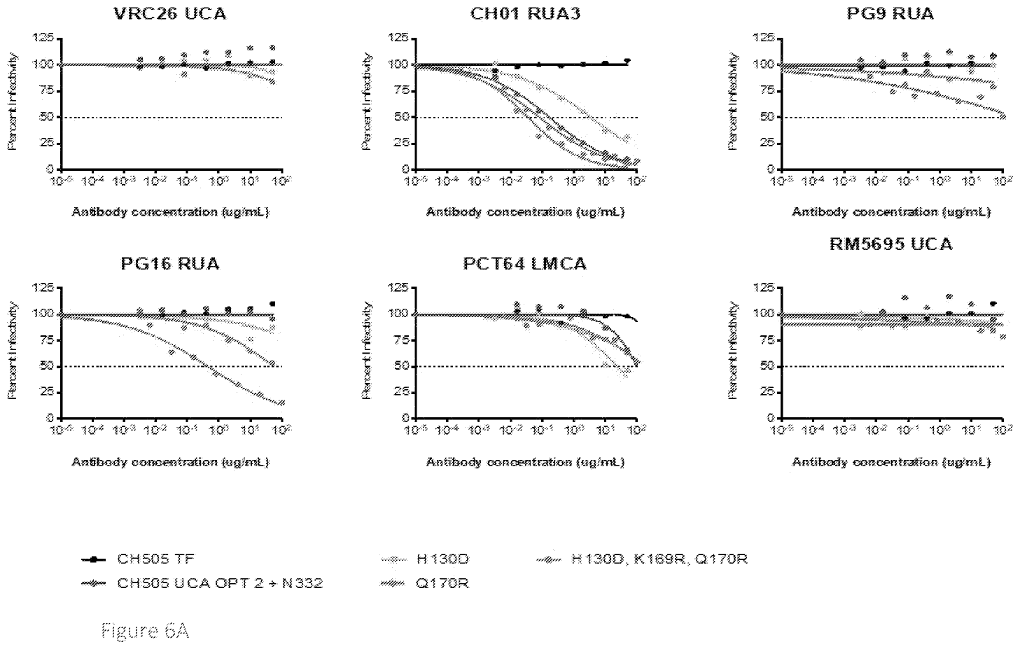

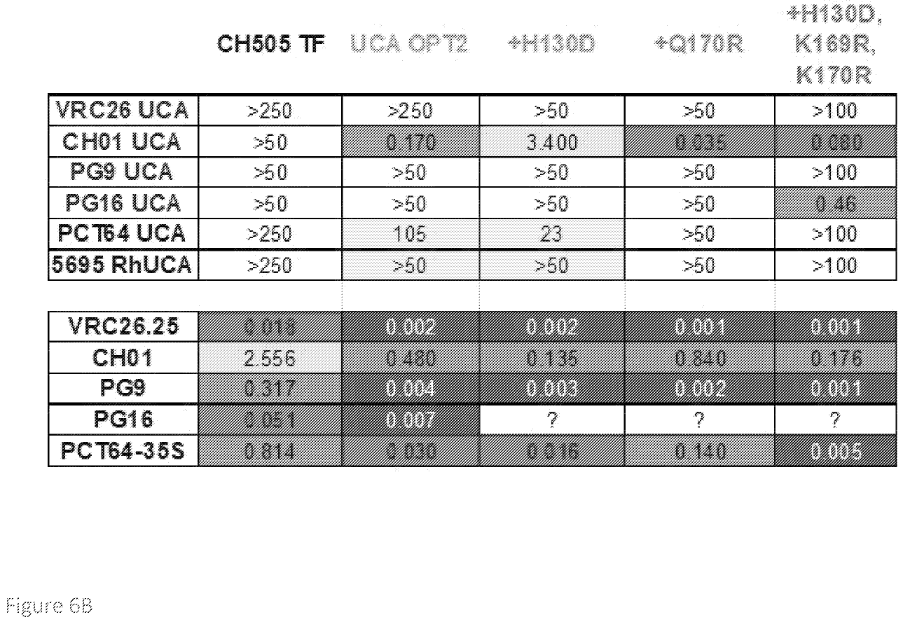

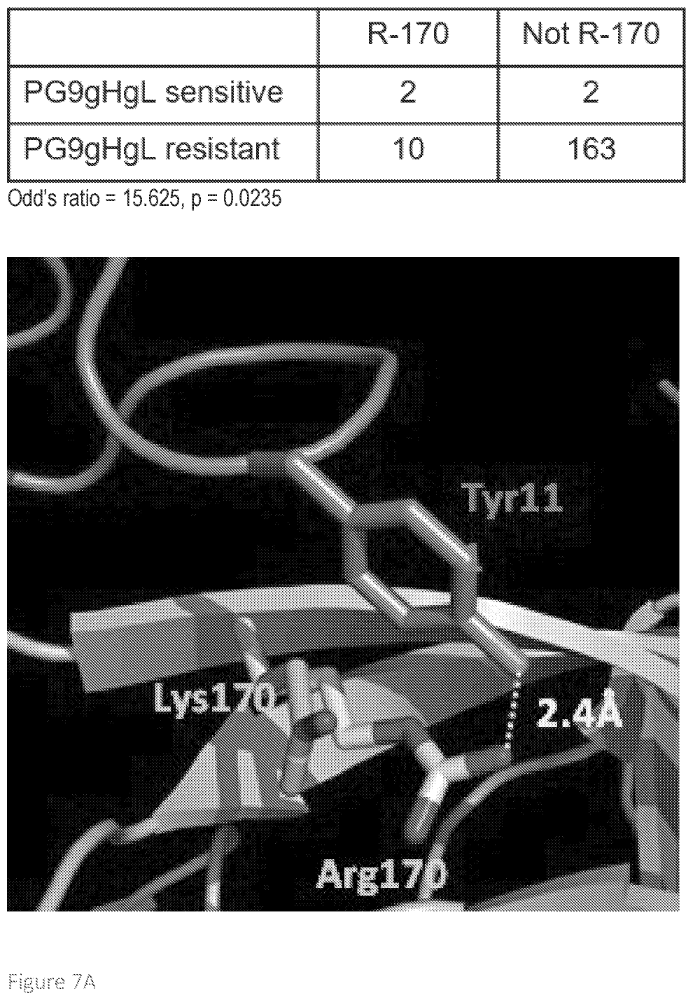

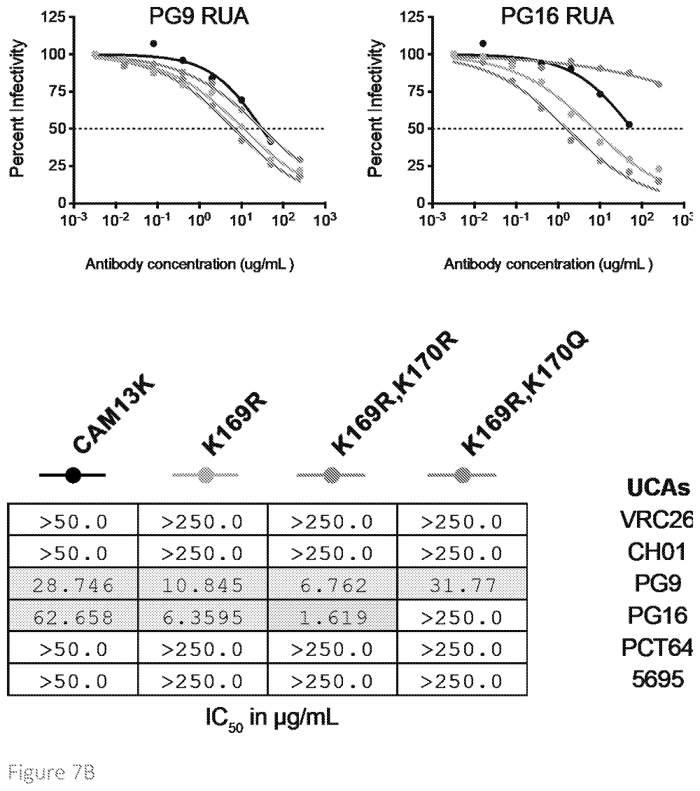

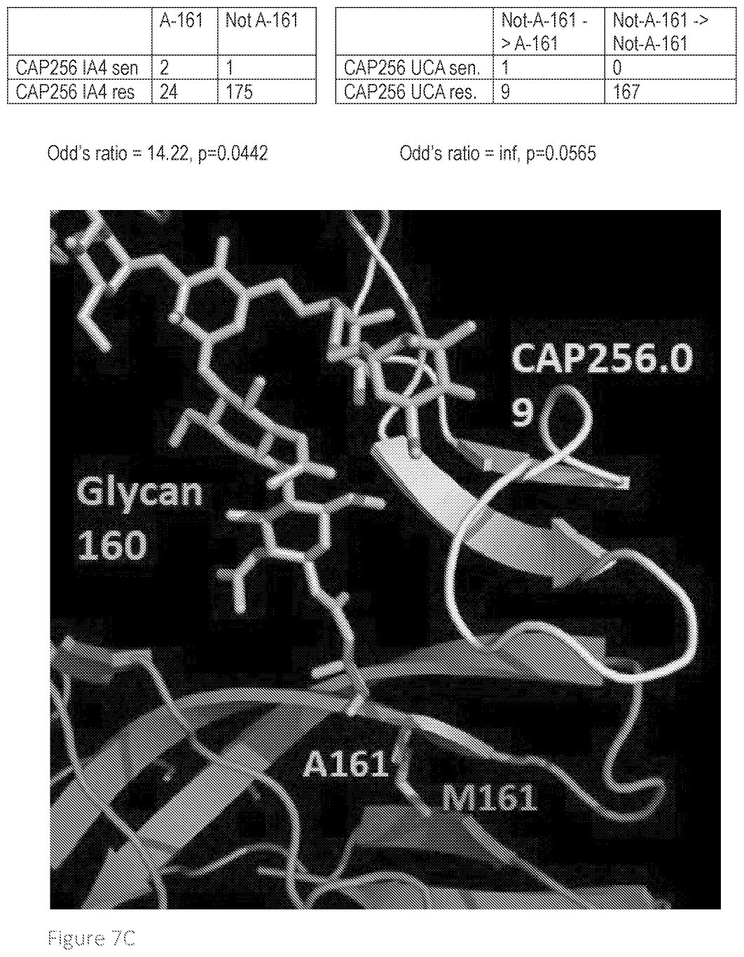

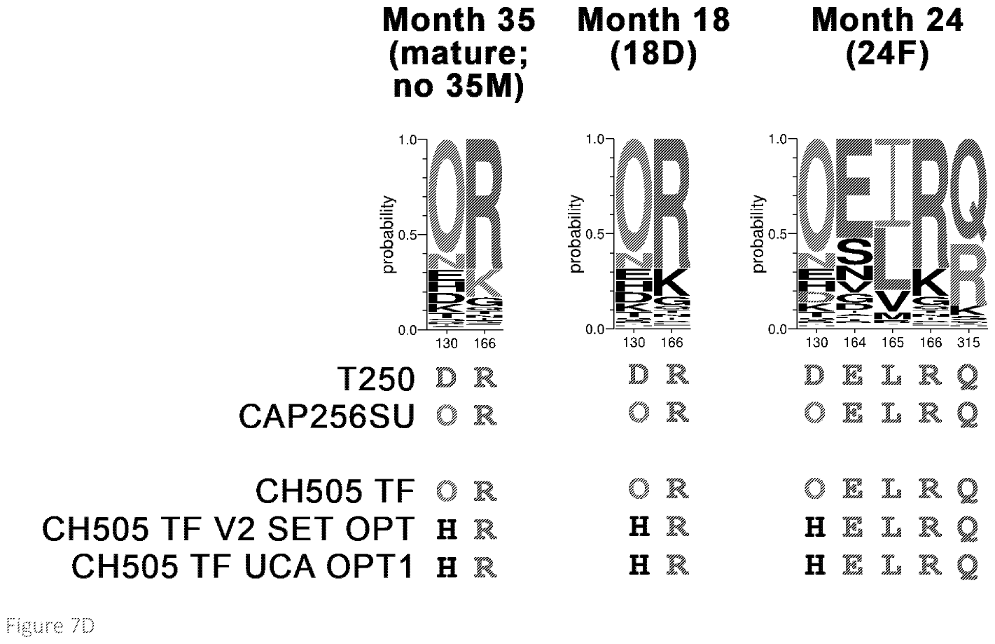

Another approach to multimerize expression constructs uses staphylococcus Sortase A transpeptidase ligation to conjugate inventive envelope trimers, for e.g. but not limited to cholesterol. Non-limiting embodiments of envelope designs for use in Sortase A reaction are shown in FIGS. 5A-B and FIG. 14. The trimers can then be embedded into liposomes via the conjugated cholesterol. To conjugate the trimer to cholesterol either a C-terminal LPXTG tag or a N-terminal pentaglycine repeat tag is added to the envelope trimer gene. Cholesterol is also synthesized with these two tags. Sortase A is then used to covalently bond the tagged envelope to the cholesterol. The sortase A-tagged trimer protein can also be used to conjugate the trimer to other peptides, proteins, or fluorescent labels. In non-limiting embodiments, the sortase A tagged trimers are conjugated to ferritin to form nanoparticles. Any suitable ferritin can be used.

The invention provides design of envelopes and trimer designs wherein the envelope comprises a linker which permits addition of a lipid, such as but not limited to cholesterol, via a Sortase A reaction. See e.g. Tsukiji, S. and Nagamune, T. (2009), Sortase-Mediated Ligation: A Gift from Gram-Positive Bacteria to Protein Engineering. ChemBioChem, 10:787-798. Doi: 10.1002/cbic.200800724; Proft, T. Sortase-mediated protein ligation: an emerging biotechnology tool for protein modification and 35mmobilization. Biotechnol Lett (2010) 32:1. Doi: 10.1007/s10529-009-0116-0; Lena Schmohl, Dirk Schwarzer, Sortase-mediated ligations for the site-specific modification of proteins, Current Opinion in Chemical Biology, Volume 22, October 2014, Pages 122-128, ISSN 1367-5931, dx.doi.org/10.1016/j.cbpa.2014.09.020; Tabata et al. Anticancer Res. 2015 August; 35 (8): 4411-7; Pritz et al. J. Org. Chem. 2007, 72, 3909-3912.

The lipid modified envelopes and trimers could be formulated as liposomes. Any suitable liposome composition is contemplated.

The lipid modified and multimerized envelopes and trimers could be formulated as liposomes. Any suitable liposome composition is contemplated.

Nomenclature for trimers: chim.6R.DS.SOSIP.664 is SOSIP.I; CHIM.6R.SOSIP.664 is SOSIP.II; CHIM.6R.SOSIP.664V4.1 is SOSIP.III.

### V2 Optimization

The CH505 HIV-I virus has been subject to intensive study as a vaccine reagent based on the observation that during the course of the natural CH505 HIV-1 infection, potent broadly neutralizing antibodies were generated by the host that targeted the CD4bs region. Here we have designed an immunogen based on the surprising finding that the HIV-1 CH505 transmitted-founder (TF) virus Envelopes, when used as vaccine, have the capacity to induce V2 apex directed heterologous neutralizing antibody responses. This has been observed in a knoc—in mice, rabbits and rhesus macaques, and in one CH505 SHIV infected macaque. These results raise the prospect of ultimately creating a dual-targeting CH505-based immunogen design that can induce both V2 apex and CD4bs broadly neutralizing antibodies (bNAbs). The designs we propose focus on enhancing both the initiation of appropriate V2 apex targeting neutralizing antibody and expand the breadth of the response.

Despite the fact the CH505 TF Envelope can elicit V2 apex neutralizing antibody responses, it is not particularly sensitive to mature V2 apex bNAbs and is not neutralized by putative V2 apex bNAb precursors. We hypothesized that these factors could be limit the successful V2 apex bNAb induction, and that CH505 TF variants with improved sensitivity to V2 apex mature and precursor antibodies might serve as better immunogens.

Thus, we used our previously published statistically robust and phylogenetically corrected strategy to compare the CH505 TF to amino acid and glycan signatures that associate with sensitivity to multiple V2 apex bNAbs (Bricault et al. Cell Host Microbe (2019) 25:59-72). We found that CH505 TF carried resistance signatures at 10 sites, and by introducing favorable mutations at these sites, we designed a variant called V2 SET OPT (signature-based epitope targeted optimized) (FIG. 1). Shorter and more positively charged bypervariable V1 and V2 loops are significantly associated with neutralization sensitivity by mature V2 apex bNAbs, so we also introduced optimal V1 and V2 hypervariable loops from two natural Envs, ZM233.6 and T250-4, respectively, into our constructs.

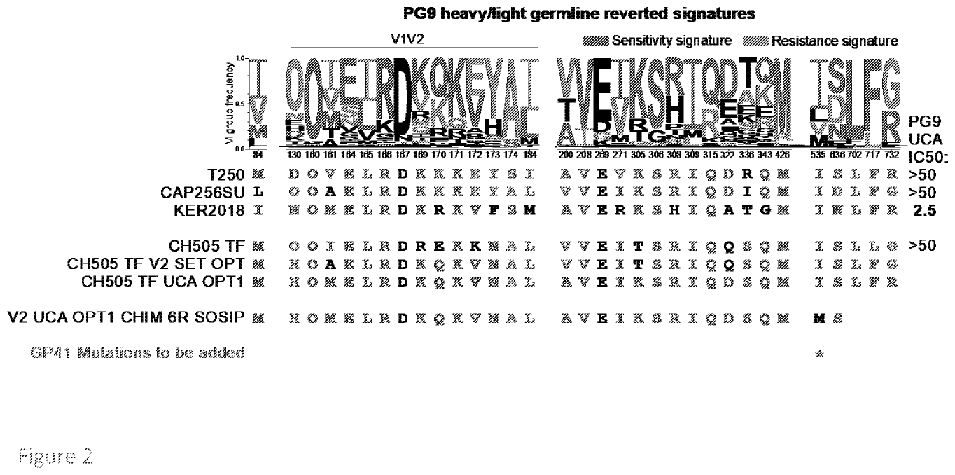

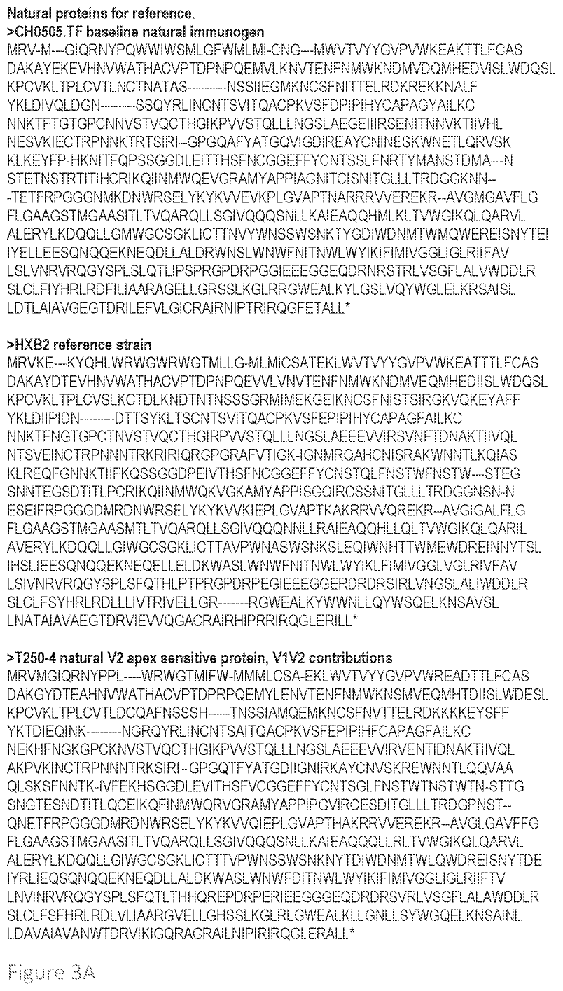

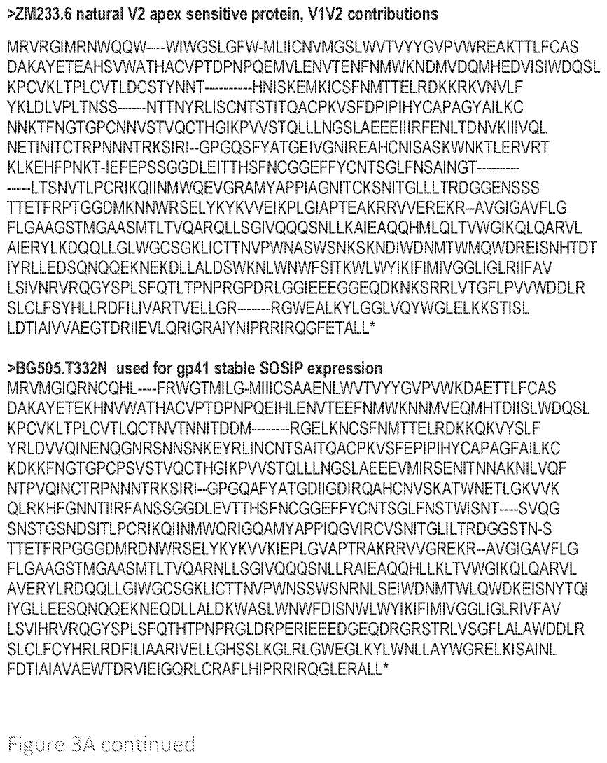

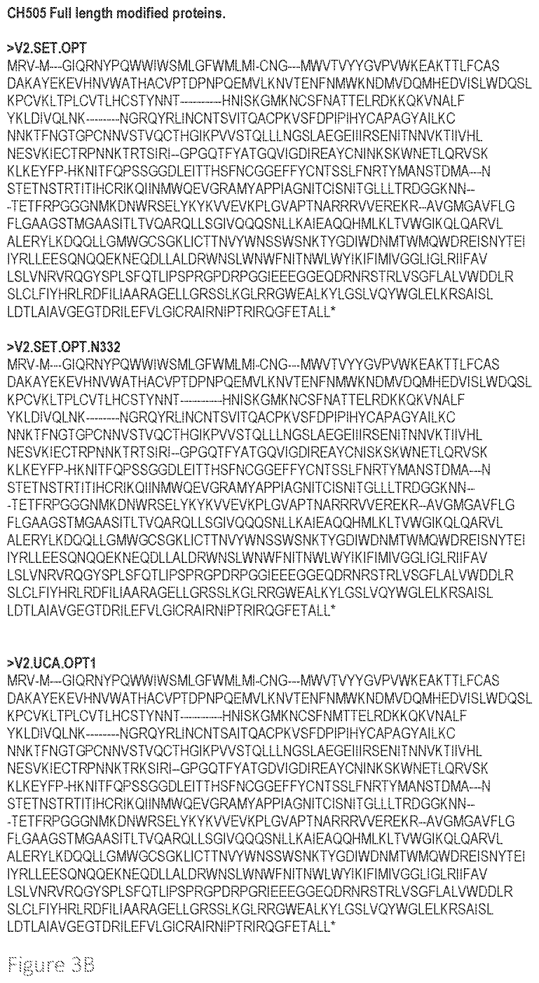

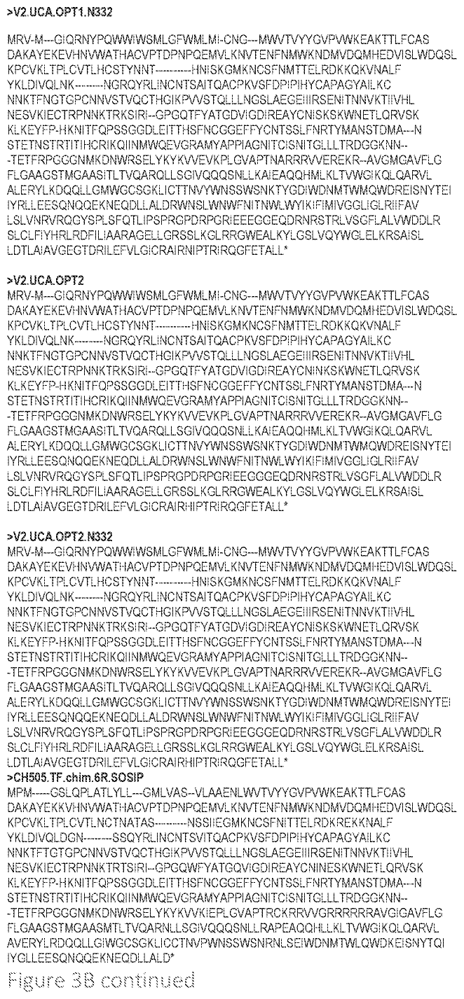

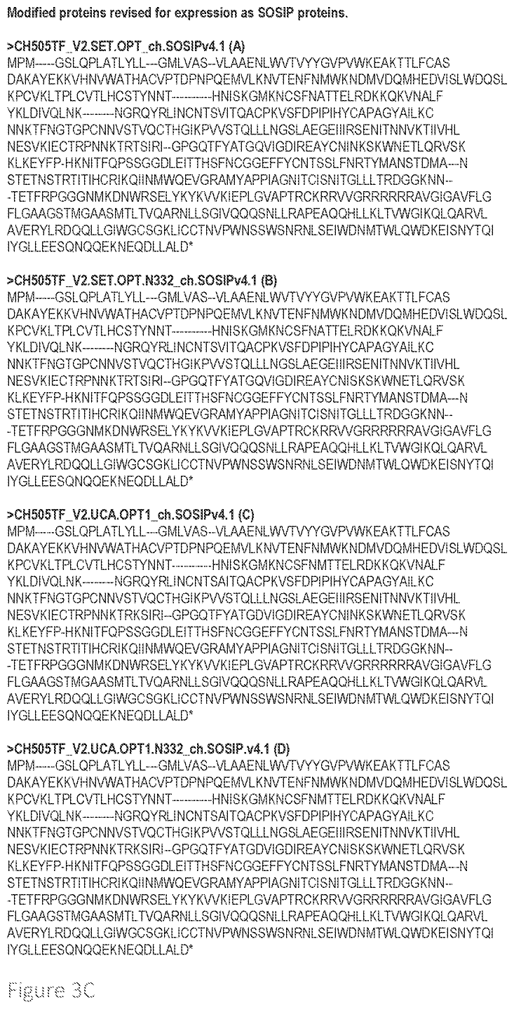

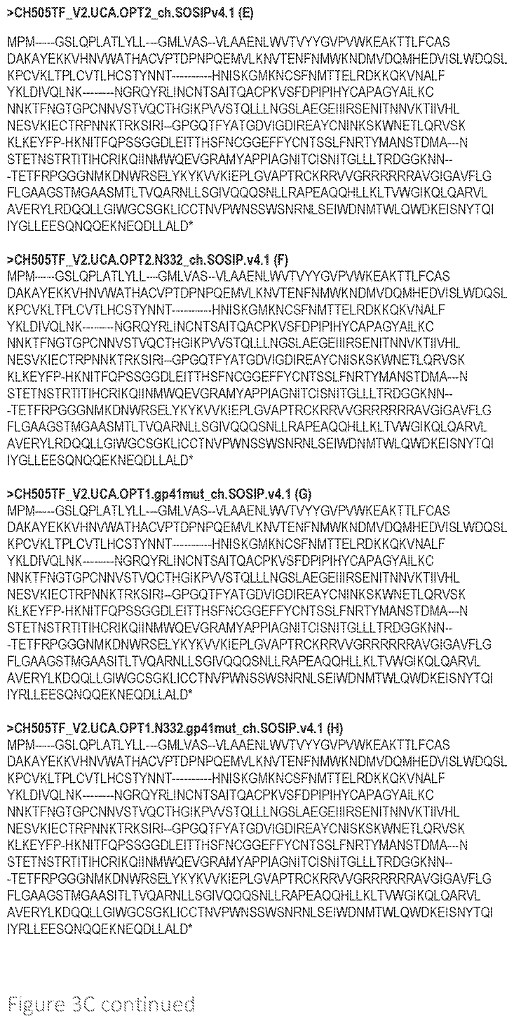

We next applied signature analyses to neutralization data for 109-208 global viruses tested against unmutated or early ancestral antibodies that ultimately gave rise to antibody lineages that targeted the V2 apex and potent broadly neutralizing antibodies: CH04 UCA, CAP256-VRC26 and PCT64 early intermediates, and heavy and/or light chain germline reverted PG9 and PGT145. Using this strategy, we identified signatures associated with sensitivity to V2 apex precursors (FIG. 2).

The hypervariable loop characteristics associated with sensitivity to V2 apex precursors were similar to those of the mature, and hence, the hypervariable V1 and V2 loop modifications from V2 SET OPT were retained.

The first round of V2 optimization was successful in improving sensitivity to all mature V2 apex bNAbs and for 2 out of 6 UCAs tested (CH01 and PCT64). A further round of iterative design optimization was carried out to improve reactivity against the remaining 4 UCAs. These designs introduced three mutations H130D, K169R and Q170R. The first mutation was based on the consideration that D-130 was a sensitivity signature for PCT64 UCA (while H-130 was sensitivity signature for CH04 UCA) and was introduced with the aim of improving sensitivity to PCT64 UCA. The K169R and Q170R mutations were introduced with the aim of improving sensitivity to PG9 and PG16 UCAs. Both of these mutations were found to improve the PG9 and PG16 UCAs in the background of an SIV strain, while the latter Q170R was also found to be the strongest sensitivity signature associated with sensitivity to fully germline reverted PG9 antibody (both heavy and light chains reverted) in the PG9 epitope. Introduction of these 3 mutations in the context of CH505.TF.V2UCA.OPT2.N332 was found to improve sensitivity to PG9 and PG16 UCAs, while retaining sensitivity to CH01 and PCT64 UCAs and to all mature V2 apex bNAbs.

In non-limiting embodiments, these vaccines are being expressed as chimeric SOSIP proteins, and so have CH505 TF gp120s, with a BG505 gp41 that end at HIV-1 HXB2 numbering position 664. SOSIP proteins are modified Env proteins that are stabilized for expression as native-like soluble trimers.

These sensitivity mutations in a CH505 TF background expressed as SOSIP proteins we propose will result in immunogens that are more susceptible to V2-apex antibodies, and thus may be better able to trigger and stimulate them.

The modified sequence we are suggesting trying as immunogens are enclosed. We start the alignment with CH505.IF as a reference, the natural transmitted founder virus that we are building mutations into. We follow with full length protein sequences that contain the amino acid modifications we believe may be advantageous. We include the natural strains ZM233.6 and T250-4 in the alignment, as we included their hypervariable regions.

Table 1 shows V2 Optimized CH505 TF immunogens

| Gene number | Protein name | Immunogen criteria |
|---|---|---|
| HV1301908 | CH505TF_V2.UCA.OPT1.gp41mut_ch.SOSIP.v4.1 | Optimized gp120 and gp41 based on |
| | | V2-glycan bnAb UCA neutralization |
| HV1301909 | CH505TF_V2.UCA.OPT1.N332.gp41mut_ch.SOSIP.v4.1 | Optimized gp120 and gp41 based on |
| | | V2-glycan bnAb UCA neutralization |
| HV1301910 | CH505TF_V2.SET.OPT_ch.SOSIPv4.1 | Optimized gp120 based on V2-glycan |
| | | bnAb neutralization |
| HV1301911 | CH505TF_V2.SET.OPT.N332_ch.SOSIPv4.1 | Optimized gp120 based on V2-glycan |
| | | bnAb neutralization with N332 |
| | | glycan hole filled |
| HV1301912 | CH505TF_V2.UCA.OPT1_ch.SOSIPv4.1 | Optimized gp120 based on V2-glycan |
| | | bnAb UCA neutralization |
| HV1301913 | CH505TF_V2.UCA.OPT1.N332_ch.SOSIP.v4.1 | Optimized gp120 based on V2-glycan |
| | | bnAb UCA neutralization with N332 |
| | | glycan hole filled |

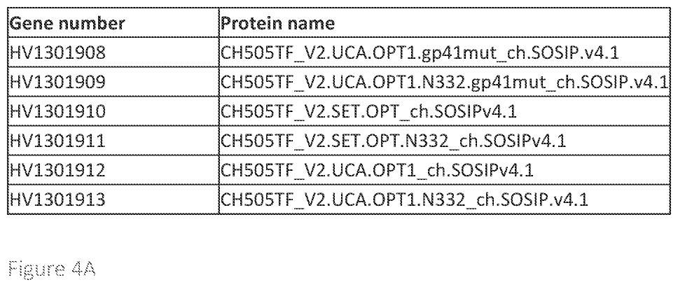

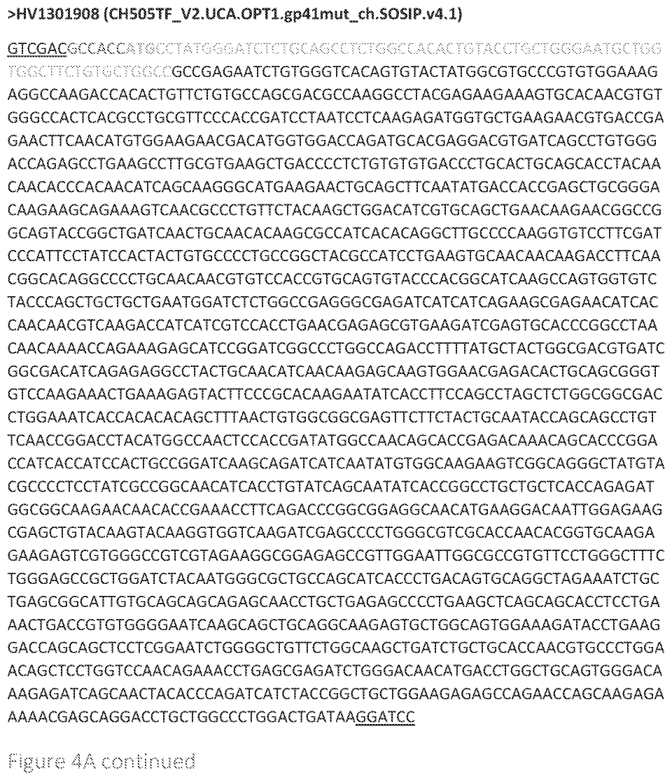

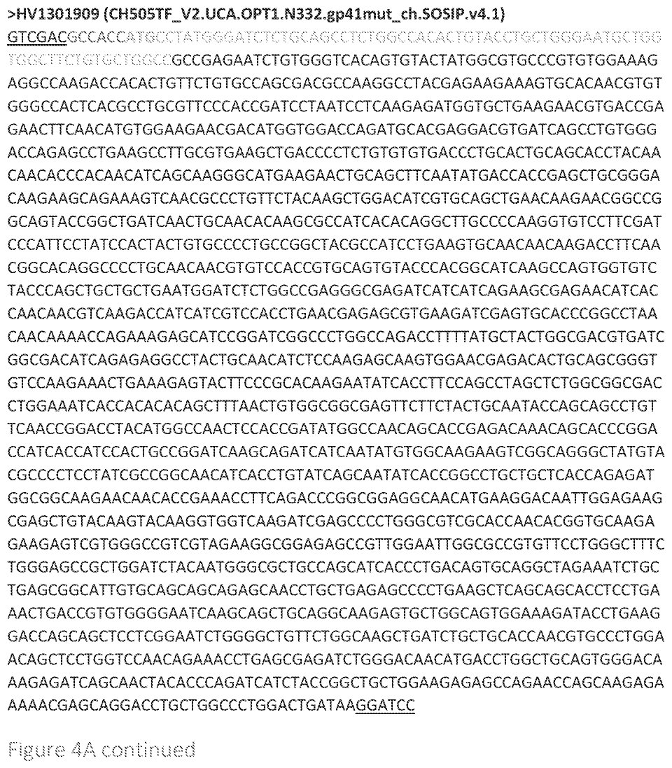

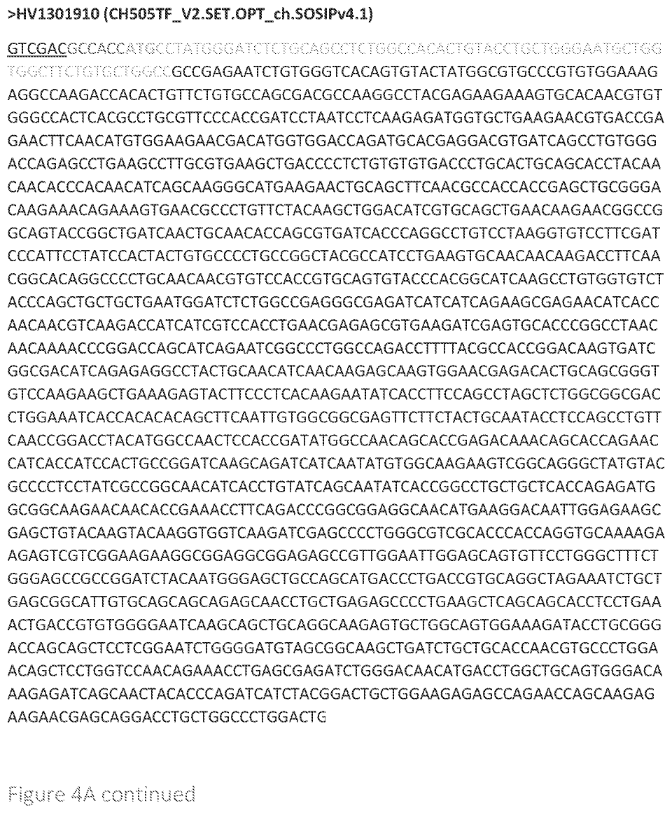

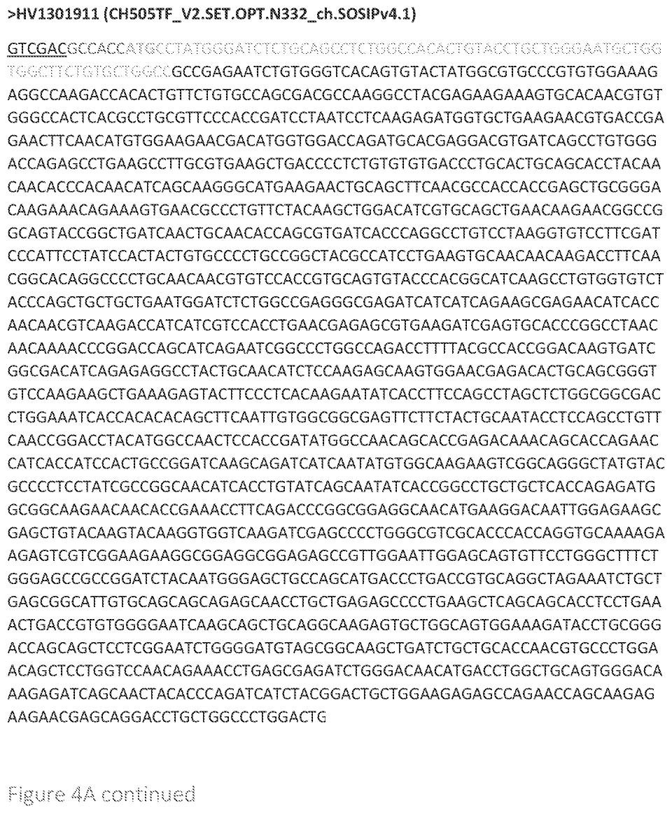

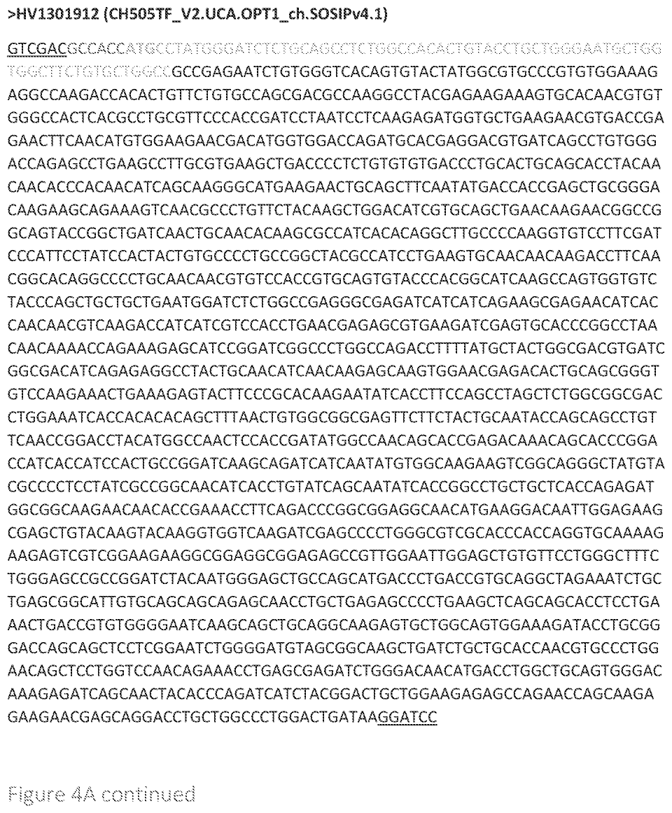

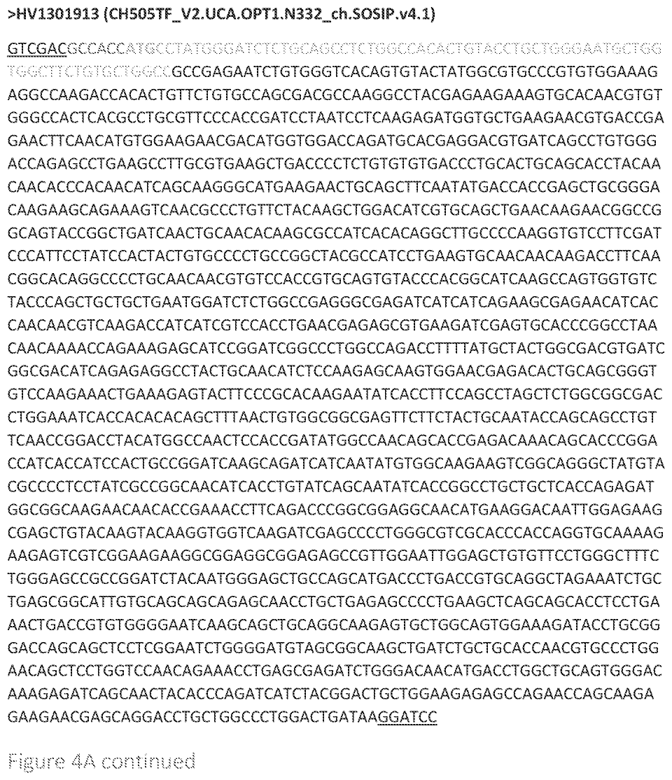

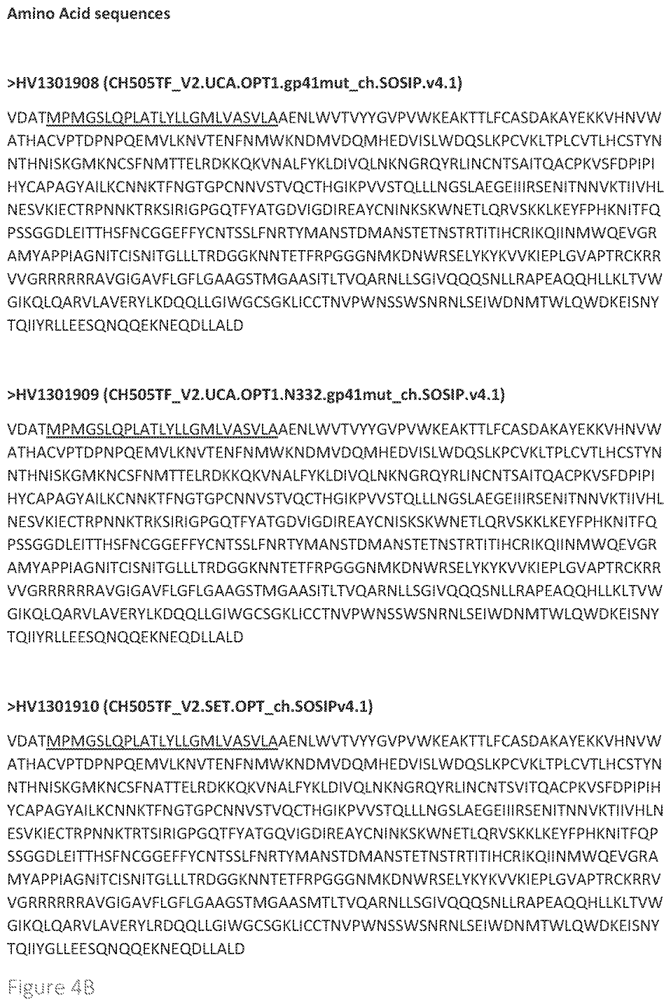

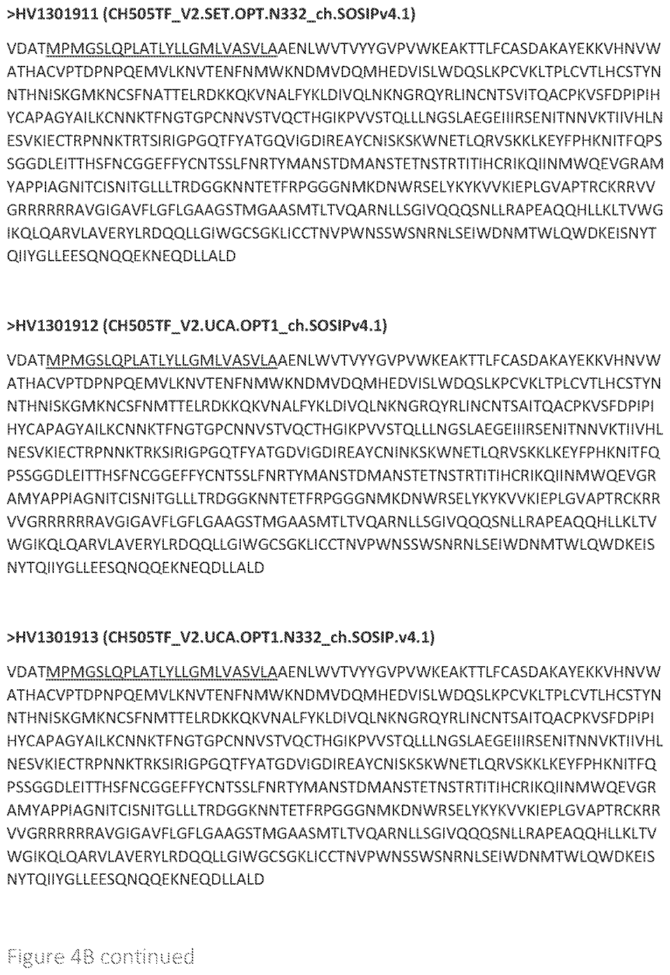

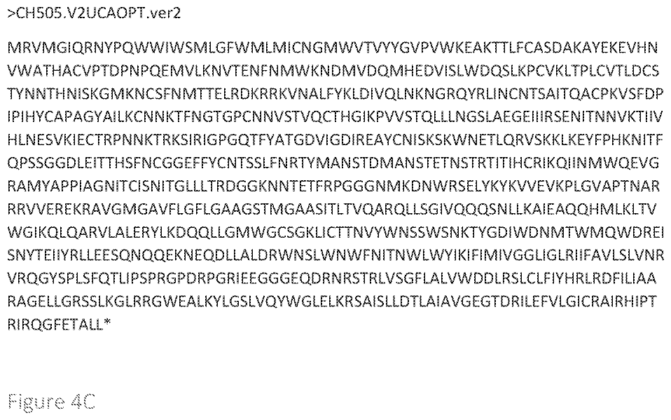

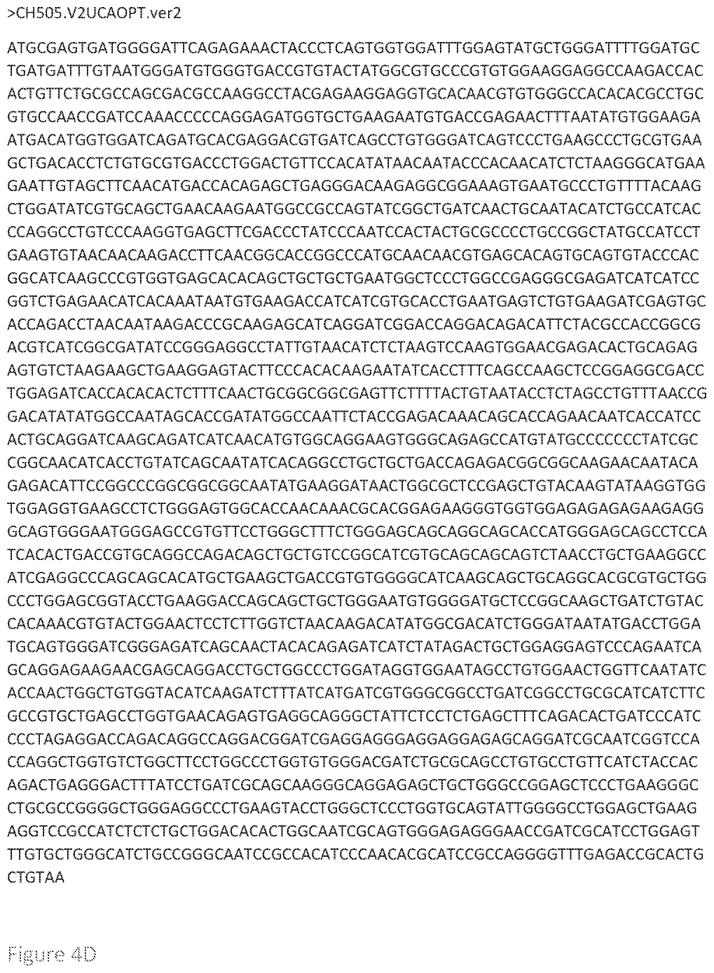

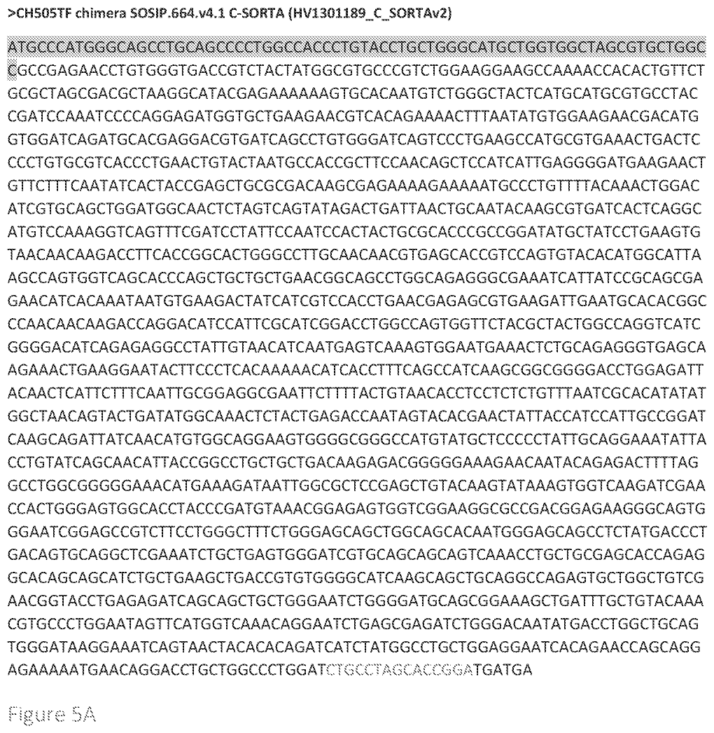

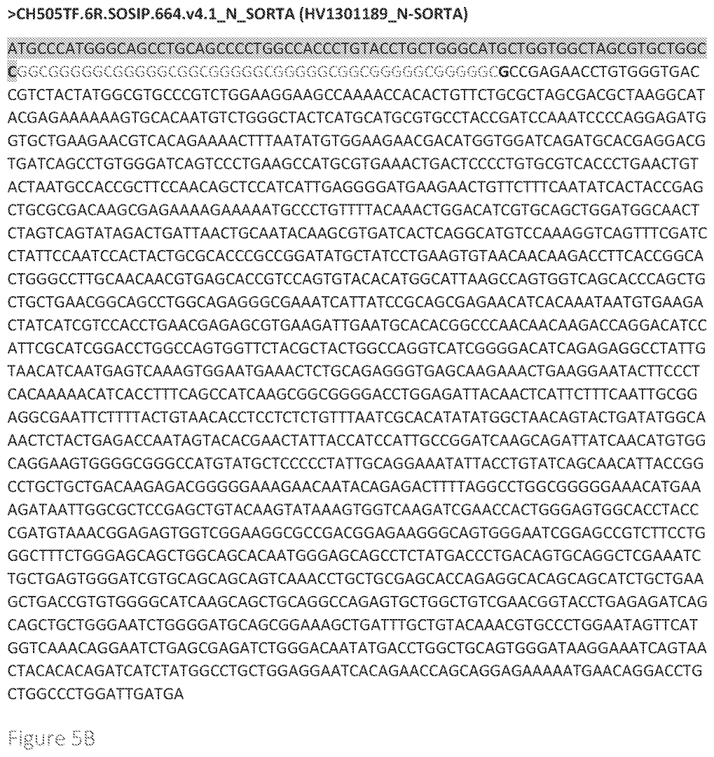

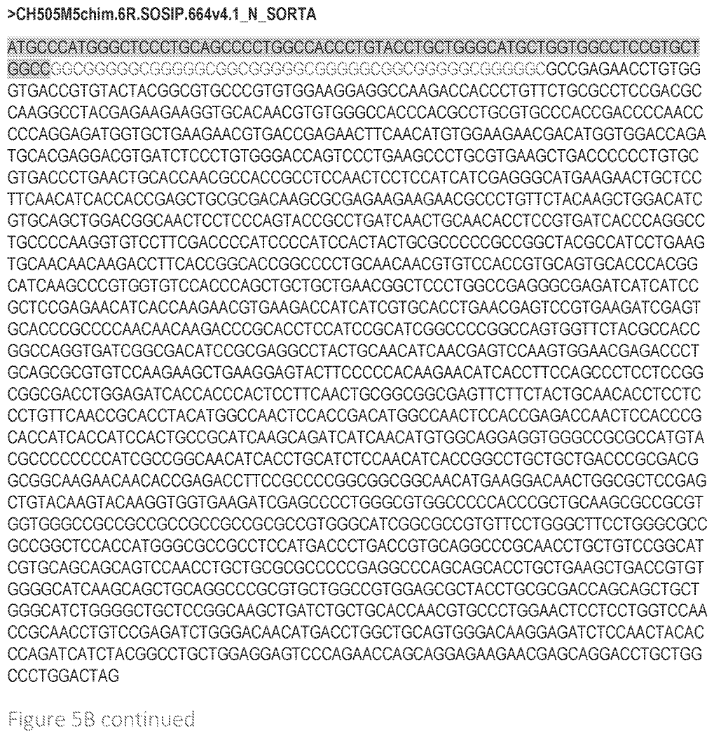

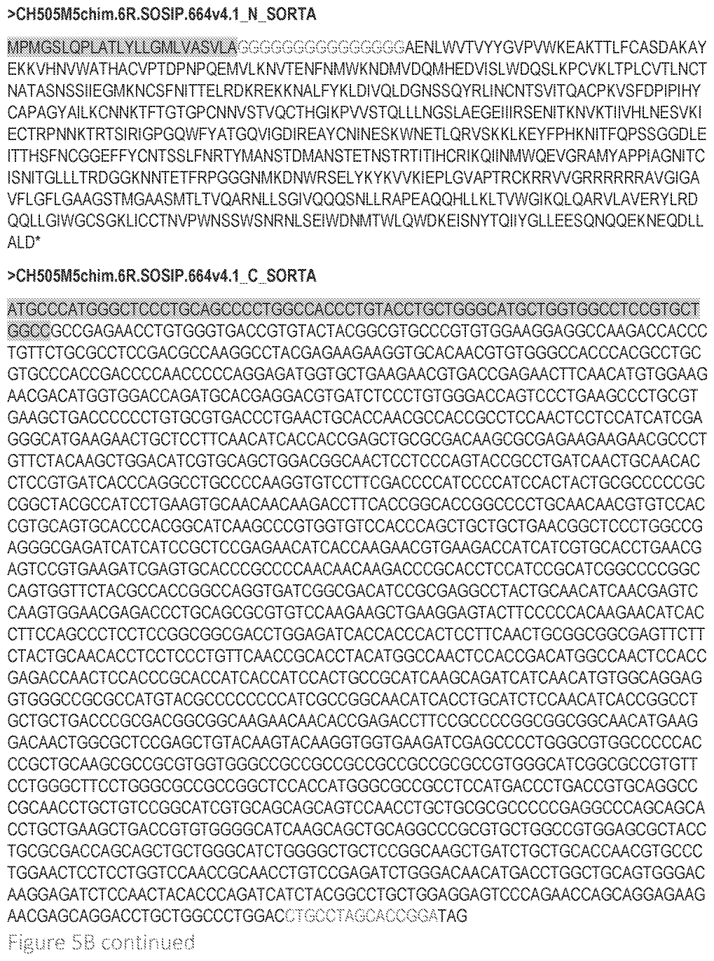

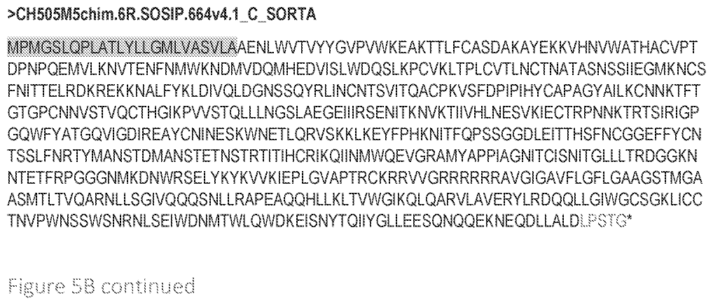

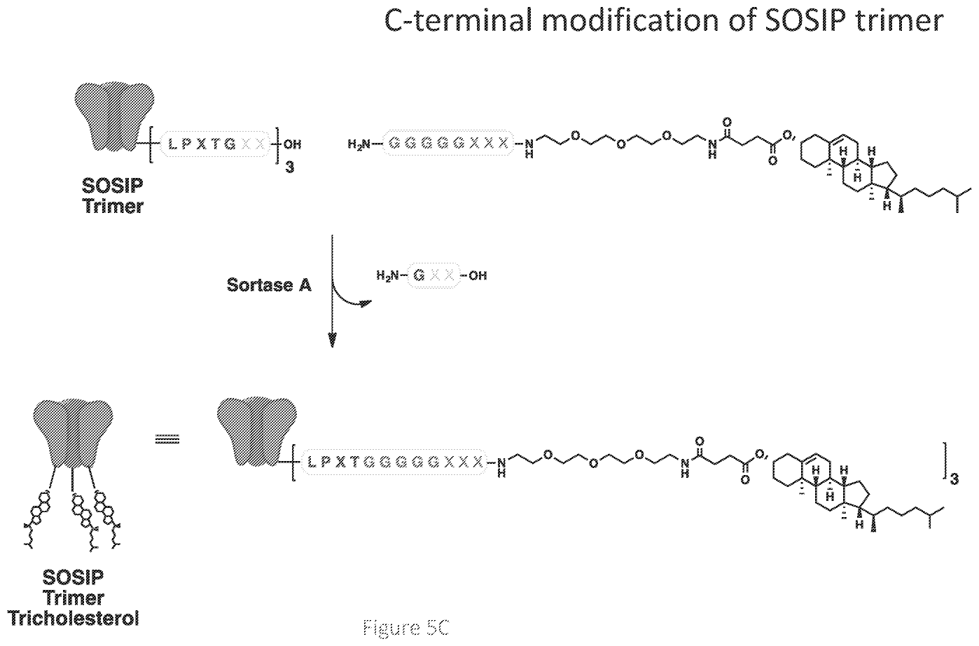

Non-limiting embodiments of sequences of the envelopes in Table I are described in FIGS. 3, 4, and 5 shows non-limiting embodiments of multimerization designs, including ferritin and/or sortase, which could be used as guidance to design V2OPT_CH505T/F designs. In FIGS. 4C-D (see Table 2 below), CH505. V2UCAOPT.ver2 envelope sequence is shown as a gp160 envelope. This V2 optimized design could be used as the basis to design any suitable protomer, wherein in non-limiting embodiments the protomer can form stabilized trimer. Non-limiting designs of envelope protomers include SOSIP designs, designs comprising F14 mutations (See US Pub 20210379177, incorporated herein by reference), and so forth. In non-limiting embodiments, any of the envelope designs, including without limitation designs in FIG. 3, 4, or 5, could comprise mutations H130D, K169R and/or Q170R.

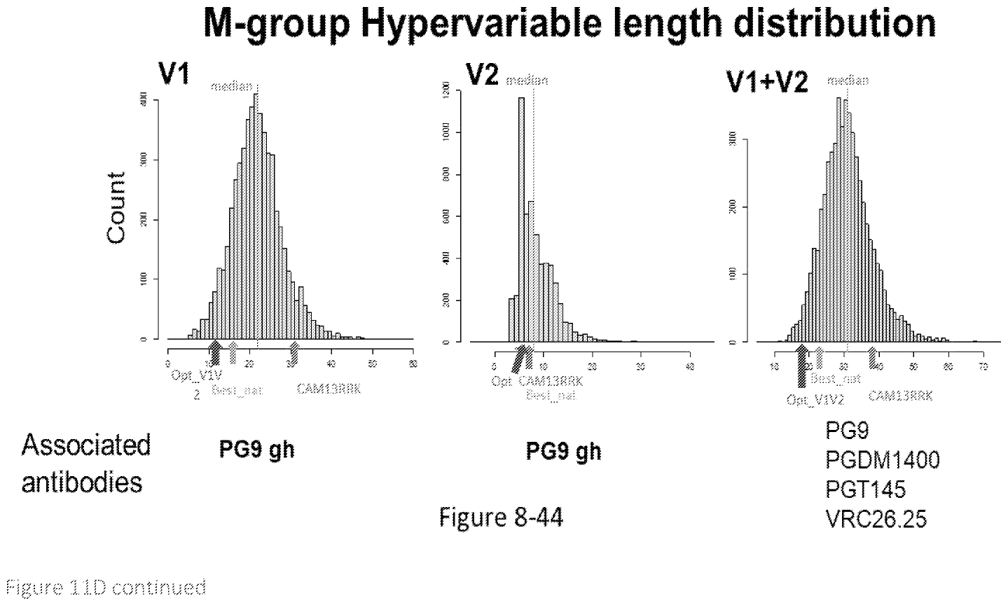

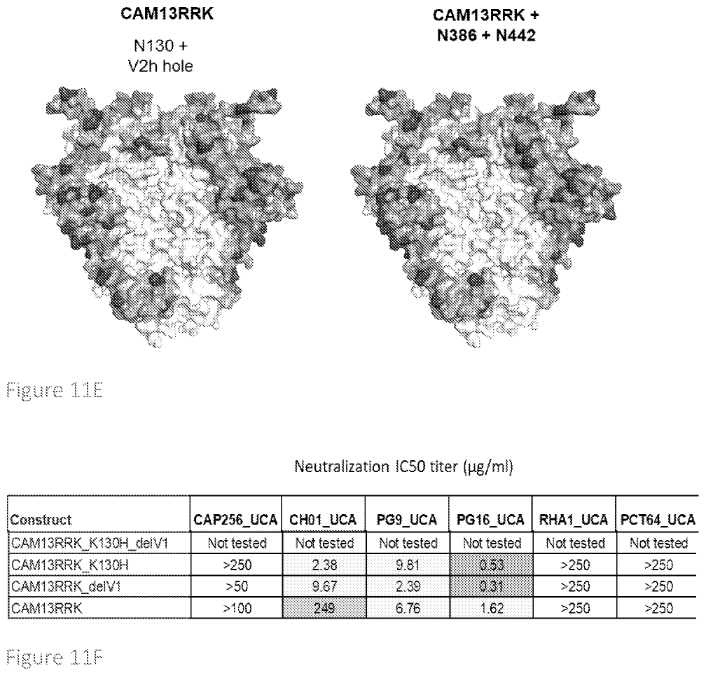

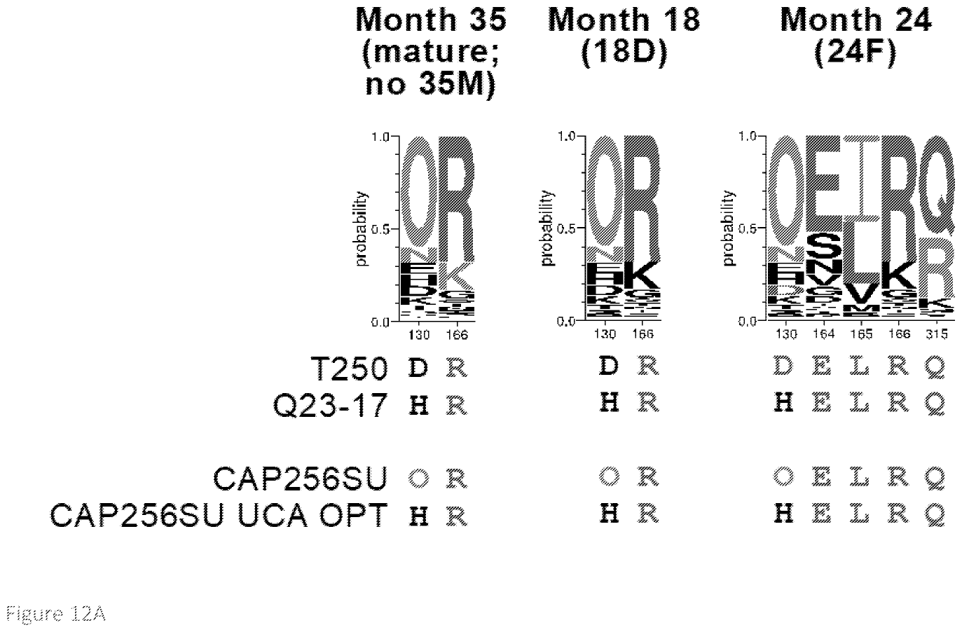

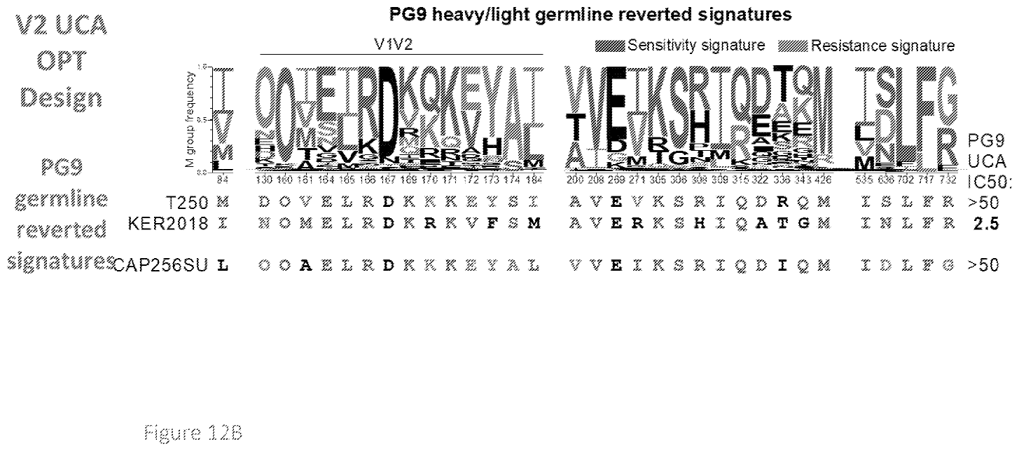

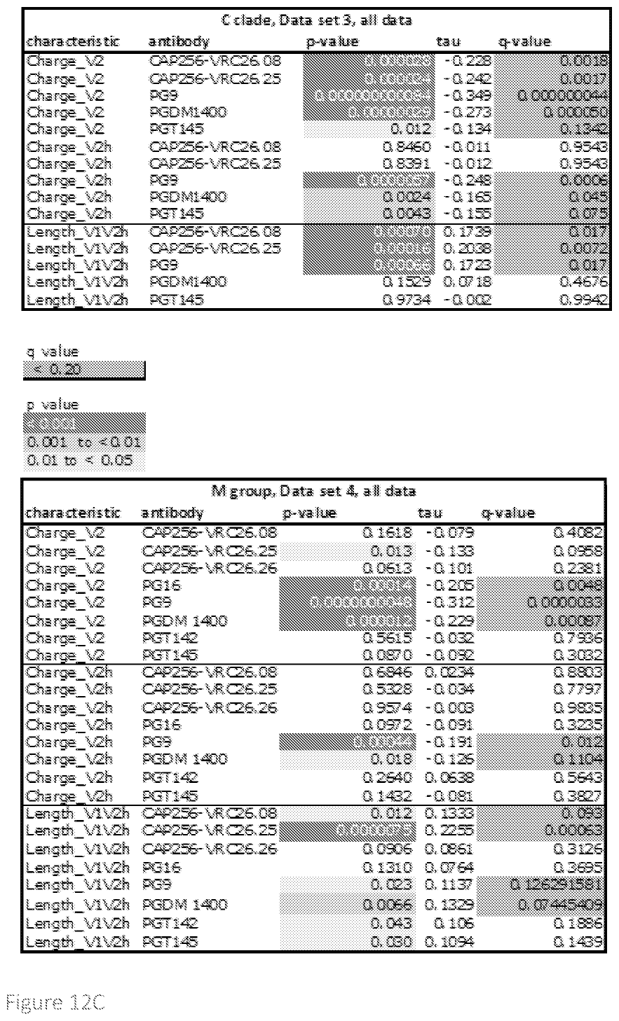

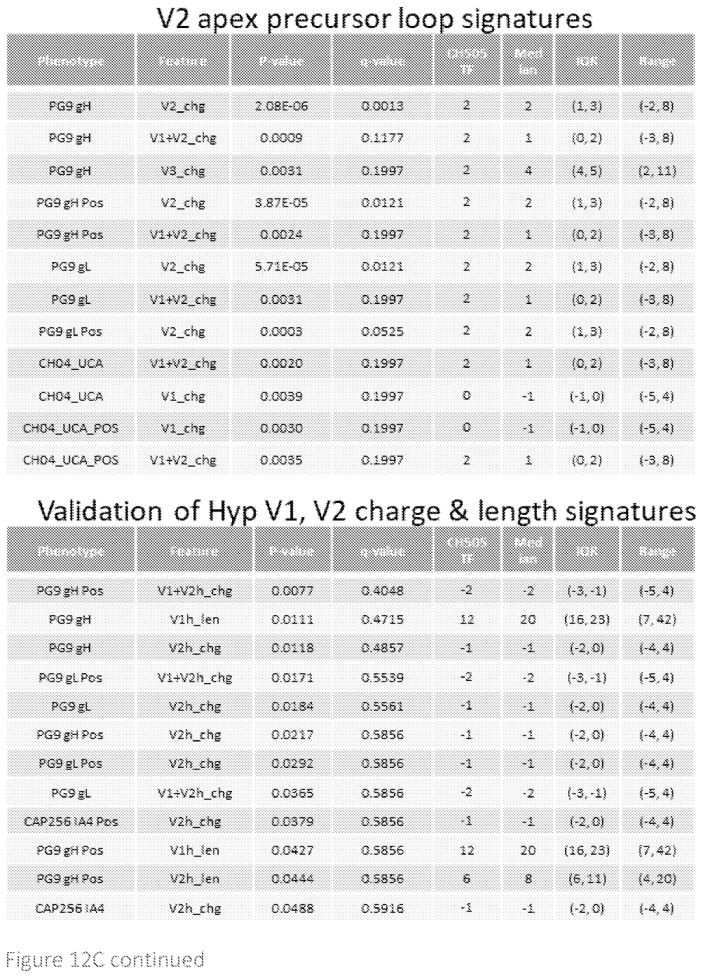

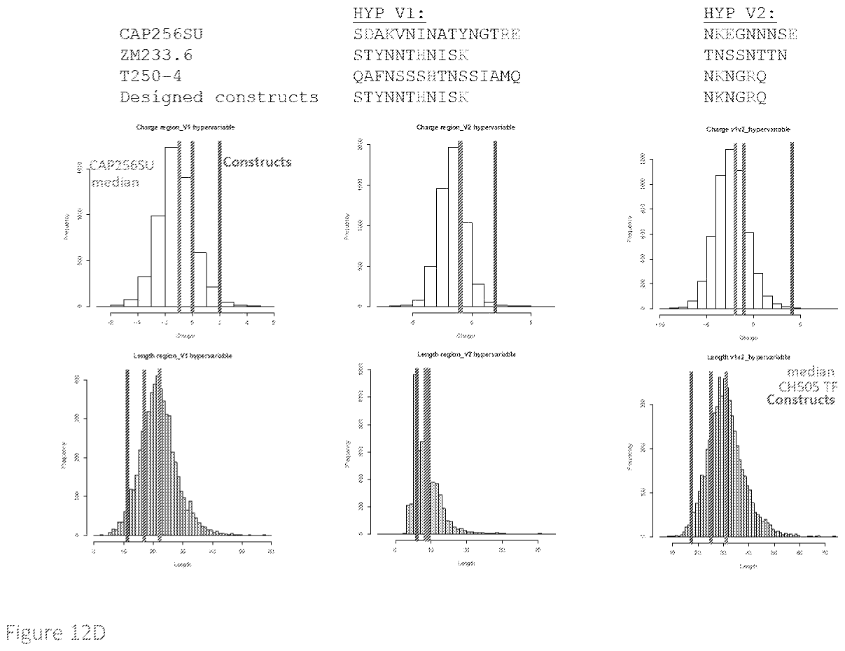

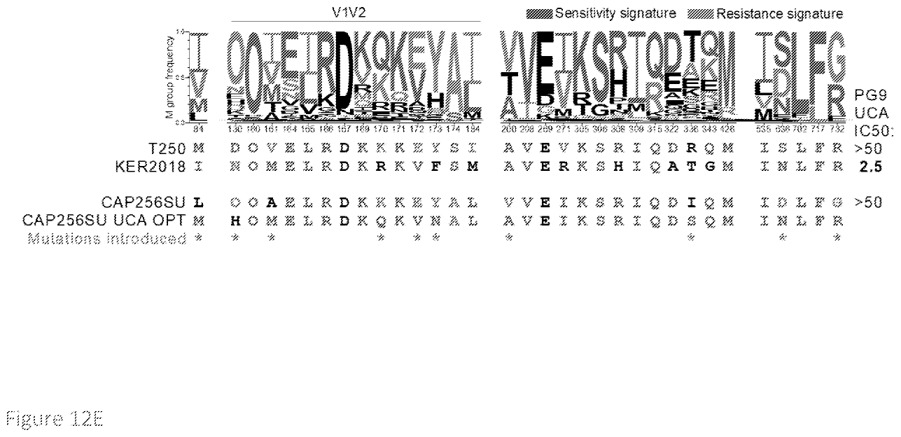

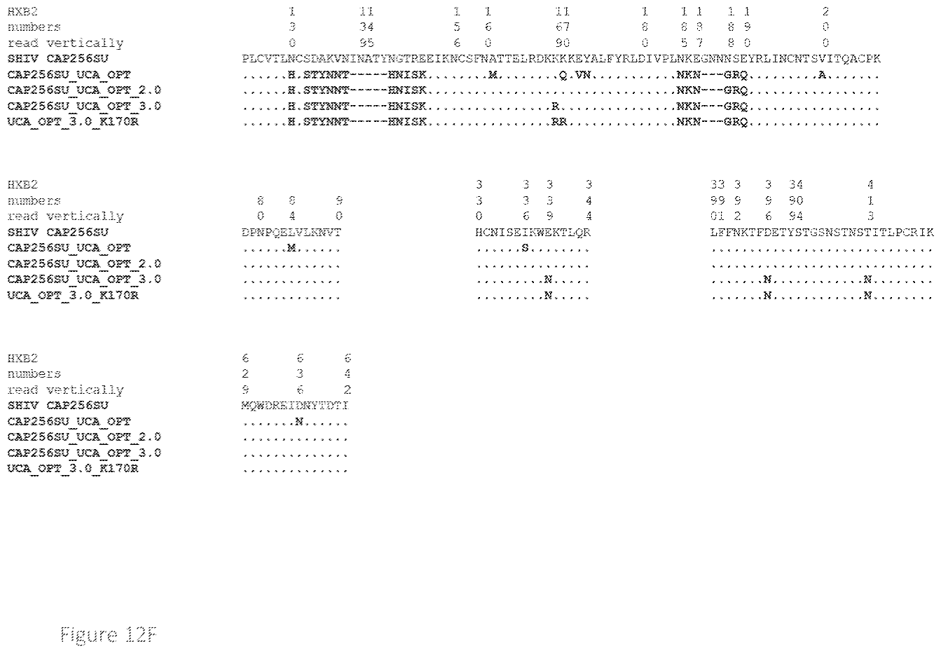

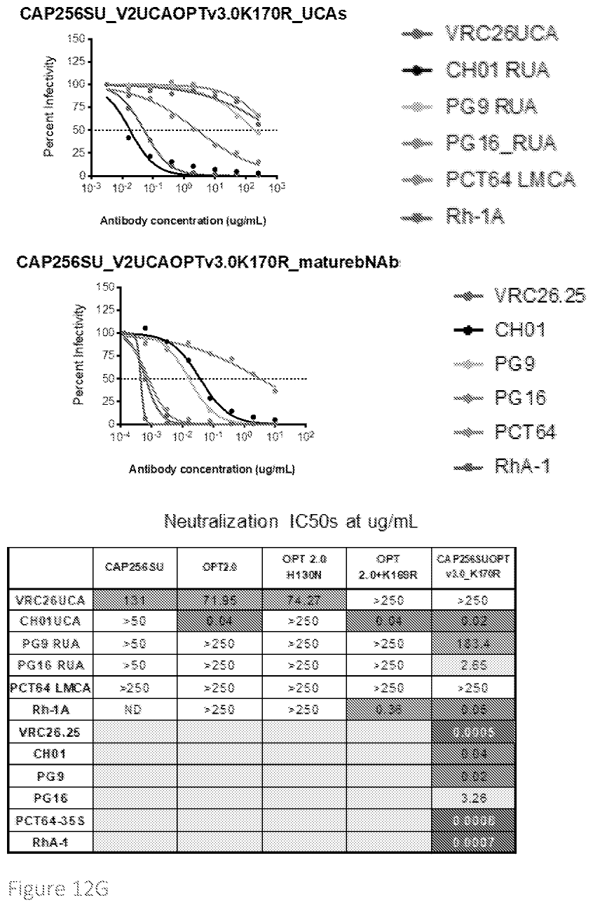

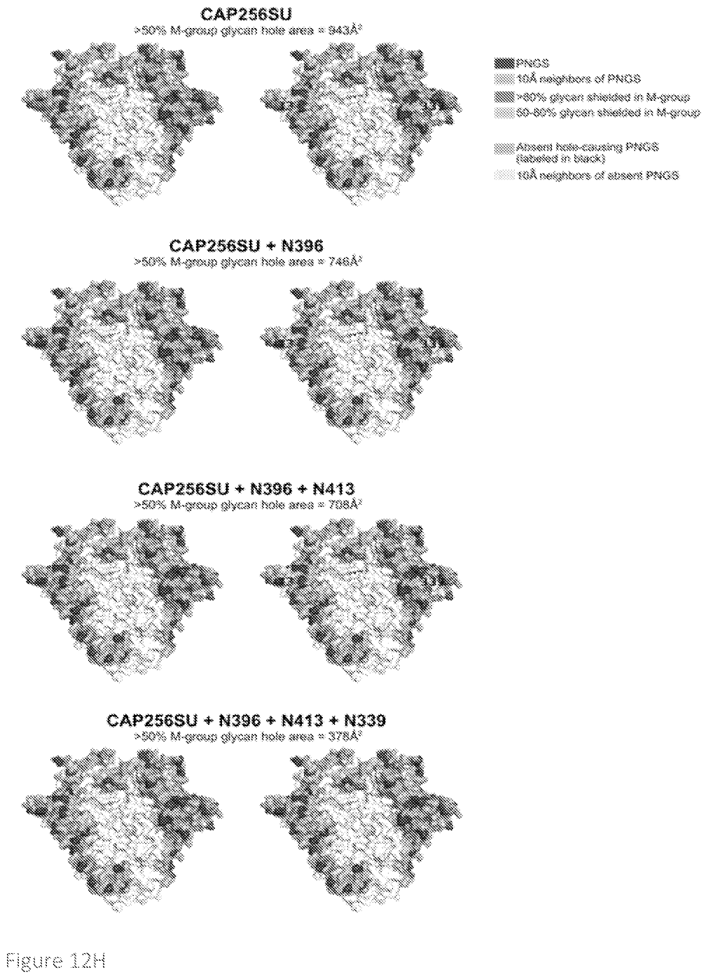

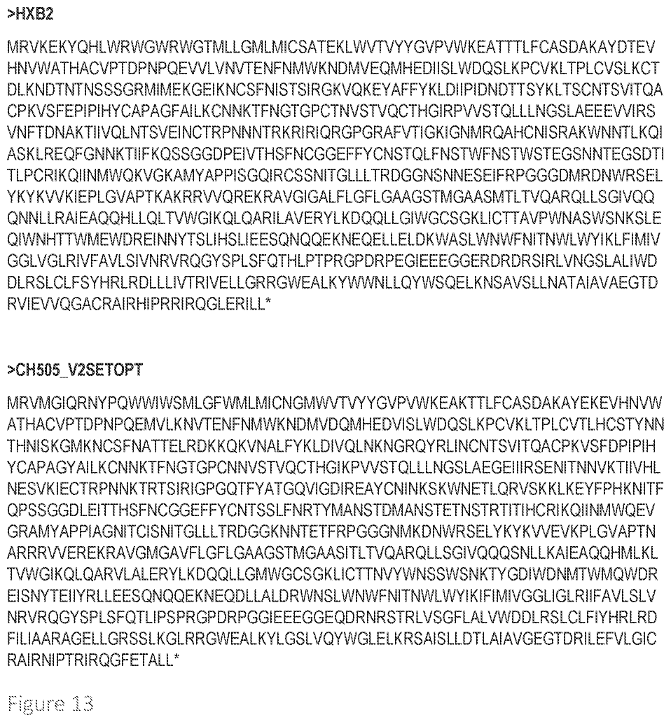

Table 2 shows non-limiting embodiments of optimized immunogens. See FIG. 4C-4D, 13, 16, and 17.

| Gene | Protein name-see | Non-limiting embodiments |
|---|---|---|
| number | FIGS. 8-12, 16, 17 | are shown in Figs. |
| | CH505.V2UCAOPT.ver2 (Optimized | FIG. 4C-4D |
| | CH505 T/F envelope) | |
| | CH505_V2SETOPT | FIG. 13 |
| | CH505_V2SETOPT_N332 | FIG. 13 |
| | CH505_V2UCAOPT1 | FIG. 13 |
| | CH505_V2UCAOPT1_N332 | FIG. 13 |
| | CH505_V2UCAOPT2 | FIG. 13 |
| | CH505_V2UCAOPT2_N332 | FIG. 13 |
| | CH505_V2UCAOPT_v3.0 | FIG. 13 |
| | CH505_UCA_OPT2_N332_H130D_K169R_K170R is called | |
| | CH505_V2UCA_OPT_v3.0 | |
| | CH505_V2UCAOPT_v3.0_D167N | FIG. 13 |
| | CAP256SU_UCA_OPT_2.0 | FIG. 13 |
| | CAP256SU_UCA_OPT_3.0 | FIG. 13 |
| | CAP256SU_UCA_OPT_3.0_K170R | FIG. 13 |
| | CAM13RRK | FIG. 13 |
| | CAM13RRK_K130H | FIG. 13 |
| | CAM13RRK_ΔV1 | FIG. 13 |
| | CAM13RRK_ΔV1_K130H | FIG. 13 |
| | CAM13RRK_K130H_natV1hV2hswap_natgly | FIG. 13 |
| | CAM13RRK_K130H_optV1hV2hswap_optgly | FIG. 13 |
| | CAP256_wk34.80_V2UCA_OPT | FIG. 16 |
| | CAP256_wk34.80_V2UCA_OPT_R171K | FIG. 16 |
| | CAP256_wk34.80_PCT64UCA_OPT | FIG. 16 |
| | CAP256SU_UCA_OPT_4.0_D167N | FIG. 16 |
| | CAM13RRRK | FIG. 17 |
| | HIV_CAP256SU_UCA_OPT_4.0 | FIG. 17 |
| | CAP256SU_UCA_OPT_4.0_375S | FIG. 17 |
| | CAP256SU_UCA_OPT_4.0_Y375S_D167N | FIG. 17 |
| | CAP256_wk34.80_V2UCA_OPT_RRK | FIG. 17 |
| | CAP256_wk34.80_V2UCA_OPT_RRK_D167N | FIG. 17 |
| | mrna1_CAP256SU_UCA_OPT_4.0 | FIG. 17 |
| | mrna2_CAP256SU_UCA_OPT_4.0 | FIG. 17 |
| HV1303230 | HIV_CAP256SU_UCA_OPT_4.0 | FIG. 17 |
| | F14(A204V_V208L_V68I_V2SSL)_gp160_{—} | |
| | IGHVss_deltaG_SOSL.GS_I535M_Y712I | |
| HV1303231 | HIV_CAP256SU_UCA_OPT_4.0 | FIG. 17 |
| | F14(A204V_V208L_V68I_V255L)_gp160_{—} | |
| | IGHVss_deltaG_DS.SOSL.GS_I535M_{—} | |
| | Y712I_LL855/6AA | |
| HV1303232 | HIV_CAP256SU_UCA_OPT_4.0 | FIG. 17 |
| | F14(A204V_V208L_V68I_V255L)_{—} | |
| | RnS3mut2G_gp160_IGHVss_deltaG_{—} | |
| | DS.SOSL.GS_I535M_Y712I_LL855/6AA | |
| HV1303233 | HIV_CAP256SU_UCA_OPT_4.0_F14(A204V_{—} | FIG. 17 |
| | V208L_V68I_V255L)_gp160_IGHVss_{—} | |
| | deltaG_DS.SOSL.GS_I535M.PC._Y712I_{—} | |
| | H66A_T316W_LL855/6AA | |
| HV1303234 | HIV_CAP256SU_UCA_OPT_4.0_F14(A204V_{—} | FIG. 17 |
| | V208L_V68I_V255L)_RnS3mut2G_{—} | |
| | gp160_IGHVss_deltaG_DS.SOSL.GS_{—} | |
| | I535M.PC._Y712I_H66A_T316W_LL855/6AA | |
| HV1303235 | HIV_CAP256SU_UCA_OPT_4.0 | FIG. 17 |
| | F14(A204V_V208L_V68I_V2SSL)_IGHVss_{—} | |
| | deltaG_SOSL.GS_I535M_gp145.712 | |
| HV1303236 | HIV_CAP256SU_UCA_OPT_4.0 | FIG. 17 |
| | F14(A204V_V208L_V68I_V255L)_IGHVss_{—} | |
| | deltaG_DS.SOSL.GS_I535M_gp145.712 | |
| HV1303237 | HIV_CAP256SU_UCA_OPT_4.0 | FIG. 17 |
| | F14(A204V_V208L_V68I_V255L)_{—} | |
| | RnS3mut2G_IGHVss_deltaG_DS.SOSL.GS_{—} | |
| | I535M_gp145.712 | |
| HV1303238 | HIV_CAP256SU_UCA_OPT_4.0 | FIG. 17 |
| | F14(A204V_V208L_V68I_V255L)_{—} | |
| | IGHVss_deltaG_DS.SOSL.GS_{—} | |
| | I535M.PC.H66A_T316W_gp145.712 | |
| HV1303239 | HIV_CAP256SU_UCA_OPT_4.0F14(A204V_{—} | FIG. 17 |
| | V208L_V68I_V255L)_RnS3mut2G_{—} | |
| | IGHVss_deltaG_DS.SOSL.GS_{—} | |
| | I535M.PC.H66A_T316W_gp145.712 | |
| HV1303240 | HIV_CAP256SU_UCA_OPT_4.0 | FIG. 17 |
| | F14(A204V_V208L_V68I_V255L)_{—} | |
| | IGHVss_deltaG_SOSL.GS_I535M_{—} | |
| | Y712I_gp150.755 | |
| HV1303241 | HIV_CAP256SU_UCA_OPT_4.0 | FIG. 17 |
| | F14(A204V_V208L_V68I_V255L)_{—} | |
| | IGHVss_deltaG_DS.SOSL.GS_I535M_{—} | |
| | Y712I_gp150.755 | |
| HV1303242 | HIV_CAP256SU_UCA_OPT_4.0 | FIG. 17 |
| | F14(A204V_V208L_V68I_V255L)_{—} | |
| | RnS3mut2G_IGHVss_deltaG_DS.SOSL.GS_{—} | |
| | I535M_Y712I_gp150.755 | |
| HV1303243 | HIV_CAP256SU_UCA_OPT_4.0 | FIG. 17 |
| | F14(A204V_V208L_V68I_V255L)_{—} | |
| | IGHVss_deltaG_DS.SOSL.GS_I535M.PC._{—} | |
| | Y712I_H66A_T316W_gp150.755 | |
| HV1303244 | HIV_CAP256SU_UCA_OPT_4.0F14(A204V_{—} | FIG. 17 |
| | V208L_V68I_V255L)_RnS3mut2G_{—} | |
| | IGHVss_deltaG_DS.SOSL.GS_I535M.PC._{—} | |
| | Y712I_H66A_T316W_gp150.755 | |
| HV1303245 | HIV_CAP256SU_UCA_OPT_4.0 | FIG. 17 |
| | F14(A204V_V208L_V68I_V255L)_{—} | |
| | IGHVss_deltaG_SOSL.GS_I535M_Y712I_{—} | |
| | gp150.750 | |
| HV1303246 | HIV_CAP256SU_UCA_OPT_4.0 | FIG. 17 |
| | F14(A204V_V208L_V68I_V255L)_{—} | |
| | IGHVss_deltaG_DS.SOSL.GS_I535M_{—} | |
| | Y712I_gp150.750 | |
| HV1303247 | HIV_CAP256SU_UCA_OPT_4.0 | FIG. 17 |
| | F14(A204V_V208L_V68I_V255L)_{—} | |
| | RnS3mut2G_IGHVss_deltaG_DS.SOSL.GS_{—} | |
| | I535M_Y712I_gp150.750 | |
| HV1303248 | HIV_CAP256SU_UCA_OPT_4.0 | FIG. 17 |
| | F14(A204V_V208L_V68I_V255L)_{—} | |
| | IGHVss_deltaG_DS.SOSL.GS_I535M.PC._{—} | |
| | Y712I_H66A_T316W_gp150.750 | |
| HV1303249 | HIV_CAP256SU_UCA_OPT_4.0F14(A204V_{—} | FIG. 17 |
| | V208L_V68I_V255L)_RnS3mut2G_{—} | |
| | IGHVss_deltaG_DS.SOSL.GS_I535M.PC._{—} | |
| | Y712I_H66A_T316W_gp150.750 | |
| HV1303250 | HIV_CAP256SU_UCA_OPT_4.0 | FIG. 17 |
| | F14(A204V_V208L_V68I_V255L)_gp160_{—} | |
| | BPrlss_deltaG_SOSL.GS_I535M_Y712I | |
| HV1303251 | HIV_CAP256SU_UCA_OPT_4.0 | FIG. 17 |
| | F14(A204V_V208L_V68I_V255L)_BPrlss_{—} | |
| | deltaG_SOSL.GS_I535M_gp145.712 | |
| HV1303252 | HIV_CAP256SU_UCA_OPT_4.0 | FIG. 17 |
| | F14(A204V_V208L_V68I_V255L)_BPrlss_{—} | |
| | deltaG_SOSL.GS_I535M_Y712I_{—} | |
| | gp150.755 | |
| HV1303253 | HIV_CAP256SU_UCA_OPT_4.0 | FIG. 17 |
| | F14(A204V_V208L_V68I_V255L)_BPrlss_{—} | |
| | deltaG_SOSL.GS_I535M_Y712I_{—} | |
| | gp150.750 | |
| HV1303254 | HIV_CAP256SU_UCA_OPT_4.0 | FIG. 17 |
| | F14(A204V_V208L_V68I_V255L)_gp160_{—} | |
| | BPrlss_deltaG_DS.SOSL.GS_I535M_{—} | |
| | Y712I_LL855/6AA | |
| HV1303049 | CAP256.wk34.c80 SOSIP.RnS2 | FIG. 17 |
| HV1303050 | CAP256.wk34.c80_V2UCAOPT_v3.0 | FIG. 17 |
| | SOSIP.RnS2 | |
| | Q23.17_V2UCAOPT | FIG. 17 |
| | Q23.17_V2UCAOPT_GLY | FIG. 17 |
| | Q23.17_V2UCAOPT_ALT | FIG. 17 |
| | Q23.17_V2UCAOPT_GLY_ALT | FIG. 17 |
| HV1301552 | A.Q23_17CHIM.SOSIPV5.2.8/293F | FIG. 17 |

FIG. 5 shows non-limiting embodiments of multimerization designs, including ferritin and/or sortase, which could be used as guidance to design any of the envelopes in Table 2 or 4 as multimeric designs. In FIGS. 4C-D, CH505 V2UCAOPT.ver2 envelope sequence is shown as a gp160 envelope. This V2 optimized design could be used as the basis to design any suitable protomer, wherein in non-limiting embodiments the protomer can form a stabilized trimer. Non-limiting designs of envelope protomers include SOSIP designs, designs comprising F14 mutations (See US Pub 20210379177, incorporated herein by reference), and so forth.

Table 3 shows non-limiting embodiments of optimized immunogens-sortase design, See FIG. 14.

| Plasmid ID | |
|---|---|
| gene number | Protein name |
| HV1302426 | T250_V2UCAOPT_v3.DS.SOSIP_TPAss_cSorta |
| HV1302427 | T250_V2UCAOPT_v3.DS.SOSIP_CDSss_cSorta |
| HV1302428 | T250_V2UCAOPT_v3.DS.SOSIP_Abss_cSorta |
| HV1302429 | CH505_V2UCAOPT_v3.0_cSORTA |
| HV1302430 | CAP256SU_V2UCAOPT_v3.0.DS.6R.SOSIP.664_TPAss_cSORTA |
| HV1302431 | CAP256SU_V2UCAOPT_v3.0.DS.6R.SOSIP.664_CDSss_cSORTA |
| HV1302432 | CAP256SU_V2UCAOPT_v3.0.DS.6R.SOSIP.664_mVHss_cSORTA |
| HV1302433 | CAP256SU_V2UCAOPT_v3.0.DS.6R.SOSIPv6.664_mVHss_cSORTA |
| HV1302434 | CAP256SU_V2UCAOPT_v3.0.DS.6R.SOSIPv6.664_CD5ss_cSORTA |
| HV1302435 | CAP256SU_V2UCAOPT_v3.0.DS.6R.SOSIPv6.664_TPAss_cSORTA |
| HV1302436 | CAM13.RRK.6R.DS.SOSIPgp140.664.CD5ss_opt_cSORTA |
| HV1302437 | CAM13.RRK.6R.DS.SOSIP.UFO_cSORTA |
| HV1302438 | CAM13.RRK.6R.DS.SOSIPv6_cSORTA |

The trimer could be incorporated in a nanoparticle, including without limitation any ferritin based nanoparticle.

Throughout the application amino acid positions numbers refer to HXB2 numbering.

Any of the immunogens herein may be encoded by a nucleic acid. FIGS. 4A, 4D, 5A, 5B, 5E, 14 and 17 provide non-limiting examples of nucleic acids encoding an immunogen. In certain embodiments, the nucleic acid may be a DNA, an RNA, or an mRNA. Non-limiting examples of mRNA nucleic acids encoding an immunogen of the present technology include, but are not limited to, 2560_pUC-ccTEV-co.mRNAI_CAP256SU_UCA_OPT_4.0.A101, 2560_pUC-ccTEV-co.mRNA2_CAP256SU_UCA_OPT_4.0A101, 2560_pUC-ccTEV-coA.Q23_17CHIM.SOSIPV5.2.8 293F (HV1301552)-A101, 2560_pUC-ccTEV-coCAP256.wk34.c80 SOSIP.RnS2 (HV1303049)-A101, 2560_pUC-ccTEV-coCAP256.wk34.c80_V2UCAOPT_v3.0 SOSIP.RnS2 (HV1303050)-A101, 2560_pUC-ccTEV-coHV1303230-A101, 2560_pUC-ccTEV-coHV1303231-A101, 2560_pUC-ccTEV-coHV1303232-A101, 2560_pUC-ccTEV-coHV1303233-A101, 2560_pUC-ccTEV-coHV1303234-A101, 2560_pUC-ccTEV-coHV1303235-A101, 2560_pUC-ccTEV-coHV1303236-A101, 2560_pUC-ccTEV-coHV1303237-A101, 2560_pUC-ccTEV-coHV1303238-A101, 2560_pUC-ccTEV-coHV1303239-A101, 2560_pUC-ccTEV-coHV1303240-A101, 2560_pUC-ccTEV-coHV1303241-A101, 2560_pUC-ccTEV-coHV1303242-A101, 2560_pUC-ccTEV-coHV1303243-A101, 2560_pUC-ccTEV-coHV1303244-A101, 2560_pUC-ccTEV-coHV1303245-A101, 2560_pUC-ccTEV-coHV1303246-A101, 2560_pUC-ccTEV-coHV1303247-A101, 2560_pUC-ccTEV-coHV1303248-A101, 2560_pUC-ccTEV-coHV1303249-A101, 2560_pUC-ccTEV-coHV1303250-A101, 2560_pUC-ccTEV-coHV1303251-A101, 2560_pUC-ccTEV-coHV1303252-A101, 2560 pUC-ccTEV-coHV1303253-A101, 2560_pUC-ccTEV-coHV1303254-A101, 2560_pUC-ccTEV-coHV1303326,A.Q23.6R.DS.SOS.GS.I535M.K658Q_E659D_Igss-A101, 2560_pUC-ccTEV-coHV1303327,A.Q23.DS.SOSL.GS.1535M.K658Q_E659D_Igss-A101, 2560_pUC-ccTEV-coHV1303328,A.Q23.6R.DS.SOS.GS.I535M.K658Q_E659D_Igss-A101, and 2560_pUC-ccTEV-coHV1303329,A.Q23.DS.SOSL.GS.1535M.K658Q_E659D_Igss-A101. It will be understood that non-identical nucleic acid sequences may encode the same amino acid sequence. As such these examples do not exclude nucleic acid sequences that encode immunogens with the same amino acid sequence but possess different nucleic acid sequences.

Exemplary constructs are provided in Table 4.

| | | Soluble or | Stabilization/ | | Furin | |
|---|---|---|---|---|---|---|
| | | membrane | Expression | Signal | Cleavage | Cytoplasmic |
| Construct | Description | anchored | mutations | Peptide | Site | tail (CT) |
| HIV_CAP256SU_{—} | Full length | Membrane | None | Wildtype | Wildtype | Wildtype |
| UCA_OPT_4.0 | Env | anchored | | | | |
| mmal_CAP256SU_{—} | Protein | Membrane | Sodroski; | Signal | Glycine- | SIVMac CT |
| UCA_OPT_4.0 | | anchored | Y712I | peptide #1 | Serine | RPVFSSPPSYFQ |
| | | | | MAISGV | linker #1 | |
| | | | | PVLGFFI | | |
| | | | | IAVLMS | | |
| | | | | AQESWA | | |
| mma2_CAP256SU_{—} | Protein | Membrane | Sodroski; | Signal | Glycine- | SIVMac CT |
| UCA_OPT_4.0 | | anchored | Y712I; F14 | peptide #1 | Serine | RPVFSSPPSYFQ |
| | | | | MAISGV | linker #1 | |
| | | | | PVLGFFI | | |
| | | | | IAVLMS | | |
| | | | | AQESWA | | |
| CAP256.wk34.c80_{—} | Protein | Soluble | SOSIP; DS; | IGHVss | RRRRRR | N/A |
| V2UCAOPT_v3.0 | | | 3mut; 2G; | | | |
| SOSIP.RnS2(HV1303050) | | | RnS | | | |
| HV1303230, HIV_{—} | Protein | Membrane | F14, deltaG, SOS, | IGHVss | Glycine- | Wildtype |
| CAP256SU_UCA_OPT_4.0 | | anchored | GS, I535M, | | Serine | |
| F14(A204V_V208L_{—} | | | Y712I | | linker #1 | |
| V68I_V255L)_gp160_{—} | | | | | (also | |
| IGHVss_deltaG_{—} | | | | | labeled | |
| SOSL.GS_I535M_Y712I | | | | | as “L”) | |
| HV1303231, HIV_{—} | Protein | Membrane | F14, deltaG, | IGHVss | Glycine- | Wildtype |
| CAP256SU_UCA_OPT_4.0 | | anchored | SOS, GS, | | Serine | |
| F14(A204V_V208L_{—} | | | I535M, Y712I, | | linker #1 | |
| V68I_V255L)_gp160_{—} | | | LL855/6AA | | (also | |
| IGHVss_deltaG_{—} | | | | | labeled | |
| DS.SOSL.GS_I535M_{—} | | | | | as “L”) | |
| Y712I_LL855/6AA | | | | | | |
| HV1303232, HIV_{—} | Protein | Membrane | F14, RnS, | IGHVss | Glycine- | Wildtype |
| CAP256SU_UCA_OPT_4.0 | | anchored | 3mut, 2G, | | Serine | |
| F14(A204V_V208L_{—} | | | deltaG, SOS, | | linker #1 | |
| V68I_V255L)_{—} | | | GS, I535M, | | (also | |
| RnS3mut2G_gp160_{—} | | | Y712I, | | labeled | |
| IGHVss_deltaG_{—} | | | LL855/6AA | | as “L”) | |
| DS.SOSL.GS_I535M_{—} | | | | | | |
| Y712I_LL855/6AA | | | | | | |
| HV1303233, HIV_{—} | Protein | Membrane | F14, deltaG, | IGHVss | Glycine- | Wildtype |
| CAP256SU_UCA_OPT_{—} | | anchored | DS, SOS, GS, | | Serine | |
| 4.0 F14(A204V_{—} | | | I535M, PC,, | | linker #1 | |
| V208L_V68I_V255L)_{—} | | | Y712I, H66A, | | (also | |
| gp160_IGHVss_{—} | | | T316W, | | labeled | |
| deltaG_DS.SOSL.GS_{—} | | | LL855/6AA | | as “L”) | |
| I535M.PC._Y712I_{—} | | | | | | |
| H66A_T316W_LL855/ | | | | | | |
| 6AA | | | | | | |
| HV1303234, HIV_{—} | Protein | Membrane | F14, RnS, | IGHVss | Glycine- | Wildtype |
| CAP256SU_UCA_OPT_{—} | | anchored | 3mut, 2G, | | Serine | |
| 4.0_F14(A204V_{—} | | | deltaG, DS, | | linker #1 | |
| V208L_V68I_V255L)_{—} | | | SOS, GS, | | (also | |
| RS3mut2G_gp160_{—} | | | I535M, PC,, | | labeled | |
| IGHVss_deltaG_{—} | | | Y712I, H66A, | | as “L”) | |
| DS.SOSL.GS_I535M.PC._{—} | | | T316W, | | | |
| Y712I_H66A_T316W_{—} | | | LL855/6AA | | | |
| LL855/6AA | | | | | | |
| HV1303235, HIV_{—} | Protein | Membrane | F14, deltaG, | IGHVss | Glycine- | Truncated |
| CAP256SU_UCA_OPT_4.0 | | anchored | SOS, GS, | | Serine | beyond HXB2 |
| F14(A204V_V208L_{—} | | | I535M | | linker #1 | position 712 |
| V68I_V255L)_IGHVss_{—} | | | | | (also | |
| deltaG_SOSL.GS | | | | | labeled | |
| I535M_gp145.712 | | | | | as “L”) | |
| HV1303236, HIV_{—} | Protein | Membrane | F14, deltaG, | IGHVss | Glycine- | Truncated |
| CAP256SU_UCA_OPT_4.0 | | anchored | DS, SOS, GS, | | Serine | beyond HXB2 |
| F14(A204V_V208L_{—} | | | I535M | | linker #1 | position 712 |
| V68I_V255L)_IGHVss_{—} | | | | | (also | |
| deltaG_DS.SOSL.GS_{—} | | | | | labeled | |
| I535M_gp145.712 | | | | | as “L”) | |
| HV1303237, HIV_{—} | Protein | Membrane | F14, RnS, | IGHVss | Glycine- | Truncated |
| CAP256SU_UCA_OPT_4.0 | | anchored | 3mut, 2G, | | Serine | beyond HXB2 |
| F14(A204V_V208L_{—} | | | deltaG, DS, | | linker #1 | position 712 |
| V68I_V255L)_RnS3mut2G_{—} | | | SOS, GS, | | (also | |
| IGHVss_deltaG_{—} | | | I535M | | labeled | |
| DS.SOSL.GS_I535M_{—} | | | | | as “L”) | |
| gp145.712 | | | | | | |
| HV1303238, HIV_{—} | Protein | Membrane | F14, deltaG, | IGHVss | Glycine- | Truncated |
| CAP256SU_UCA_OPT_4.0 | | anchored | DS, SOS, GS, | | Serine | beyond HXB2 |
| F14(A204V_V208L_{—} | | | I535M, PC, | | linker #1 | position 712 |
| V68I_V255L)_IGHVss_{—} | | | H66A, T316W | | (also | |
| deltaG_DS.SOSL.GS_{—} | | | | | labeled | |
| I535M.PC.H66A_{—} | | | | | as “L”) | |
| T316W_gp145.712 | | | | | | |
| HV1303239, HIV_{—} | Protein | Membrane | F14, RnS, | IGHVss | Glycine- | Truncated |
| CAP256SU_UCA_OPT_{—} | | anchored | 3mut, 2G, | | Serine | beyond HXB2 |
| 4.0F14(A204V_V208L_{—} | | | deltaG, DS, | | linker #1 | position 712 |
| V68I_V255L)_{—} | | | SOS, GS, | | (also | |
| RnS3mut2G_IGHVss_{—} | | | I535M, PC, | | labeled | |
| deltaG_DS.SOSL.GS_{—} | | | H66A, T316W | | as “L”) | |
| I535M.PC.H66A_{—} | | | | | | |
| T316W_gp145.712 | | | | | | |
| HV1303240, HIV_{—} | Protein | Membrane | F14, deltaG, | IGHVss | Glycine- | Truncated |
| CAP256SU_UCA_OPT_4.0 | | anchored | SOS, GS, | | Serine | beyond HXB2 |
| F14(A204V_V208L_{—} | | | I535M, Y712I | | linker #1 | position 755 |
| V68I_V255L)_IGHVss_{—} | | | | | (also | |
| deltaG_SOSL.GS_{—} | | | | | labeled | |
| I535M_Y712I_gp150.755 | | | | | as “L”) | |
| HV1303241, HIV_{—} | Protein | Membrane | F14, deltaG, | IGHVss | Glycine- | Truncated |
| CAP256SU_UCA_OPT_4.0 | | anchored | DS, SOS, GS, | | Serine | beyond HXB2 |
| F14(A204V_V208L_{—} | | | I535M, Y712I | | linker #1 | position 755 |
| V68I_V255L)_IGHVss_{—} | | | | | (also | |
| deltaG_DS.SOSL.GS_{—} | | | | | labeled | |
| I535M_Y712I_gp150.755 | | | | | as “L”) | |
| HV1303242, HIV_{—} | Protein | Membrane | F14, RnS, | IGHVss | Glycine- | Truncated |
| CAP256SU_UCA_OPT_4.0 | | anchored | 3mut, 2G, | | Serine | beyond HXB2 |
| F14(A204V_V208L_{—} | | | deltaG, DS, | | linker #1 | position 755 |
| V68I_V255L)_RnS3mut2G_{—} | | | SOS, GS, | | (also | |
| IGHVss_deltaG_{—} | | | I535M, Y712I | | labeled | |
| DS.SOSL.GS_I535M_{—} | | | | | as “L”) | |
| Y712I_gp150.755 | | | | | | |
| HV1303243, HIV_{—} | Protein | Membrane | F14, deltaG, | IGHVss | Glycine- | Truncated |
| CAP256SU_UCA_OPT_4.0 | | anchored | DS, SOS, GS, | | Serine | beyond HXB2 |
| F14(A204V_V208L_{—} | | | I535M, PC,, | | linker #1 | position 755 |
| V68I_V255L)_IGHVss_{—} | | | Y712I, H66A, | | (also | |
| deltaG_DS.SOSL.GS_{—} | | | T316W | | labeled | |
| I535M.PC._Y712I_{—} | | | | | as “L”) | |
| H66A_T316W_gp150.755 | | | | | | |
| HV1303244, HIV_{—} | Protein | Membrane | F14, RnS, | IGHVss | Glycine- | Truncated |
| CAP256SU_UCA_OPT_{—} | | anchored | 3mut, 2G, | | Serine | beyond HXB2 |
| 4.0F14(A204V_V208L_{—} | | | deltaG, DS, | | linker #1 | position 755 |
| V68I_V255L)_{—} | | | SOS, GS, | | (also | |
| RnS3mut2G_IGHVss_{—} | | | I535M, PC,, | | labeled | |
| deltaG_DS.SOSL.GS_{—} | | | Y712I, H66A, | | as “L”) | |
| I535M.PC._Y712I_{—} | | | T316W | | | |
| H66A_T316W_gp150.755 | | | | | | |
| HV1303245, HIV_{—} | Protein | Membrane | F14, deltaG, | IGHVss | Glycine- | Truncated |
| CAP256SU_UCA_OPT_4.0 | | anchored | SOS, GS, | | Serine | beyond HXB2 |
| F14(A204V_V208L_{—} | | | I535M, Y712I | | linker #1 | position 750 |
| V68I_V255L)_IGHVss_{—} | | | | | (also | |
| deltaG_SOSL.GS_{—} | | | | | labeled | |
| I535M_Y712I_gp150.750 | | | | | as “L”) | |
| HV1303246, HIV_{—} | Protein | Membrane | F14, deltaG, | IGHVss | Glycine- | Truncated |
| CAP256SU_UCA_OPT_4.0 | | anchored | DS, SOS, GS, | | Serine | beyond HXB2 |
| F14(A204V_V208L_{—} | | | I535M, Y712I | | linker #1 | position 750 |
| V68I_V255L)_IGHVss_{—} | | | | | (also | |
| deltaG_DS.SOSL.GS_{—} | | | | | labeled | |
| I535M_Y712I_gp150.750 | | | | | as “L”) | |
| HV1303247, HIV_{—} | Protein | Membrane | F14, RnS, | IGHVss | Glycine- | Truncated |
| CAP256SU_UCA_OPT_4.0 | | anchored | 3mut, 2G, | | Serine | beyond HXB2 |
| F14(A204V_V208L_{—} | | | deltaG, DS, | | linker #1 | position 750 |
| V68I_V255L)_RnS3mut2G_{—} | | | SOS, GS, | | (also | |
| IGHVss_deltaG_{—} | | | I535M, Y712I | | labeled | |
| DS.SOSL.GS_I535M_{—} | | | | | as “L”) | |
| Y712I_gp150.750 | | | | | | |
| HV1303248, HIV_{—} | Protein | Membrane | F14, deltaG, | IGHVss | Glycine- | Truncated |
| CAP256SU_UCA_OPT_4.0 | | anchored | DS, SOS, GS, | | Serine | beyond HXB2 |
| F14(A204V_V208L_{—} | | | I535M, PC,, | | linker #1 | position 750 |
| V68I_V255L)_IGHVss_{—} | | | Y712I, H66A, | | (also | |
| deltaG_DS.SOSL.GS_{—} | | | T316W | | labeled | |
| I535M.PC._Y712I_{—} | | | | | as “L”) | |
| H66A_T316W_gp150.750 | | | | | | |
| HV1303249, HIV_{—} | Protein | Membrane | F14, RnS, | IGHVss | Glycine- | Truncated |
| CAP256SU_UCA_OPT_{—} | | anchored | 3mut, 2G, | | Serine | beyond HXB2 |
| 4.0F14(A204V_V208L_{—} | | | deltaG, DS, | | linker #1 | position 750 |
| V68I_V255L)_{—} | | | SOS, GS, | | (also | |
| RnS3mut2G_IGHVss_{—} | | | I535M, PC, | | labeled | |
| deltaG_DS.SOSL.GS_{—} | | | Y712I, H66A, | | as “L”) | |
| I535M.PC._Y712I_{—} | | | T316W | | | |
| H66A_T316W_gp150.750 | | | | | | |
| HV1303250, HIV_{—} | Protein | Membrane | F14, deltaG, | BPrlss | Glycine- | Truncated |
| CAP256SU_UCA_OPT_4.0 | | anchored | SOS, GS, | | Serine | beyond HXB2 |
| F14(A204V_V208L_{—} | | | I535M, Y712I | | linker #1 | position 712 |
| V68I_V255L)_gp160_{—} | | | | | (also | |
| BPrlss_deltaG_{—} | | | | | labeled | |
| SOSL.GS_I535M_Y712I | | | | | as “L”) | |
| HV1303251, HIV_{—} | Protein | Membrane | F14, deltaG, | BPrlss | Glycine- | Truncated |
| CAP256SU_UCA_OPT_4.0 | | anchored | SOS, GS, | | Serine | beyond HXB2 |
| F14(A204V_V208L_{—} | | | I535M | | linker #1 | position 712 |
| V68I_V255L)_BPrlss_{—} | | | | | (also | |
| deltaG_SOSL.GS_{—} | | | | | labeled | |
| I535M_gp145.712 | | | | | as “L”) | |
| HV1303252, HIV_{—} | Protein | Membrane | F14, deltaG, | BPrlss | Glycine- | Truncated |
| CAP256SU_UCA_OPT_4.0 | | anchored | SOS, GS, | | Serine | beyond HXB2 |
| F14(A204V_V208L_{—} | | | I535M, Y712I | | linker #1 | position 755 |
| V68I_V255L)_BPrlss_{—} | | | | | (also | |
| deltaG_SOSL.GS_{—} | | | | | labeled | |
| I535M_Y712I_gp150.755 | | | | | as “L”) | |
| HV1303253, HIV_{—} | Protein | Membrane | F14, deltaG, | BPrlss | Glycine- | Truncated |
| CAP256SU_UCA_OPT_4.0 | | anchored | SOS, GS, | | Serine | beyond HXB2 |
| F14(A204V_V208L_{—} | | | I535M, Y712I | | linker #1 | position 750 |
| V68I_V255L)_BPrlss_{—} | | | | | (also | |
| deltaG_SOSL.GS_{—} | | | | | labeled | |
| I535M_Y712I_gp150.750 | | | | | as “L”) | |
| HV1303254, HIV_{—} | Protein | Membrane | F14, deltaG, | BPrlss | Glycine- | Wildtype |
| CAP256SU_UCA_OPT_4.0 | | anchored | DS, SOS, GS, | | Serine | |
| F14(A204V_V208L_{—} | | | I535M, Y712I, | | linker #1 | |
| V68I_V255L)_gp160_{—} | | | LL855/6AA | | (also | |
| BPrlss_deltaG_{—} | | | | | labeled | |
| DS.SOSL.GS_I535M_{—} | | | | | as “L”) | |
| Y712I_LL855/6AA | | | | | | |
| HV1303326, A.Q23.6R. | Protein | Soluble | 6R; DS; SOS; | IGHVss | RRRRRR | N/A |
| DS.SOS.GS.I535M. | | | GS; I535M; | | | |
| K658Q_E659D_Igss | | | K658Q; | | | |
| | | | E659D; Igss | | | |
| HV1303327, A.Q23. | Protein | Soluble | DS; SOSL; GS; | IGHVss | Glycine- | N/A |
| DS.SOSL.GS.I535M. | | | I535M; K658Q; | | Serine | |
| K658Q_E659D_Igss | | | E659D; Igss | | linker #1 | |
| | | | | | (also | |
| | | | | | labeled | |
| | | | | | as ‘L’) | |
| HV1303328, A.Q23.6R. | Protein | Soluble | 6R; DS; SOS; | IGHVss | RRRRRR | N/A |
| DS.SOS.GS.I535M. | | | GS; I535M; | | | |
| K658Q_E659D_Igss_cSorta | | | K658Q; E659D; | | | |
| | | | Igss; cSorta | | | |
| HV1303329, A.Q23. | Protein | Soluble | DS; SOSL; GS; | IGHVss | Glycine- | N/A |
| DS.SOSL.GS.I535M. | | | I535M; K658Q; | | Serine | |
| K658Q_E659D_Igss_cSorta | | | E659D; Igss; | | linker #1 | |
| | | | cSorta | | (also | |
| | | | | | labeled | |
| | | | | | as ‘L’) | |
| 2560_pUC-ccTEV- | mRNA | Membrane | mRNA immunogen | See protein | See protein | See protein |
| co.mRNA1_CAP256SU_{—} | | anchored | construct for | construct | construct | construct |
| UCA_OPT_4.0.A101 | | | mma1_CAP256SU | above. | above. | above. |
| | | | UCA_OPT_4.0. | | | |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV- | mRNA | Membrane | mRNA immunogen | See protein | See protein | See protein |
| co.mRNA2_CAP256SU_{—} | | anchored | construct for | construct | construct | construct |
| UCA_OPT_4.0A101 | | | mrna2_CAP256SU_{—} | above. | above. | above. |
| | | | UCA_OPT_4.0. | | | |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV-coA.Q23_{—} | mRNA | Membrane | mRNA immunogen | See protein | See protein | See protein |
| 17CHIM.SOSIPV5.2.8_293F | | anchored | construct for Q23_{—} | construct | construct | construct |
| (HV1301552)-A101 | | | 17CHIM.SOSIPV5.2.8 | above. | above. | above. |
| | | | 293F (HV1301552). | | | |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV- | mRNA | Membrane | mRNA immunogen | See protein | See protein | See protein |
| coCAP256.wk34.c80 | | anchored | construct for | construct | construct | construct |
| SOSIP.RnS2 | | | CAP256.wk34.c80 | above. | above. | above. |
| (HV1303049)-A101 | | | SOSIP.RnS2 | | | |
| | | | (HV1303049). | | | |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV- | mRNA | Membrane | mRNA immunogen | See protein | See protein | See protein |
| coCAP256.wk34.c80_{—} | | anchored | construct for | construct | construct | construct |
| V2UCAOPT_v3.0 | | | CAP256.wk34.c80_{—} | above. | above. | above. |
| SOSIP.RnS2(HV1303050)- | | | V2UCAOPT_v3.0 | | | |
| A101 | | | SOSIP.RnS2 | | | |
| | | | (HV1303050). | | | |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV- | mRNA | Membrane | mRNA immunogen | See protein | See protein | See protein |
| coHV1303230-A101 | | anchored | construct for | construct | construct | construct |
| | | | HV1303230 above. | above. | above. | above. |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV- | mRNA | Membrane | mRNA immunogen | See protein | See protein | See protein |
| coHV1303231-A101 | | anchored | construct for | construct | construct | construct |
| | | | HV1303231 above. | above. | above. | above. |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV- | mRNA | Membrane | mRNA immunogen | See protein | See protein | See protein |
| coHV1303232-A101 | | anchored | construct for | construct | construct | construct |
| | | | HV1303232 above. | above. | above. | above. |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV- | mRNA | Membrane | mRNA immunogen | See protein | See protein | See protein |
| coHV1303233-A101 | | anchored | construct for | construct | construct | construct |
| | | | HV1303233 above. | above. | above. | above. |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV- | mRNA | Membrane | mRNA immunogen | See protein | See protein | See protein |
| coHV1303234-A101 | | anchored | construct for | construct | construct | construct |
| | | | HV1303234 above. | above. | above. | above. |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV- | mRNA | Membrane | mRNA immunogen | See protein | See protein | See protein |
| coHV1303235-A101 | | anchored | construct for | construct | construct | construct |
| | | | HV1303235 above. | above. | above. | above. |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV- | mRNA | Membrane | mRNA immunogen | See protein | See protein | See protein |
| coHV1303236-A101 | | anchored | construct for | construct | construct | construct |
| | | | HV1303236 above. | above. | above. | above. |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV- | mRNA | Membrane | mRNA immunogen | See protein | See protein | See protein |
| coHV1303237-A101 | | anchored | construct for | construct | construct | construct |
| | | | HV1303237 above. | above. | above. | above. |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV- | mRNA | Membrane | mRNA immunogen | See protein | See protein | See protein |
| coHV1303238-A101 | | anchored | construct for | construct | construct | construct |
| | | | HV1303238 above. | above. | above. | above. |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV- | mRNA | Membrane | mRNA immunogen | See protein | See protein | See protein |
| coHV1303239-A101 | | anchored | construct for | construct | construct | construct |
| | | | HV1303239 above. | above. | above. | above. |
| | | | Modifications; | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV- | mRNA | Membrane | mRNA immunogen | See protein | See protein | See protein |
| coHV1303240-A101 | | anchored | construct for | construct | construct | construct |
| | | | HV1303240 above. | above. | above. | above. |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV- | mRNA | Membrane | mRNA immunogen | See protein | See protein | See protein |
| coHV1303241-A101 | | anchored | construct for | construct | construct | construct |
| | | | HV1303241 above. | above. | above. | above. |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV- | mRNA | Membrane | mRNA immunogen | See protein | See protein | See protein |
| coHV1303242-A101 | | anchored | construct for | construct | construct | construct |
| | | | HV1303242 above. | above. | above. | above. |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV- | mRNA | Membrane | mRNA immunogen | See protein | See protein | See protein |
| coHV1303243-A101 | | anchored | construct for | construct | construct | construct |
| | | | HV1303243 above. | above. | above. | above. |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV- | mRNA | Membrane | mRNA immunogen | See protein | See protein | See protein |
| coHV1303244-A101 | | anchored | construct for | construct | construct | construct |
| | | | HV1303244 above. | above. | above. | above. |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV- | mRNA | Membrane | mRNA immunogen | See protein | See protein | See protein |
| coHV1303245-A101 | | anchored | construct for | construct | construct | construct |
| | | | HV1303245 above. | above. | above. | above. |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV- | mRNA | Membrane | mRNA immunogen | See protein | See protein | See protein |
| coHV1303246-A101 | | anchored | construct for | construct | construct | construct |
| | | | HV1303246 above. | above. | above. | above. |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV- | mRNA | Membrane | mRNA immunogen | See protein | See protein | See protein |
| coHV1303247-A101 | | anchored | construct for | construct | construct | construct |
| | | | HV1303247 above. | above. | above. | above. |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV- | mRNA | Membrane | mRNA immunogen | Sec protein | See protein | See protein |
| coHV1303248-A101 | | anchored | construct for | construct | construct | construct |
| | | | HV1303248 above. | above. | above. | above. |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV- | mRNA | Membrane | mRNA immunogen | See protein | See protein | See protein |
| coHV1303249-A101 | | anchored | construct for | construct | construct | construct |
| | | | HV1303249 above. | above. | above. | above. |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV- | mRNA | Membrane | mRNA immunogen | See protein | See protein | See protein |
| coHV1303250-A101 | | anchored | construct for | construct | construct | construct |
| | | | HV1303250 above. | above. | above. | above. |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV- | mRNA | Membrane | mRNA immunogen | See protein | See protein | See protein |
| coHV1303251-A101 | | anchored | construct for | construct | construct | construct |
| | | | HV1303251 above. | above. | above. | above. |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV- | mRNA | Membrane | mRNA immunogen | See protein | See protein | See protein |
| coHV1303252-A101 | | anchored | construct for | construct | construct | construct |
| | | | HV1303252 above. | above. | above. | above. |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV- | mRNA | Membrane | mRNA immunogen | See protein | See protein | See protein |
| coHV1303253-A101 | | anchored | construct for | construct | construct | construct |
| | | | HV1303253 above. | above. | above. | above. |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV- | mRNA | Membrane | mRNA immunogen | See protein | See protein | See protein |
| coHV1303254-A101 | | anchored | construct for | construct | construct | construct |
| | | | HV1303254 above. | above. | above. | above. |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV- | mRNA | Membrane | mRNA immunogen | See protein | See protein | See protein |
| coHV1303326, A.Q23. | | anchored | construct for | construct | construct | construct |
| 6R.DS.SOS.GS.I535M. | | | HV1303326, | above. | above. | above. |
| K658Q_E659D_Igss-A101 | | | A.Q23.6R.DS. | | | |
| | | | SOS.GS.I535M. | | | |
| | | | K658Q_E659D. | | | |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV- | mRNA | Membrane | mRNA immunogen | See protein | See protein | See protein |
| coHV1303327, | | anchored | construct for | construct | construct | construct |
| A.Q23.DS.SOSL.GS. | | | HV1303327, | above. | above. | above. |
| I535M.K658Q_E659D_{—} | | | A.Q23.DS.SOSL. | | | |
| Igss-A101 | | | GS.I535M.K658Q_{—} | | | |
| | | | E659D. | | | |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV- | mRNA | Membrane | mRNA immunogen | See protein | See protein | See protein |
| coHV1303328, | | anchored | construct | construct | construct | construct |
| A.Q23.6R.DS.SOS.GS. | | | forHV1303328, | above. | above. | above. |
| I535M.K658Q_E659D_{—} | | | A.Q23.6R.DS.SOS. | | | |
| Igss-A101 | | | GS.I535M.K658Q_{—} | | | |
| | | | E659D. | | | |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| 2560_pUC-ccTEV- | mRNA | Membrane | mRNA immunogen | See protein | See protein | See protein |
| coHV1303329, | | anchored | construct for | construct | construct | construct |
| A.Q23.DS.SOSL.GS. | | | HV1303329, | above. | above. | above. |
| I535M.K658Q_E659D_{—} | | | A.Q23.DS.SOSL.GS. | | | |
| Igss-A101 | | | I535M.K658Q_E659D. | | | |
| | | | Modifications: | | | |
| | | | mRNA codon | | | |
| | | | optimization; | | | |
| | | | 2560_pUC-ccTEV- | | | |
| | | | A101 5′UTR; | | | |
| | | | 2560_pUC-ccTEV- | | | |
| | | | A101 3′UTR; | | | |
| | | | Poly A | | | |
| HV1302796, | Protein | Soluble | DS; 6R; SOS; | IGHVss | RRRRRR | N/A |
| CAP256SU_V2UCAOPT_{—} | | | GS; v6; I535M; | | | |
| v3.0.DS.6R.SOS.GS.v6_{—} | | | K658Q; | | | |
| I535M_K658Q_mIgss | | | | | | |
| HV1302797, | Protein | Soluble | DS; 6R; SOS; | IGHVss | RRRRRR | N/A |
| CAP256SU_V2UCAOPT_{—} | | | GS; I535M; | | | |
| v3.0.DS.6R.SOS.GS_{—} | | | K658Q; | | | |
| I535M_K658Q_mIgss | | | | | | |
| HV1302798, | Protein | Soluble | DS; 6R; SOS; | IGHVss | RRRRRR | N/A |
| CAP256SU_V2UCAOPT_{—} | | | UFO; v6; I535M; | | | |
| v3.0.DS.6R.SOS.UFO.v6_{—} | | | K658Q; | | | |
| I535M_K658Q_mIgss | | | | | | |
| HV1302799, | Protein | Soluble | DS; 6R; SOS; | IGHVss | RRRRRR | N/A |
| CAP256SU_V2UCAOPT_{—} | | | UFO; I535M; | | | |
| v3.0.DS.6R.SOS.UFO_{—} | | | K658Q; | | | |
| I535M_K658Q_mIgss | | | | | | |
| HV1302800, | Protein | Soluble | DS; 6R; | CD5ss | RRRRRR | N/A |
| CAP256SU_V2UCAOPT_{—} | | | chimSOSIP; | | | |
| v3.0.DS.6R.chimSOSIP.664_{—} | | | 664; cSORTA | | | |
| CD5ss_cSORTA | | | | | | |
| HV1303209, | Protein | Soluble | SOSIP; RnS2; | IGHVss | RRRRRR | N/A |
| HIV_CAP256.wk34.c80_{—} | | | c-sorta | | | |
| V2UCA_OPT_4.0_{—} | | | | | | |
| SOSIP.RnS2_c-sorta | | | | | | |
| HV1303210, | Protein | Soluble | SOSIP; RnS2; | IGHVss | RRRRRR | N/A |
| HIV_CAP256.wk34.c80_{—} | | | c-sorta | | | |
| V2UCA_OPT_4.0_D167N | | | | | | |
| SOSIP.RnS2_c-sorta | | | | | | |
| HV1303211, | Protein | Soluble | SOSIP; RnS2; | IGHVss | RRRRRR | N/A |
| HIV_CAP256.wk34.c80_{—} | | | c-sorta | | | |
| V2UCA_OPT_4.0_R171K | | | | | | |
| SOSIP.RnS2_c-sorta | | | | | | |
| HV1303212, | Protein | Soluble | SOSIP; RnS2; | IGHVss | RRRRRR | N/A |
| HIV_CAP256.wk34.c80_{—} | | | 10lnQQavi | | | |
| V2UCA_OPT_4.0SOSIP. | | | | | | |
| RnS2_10lnQQavi | | | | | | |
| HV1303213, | Protein | Soluble | SOSIP; RnS2; | IGHVss | RRRRRR | N/A |
| HIV_CAP256.wk34.c80_{—} | | | 10lnQQavi | | | |
| V2UCA_OPT_4.0_D167N | | | | | | |
| SOSIP.RnS2_10lnQQavi | | | | | | |
| HV1303214, | Protein | Soluble | SOSIP; RnS2; | IGHVss | RRRRRR | N/A |
| HIV_CAP256.wk34.c80_{—} | | | 10lnQQavi | | | |
| V2UCA_OPT_4.0_R171K | | | | | | |
| SOSIP.RnS2_10lnQQavi | | | | | | |
| HV1303332, HIV_{—} | Protein | Membrane | F14; RnS; | BPrlss | Glycine- | Truncated |
| CAP256SU_UCA_OPT_{—} | | anchored | 3mut; 2G; | | Serine | beyond HXB2 |
| 4.0F14(A204V_V208L_{—} | | | deltaG; DS; | | linker #1 | position 750 |
| V68I_V255L)_{—} | | | SOSL; GS; | | (also | |
| RnS3mut2G_BPrlss_{—} | | | I535M; PC; | | labeled | |
| deltaG_DS.SOSL.GS_{—} | | | Y712I; H66A; | | as ‘L’) | |
| I535M.PC._Y712I_{—} | | | T316W | | | |
| H66A_T316W_gp150.750 | | | | | | |
| HV1303333, HIV_{—} | Protein | Membrane | F14; deltaG; | IGHVss | Glycine- | Wildtype |
| CAP256SU_UCA_OPT_4.0 | | anchored | SOSL; GS; | | Serine | |
| F14(A204V_V208L_{—} | | | I535M; Y712I | | linker #1 | |
| V68I_V255L)_gp160_{—} | | | | | (also | |
| IGHVss_deltaG_{—} | | | | | labeled | |
| SOSL.GS_I535M_Y712I_{—} | | | | | as ‘L’) | |
| D167N | | | | | | |
| HV1303334, HIV_{—} | Protein | Membrane | F14; deltaG; | IGHVss | Glycine- | Wildtype |
| CAP256SU_UCA_OPT_4.0 | | anchored | DS; SOSL; | | Serine | |
| F14(A204V_V208L_{—} | | | GS; I535M; | | linker #1 | |
| V68I_V255L)_gp160_{—} | | | Y712I; | | (also | |
| IGHVss_deltaG_{—} | | | LL855/6AA | | labeled | |
| DS.SOSL.GS_I535M_{—} | | | | | as ‘L’) | |
| Y712I_D167N_LL855/ | | | | | | |
| 6AA | | | | | | |
| HV1303335, HIV_{—} | Protein | Membrane | F14; RnS; | IGHVss | Glycine- | Wildtype |
| CAP256SU_UCA_OPT_4.0 | | anchored | 3mut; 2G; | | Serine | |
| F14(A204V_V208L_{—} | | | deltaG; DS; | | linker #1 | |
| V68I_V255L)_RnS3mut2G_{—} | | | SOSL; GS; | | (also | |
| gp160_IGHVss_deltaG_{—} | | | I535M; | | labeled | |
| DS.SOSL.GS_I535M_{—} | | | Y712I; | | as ‘L’) | |
| Y712I_D167N_LL855/ | | | LL855/6AA | | | |
| 6AA | | | | | | |
| HV1303336, HIV_{—} | Protein | Membrane | F14; deltaG; | IGHVss | Glycine- | Wildtype |
| CAP256SU_UCA_OPT_4.0 | | anchored | DS; SOSL; | | Serine | |
| F14(A204V_V208L_{—} | | | GS; I535M; | | linker #1 | |
| V68I_V255L)_gp160_{—} | | | PC; Y712I; | | (also | |
| IGHVss_deltaG_{—} | | | H66A; T316W; | | labeled | |
| DS.SOSL.GS_I535M.PC_{—} | | | LL855/6AA | | as ‘L’) | |
| Y712I_D167N_H66A_{—} | | | | | | |
| T316W_LL855/6AA | | | | | | |
| HV1303337, HIV_{—} | Protein | Membrane | F14; RnS; | IGHVss | Glycine- | Wildtype |
| CAP256SU_UCA_OPT_{—} | | anchored | 3mut; 2G; | | Serine | |
| 4.0F14(A204V_V208L_{—} | | | deltaG; DS; | | linker #1 | |
| V68I_V255L)_{—} | | | SOSL; GS; | | (also | |
| RnS3mut2G_gp160_{—} | | | I535M; PC; | | labeled | |
| IGHVss_deltaG_{—} | | | Y712I; H66A; | | as ‘L’) | |
| DS.SOSL.GS_I535M.PC_{—} | | | T316W; | | | |
| Y712I_D167N_H66A_{—} | | | LL855/6AA | | | |
| T316W_LL855/6AA | | | | | | |
| HV1303338, HIV_{—} | Protein | Membrane | F14; deltaG; | IGHVss | Glycine- | Truncated |
| CAP256SU_UCA_OPT_4.0 | | anchored | SOSL; GS; | | Serine | beyond HXB2 |
| F14(A204V_V208L_{—} | | | I535M | | linker #1 | position 712 |
| V68I_V255L)_IGHVss_{—} | | | | | (also | |
| deltaG_SOSL.GS_{—} | | | | | labeled | |
| I535M_D167N_gp145.712 | | | | | as ‘L’) | |
| HV1303339, HIV_{—} | Protein | Membrane | F14; deltaG; | IGHVss | Glycine- | Truncated |
| CAP256SU_UCA_OPT_4.0 | | anchored | DS; SOSL; | | Serine | beyond HXB2 |
| F14(A204V_V208L_{—} | | | GS; I535M | | linker #1 | position 712 |
| V68I_V255L)_IGHVss_{—} | | | | | (also | |
| deltaG_DS.SOSL.GS_{—} | | | | | labeled | |
| I535M_D167N_gp145.712 | | | | | as ‘L’) | |
| HV1303340, HIV_{—} | Protein | Membrane | F14; RnS; | IGHVss | Glycine- | Truncated |
| CAP256SU_UCA_OPT_4.0 | | anchored | 3mut; 2G; | | Serine | beyond HXB2 |
| F14(A204V_V208L_{—} | | | deltaG; DS; | | linker #1 | position 712 |
| V68I_V255L)_RnS3mut2G_{—} | | | SOSL; GS; | | (also | |
| IGHVss_deltaG_{—} | | | I535M | | labeled | |
| DS.SOSL.GS_I535M_{—} | | | | | as ‘L’) | |
| D167N_gp145.712 | | | | | | |
| HV1303341, HIV_{—} | Protein | Membrane | F14; deltaG; | IGHVss | Glycine- | Truncated |
| CAP256SU_UCA_OPT_4.0 | | anchored | DS; SOSL; | | Serine | beyond HXB2 |
| F14(A204V_V208L_{—} | | | GS; I535M; | | linker #1 | position 712 |
| V68I_V255L)_IGHVss_{—} | | | PC; H66A; | | (also | |
| deltaG_DS.SOSL.GS_{—} | | | T316W | | labeled | |
| I535M.PC.H66A_{—} | | | | | as ‘L’) | |
| T316W_D167N_gp145.712 | | | | | | |
| HV1303342, HIV_{—} | Protein | Membrane | F14; RnS; | IGHVss | Glycine- | Truncated |
| CAP256SU_UCA_OPT_{—} | | anchored | 3mut; 2G; | | Serine | beyond HXB2 |
| 4.0F14(A204V_V208L_{—} | | | deltaG; DS; | | linker #1 | position 712 |
| V68I_V255L)_{—} | | | SOSL; GS; | | (also | |
| RnS3mut2G_IGHVss_{—} | | | I535M; PC; | | labeled | |
| deltaG_DS.SOSL.GS_{—} | | | H66A; T316W | | as ‘L’) | |
| I535M.PC.H66A_{—} | | | | | | |
| T316W_D167N_gp145.712 | | | | | | |
| HV1303343, HIV_{—} | Protein | Membrane | F14; deltaG; | IGHVss | Glycine- | Truncated |
| CAP256SU_UCA_OPT_4.0 | | anchored | SOSL; GS; | | Serine | beyond HXB2 |
| F14(A204V_V208L_{—} | | | I535M; Y712I | | linker #1 | position 755 |
| V68I_V255L)_IGHVss_{—} | | | | | (also | |
| deltaG_SOSL.GS_{—} | | | | | labeled | |
| I535M_D167N_Y712I_{—} | | | | | as ‘L’) | |
| gp150.755 | | | | | | |
| HV1303344, HIV_{—} | Protein | Membrane | F14; deltaG; | IGHVss | Glycine- | Truncated |
| CAP256SU_UCA_OPT_4.0 | | anchored | DS; SOSL; | | Serine | beyond HXB2 |
| F14(A204V_V208L_{—} | | | GS; I535M; | | linker #1 | position 755 |
| V68I_V255L)_IGHVss_{—} | | | Y712I | | (also | |
| deltaG_DS.SOSL.GS_{—} | | | | | labeled | |
| I535M_D167N_{—} | | | | | as ‘L’) | |
| Y712I_gp150.755 | | | | | | |
| HV1303345, HIV_{—} | Protein | Membrane | F14; RnS; | IGHVss | Glycine- | Truncated |
| CAP256SU_UCA_OPT_4.0 | | anchored | 3mut; 2G; | | Serine | beyond HXB2 |
| F14(A204V_V208L_{—} | | | deltaG; DS; | | linker #1 | position 755 |
| V68I_V255L)_RnS3mut2G_{—} | | | SOSL; GS; | | (also | |
| IGHVss_deltaG_{—} | | | I535M; Y712I | | labeled | |
| DS.SOSL.GS_I535M_{—} | | | | | as ‘L’) | |
| DI67N_Y712I_gp150.755 | | | | | | |
| HV1303346, HIV_{—} | Protein | Membrane | F14; deltaG; | IGHVss | Glycine- | Truncated |
| CAP256SU_UCA_OPT_4.0 | | anchored | DS; SOSL; | | Serine | beyond HXB2 |
| F14(A204V_V208L_{—} | | | GS; I535M; | | linker #1 | position 755 |
| V68I_V255L)_IGHVss_{—} | | | PC; Y712I; | | (also | |
| deltaG_DS.SOSL.GS_{—} | | | H66A; T316W | | labeled | |
| I535M.PC_D167N_Y712I_{—} | | | | | as ‘L’) | |
| H66A_T316W_gp150.755 | | | | | | |
| HV1303347, HIV_{—} | Protein | Membrane | F14; RnS; | IGHVss | Glycine- | Truncated |
| CAP256SU_UCA_OPT_{—} | | anchored | 3mut; 2G; | | Serine | beyond HXB2 |
| 4.0F14(A204V_V208L_{—} | | | deltaG; DS; | | linker #1 | position 755 |
| V68I_V255L)_{—} | | | SOSL; GS; | | (also | |
| RnS3mut2G_IGHVss_{—} | | | I535M; PC; | | labeled | |
| deltaG_DS.SOSL.GS_{—} | | | Y712I; H66A; | | as ‘L’) | |
| I535M.PC_D167N_{—} | | | T316W | | | |
| Y712I_H66A_T316W_{—} | | | | | | |
| gp150.755 | | | | | | |
| HV1303348, HIV_{—} | Protein | Membrane | F14; deltaG; | IGHVss | Glycine- | Truncated |
| CAP256SU_UCA_OPT_4.0 | | anchored | SOSL; GS; | | Serine | beyond HXB2 |
| F14(A204V_V208L_{—} | | | I535M; | | linker #1 | position 750 |
| V68I_V255L)_IGHVss_{—} | | | Y712I | | (also | |
| deltaG_SOSL.GS_{—} | | | | | labeled | |
| I535M_D167N_Y712I_{—} | | | | | as ‘L’) | |
| gp150.750 | | | | | | |
| HV1303349, HIV_{—} | Protein | Membrane | F14; deltaG; | IGHVss | Glycine- | Truncated |
| CAP256SU_UCA_OPT_4.0 | | anchored | DS; SOSL; | | Serine | beyond HXB2 |
| F14(A204V_V208L_{—} | | | GS; I535M; | | linker #1 | position 750 |
| V68I_V255L)_IGHVss_{—} | | | Y712I | | (also | |
| deltaG_DS.SOSL.GS_{—} | | | | | labeled | |
| I535M_D167N_Y712I_{—} | | | | | as ‘L’) | |
| gp150.750 | | | | | | |
| HV1303350, HIV_{—} | Protein | Membrane | F14; RnS; | IGHVss | Glycine- | Truncated |
| CAP256SU_UCA_OPT_4.0 | | anchored | 3mut; 2G; | | Serine | beyond HXB2 |
| F14(A204V_V208L_{—} | | | deltaG; DS; | | linker #1 | position 750 |
| V68I_V255L)_RnS3mut2G_{—} | | | SOSL; GS; | | (also | |
| IGHVss_deltaG_{—} | | | I535M; Y712I | | labeled | |
| DS.SOSL.GS_I535M_{—} | | | | | as ‘L’) | |
| D167N_Y712I_gp150.750 | | | | | | |
| HV1303351, HIV_{—} | Protein | Membrane | F14; deltaG; | IGHVss | Glycine- | Truncated |
| CAP256SU_UCA_OPT_4.0 | | anchored | DS; SOSL; | | Serine | beyond HXB2 |
| F14(A204V_V208L_{—} | | | GS; I535M; | | linker #1 | position 750 |
| V68I_V255L)_IGHVss_{—} | | | PC; Y712I; | | (also | |
| deltaG_DS.SOSL.GS_{—} | | | H66A; T316W | | labeled | |
| I535M.PC_D167N_{—} | | | | | as ‘L’) | |
| Y712I_H66A_T316W_{—} | | | | | | |
| gp150.750 | | | | | | |
| HV1303352, HIV_{—} | Protein | Membrane | F14; RnS; | IGHVss | Glycine- | Truncated |
| CAP256SU_UCA_OPT_{—} | | anchored | 3mut; 2G; | | Serine | beyond HXB2 |
| 4.0F14(A204V_V208L_{—} | | | deltaG; DS; | | linker #1 | position 750 |
| V68I_V255L)_{—} | | | SOSL; GS; | | (also | |
| RnS3mut2G_IGHVss_{—} | | | I535M; PC; | | labeled | |
| deltaG_DS.SOSL.GS_{—} | | | Y712I; H66A; | | as ‘L’) | |
| I535M.PC_D167N_{—} | | | T316W | | | |
| Y712I_H66A_T316W_{—} | | | | | | |
| gp150.750 | | | | | | |
| HV1303353, HIV_{—} | Protein | Membrane | F14; deltaG; | BPrlss | Glycine- | Wildtype |
| CAP256SU_UCA_OPT_4.0 | | anchored | SOSL; GS; | | Serine | |
| F14(A204V_V208L_{—} | | | I535M; | | linker #1 | |
| V68I_V255L)_gp160_{—} | | | Y712I | | (also | |
| BPrlss_deltaG_{—} | | | | | labeled | |
| SOSL.GS_I535M_D167N_{—} | | | | | as ‘L’) | |
| Y712I | | | | | | |
| HV1303354, HIV_{—} | Protein | Membrane | F14; deltaG; | BPrlss | Glycine- | Truncated |
| CAP256SU_UCA_OPT_4.0 | | anchored | SOSL; GS; | | Serine | beyond HXB2 |
| F14(A204V_V208L_{—} | | | I535M | | linker #1 | position 712 |
| V68I_V255L)_BPrlss_{—} | | | | | (also | |
| deltaG_SOSL.GS_{—} | | | | | labeled | |
| I535M_D167N_gp145.712 | | | | | as ‘L’) | |
| HV1303355, HIV_{—} | Protein | Membrane | F14; deltaG; | BPrlss | Glycine- | Truncated |
| CAP256SU_UCA_OPT_4.0 | | anchored | SOSL; GS; | | Serine | beyond HXB2 |
| F14(A204V_V208L_{—} | | | I535M; Y712I | | linker #1 | position 755 |
| V68I_V255L)_BPrlss_{—} | | | | | (also | |
| deltaG_SOSL.GS_{—} | | | | | labeled | |
| I535M_D167N_Y712I_{—} | | | | | as ‘L’) | |
| gp150.755 | | | | | | |
| HV1303356, HIV_{—} | Protein | Membrane | F14; deltaG; | BPrlss | Glycine- | Truncated |
| CAP256SU_UCA_OPT_4.0 | | anchored | SOSL; GS; | | Serine | beyond HXB2 |
| F14(A204V_V208L_{—} | | | I535M; Y712I | | linker #1 | position 750 |
| V68I_V255L)_BPrlss_{—} | | | | | (also | |
| deltaG_SOSL.GS_{—} | | | | | labeled | |
| I535M_D167N_Y712I_{—} | | | | | as ‘L’) | |
| gp150.750 | | | | | | |
| HV1303357, HIV_{—} | Protein | Membrane | F14; deltaG; | BPrlss | Glycine- | Wildtype |
| CAP256SU_UCA_OPT_4.0 | | anchored | DS; SOSL; | | Serine | |
| F14(A204V_V208L_{—} | | | GS; I535M; | | linker #1 | |
| V68I_V255L)_gp160_{—} | | | Y712I; | | (also | |
| BPrlss_deltaG_{—} | | | LL855/6AA | | labeled | |
| DS.SOSL.GS_I535M_{—} | | | | | as ‘L’) | |
| D167N_Y712I_LL855/ | | | | | | |
| 6AA | | | | | | |
| HV1303358, HIV_{—} | Protein | Membrane | F14; RnS; | BPrlss | Glycine- | Wildtype |
| CAP256SU_UCA_OPT_4.0 | | anchored | 3mut; 2G; | | Serine | |
| F14(A204V_V208L_{—} | | | deltaG; DS; | | linker #1 | |
| V68I_V255L)_RnS3mut2G_{—} | | | SOSL; GS; | | (also | |
| gp160_BPrlss_deltaG_{—} | | | I535M; Y712I, | | labeled | |
| DS.SOSL.GS_I535M_D167N_{—} | | | LL855/6AA | | as ‘L’) | |
| Y712I_LL855/6AA | | | | | | |
| HV1303359, HIV_{—} | Protein | Membrane | F14; deltaG; | BPrlss | Glycine- | Wildtype |
| CAP256SU_UCA_OPT_4.0 | | anchored | DS; SOSL; | | Serine | |
| F14(A204V_V208L_{—} | | | GS; I535M; | | linker #1 | |
| V68I_V255L)_gp160_{—} | | | PC; Y712I; | | (also | |
| BPrlss_deltaG_{—} | | | H66A; T316W; | | labeled | |
| DS.SOSL.GS_I535M.PC_{—} | | | LL855/6AA | | as ‘L’) | |
| D167N_Y712I_H66A_{—} | | | | | | |
| T316W_LL855/6AA | | | | | | |
| HV1303360, HIV_{—} | Protein | Membrane | F14; RnS; | BPrlss | Glycine- | Wildtype |
| CAP256SU_UCA_OPT_{—} | | anchored | 3mut; 2G; | | Serine | |
| 4.0F14(A204V_V208L_{—} | | | deltaG; DS; | | linker #1 | |
| V68I_V255L)_{—} | | | SOSL; GS; | | (also | |
| RnS3mut2G_gp160_{—} | | | I535M; PC; | | labeled | |
| BPrlss_deltaG_{—} | | | Y712I; H66A; | | as ‘L’) | |
| DS.SOSL.GS_I535M.PC_{—} | | | T316W; | | | |
| D167N_Y712I_H66A_{—} | | | LL855/6AA | | | |
| T316W_LL855/6AA | | | | | | |
| HV1303361, HIV_{—} | Protein | Membrane | F14; deltaG; | BPrlss | Glycine- | Truncated |
| CAP256SU_UCA_OPT_4.0 | | anchored | DS; SOSL; | | Serine | beyond HXB2 |
| F14(A204V_V208L_{—} | | | GS; I535M | | linker #1 | position 712 |
| V68I_V255L)_BPrlss_{—} | | | | | (also | |
| deltaG_DS.SOSL.GS_{—} | | | | | labeled | |
| I535M_D167N_gp145.712 | | | | | as ‘L’) | |
| HV1303362, HIV_{—} | Protein | Membrane | F14; RnS; | BPrlss | Glycine- | Truncated |
| CAP256SU_UCA_OPT_4.0 | | anchored | 3mut; 2G; | | Serine | beyond HXB2 |
| F14(A204V_V208L_{—} | | | deltaG; DS; | | linker #1 | position 712 |
| V68I_V255L)_RnS3mut2G_{—} | | | SOSL; GS; | | (also | |
| BPrlss_deltaG_{—} | | | I535M | | labeled | |
| DS.SOSL.GS_I535M_{—} | | | | | as ‘L’) | |
| D167N_gp145.712 | | | | | | |
| HV1303363, HIV_{—} | Protein | Membrane | F14; deltaG; | BPrlss | Glycine- | Truncated |
| CAP256SU_UCA_OPT_4.0 | | anchored | DS; SOSL; | | Serine | beyond HXB2 |
| F14(A204V_V208L_{—} | | | GS; I535M; | | linker #1 | position 712 |
| V68I_V255L)_BPrlss_{—} | | | PC; H66A; | | (also | |
| deltaG_DS.SOSL.GS_{—} | | | T316W | | labeled | |
| I535M.PC.H66A_T316W_{—} | | | | | as ‘L’) | |
| D167N_gp145.712 | | | | | | |
| HV1303364, HIV_{—} | Protein | Membrane | F14; RnS; | BPrlss | Glycine- | Truncated |
| CAP256SU_UCA_OPT_{—} | | anchored | 3mut; 2G; | | Serine | beyond HXB2 |
| 4.0F14(A204V_V208L_{—} | | | deltaG; DS; | | linker #1 | position 712 |
| V68I_V255L)_{—} | | | SOSL; GS; | | (also | |
| RnS3mut2G_BPrlss_{—} | | | I535M; PC; | | labeled | |
| deltaG_DS.SOSL.GS_{—} | | | H66A; T316W | | as ‘L’) | |
| I535M.PC.H66A_T316W_{—} | | | | | | |
| D167N_gp145.712 | | | | | | |
| HV1303365, HIV_{—} | Protein | Membrane | F14; deltaG; | BPrlss | Glycine- | Truncated |
| CAP256SU_UCA_OPT_4.0 | | anchored | DS; SOSL; | | Serine | beyond HXB2 |
| F14(A204V_V208L_{—} | | | GS; I535M; | | linker #1 | position 755 |
| V68I_V255L)_BPrlss_{—} | | | Y712I | | (also | |
| deltaG_DS.SOSL.GS_{—} | | | | | labeled | |
| I535M_D167N_Y712I_{—} | | | | | as ‘L’) | |
| gp150.755 | | | | | | |
| HV1303366, HIV_{—} | Protein | Membrane | F14; RnS; | BPrlss | Glycine- | Truncated |
| CAP256SU_UCA_OPT_4.0 | | anchored | 3mut; 2G; | | Serine | beyond HXB2 |
| F14(A204V_V208L_{—} | | | deltaG; DS; | | linker #1 | position 755 |
| V68I_V255L)_RnS3mut2G_{—} | | | SOSL; GS; | | (also | |
| BPrlss_deltaG_{—} | | | I535M; Y712I | | labeled | |
| DS.SOSL.GS_I535M_{—} | | | | | as ‘L’) | |
| D167N_Y712I_gp150.755 | | | | | | |
| HV1303367, HIV_{—} | Protein | Membrane | F14; deltaG; | BPrlss | Glycine- | Truncated |
| CAP256SU_UCA_OPT_4.0 | | anchored | DS; SOSL; | | Serine | beyond HXB2 |
| F14(A204V_V208L_{—} | | | GS; I535M; | | linker #1 | position 755 |
| V68I_V255L)_BPrlss_{—} | | | PC; Y712I; | | (also | |
| deltaG_DS.SOSL.GS_{—} | | | H66A; T316W | | labeled | |
| I535M.PC_D167N_Y712I_{—} | | | | | as ‘L’) | |
| H66A_T316W_gp150.755 | | | | | | |
| HV1303368, HIV_{—} | Protein | Membrane | F14; RnS; | BPrlss | Glycine- | Truncated |
| CAP256SU_UCA_OPT_{—} | | anchored | 3mut; 2G; | | Serine | beyond HXB2 |
| 4.0F14(A204V_V208L_{—} | | | deltaG; DS; | | linker #1 | position 755 |
| V68I_V255L)_{—} | | | SOSL; GS; | | (also | |
| RnS3mut2G_BPrlss_{—} | | | I535M; PC; | | labeled | |
| deltaG_DS.SOSL.GS_{—} | | | Y712I; H66A; | | as ‘L’) | |
| I535M.PC_D167N_{—} | | | T316W | | | |
| Y712I_H66A_T316W_{—} | | | | | | |
| gp150.755 | | | | | | |
| HV1303369, HIV_{—} | Protein | Membrane | F14; deltaG; | BPrlss | Glycine- | Truncated |
| CAP256SU_UCA_OPT_4.0 | | anchored | DS; SOSL; | | Serine | beyond HXB2 |
| F14(A204V_V208L_{—} | | | GS; I535M; | | linker #1 | position 750 |
| V68I_V255L)_BPrlss_{—} | | | Y712I | | (also | |
| deltaG_DS.SOSL.GS_{—} | | | | | labeled | |
| I535M_D167N_Y712I_{—} | | | | | as ‘L’) | |
| gp150.750 | | | | | | |
| HV1303370, HIV_{—} | Protein | Membrane | F14; RnS; | BPrlss | Glycine- | Truncated |
| CAP256SU_UCA_OPT_4.0 | | anchored | 3mut; 2G; | | Serine | beyond HXB2 |
| F14(A204V_V208L_{—} | | | deltaG; DS; | | linker #1 | position 750 |
| V68I_V255L)_RnS3mut2G_{—} | | | SOSL; GS; | | (also | |
| BPrlss_deltaG_{—} | | | I535M; Y712I | | labeled | |
| DS.SOSL.GS_I535M_{—} | | | | | as ‘L’) | |
| D167N_Y712I_gp150.750 | | | | | | |
| HV1303371, HIV_{—} | Protein | Membrane | F14; deltaG; | BPrlss | Glycine- | Truncated |
| CAP256SU_UCA_OPT_4.0 | | anchored | DS; SOSL; | | Serine | beyond HXB2 |
| F14(A204V_V208L_{—} | | | GS; I535M; | | linker #1 | position 750 |
| V68I_V255L)_BPrlss_{—} | | | PC; Y712I; | | (also | |
| deltaG_DS.SOSL.GS_{—} | | | H66A; T316W | | labeled | |
| I535M.PC_D167N_Y712I_{—} | | | | | as ‘L’) | |
| H66A_T316W_gp150.750 | | | | | | |
| HV1303372, HIV_{—} | Protein | Membrane | F14; RnS; | BPrlss | Glycine- | Truncated |
| CAP256SU_UCA_OPT_{—} | | anchored | 3mut; 2G; | | Serine | beyond HXB2 |
| 4.0F14(A204V_V208L_{—} | | | deltaG; DS; | | linker #1 | position 750 |
| V68I_V255L)_{—} | | | SOSL; GS; | | (also | |
| RnS3mut2G_BPrlss_{—} | | | I535M; PC; | | labeled | |
| deltaG_DS.SOSL.GS_{—} | | | Y712I; H66A; | | as ‘L’) | |
| I535M.PC_D167N_{—} | | | T316W | | | |
| Y712I_H66A_T316W_{—} | | | | | | |
| gp150.750 | | | | | | |
| HV1303373, HIV_{—} | Protein | Membrane | F14; RnS; | BPrlss | Glycine- | Wildtype |
| CAP256SU_UCA_OPT_4.0 | | anchored | 3mut; 2G; | | Serine | |
| F14(A204V_V208L_{—} | | | deltaG; DS; | | linker #1 | |
| V68I_V255L)_RnS3mut2G_{—} | | | SOSL; GS; | | (also | |
| gp160_BPrlss_deltaG_{—} | | | I535M; Y712I; | | labeled | |
| DS.SOSL.GS_I535M_{—} | | | LL855/6AA | | as ‘L’) | |
| Y712I_LL855/6AA | | | | | | |
| HV1303374, HIV_{—} | Protein | Membrane | F14; deltaG; | BPrlss | Glycine- | Wildtype |
| CAP256SU_UCA_OPT_4.0 | | anchored | DS; SOSL; | | Serine | |
| F14(A204V_V208L_{—} | | | GS; I535M; | | linker #1 | |
| V68I_V255L)_gp160_{—} | | | PC; Y712I; | | (also | |
| BPrlss_deltaG_{—} | | | H66A; T316W; | | labeled | |
| DS.SOSL.GS_I535M.PC._{—} | | | LL855/6AA | | as ‘L’) | |
| Y712I_H66A_T316W_{—} | | | | | | |
| LL855/6AA | | | | | | |
| HV1303375, HIV_{—} | Protein | Membrane | F14; RnS; | BPrlss | Glycine- | Wildtype |
| CAP256SU_UCA_OPT_{—} | | anchored | 3mut; 2G; | | Serine | |
| 4.0F14(A204V_V208L_{—} | | | deltaG; DS; | | linker #1 | |
| V68I_V255L)_{—} | | | SOSL; GS; | | (also | |
| RnS3mut2G_gp160_{—} | | | I535M; PC; | | labeled | |
| BPrlss_deltaG_{—} | | | Y712I; H66A; | | as ‘L’) | |
| DS.SOSL.GS_I535M.PC._{—} | | | T316W; | | | |
| Y712I_H66A_T316W_{—} | | | LL855/6AA | | | |
| LL855/6AA | | | | | | |
| HV1303376, HIV_{—} | Protein | Membrane | F14; deltaG; | BPrlss | Glycine- | Truncated |
| CAP256SU_UCA_OPT_4.0 | | anchored | DS; SOSL; | | Serine | beyond HXB2 |
| F14(A204V_V208L_{—} | | | GS; I535M | | linker #1 | position 712 |
| V68I_V255L)_BPrlss_{—} | | | | | (also | |
| deltaG_DS.SOSL.GS_{—} | | | | | labeled | |
| I535M_gp145.712 | | | | | as ‘L’) | |
| HV1303377, HIV_{—} | Protein | Membrane | F14; RnS; | BPrlss | Glycine- | Truncated |
| CAP256SU_UCA_OPT_4.0 | | anchored | 3mut; 2G; | | Serine | beyond HXB2 |
| F14(A204V_V208L_{—} | | | deltaG; DS; | | linker #1 | position 712 |
| V68I_V255L)_RnS3mut2G_{—} | | | SOSL; GS; | | (also | |
| BPrlss_deltaG_{—} | | | I535M | | labeled | |
| DS.SOSL.GS_I535M_{—} | | | | | as ‘L’) | |
| gp145.712 | | | | | | |
| HV1303378, HIV_{—} | Protein | Membrane | F14; deltaG; | BPrlss | Glycine- | Truncated |
| CAP256SU_UCA_OPT_4.0 | | anchored | DS; SOSL; | | Serine | beyond HXB2 |
| F14(A204V_V208L_{—} | | | GS; I535M; | | linker #1 | position 712 |
| V68I_V255L)_BPrlss_{—} | | | PC; H66A; | | (also | |
| deltaG_DS.SOSL.GS_{—} | | | T316W | | labeled | |
| I535M.PC.H66A_{—} | | | | | as ‘L’) | |
| T316W_gp145.712 | | | | | | |
| HV1303379, HIV_{—} | Protein | Membrane | F14; RnS; | BPrlss | Glycine- | Truncated |
| CAP256SU_UCA_OPT_{—} | | anchored | 3mut; 2G; | | Serine | beyond HXB2 |
| 4.0F14(A204V_V208L_{—} | | | deltaG; DS; | | linker #1 | position 712 |
| V68I_V255L)_{—} | | | SOSL; GS; | | (also | |
| RnS3mut2G_BPrlss_{—} | | | I535M; PC; | | labeled | |
| deltaG_DS.SOSL.GS_{—} | | | H66A; T316W | | as ‘L’) | |
| I535M.PC.H66A_{—} | | | | | | |
| T316W_gp145.712 | | | | | | |
| HV1303380, HIV_{—} | Protein | Membrane | F14; deltaG; | BPrlss | Glycine- | Truncated |
| CAP256SU_UCA_OPT_4.0 | | anchored | DS; SOSL; | | Serine | beyond HXB2 |
| F14(A204V_V208L_{—} | | | GS; I535M; | | linker #1 | position 755 |
| V68I_V255L)_BPrlss_{—} | | | Y712I | | (also | |
| deltaG_DS.SOSL.GS_{—} | | | | | labeled | |
| I535M_Y712I_gp150.755 | | | | | as ‘L’) | |
| HV1303381, HIV_{—} | Protein | Membrane | F14; RnS; | BPrlss | Glycine- | Truncated |
| CAP256SU_UCA_OPT_4.0 | | anchored | 3mut; 2G; | | Serine | beyond HXB2 |
| F14(A204V_V208L_{—} | | | deltaG; DS; | | linker #1 | position 755 |
| V68I_V255L)_RnS3mut2G_{—} | | | SOSL; GS; | | (also | |
| BPrlss_deltaG_{—} | | | I535M; Y712I | | labeled | |
| DS.SOSL.GS_I535M_{—} | | | | | as ‘L’) | |
| Y712I_gp150.755 | | | | | | |
| HV1303382, HIV_{—} | Protein | Membrane | F14; deltaG; | BPrlss | Glycine- | Truncated |
| CAP256SU_UCA_OPT_4.0 | | anchored | DS; SOSL; | | Serine | beyond HXB2 |
| F14(A204V_V208L_{—} | | | GS; I535M; | | linker #1 | position 755 |
| V68I_V255L)_BPrlss_{—} | | | PC; Y712I; | | (also | |
| deltaG_DS.SOSL.GS_{—} | | | H66A; T316W | | labeled | |
| I535M.PC._Y712I_{—} | | | | | as ‘L’) | |
| H66A_T316W_gp150.755 | | | | | | |
| HV1303383, HIV_{—} | Protein | Membrane | F14; RnS; | BPrlss | Glycine- | Truncated |
| CAP256SU_UCA_OPT_{—} | | anchored | 3mut; 2G; | | Serine | beyond HXB2 |
| 4.0F14(A204V_V208L_{—} | | | deltaG; DS; | | linker #1 | position 755 |
| V68I_V255L)_{—} | | | SOSL; GS; | | (also | |
| RnS3mut2G_BPrlss_{—} | | | I535M; PC; | | labeled | |
| deltaG_DS.SOSL.GS_{—} | | | Y712I; H66A; | | as ‘L’) | |
| I535M.PC._Y712I_{—} | | | T316W | | | |
| H66A_T316W_gp150.755 | | | | | | |
| HV1303384, HIV_{—} | Protein | Membrane | F14; deltaG; | BPrlss | Glycine- | Truncated |
| CAP256SU_UCA_OPT_4.0 | | anchored | DS; SOSL; | | Serine | beyond HXB2 |
| F14(A204V_V208L_{—} | | | GS; I535M; | | linker #1 | position 750 |
| V68I_V255L)_BPrlss_{—} | | | Y712I | | (also | |
| deltaG_DS.SOSL.GS_{—} | | | | | labeled | |
| I535M_Y712I_gp150.750 | | | | | as ‘L’) | |
| HV1303385, HIV_{—} | Protein | Membrane | F14; RnS; | BPrlss | Glycine- | Truncated |
| CAP256SU_UCA_OPT_4.0 | | anchored | 3mut; 2G; | | Serine | beyond HXB2 |
| F14(A204V_V208L_{—} | | | deltaG; DS; | | linker #1 | position 750 |
| V68I_V255L)_RnS3mut2G_{—} | | | SOSL; GS; | | (also | |
| BPrlss_deltaG_{—} | | | I535M; Y712I | | labeled | |
| DS.SOSL.GS_I535M_{—} | | | | | as ‘L’) | |
| Y712I_gp150.750 | | | | | | |
| HV1303386, HIV_{—} | Protein | Membrane | F14; deltaG; | BPrlss | Glycine- | Truncated |
| CAP256SU_UCA_OPT_4.0 | | anchored | DS; SOSL; | | Serine | beyond HXB2 |
| F14(A204V_V208L_{—} | | | GS; I535M; | | linker #1 | position 750 |
| V68I_V255L)_BPrlss_{—} | | | PC; Y712I; | | (also | |
| deltaG_DS.SOSL.GS_{—} | | | H66A; T316W | | labeled | |
| I535M.PC._Y712I_{—} | | | | | as ‘L’) | |
| H66A_T316W_gp150.750 | | | | | | |

Modifications disclosed include (all positions are based on HXB2 numbering):
Y712I includes a Y712I substitution. Without being bound by theory, this modification increase expression of Env on cell surface. See Labranche et al. J. Virol. 69 (9): 5217-5227 (1995).
Sodroski includes substitutions H66A, A582T, and L587A. Without being bound by theory, this modification prevents CD4-induced conformations. See Pacheco et al. J Virol. 91 (5): e02219-16 (2017).
F14 includes substitutions A204V, V208L, V681, and V255L. Without being bound by theory, this modification prevents CD4-induced conformations. See Henderson et al. Nat Commun. 11:520 (2020).
SOSIP includes substitutions A501C, T605C, and 1559P. Without being bound by theory, this modification stabilizes prefusion conformation. See Sanders et al. J. Virol. 76, 8875-8889 (2002).
SOS includes substitutions A501C and T605C. Without being bound by theory, this modification stabilizes prefusion conformation, See Sanders et al. J. Virol. 76, 8875-8889 (2002).
DS includes substitutions I201C and A433C. Without being bound by theory, this modification fixes prefusion conformation. See Kwon et al. Nat Struct Mol Biol 22:522-531 (2015).
3mut includes substitutions N302M, T320L, and A329P. Without being bound by theory, this modification stabilizes trimer apex, improve thermostability. See Chuang, G-Y et al. J Virol 94: e00074-20 (2020)
2G includes substitutions D636G and T569G. Without being bound by theory, this modification prevents postfusion gp41 helical transition. See Guenaga J., et al. Immunity 46 (5): 792-803.e3 (2017).
RnS includes substitutions E442N, S437P, A2041, 1573F, K588E, D589V, Y609P, K651F, S6551, and 1535N. It replaces rare and/or destabilizing mutations from wildtype Env. (Specific to CAP256wk34.80 Env, but we also used for CAP256SU based on its high similarity to the former. Due to conflict with F14 mutation A204V, when RnS are combined with F14, the A204V in F14 was used.). See Gorman J., et al. Cell Reports 31 (1): 107488 (2020).
Signal Peptide #1 replaces wildtype signal peptide with MAISGVPVLGFFIIAVLMSAQESWA. Without being bound by theory, this modification improves expression of mRNA constructs.
Glycine-Serine linker #1 replace 508-REKR-511 with GGGGSGGGGS, a flexible linker between gp120 & gp41 to replace furin cleavage. Also referred to as modification “L”. See Sharma S.K., et al. Cell Reports 11 (4): 539-550 (2015).
RRRRRR replaces 508-REKR-511 with RRRRRR and replaces native furin cleavage site with a modified furin cleavage sequence between gp120 & gp41. Without being bound by theory, this modification increases cleavage frequency. Also referred to as modification “6R or R6”. See Ringe R P et al. Proc Natl Acad Sci USA. 2013 Nov. 5; 110 (45): 18256-61.
SIVMac CT replaces wildtype cytoplasmic tail (HXB2 713-854) with truncated SIVMac cytoplasmic tail RPVFSSPPSYFQ. Without being bound by theory, this modification improves expression of Env on cell-surface when expressed by mRNA immunogens. See Postler T. S., Desrosiers R. C. J. Virol. 87 (1): 2-15 (2013).
GS replaces sequence from HXB2 546-568 with GSAGSAGSGSAGSGSAGSGSAGS and replaces the unstable portion (23 amino acids) of envelope heptad repeat 1 with a flexible linker of equivalent size. See Saunders et al. Unpublished.
T316W includes substitution T316W. Without being bound by theory, this modification includes hydrophobic amino acid in the V3 loop to facilitate packing of the V3 loop and prevent unwanted exposure. See de Taeye S W, Ozorowski G, Torrents de la Peña A, et al. Cell. 2015; 163 (7): 1702-1715.
I535M includes substitution IS35M. Without being bound by theory, this modification stabilizes of the interprotomer contacts in gp41. See de Taeye SW, Ozorowski G, Torrents de la Peña A, et al. Cell. 2015; 163 (7): 1702-1715.
deltaG deletes HIV-1 envelope Glycine at position 29. Without being bound by theory, this modification presumes Glycine 29 to be the final amino acid in the signal peptide. Glycine 29 is removed when an artificial signal sequence is added. See Saunders et al. Unpublished.
LL855/6AA includes substitutions L855A and L856A. Without being bound by theory, this modification is a mutation of a conserved dileucine motif that mediates endocytosis of HIV-1 envelope. Without being bound by theory, when combined with Y712I boosts surface expression of HIV-1 envelope. See Byland R et al. Mol Biol Cell. 2007 February; 18 (2): 414-25
IGHVss replace wildtype signal peptide with MGWSCIILFLVATATGVHA, a mouse immunoglobulin heavy chain variable region signal sequence. Without being bound by theory, this modification enhances protein secretion from cells. UniProtKB/Swiss-Prot Accession Number P01750. Also referred as “mIgss”. See Cheng K W et al. Biochem J. 2021; 478 (12): 2309-2319.
BPriss replaces wildtype signal peptide with MDSKGSSQKGSRLLLLLVVSNLLLPQGVLA, a bovine prolactin signal sequence. Without being bound by theory, this modification enhances protein secretion from cells. See Saunders et al. Unpublished.
PC includes substitutions S655K and K658Q. Without being bound by theory, this modification includes contact amino acids between envelope protomers. Without being bound by theory, this modification stabilizes sequence found in BG505. Protomer contacts are referred to as “PC”. See Saunders et al. Unpublished.
H66A includes substitution H66A. Without being bound by theory, substitution in gp120 that modulates the transition from the unliganded conformation of envelope to the CD4-bound state. See Pacheco B, et al. J Virol. 2017; 91 (5): e02219-16.
2560_pUC-ccTEV-A101 5′UTR includes aGcATAAAAGTCTCAACACAACATATACAAAACAAACGAATCTCAAGCAA TCAAGCATTCTACTTCTATTGCAGCAATTTAAATCATTTCTTTTAAAGCAA AAGCAATTTTCTGAAAATTTTCACCATTTACGAACGATAGCGCT. Without being bound by theory, this modification is an improved 5′ UTR sequence for mRNA stability and half-life from screens. See Mohamad-Gabriel Alamch, Drew Weissman et al.
2560_pUC-ccTEV-A101 3′UTR includes actagtAGTGACTGACTAGGATCTGGTTACCACTAAACCAGCCTCAAGAACAC CCGAATGGAGTCTCTAAGCTACATAATACCAACTTACACTTACAAAATGTT GTCCCCCAAAATGTAGCCATTCGTATCTGCTCCTAATAAAAAGAAAGTTTC TTCACATTCT. Without being bound by theory, this modification is an improved 5′ UTR sequence for mRNA stability and half-life from screens. See Mohamad-Gabriel Alameh, Drew Weissman et al.
poly A (immediately after 3′UTR) includes AAAAAAAAAAAAAAAAAAAAAAAAAAAAAAAAAAAAAAAAAAAAA AAAAAAAAAAAAAAAAAAAAAAAAAAAAAAAAAAAAAAAAAAAAAAA AAAAAAA. Without being bound by theory, this modification is an improved polyA tail sequence for mRNA stability and half-life. See Jalkanen et al. Semin Cell Dev Biol. 34:24-32 (2014).
mRNA codon optimization includes a reverse translation of protein amino acid sequence to optimal codons. Without being bound by theory, this modification codon optimization is performed as follow: amino acid sequence is reverse translated into an DNA sequence using a modified mammalian codon usage table. The table increases both the CIA and the GC content of the mRNA. The reverse translated sequence (or mRNA sequence) is modeled into mFold and Delta H/Delta G computed, and the sequence with the lowest free energy is selected. In some cases, the codons can be replaced in specific locations to relax the tridimentional structure of the optimized mRNA. The sequence is then cloned between the 5′UTR and 3′UTR above. See Leppek et al. Nature Communications 13:1536 (2022).

The exemplary constructs provided herein, see e.g., Table 4, include various combinations of these envelope modifications. Any modification or combination of the modifications described herein, including but not limited, to different versions of soluble proteins, different versions of membrane expressed proteins, stabilization mutations, furin cleavage site mutations, signal peptides, and/or cytoplasmic tail modifications can be applied to any full-length envelopes sequence. For example, one or more of the modifications described herein can be applied to envelope CAM13RRK, CAM13RRRK, HIV_CAP256SU_UCA_OPT_4.0, CAP256SU_UCA_OPT_4.0 375S, CAP256SU_UCA_OPT_4.0 Y375S_D167N, CAP256 wk34.80_V2UCA_OPT, CAP256 wk34.80 PCT64UCA_OPT, CAP256_wk34.80_V2UCA_OPT_R17IK, CAP256 wk34.80 V2UCA_OPT_RRK, CAP256_wk34.80_V2UCA_OPT_RRK_D167N, Q23.17 (natural_wildtype), Q23.17_V2UCAOPT, Q23.17_V2UCAOPT_GLY, Q23.17_V2UCAOPT_ALT, Q23.17_V2UCAOPT_GLY_ALT, Q23.17_V2UCAOPT_GLY_ALT_R170Q, CH505_V2UCAOPT2_N332, CH505_V2UCAOPT_v3.0.

In some embodiments, envelope CAM13RRK, CAM13RRRK, HIV_CAP256SU_UCA_OPT_4.0, CAP256SU_UCA_OPT_4.0 375S, CAP256SU_UCA_OPT_4.0_Y375S_D167N, CAP256_wk34.80_V2UCA_OPT, CAP256 wk34.80 PCT64UCA_OPT, CAP256 wk34.80_V2UCA_OPT_R17IK, CAP256_wk34.80 V2UCA_OPT_RRK, CAP256_wk34.80_V2UCA_OPT_RRK_D167N, Q23.17_(natural_wildtype), Q23.17_V2UCAOPT, Q23.17_V2UCAOPT_GLY, Q23.17_V2UCAOPT_ALT, Q23.17_V2UCAOPT_GLY_ALT, Q23.17 V2UCAOPT_GLY_ALT_R170Q, CH505_V2UCAOPT2 N332, CH505_V2UCAOPT_v3.0 comprises one or more of modifications Y712I, Sodroski substitutions, F14 substitutions, SOSIP substitutions, SOS substitutions, DS substitutions, PP substitutions, 3mut substitutions, 2G substitutions, RnS substitutions, Signal Peptide #1, Glysine-Serine linker #1, RRRRRR, SIVMacCT, GS, T316W, 1535M, deltaG, LL855/6AA, IGHVss, BPriss, PC substitutions, or H66A.

The invention is described in the following non-limiting examples.

### EXAMPLES

### Example 1

Saunders et al. have reported that vaccination with stabilized CH505 SOSIP trimers elicits VIV2-glycan bnAbs. See Cell Rep. 2017 Dec. 26; 21 (13): 3681-3690, incorporated by reference in its entirety.

### Example 2

CH505-BG505 Chimeric SOSIP Redesign for V2 UCA Constructs & for V5 glycan mutants

Chimeric v4 6R SOSIP constructs have BG505 gp41 and end at HXB2 664. Thus, the SOSIP constructs have sub-optimal amino acids at some of our mature and UCA signature sites in gp41.

Since the region encompassed by the SOSIP constructs ends at 664, the UCA_OPT1 SOSIP and OPT2 SOSIP constructs are the same. Same for UCA_OPT1 N332 and UCA OPT2 N332 SOSIPs. So, skip testing the OPT2 SOSIP constructs.

Instead, we suggest testing two other constructs: with and without gp41 optimized mutations in the backbones of UCA_OPT1 and UCA_OPT1 N332—these are UCA_OPT1 gp41mut and UCA_OPT1 N332 gp41mut.

The gp41mut constructs introduce favorable amino acids at 3 sites: 588 and 644 (signature sites for mature V2 apex bNAbs) and 535 (PG9 germline reverted signature).

List of SOSIP constructs for testing:
CH50STF_V2.SET.OPT_ch.SOSIPv4.1
CH505TF_V2.SET.OPT.N332_ch.SOSIPv4.1
CH505TF_V2.UCA.OPT1_ch.SOSIPv4.1
CH505TF_V2.UCA.OPT1.N332 ch.SOSIP.v4.1

But we propose testing the following two instead of the UCA_OPT2 constructs (since they are same as UCA_OPT1 for the SOSIP constructs);
CH50STF_V2.UCA.OPT1.gp41mut_ch.SOSIP.v4.1
CH50STF_V2.UCA.OPT1.N332.gp41mut_ch.SOSIP.v4.1

The gp4 Imut constructs have 3 mutations in gp41: R->K at position 588; G->R at position 644; M->I at position 535.

Additional optimized sequences are shown in FIG. 4C, 12F, 13, 14, 16, 17, and 18F, and characterization in FIGS. 6A and 6B and 8-12, 15, and 18. Additional SOSIPs sequences are shown in FIGS. 13, 14, and 17.

### Example 3 Animal Studies

In non-limiting embodiment these immunogens can be used as either single primes and boosts in humanized mice or bnAb UCA or intermediate antibody VH+VL knockin mice, non-human primates (NHPs) or humans, or used in combinations in animal models or in humans.

Immunogens to initiate VIV2, and/or CD4 binding site and/or Fusion Peptide unmutated common ancestor (UCA) broadly neutralizing antibody (bnAbs) precursors. Non-limiting examples of immunizations are listed:
1. Prime X 3 with either A, B, C, D, G or H (listed in FIGS. 3, 12F, 13, 14, 16, 17, or 18F, Table 1). In other embodiments, these immunogens could be in any suitable envelope form.
2. Take the optimal prime for bnAbs and after priming, boost with A, B, C, D, G or H.
3. Take the optimal prime for bnAbs, and after priming boost with a mixture of A, B, C, D. G or H.
4. Prime X3 with the mixture of A, B, C, D, G and H and the boost with one of A, B, C D, D or H to focus the response on bnAb epitopes.
5. Prime as in steps #1-4 above and then boost with the CH505 Transmitted/Founder (TF) gp140 SOSIP trimer that has induced autologous neutralizing antibodies against the CH505 tier 2 TF virus.
6. Prime as in steps #1-4 above and then boost with the forms of the MT145 SIV Env (see e.g. Andrabi et al., 2019, Cell Reports27, 2426-244) or similar SIV envelope that has a VIV2 loop-glycan bnAb epitope that binds to VIV2-glycan UCAs and bnAbs.
7. Prime as in steps #1-4 above and then boost with CM244, ZM233, WITO HIV-1 envelope or other WT Envs that have binding affinity for V1V2 bnAbs and their UCAs.

In non-limiting embodiments, these are administered as recombinant protein. Any suitable adjuvant could be use. In non-limiting embodiments, these are administered as nucleic acids, DNA and/or mRNAs. In non-limiting embodiments, the mRNAs are modified mRNAs administered as LNPs.

In non-limiting embodiments, the immunogens provide optimal prime for VIV2, and/or CD4 binding site, and/or Fusion Peptide precursors. In some embodiments, an optimal prime is determined by measurement of the frequency of bnAb precursors before immunization and after each immunization to determine if the immunization has expanded the desired bnAb B cell precursor pool. This can be performed by initial B cell repertoire analysis by single cell sorting of memory or germinal center B cells (e.g. Bonsignori et al. Sci Transl Med. 2017 Mar. 15; 9 (381): eaai7514.) and then followed by next generation sequencing of either lymph node, blood or other immune organ B cells to determine if the primed B cell bnAb clones were expanded and therefore boosted.

### Example 4

This example shows information and sequences of a second design round. This second round of designs resulted in gains in sensitivity to the CAP256 and PG9/PG16 UCAs.

Several signatures had been found for PG9 with only the heavy and/or light chain reverted. However, no PG9 UCA reactivity was identified. Thus, it was hypothesized that the single chain revered PG9 is not a good mimic of the PG9 UCA.

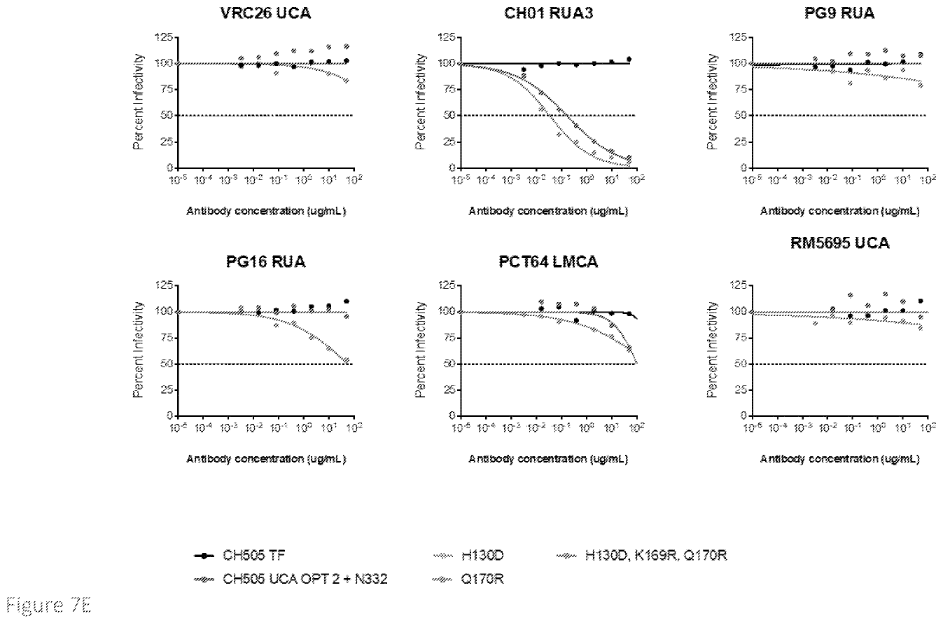

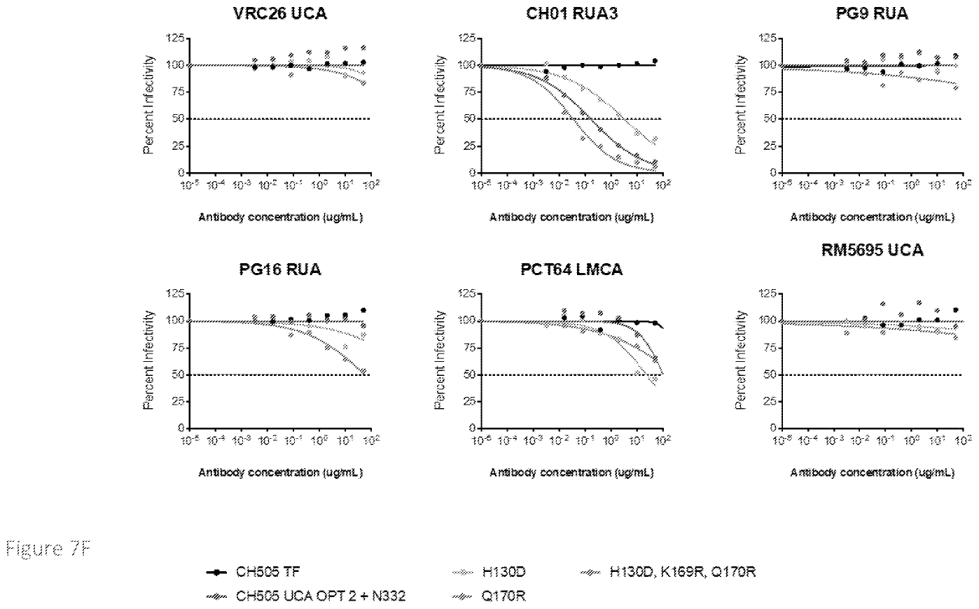

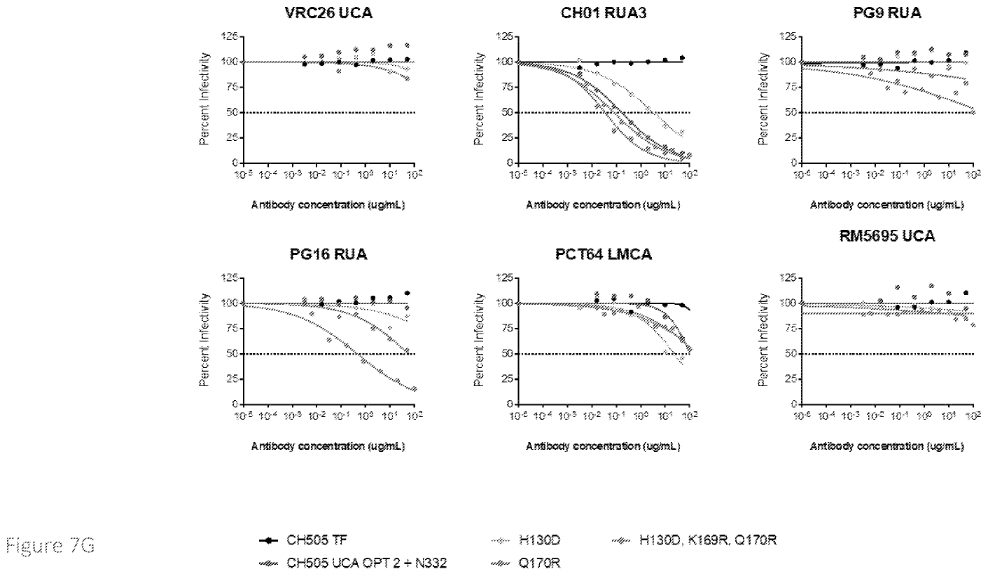

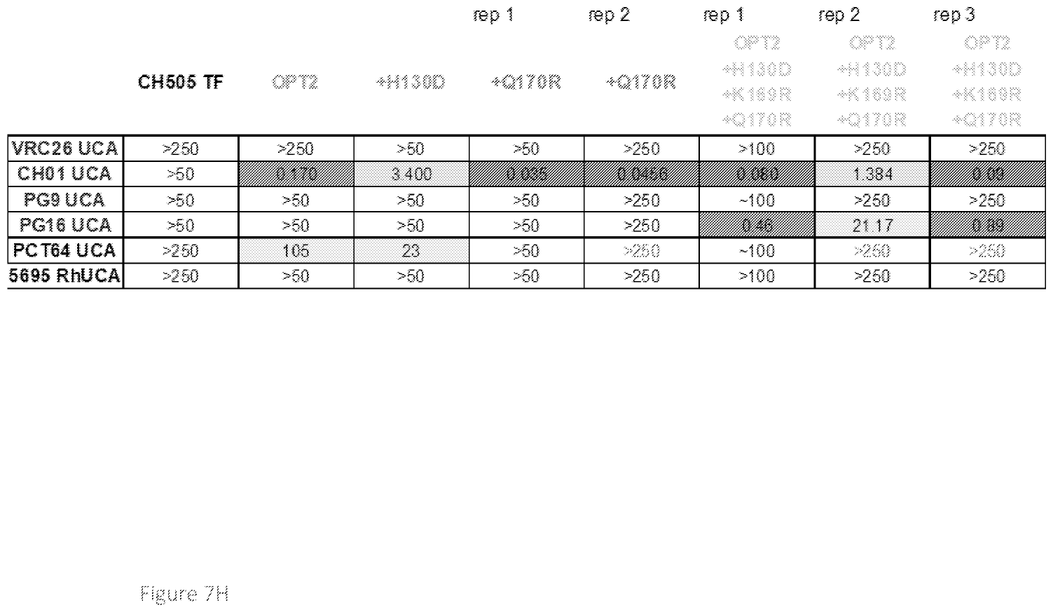

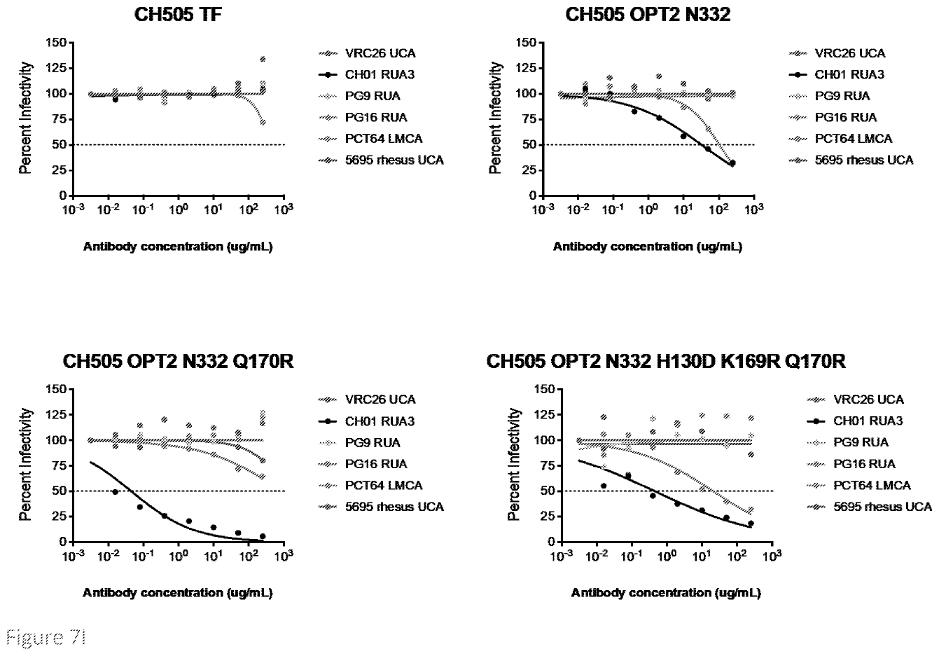

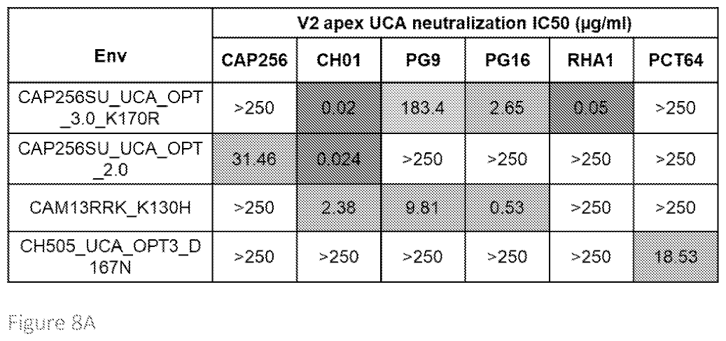

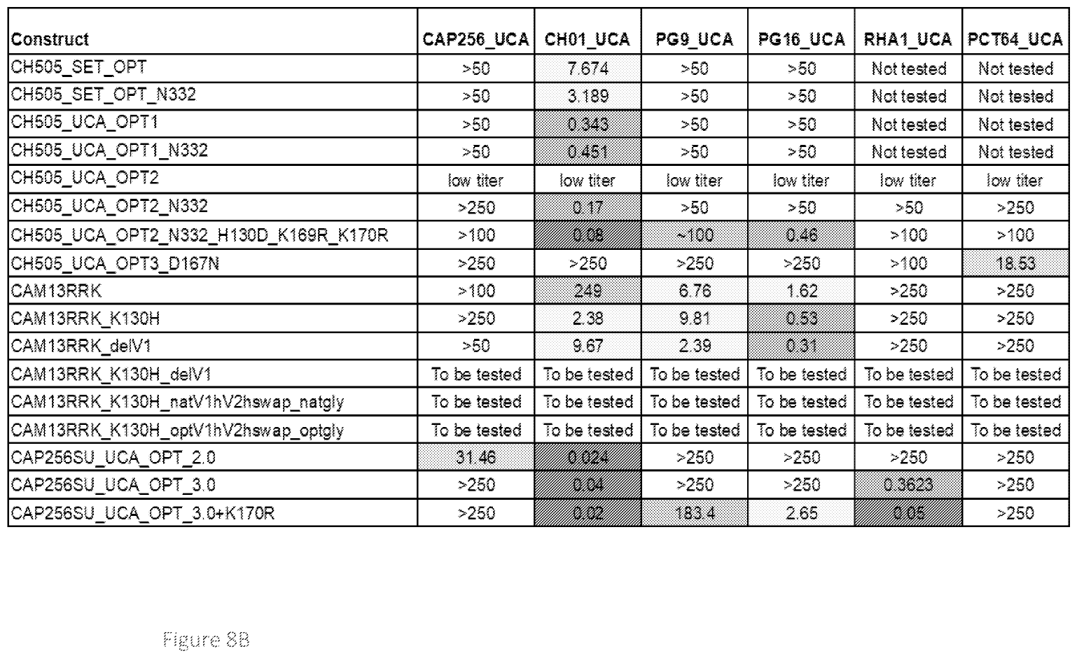

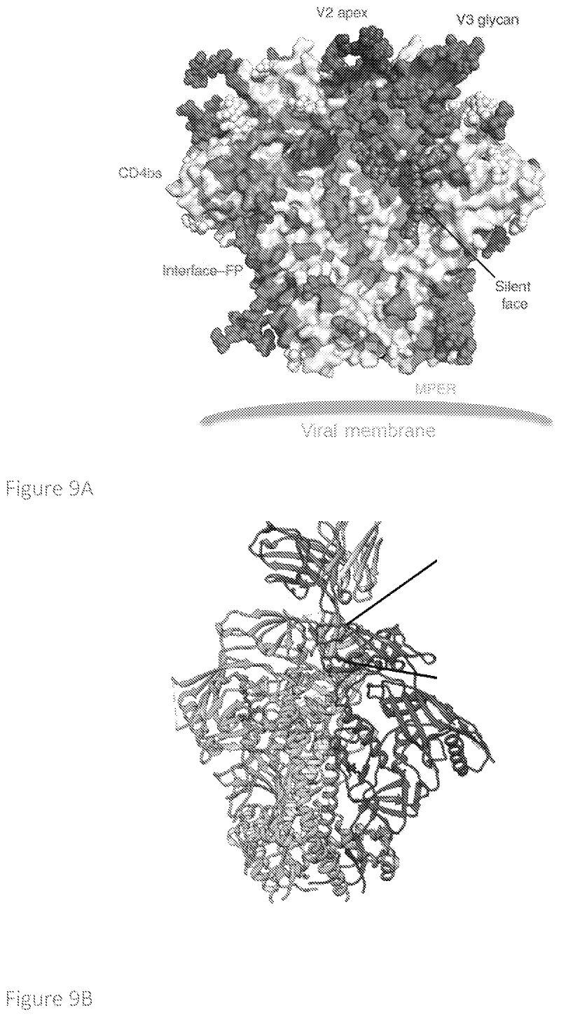

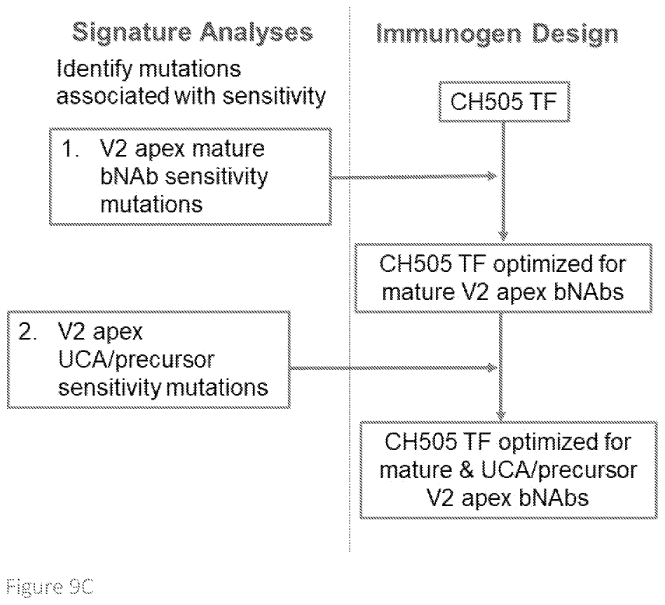

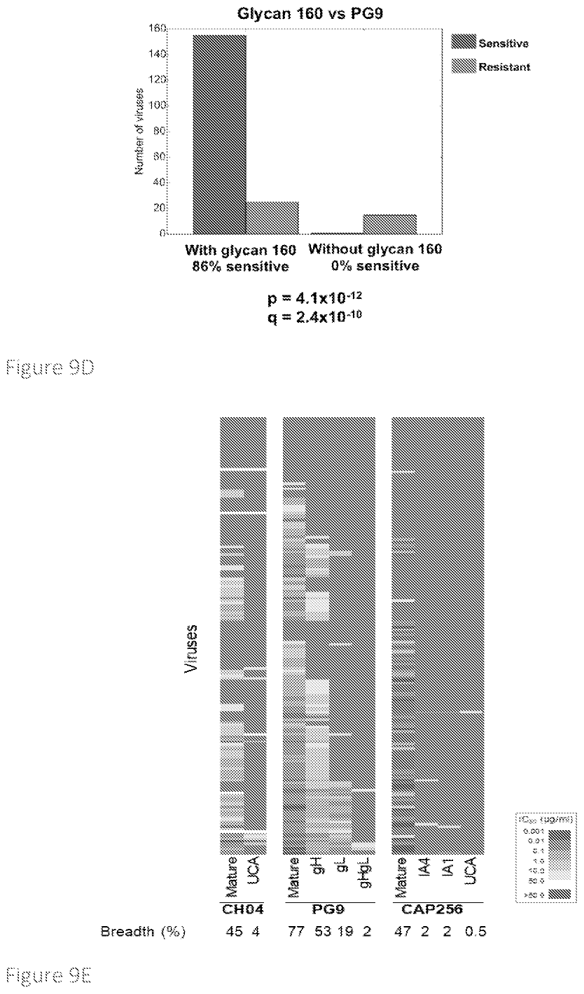

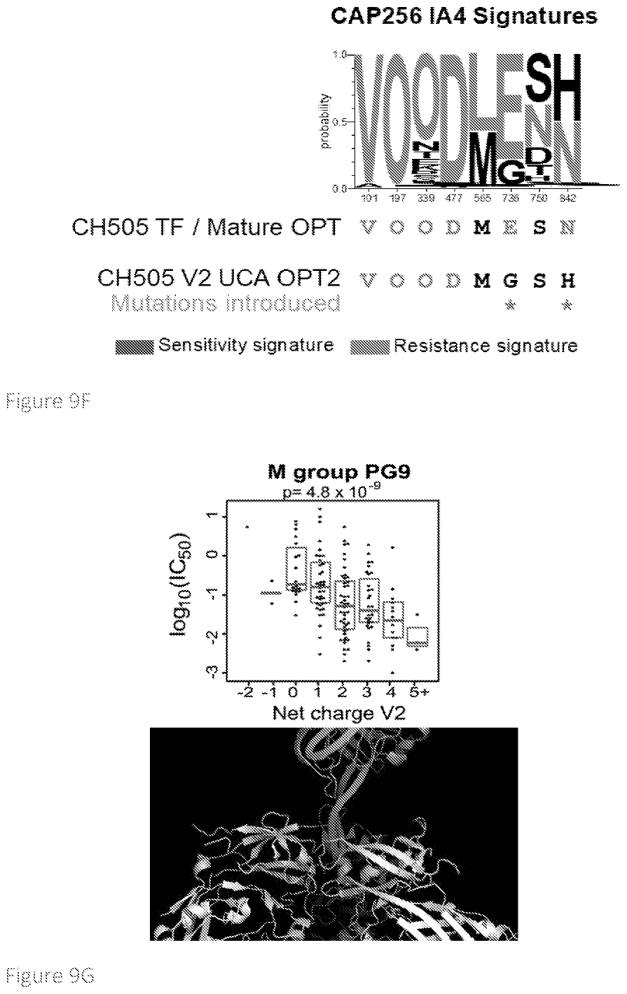

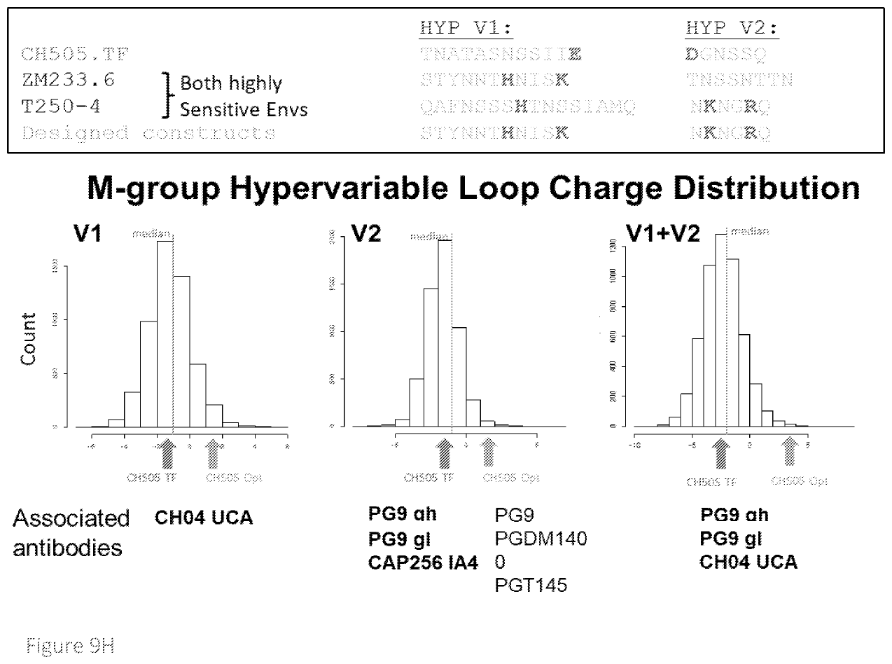

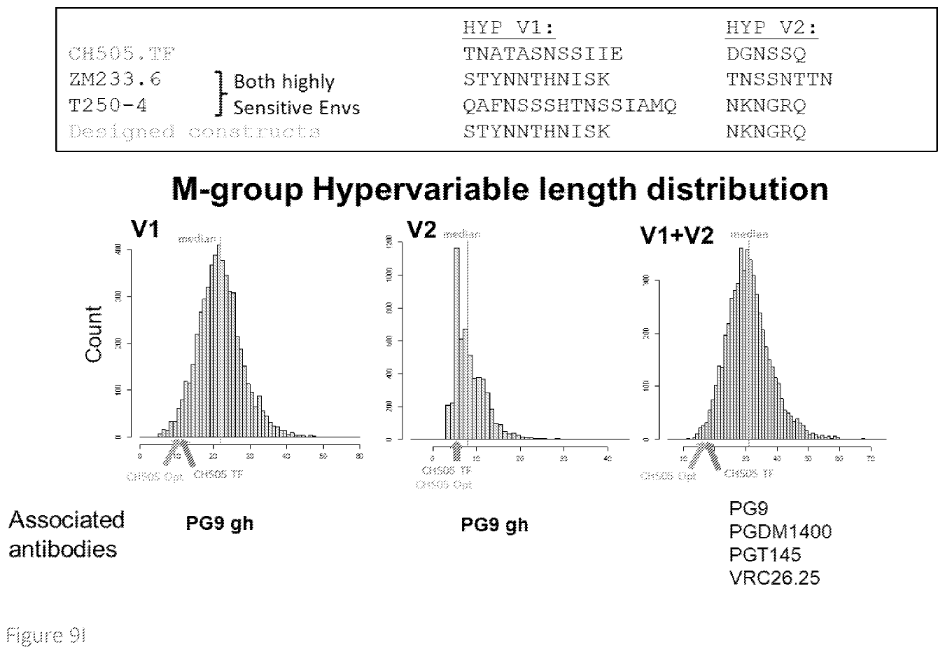

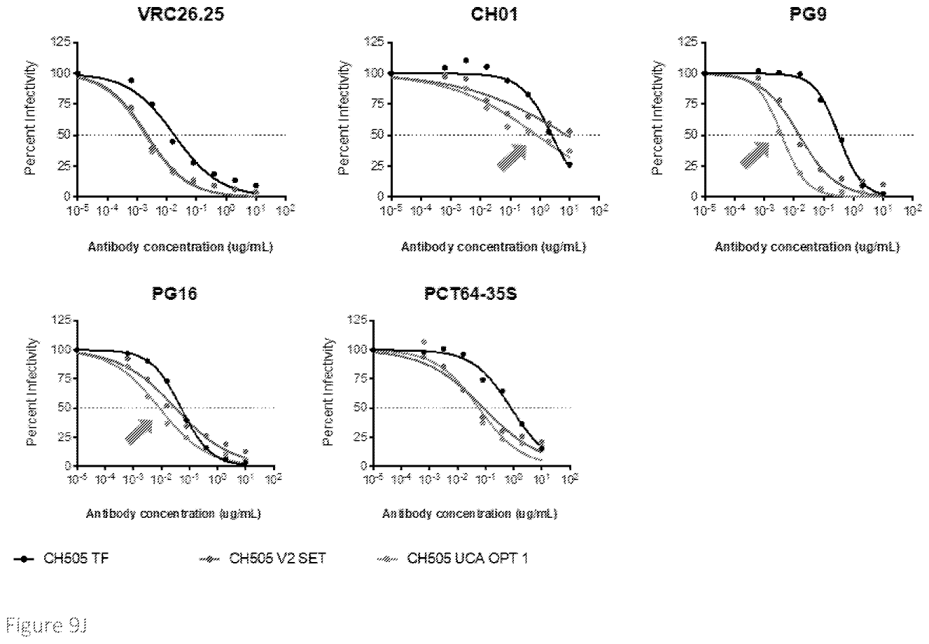

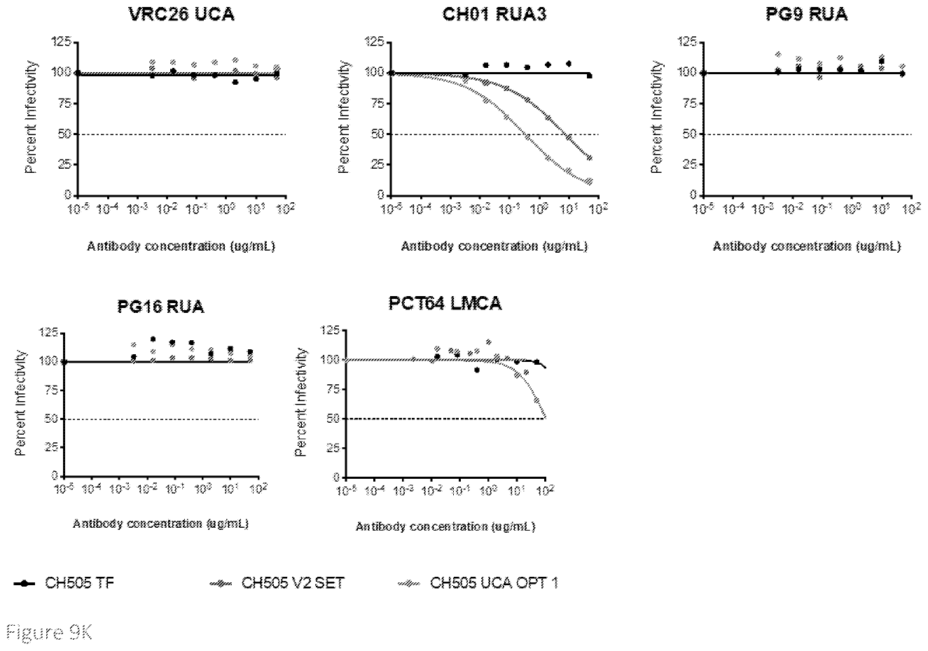

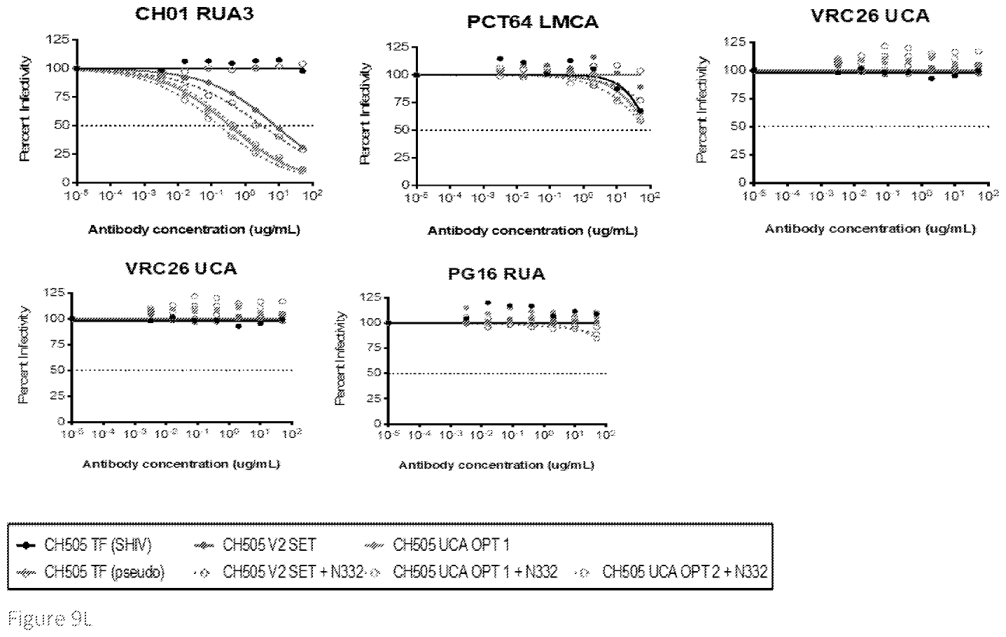

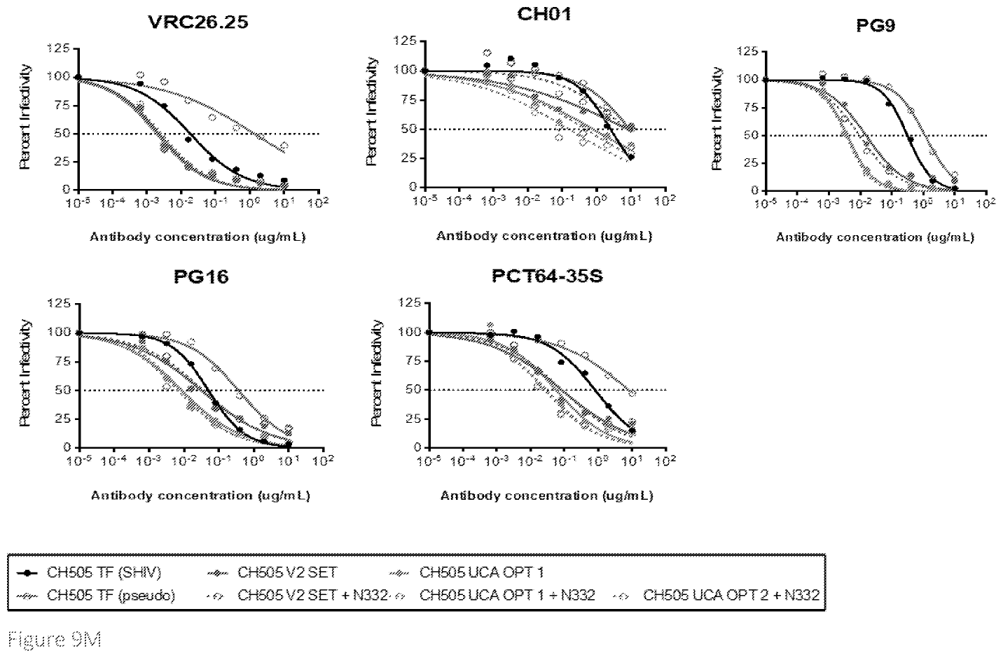

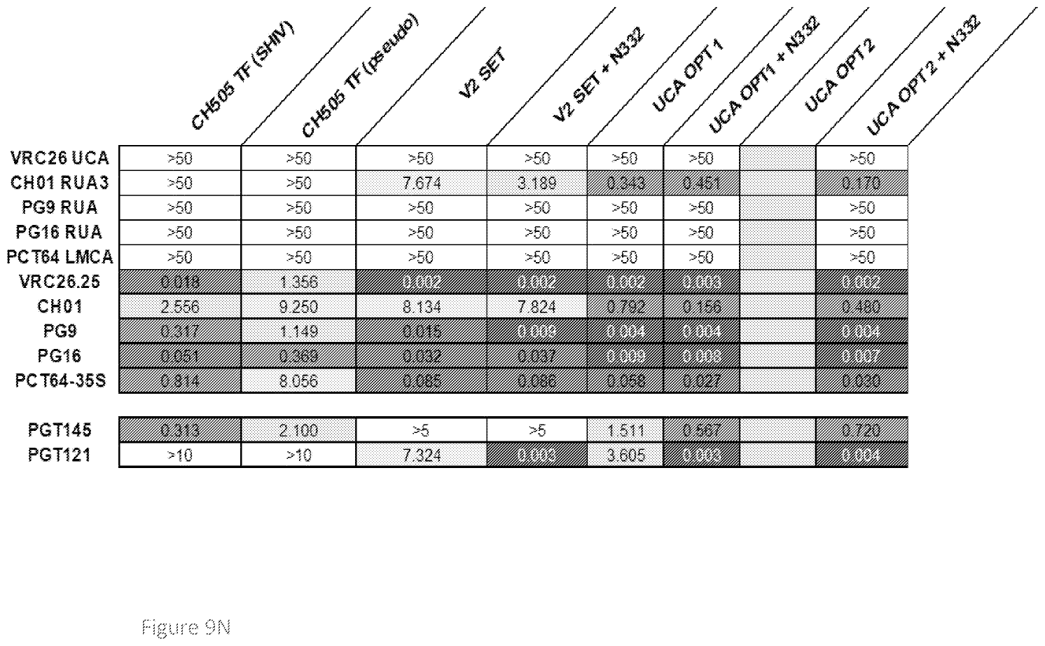

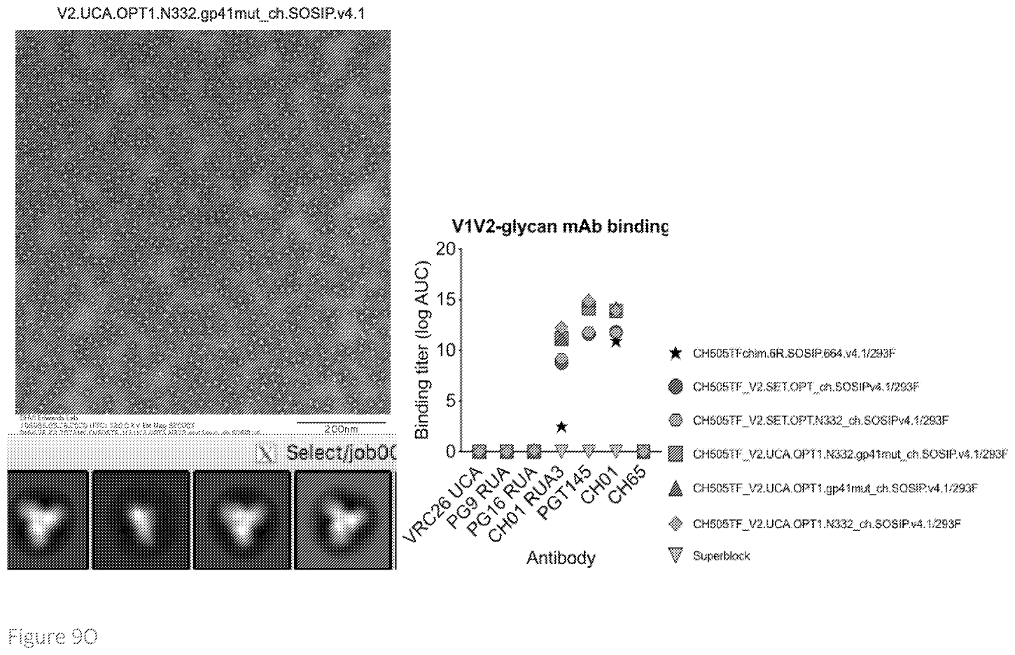

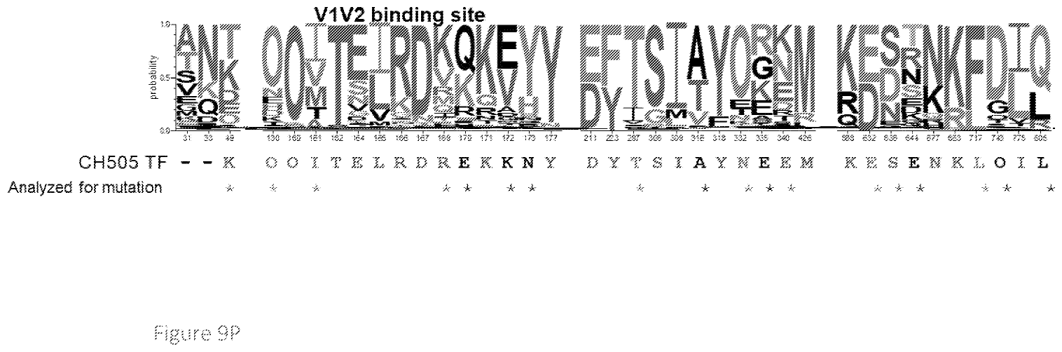

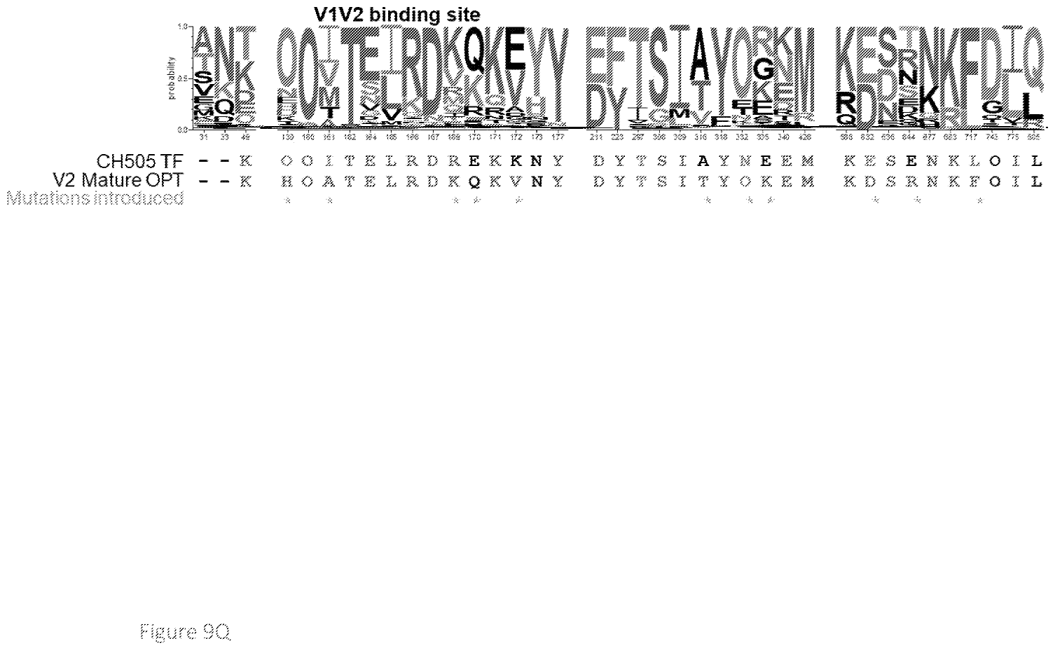

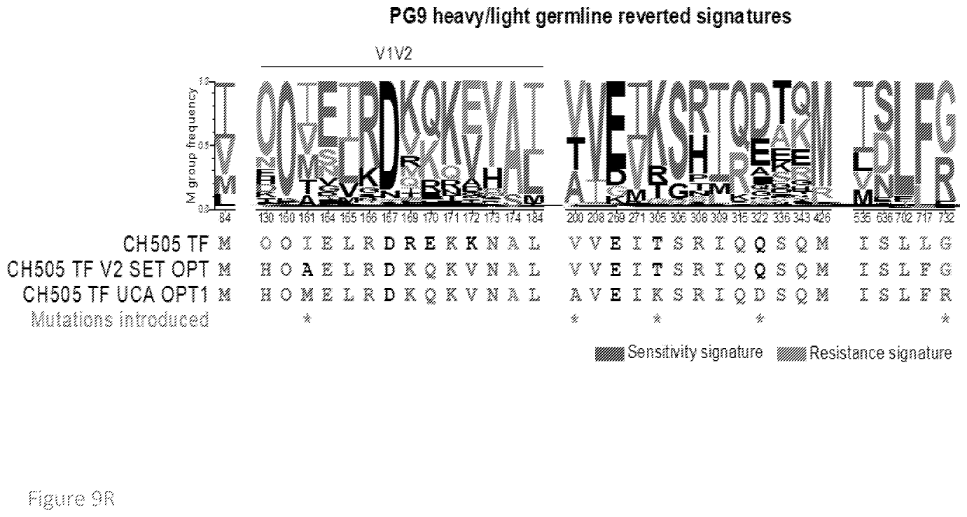

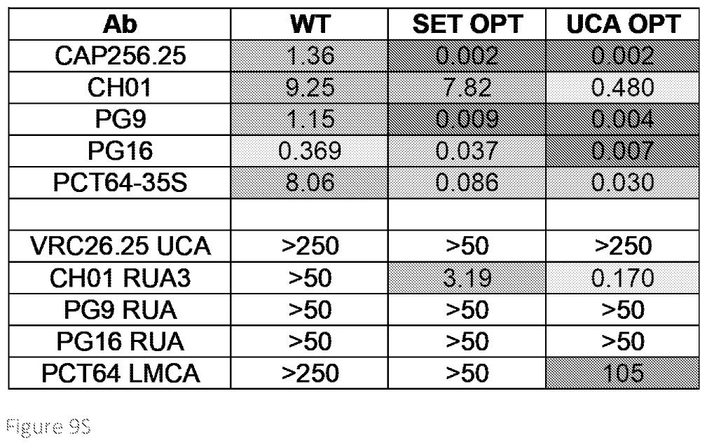

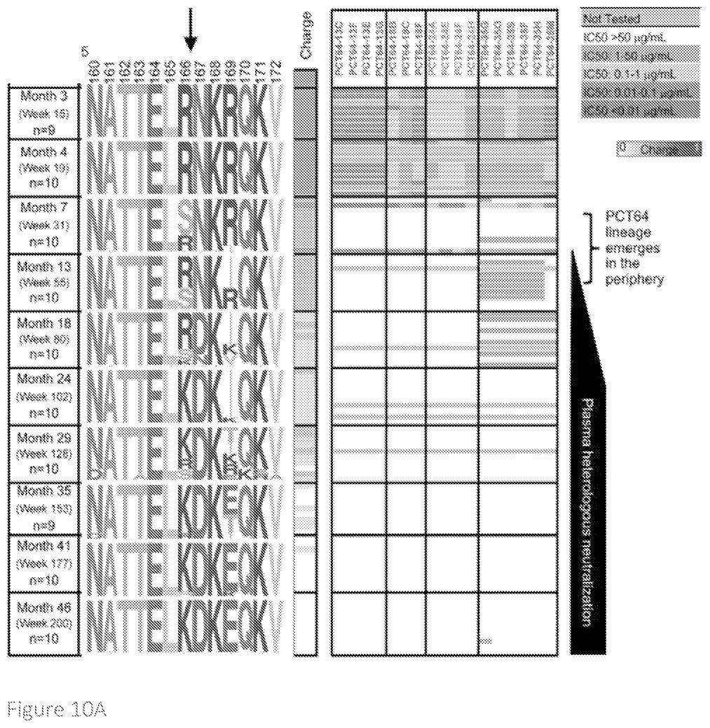

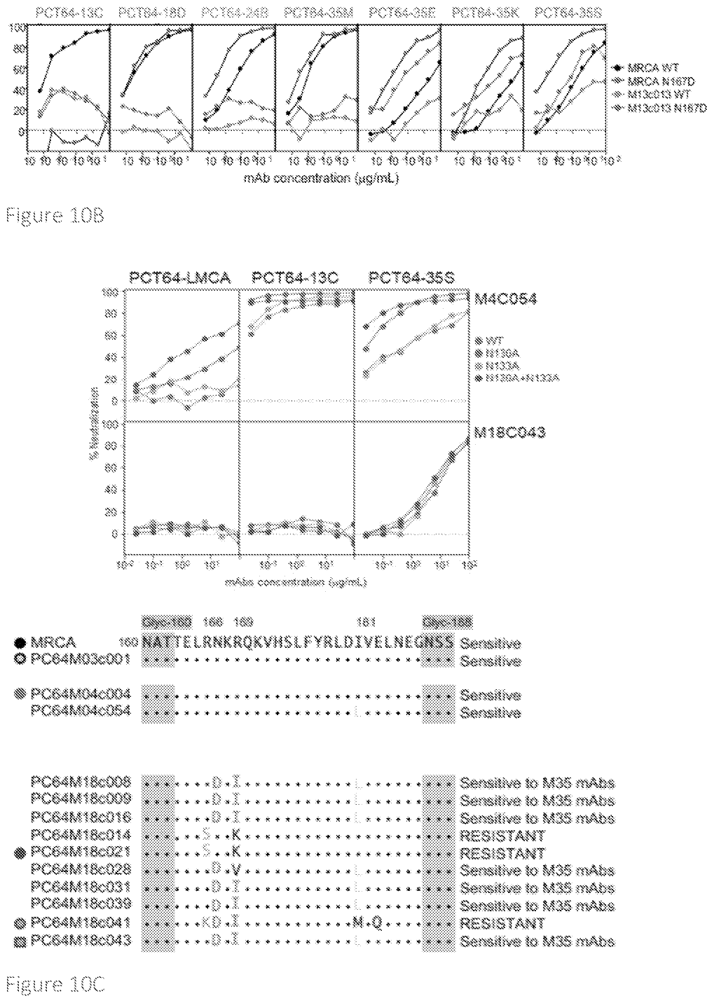

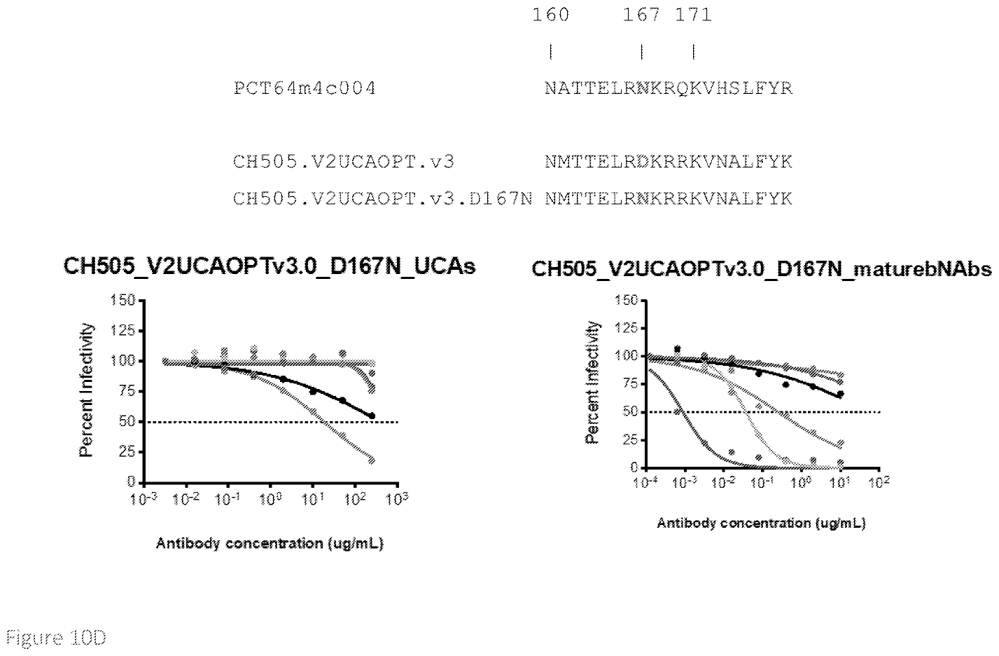

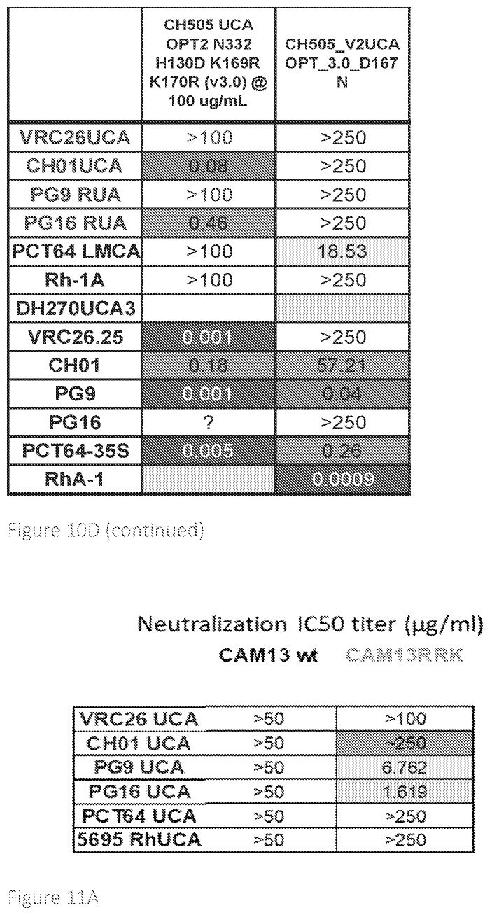

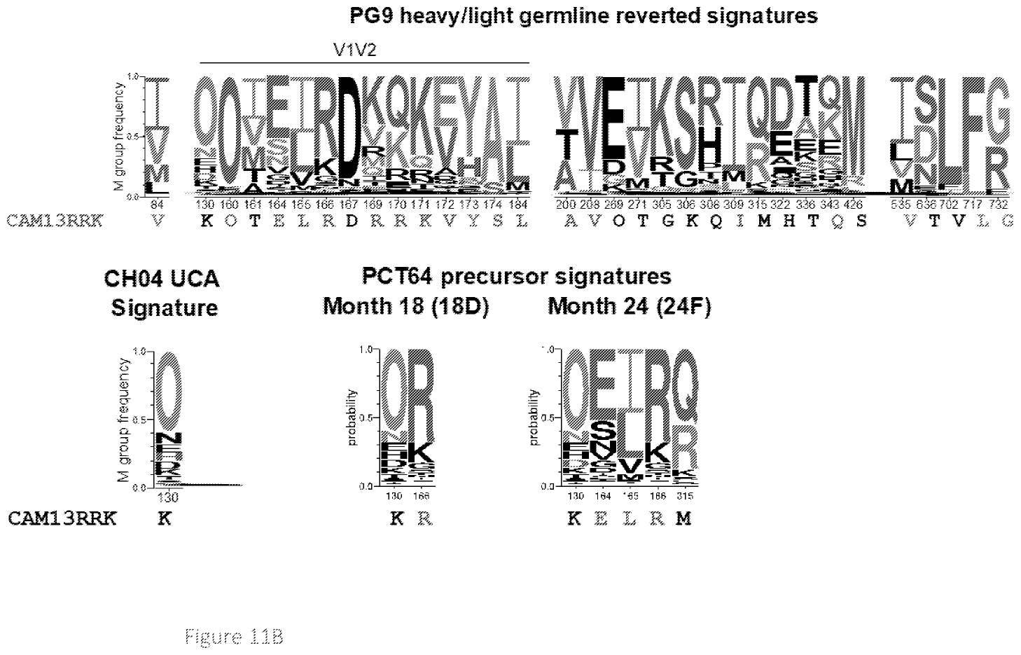

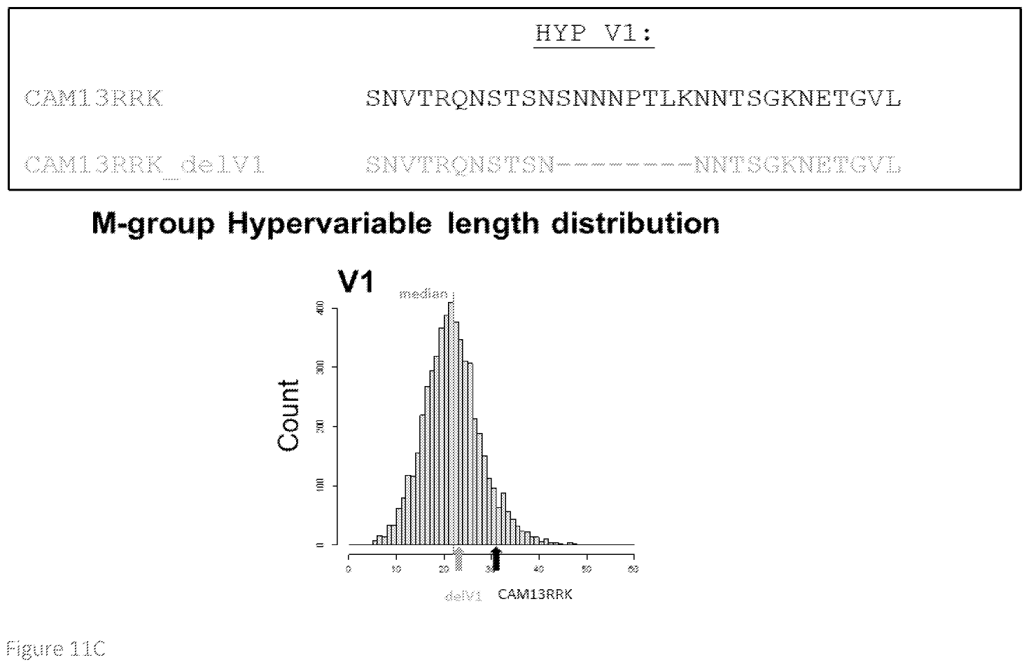

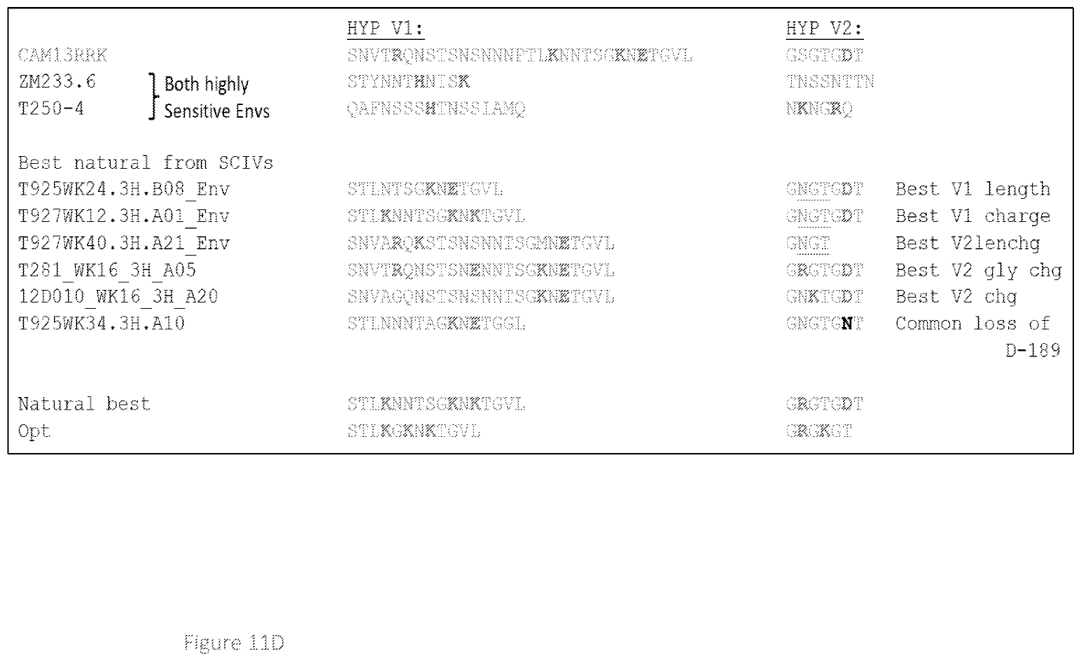

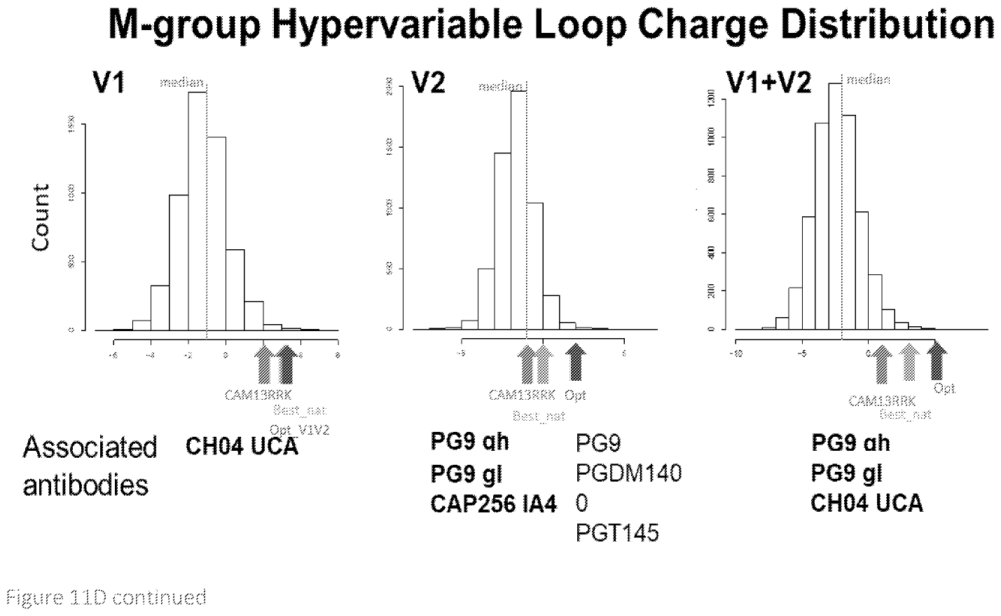

It was observed that 4 out of 177 viruses were neutralized for fully reverted PG9gHgL. Signatures were detected using the following criteria: (i) contact sites; (ii) p less than 0.05; and (iii) at least two sensitive viruses have the signature. Using these criteria, one signature was identified-Arginine 170 (i.e., Arg170 or R170). FIG. 7A. Arg170 is a polar contact with Tyr111. Lys170, however, is not a contact, as it is 4.2 Å away. PG9 UCA possesses Trp111, raising the question of whether this residue participates in cation-pi interactions with Arg170.

Mutations of K169 to arginine (K169R) resulted in enhanced PG16 RUA sensitivity of about 10-fold and double mutations at K169 and K170 (K169R and K170R) resulted in roughly a 50-fold sensitivity enhancement. There was a similar, though less pronounced improvement in PG9 with these mutations. Based on these results and signatures the following mutations were investigated: CH505 UCA_OPT+Q170R and CH505 UCA_OPT+Q170R+K169R. Results are depicted in FIG. 7B.

Previous designs relied on weak outside epitope signatures for CAP256 IA4 (breadth=3 of 202 viruses). The threshold signature was relaxed to A-161 sensitive for CAP256 IA4 & UCA. Structurally A-161 is at the base of 160 glycan, so it may impact glycan dynamics or processing. Experimental testing showed M161A did not gain CAP256 UCA sensitivity. FIG. 7C.

CH505 UCA_OPT2+N332 is weakly neutralized by PCT64 LMCA (IC50=105 ug/ml). Previous designs were most favorable for all PCT64 intermediate signatures except at 130. D-130 associated with sensitivity. H-130 was used as it was the only CH04 UCA sensitivity signature, and was not a significant signature for PCT64 intermediates. CH505 UCA_OPT+H130D was tested to determine the PCT64 LMCA signature. FIG. 7D.

Q170R improved sensitivity to PG16RUA and CH01 RUA. FIG. 7E. H130D improved sensitivity to PCT64 LMCA and reduced sensitivity to CH01 RUA. FIG. 7F. H130D+K169R+Q170R improved sensitivity to PG9 and PG16 RUAs. FIG. 7G. This was a surprising 100-fold improvement for PG16 RUA compared to the Q170R mutation.

The H130D+K169R+Q170R mutation displayed slight improvement over UCA OPT2 for CH01 UCA, but was slightly reduced compared to the Q170R mutation. H130D+K169R+Q170R sensitivity was slightly reduced for PCT64 LMCA compared to UCA OPT2. H130D sensitivity for PCT64 LMCA was also reduced, but neutralization was observed at 50% at about 100 μg/ml.

In summary, the redesign of UCA_OPT showed partial success. H130D improved sensitivity to PCT64 UCAs. Q170R and K169R improved PG9 and PG16 UCA sensitivity. Triple mutants can be potentially neutralized by CH01 and PG16 UCAs and may provide weak neutralization of PG9 and PCT64 UCAs. Leading candidates were tested for sensitivity and were found to be reactive to three out of five linage UCAs tested. FIG. 7H. This includes leading candidate CH505.V2UCAOPT.v2+H130D+K169R+Q170R (CH505.V2UCAOPT.v3). The initial round of signature based optimization of CH505 led to improved sensitivity to all V2 apex mature and gain of reactivity to UCAs of two lineages. This second round produced improvements by gaining reactivity to one more lineage's UCA.

Further development may include improving sensitivity to the three reactive lineage UCAs, developing SOSIP and/or mRNA expression and their associated immunization abilities, testing as SHIVs for accelerated V2 apex bNAb development, and co-optimizing for simultaneous targeting of CH235 and V2 apex UCAs.

Any one of these immunogens could be tested in any suitable animal study to determine immunogenicity of the envelopes.

### Example 5

This example shows information and sequences of a third design round. The design was directed towards a cocktail of pan V2 apex bNAb germline targeting envelopes.

Env signatures were used to design Envs that are sensitive to V2 apex bNAb UCA. The natural Envs CH505.TF, CAP256-SU, CAM13, T250 and Q23 were used as starting templates and improved upon. FIG. 8A shows the leading constructs that together as a cocktail are sensitive to all V2 apex UCAs. No single natural or optimized Env is sensitive to each V2 UCAs, so we want to use a cocktail of Envs for multiple V2 apex bNAb germline targeting. Other constructs are still being improved and tested. FIG. 8B. (CH505_UCA_OPT2 N332 H130D K169R K170R is also referred to as CH505_V2UCA_OPT_v3.0.)

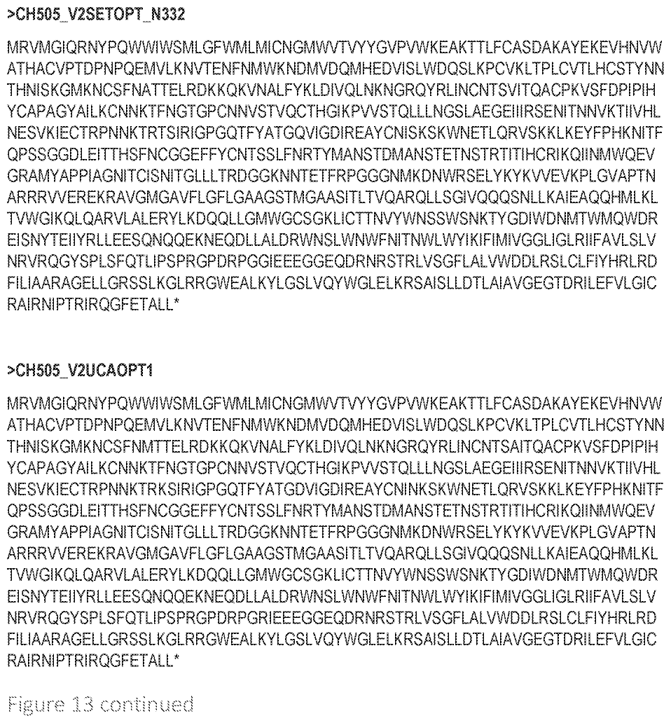

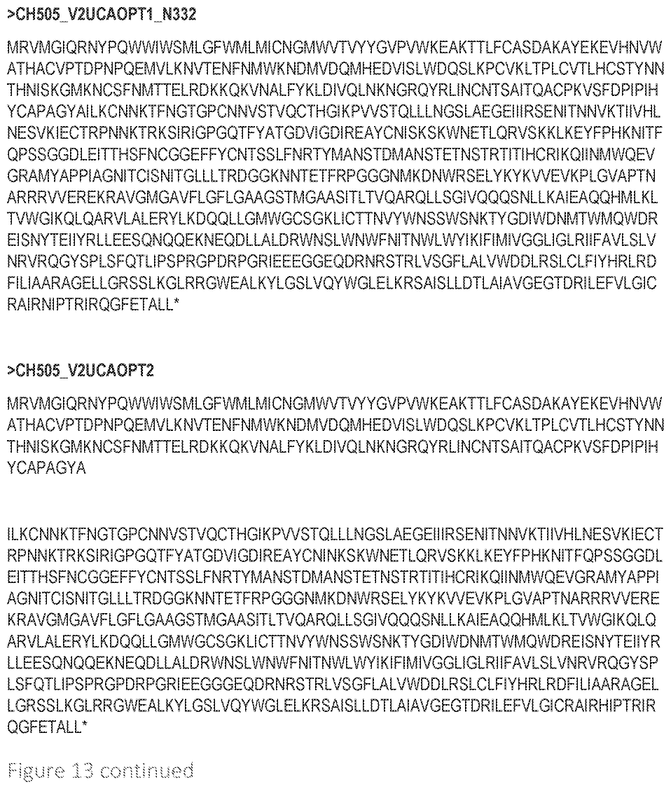

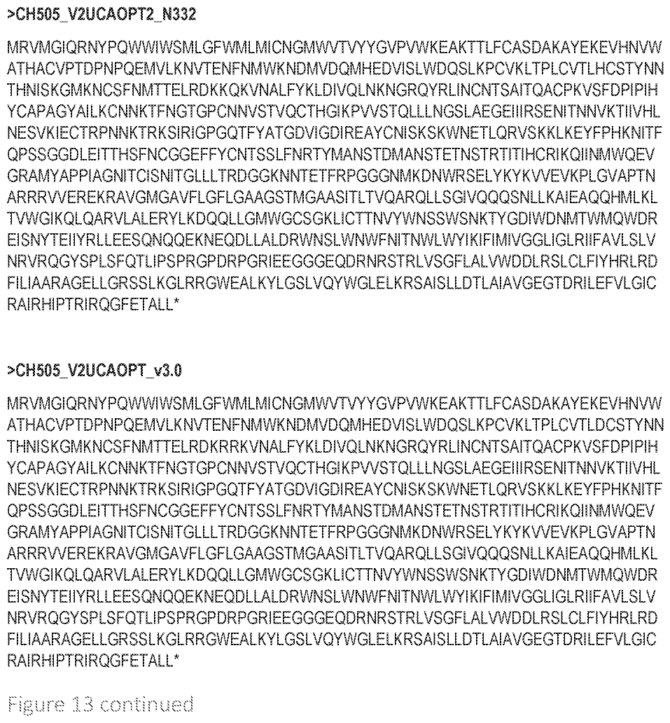

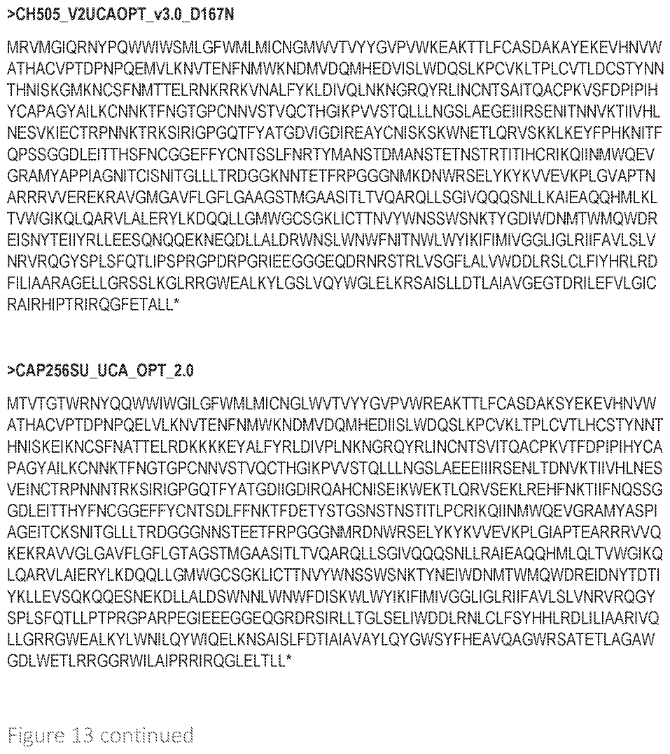

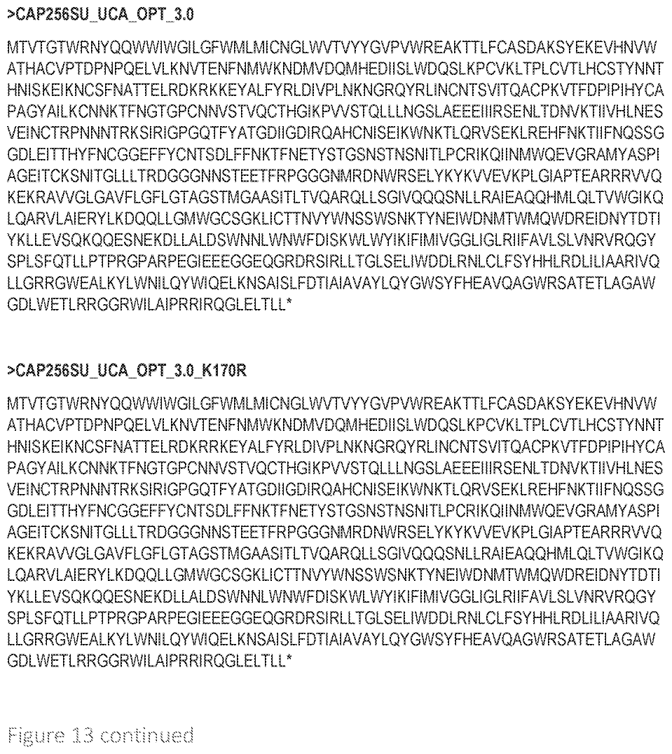

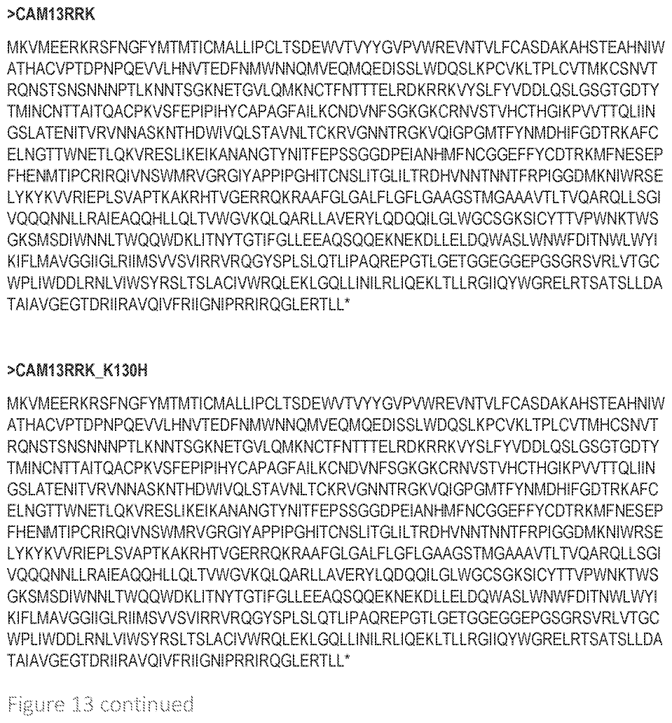

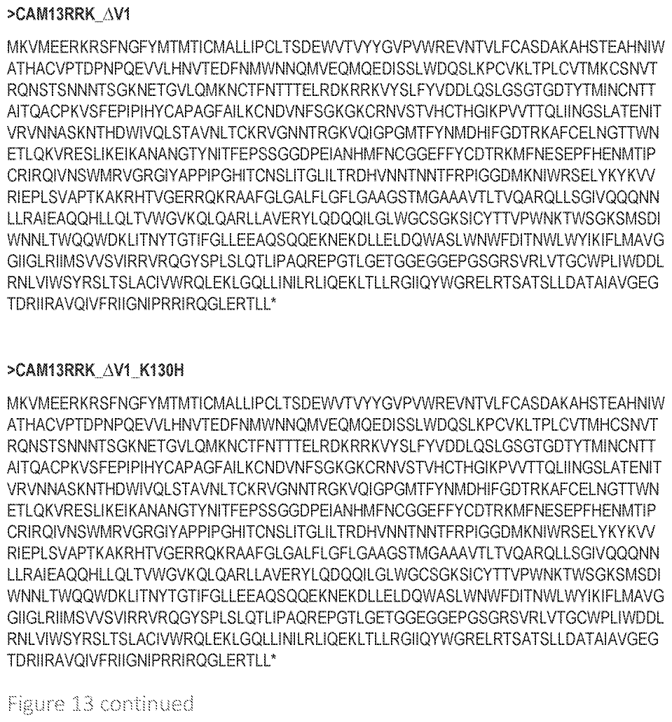

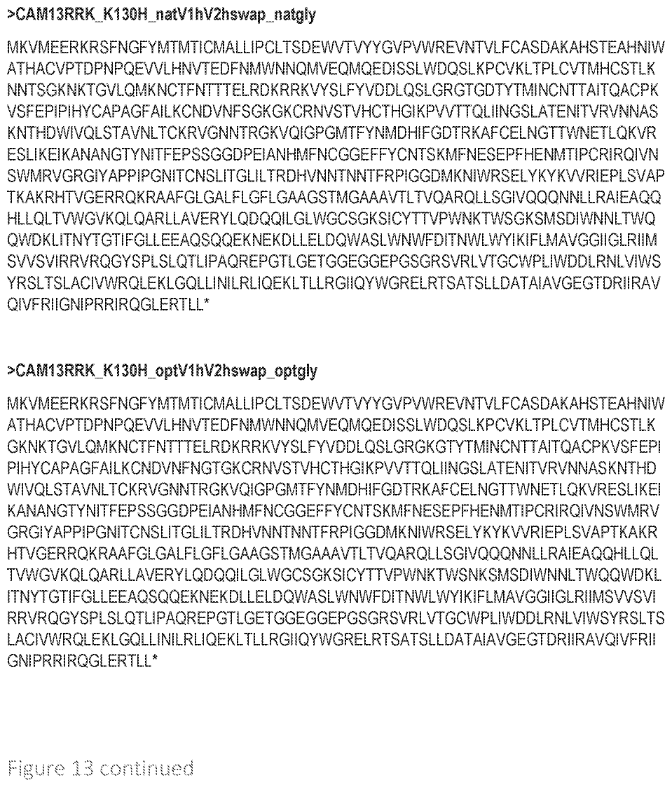

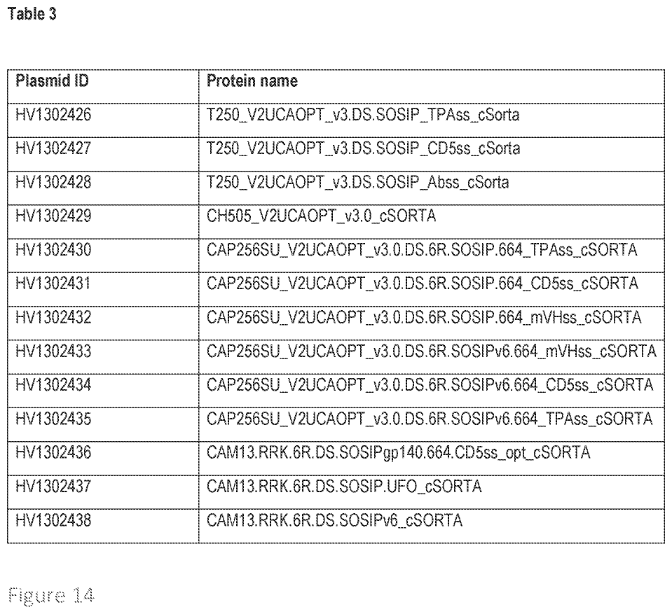

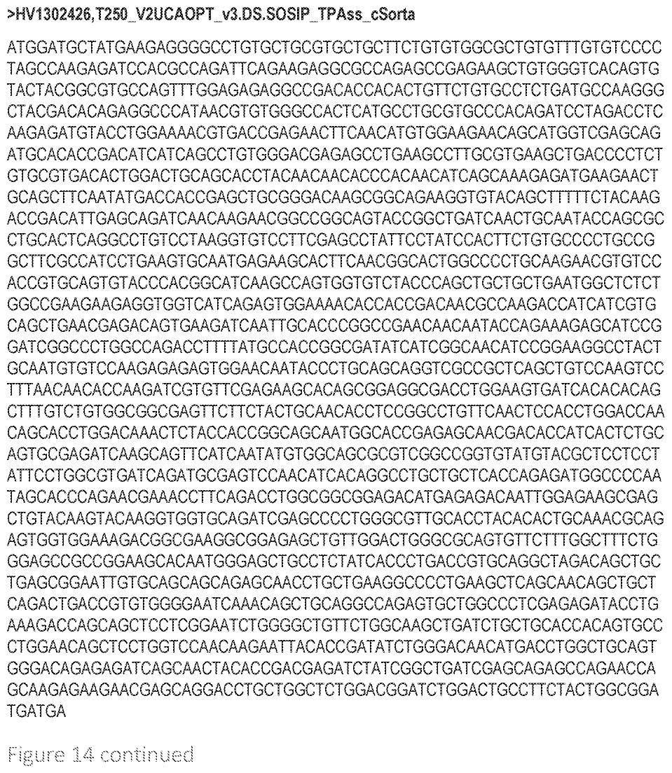

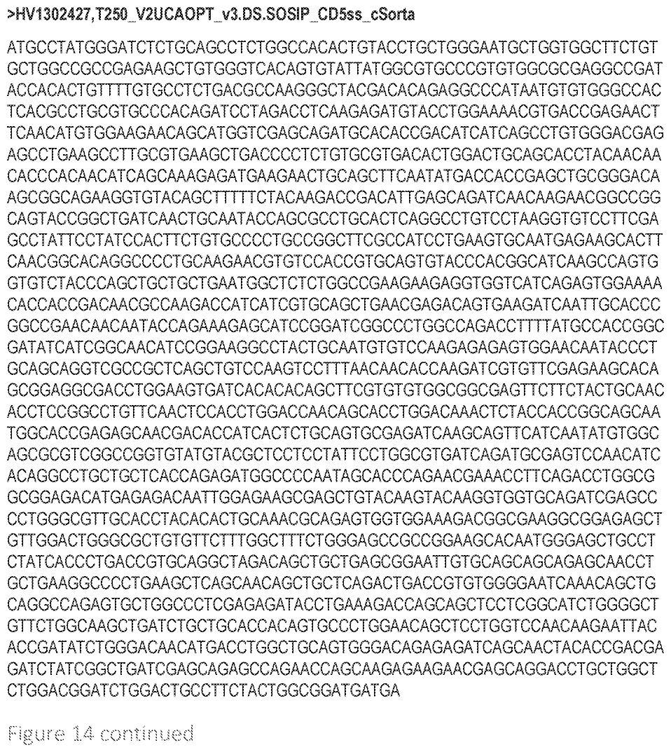

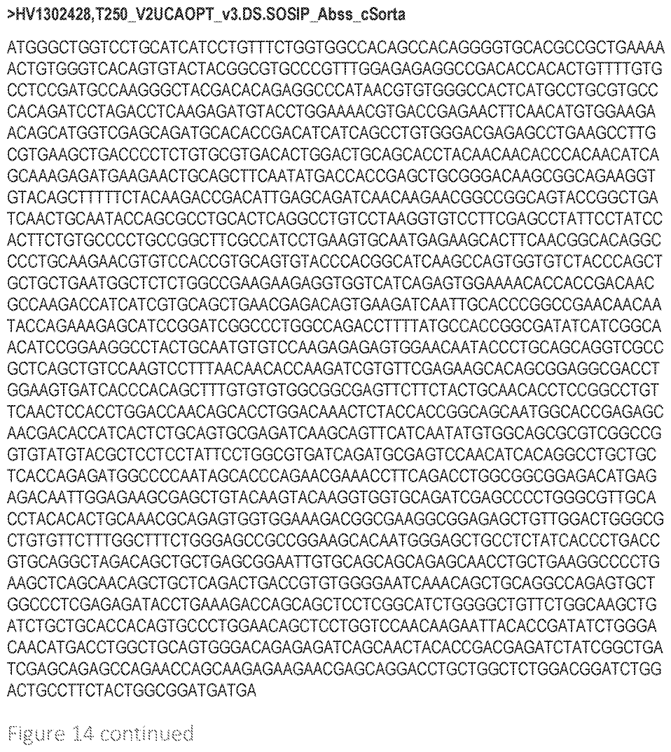

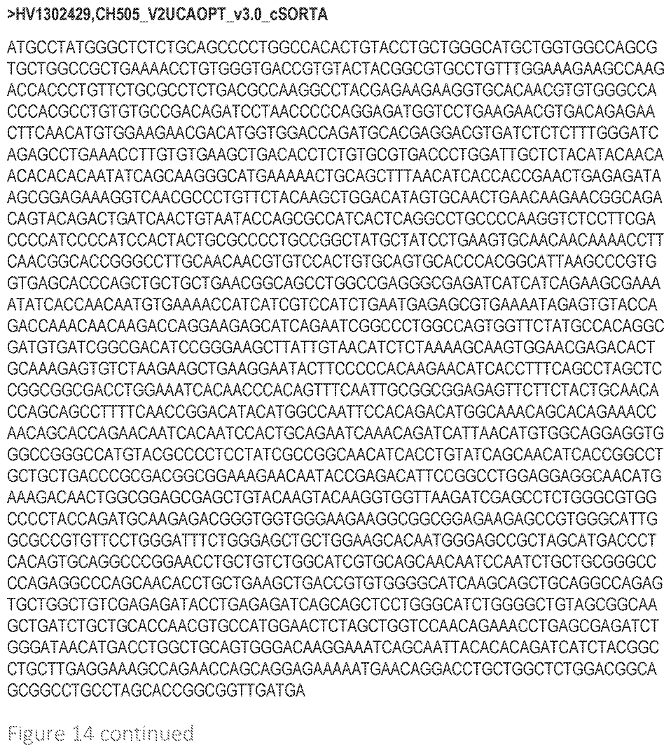

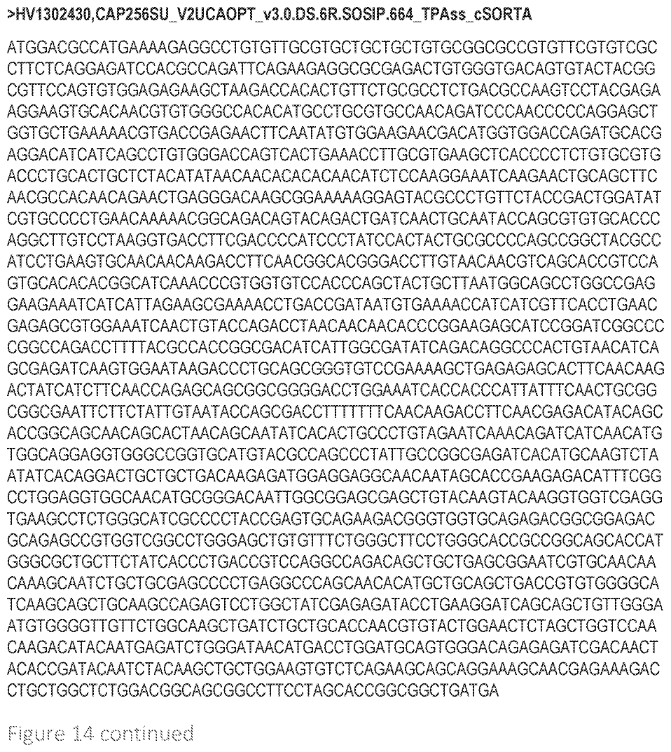

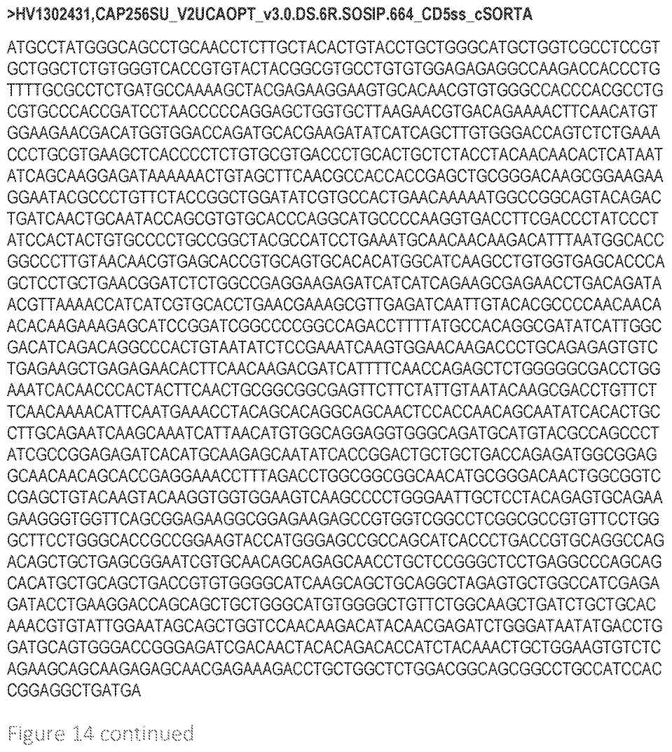

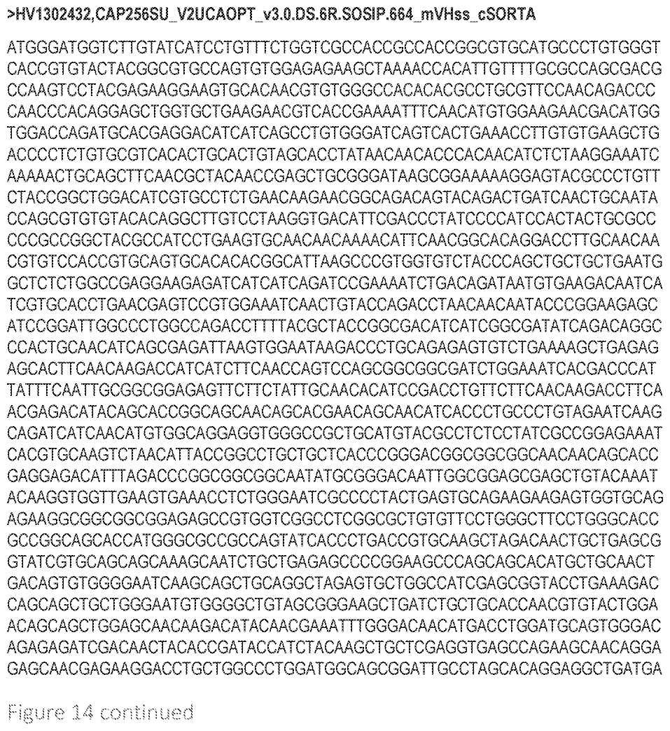

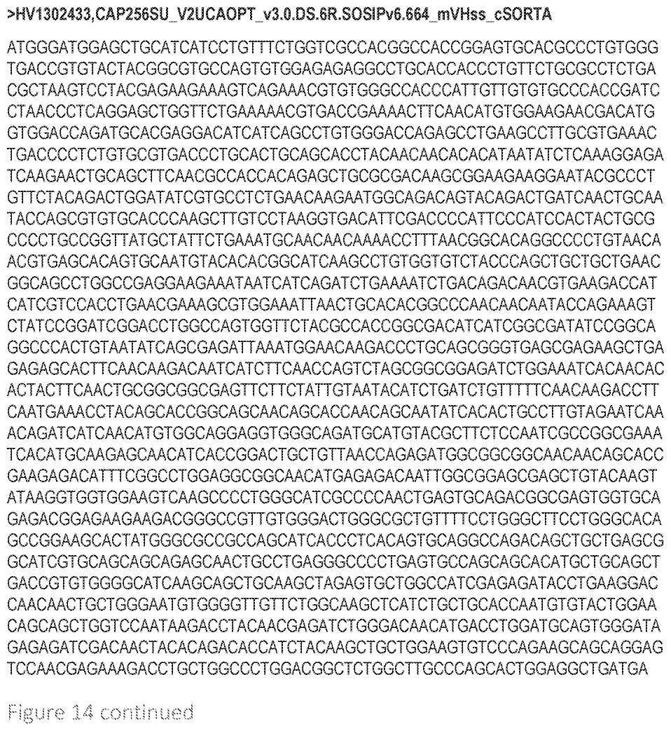

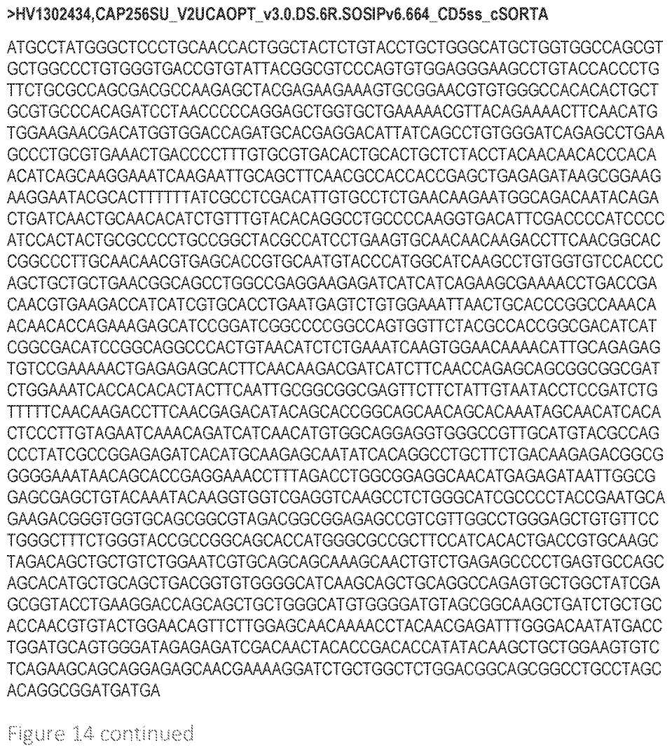

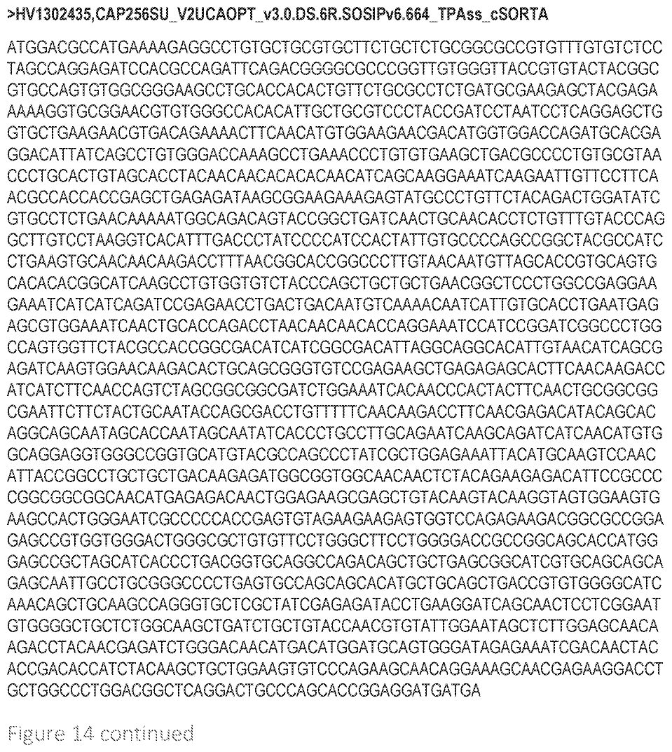

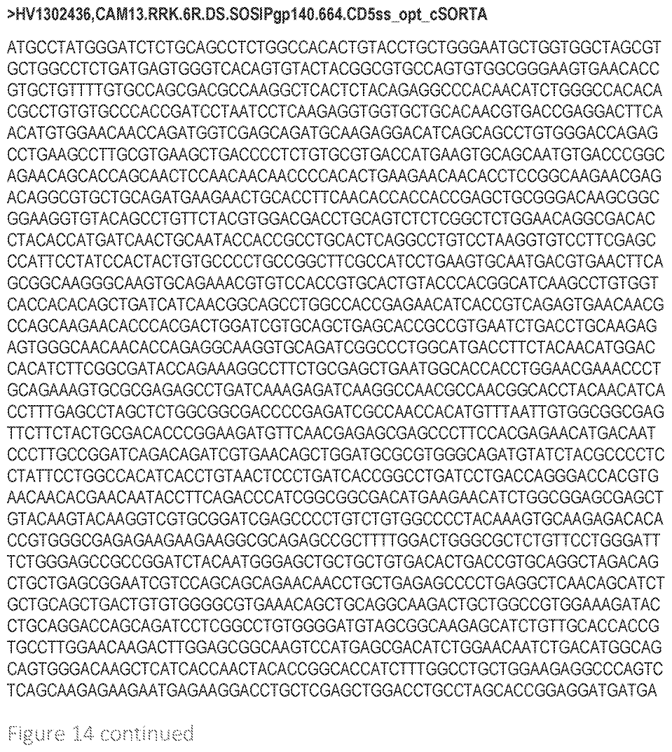

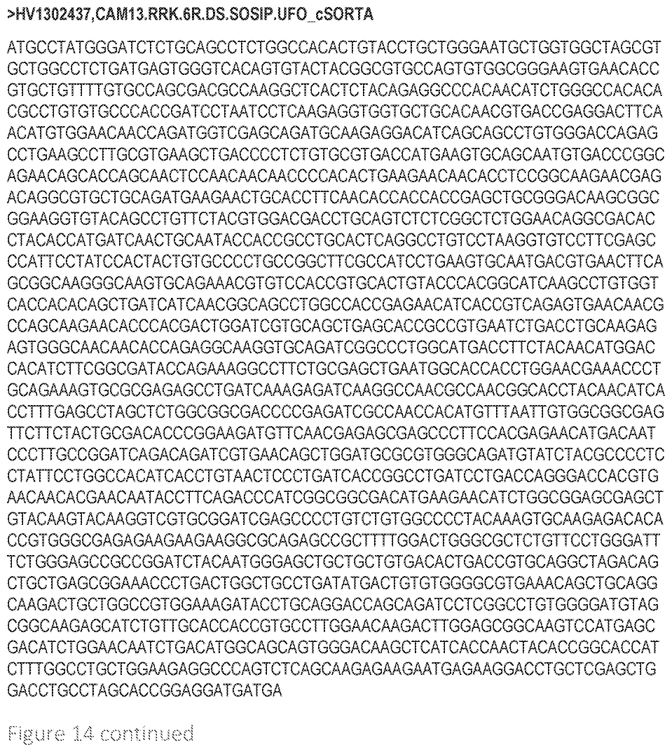

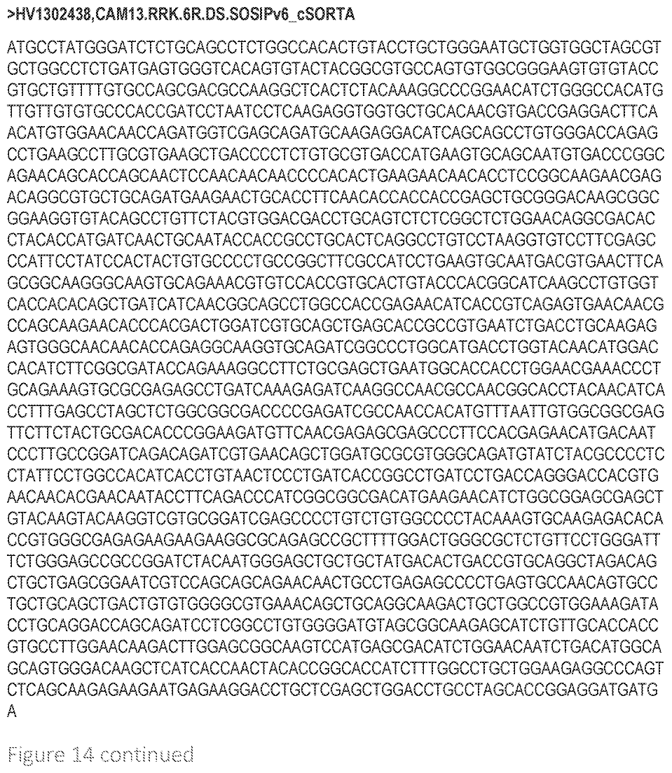

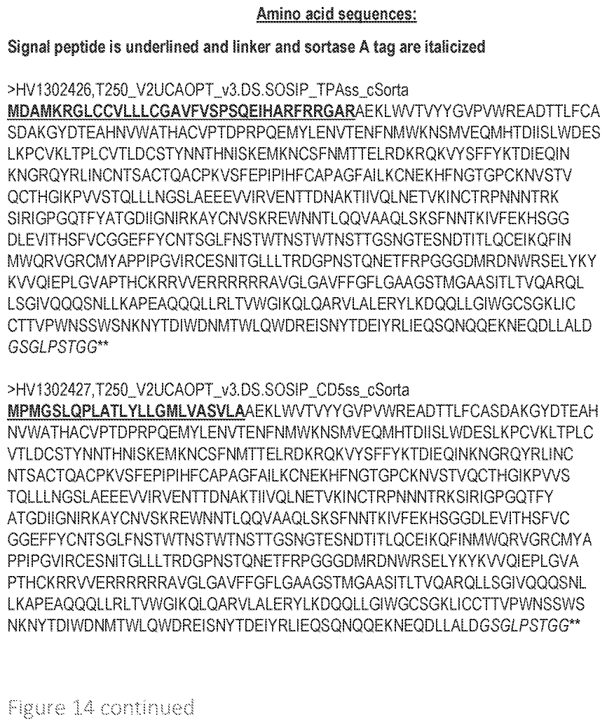

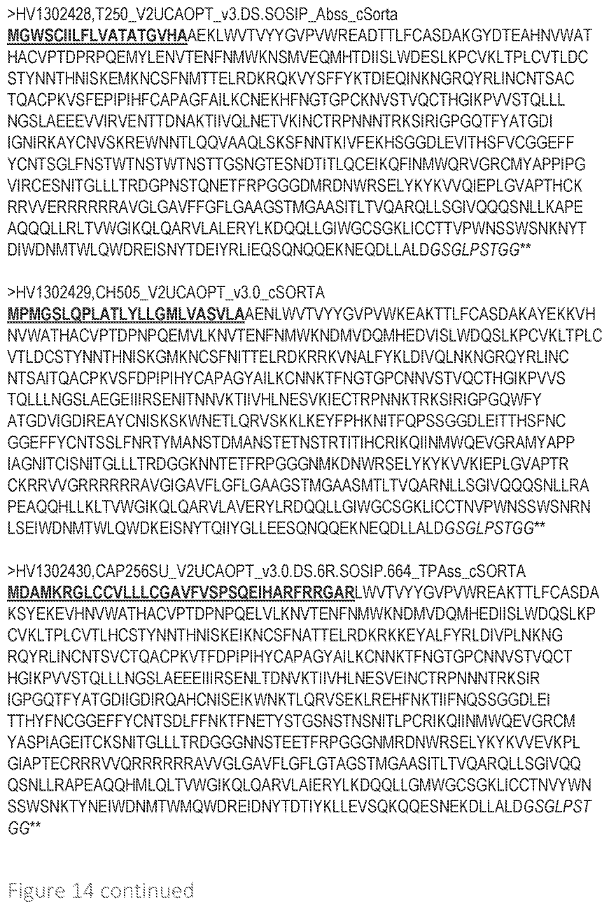

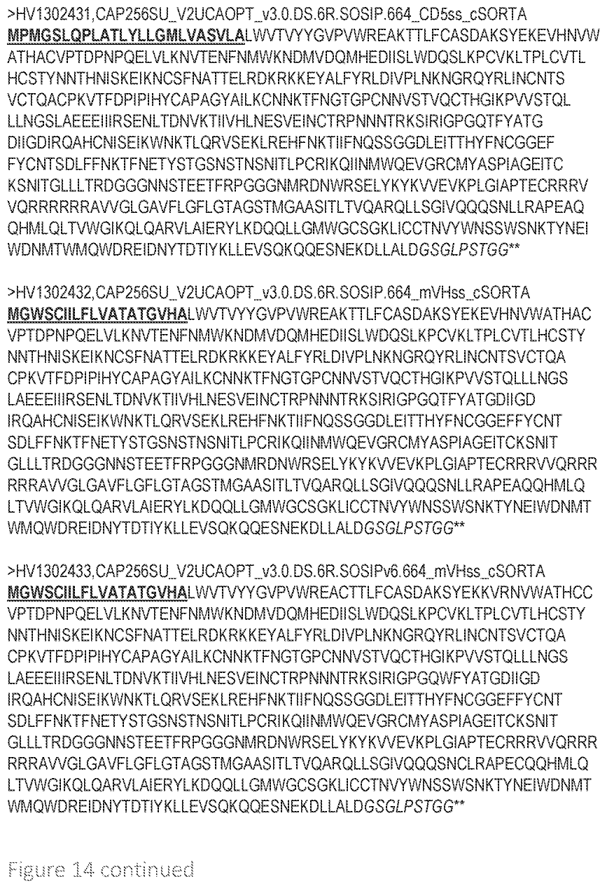

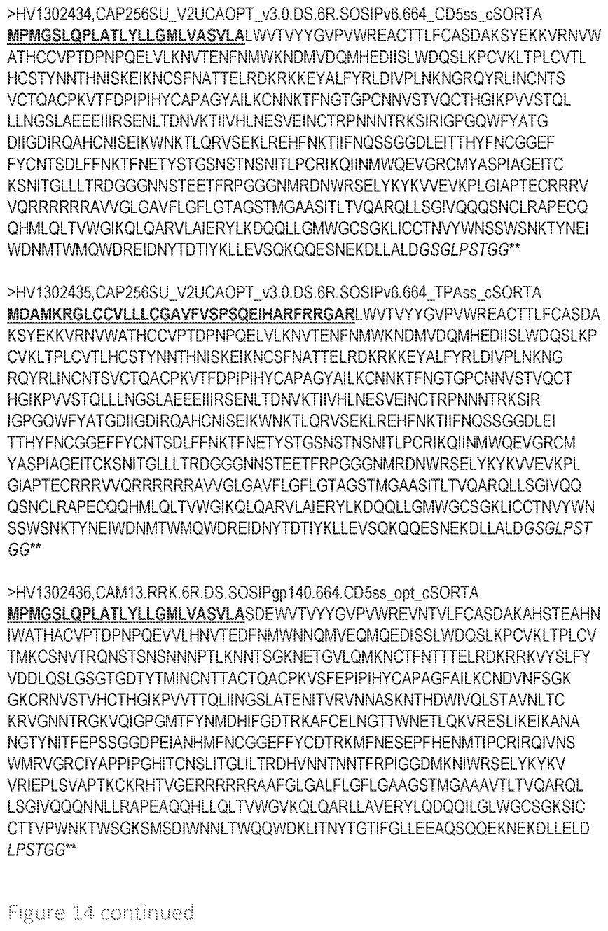

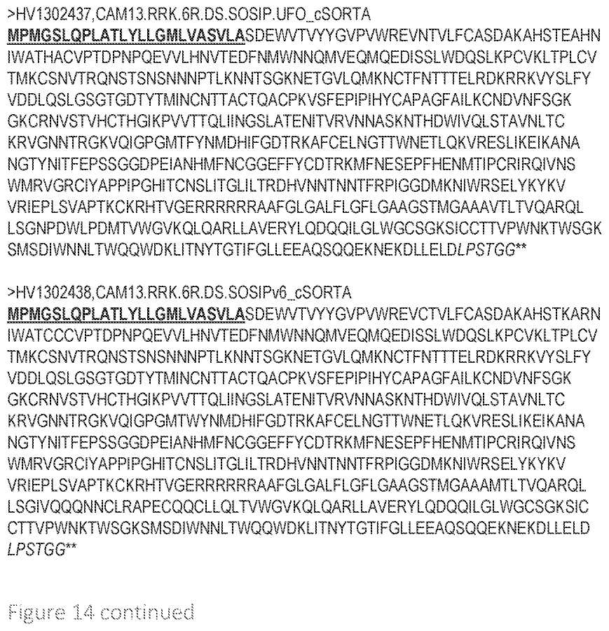

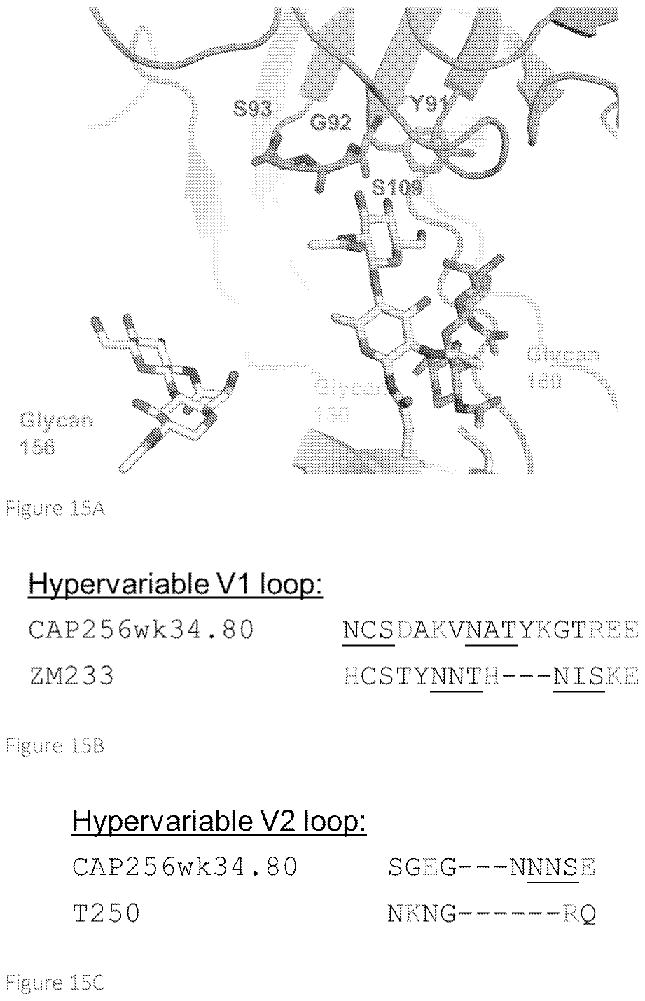

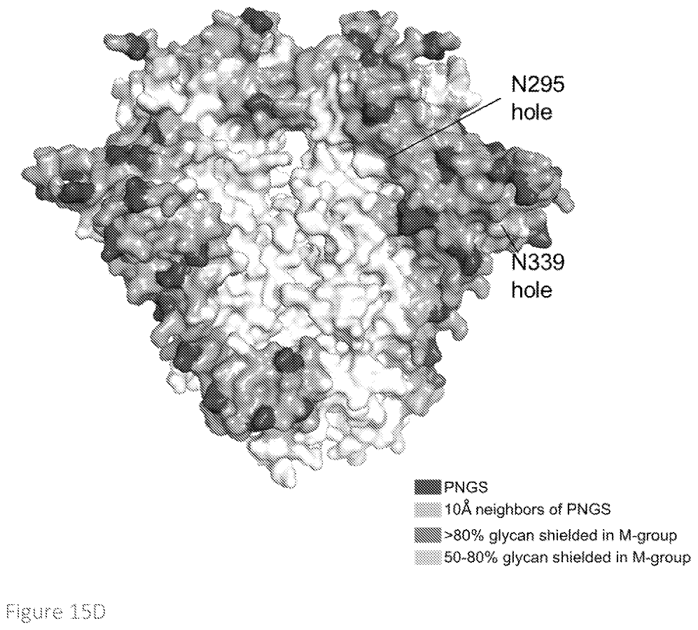

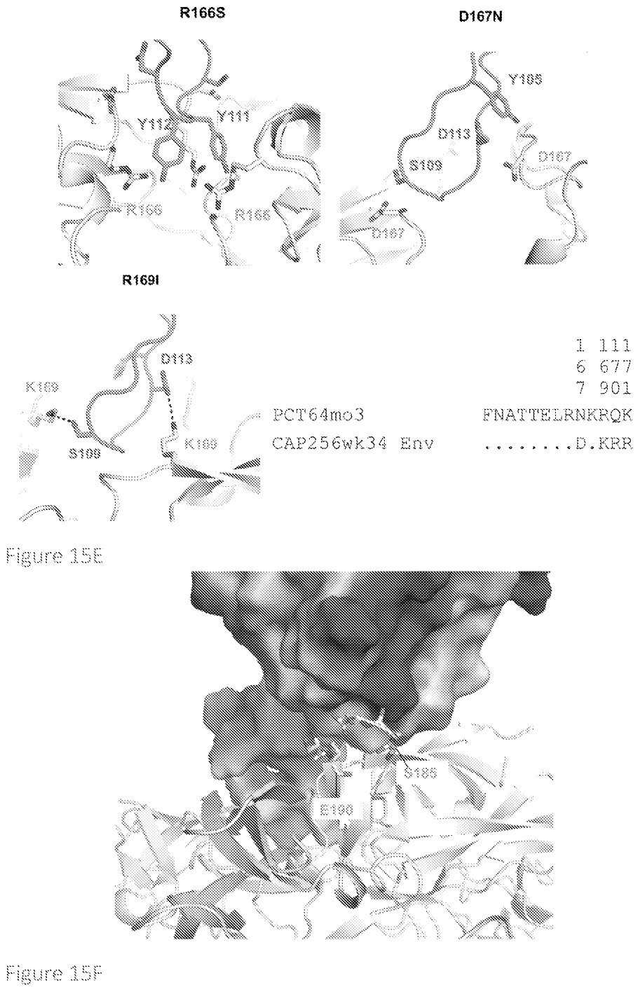

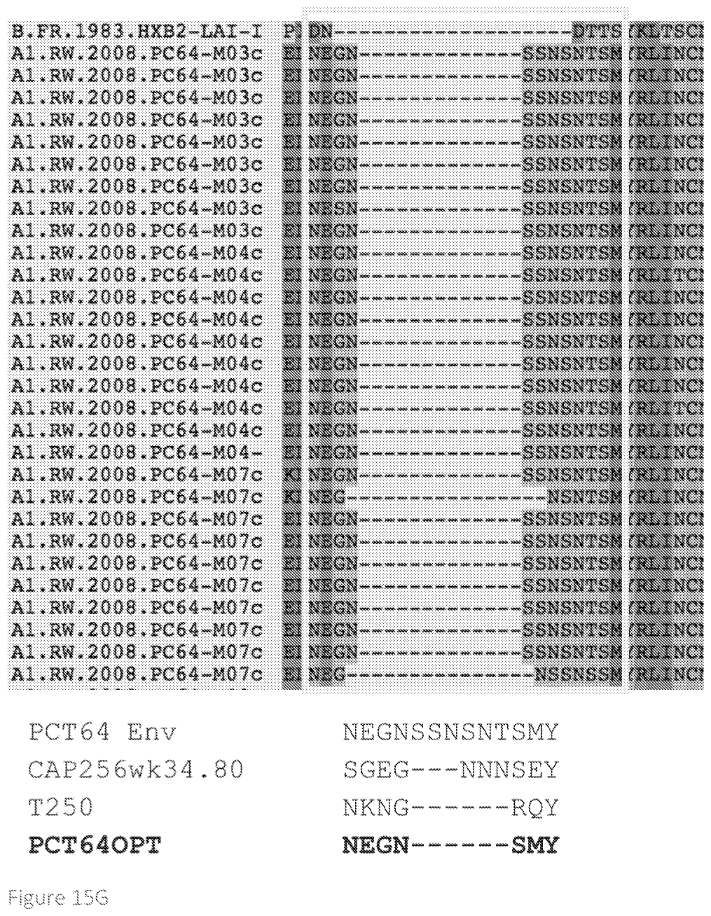

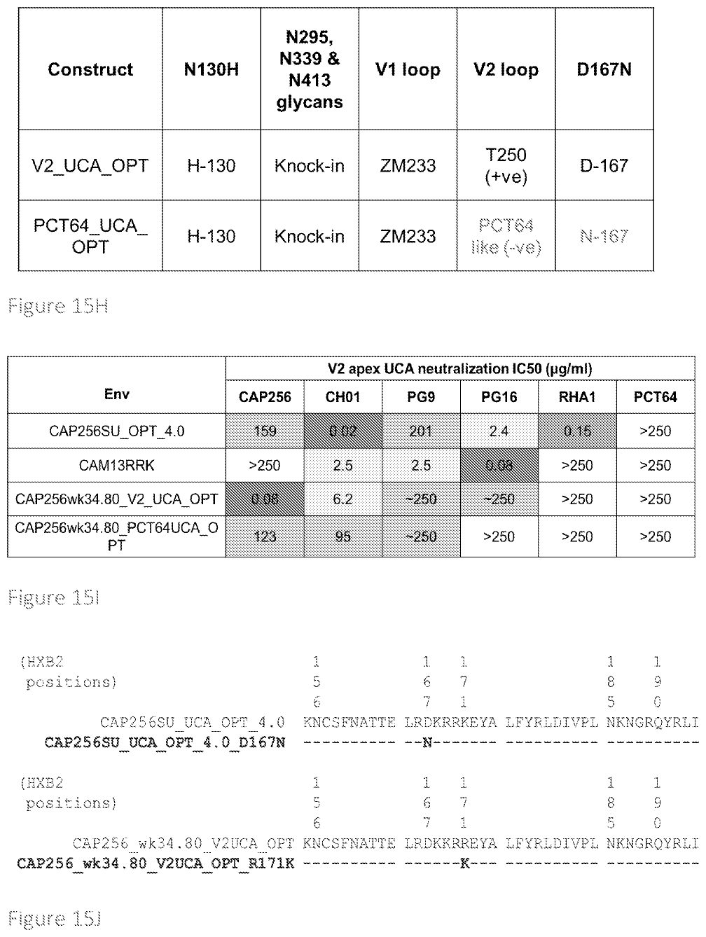

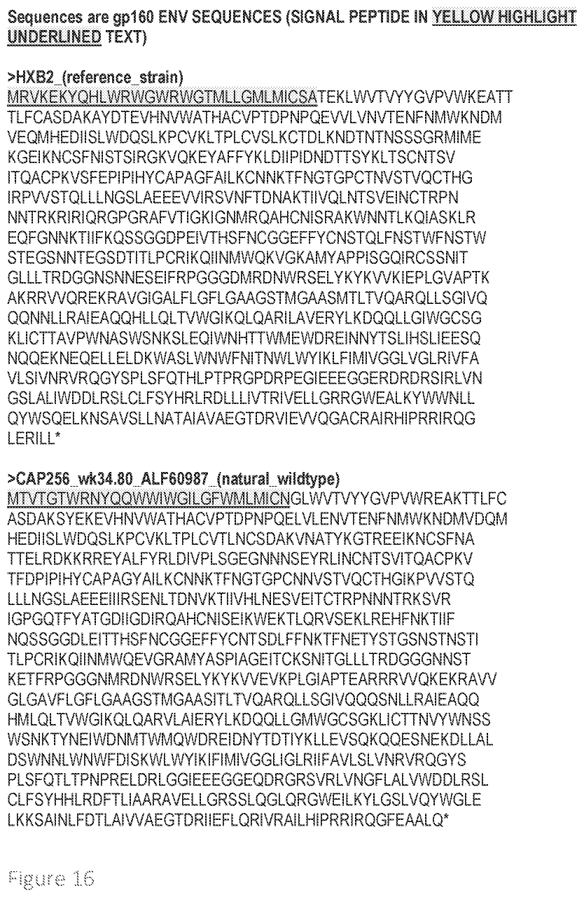

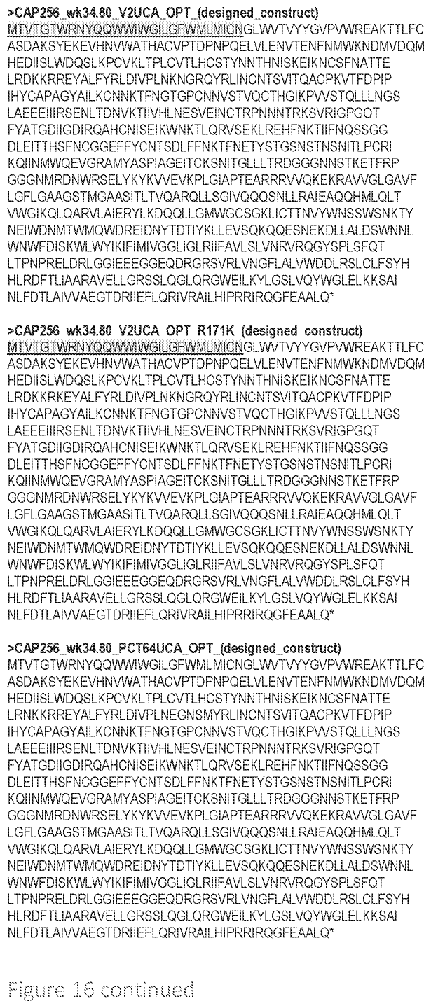

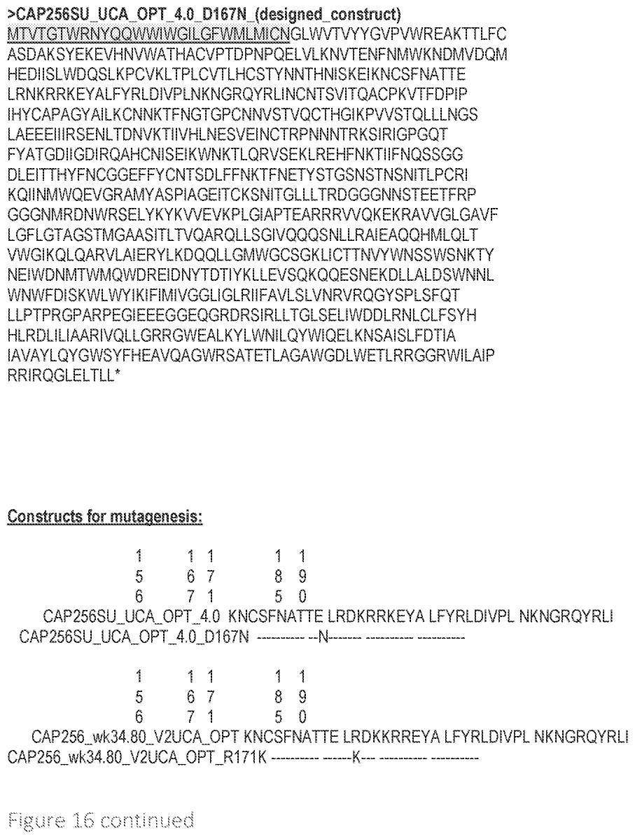

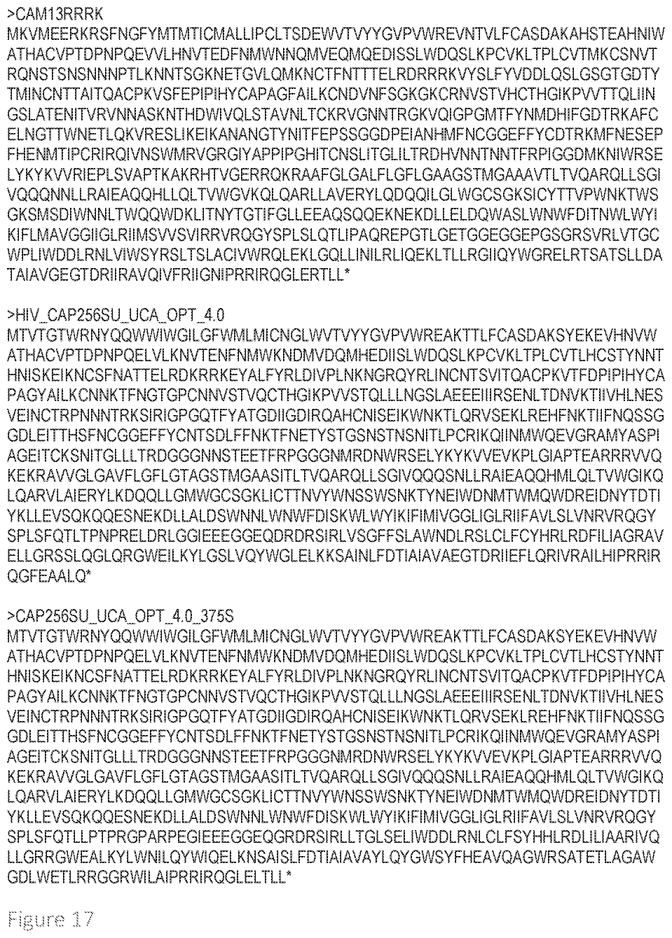

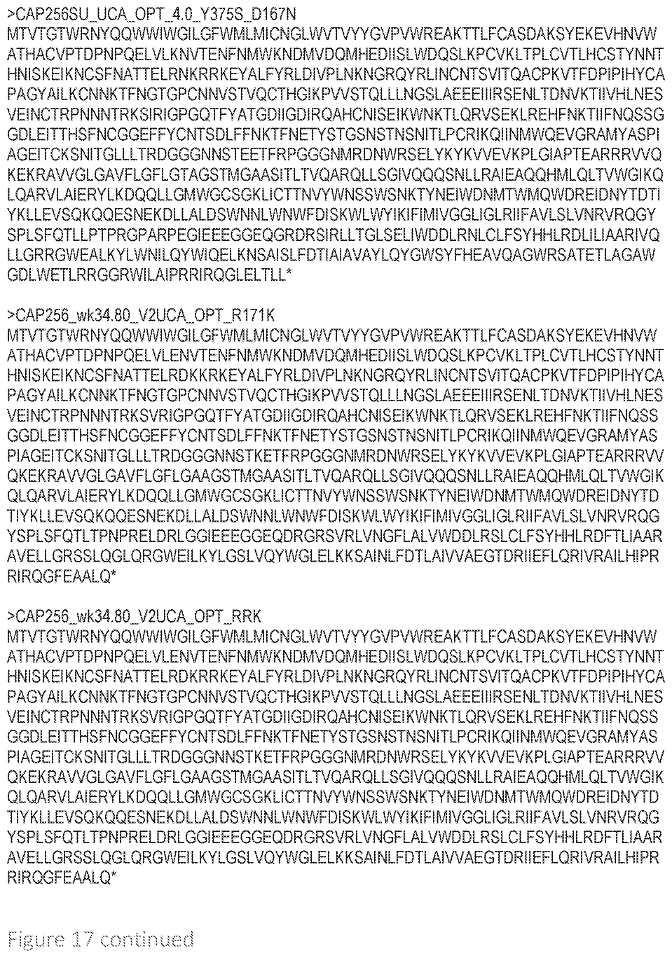

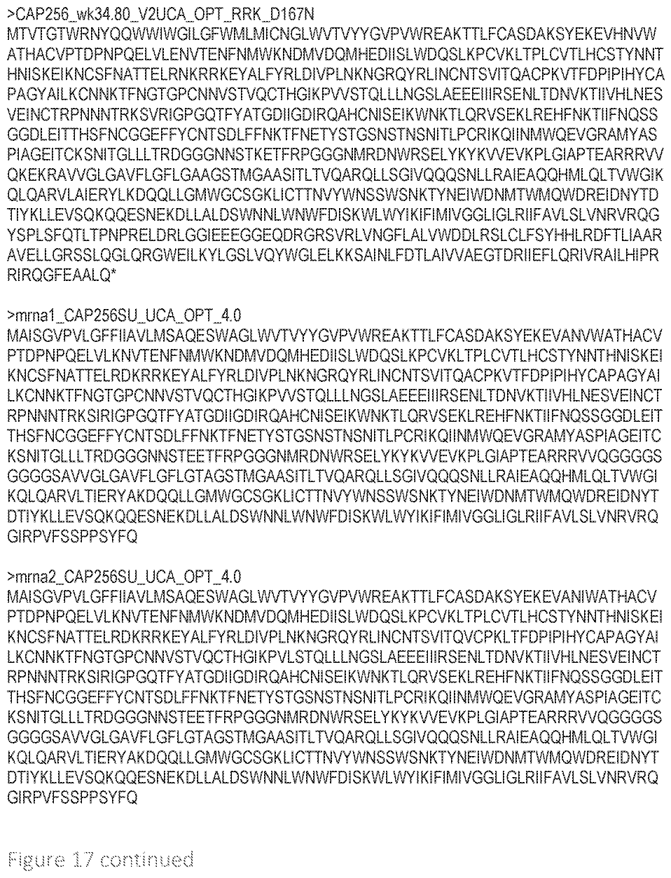

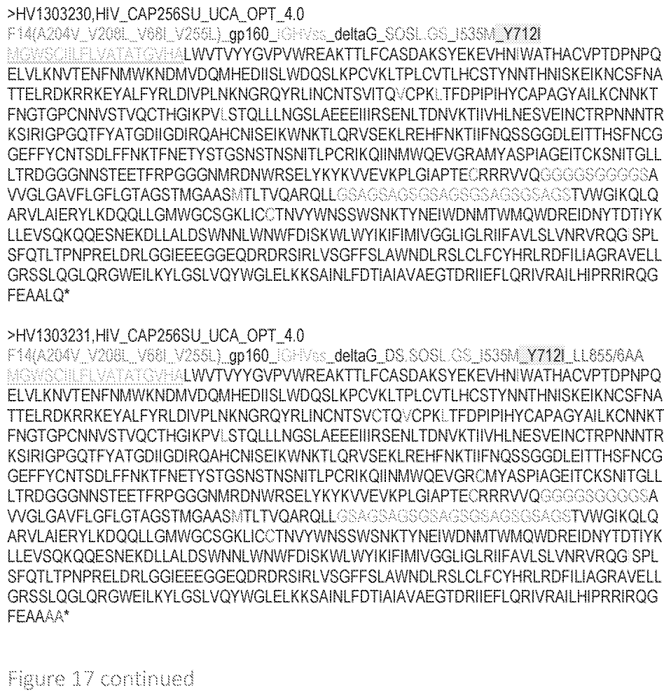

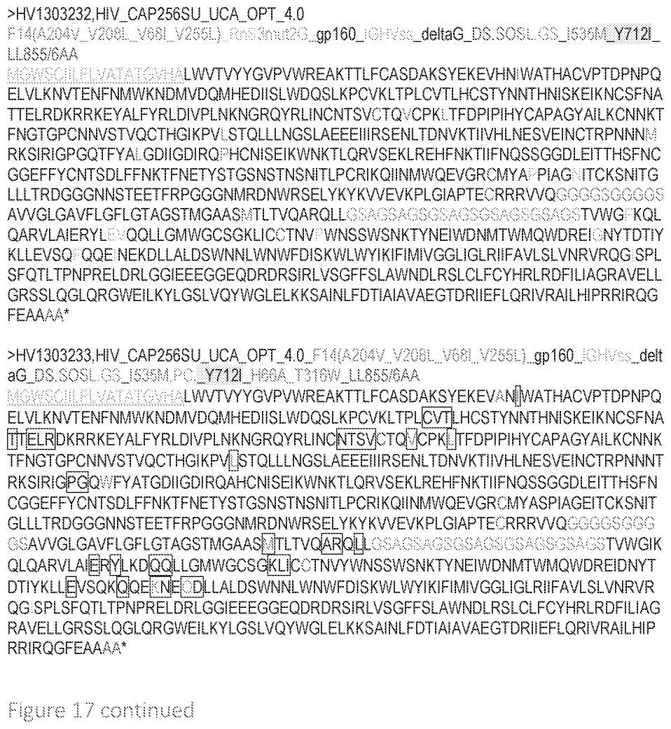

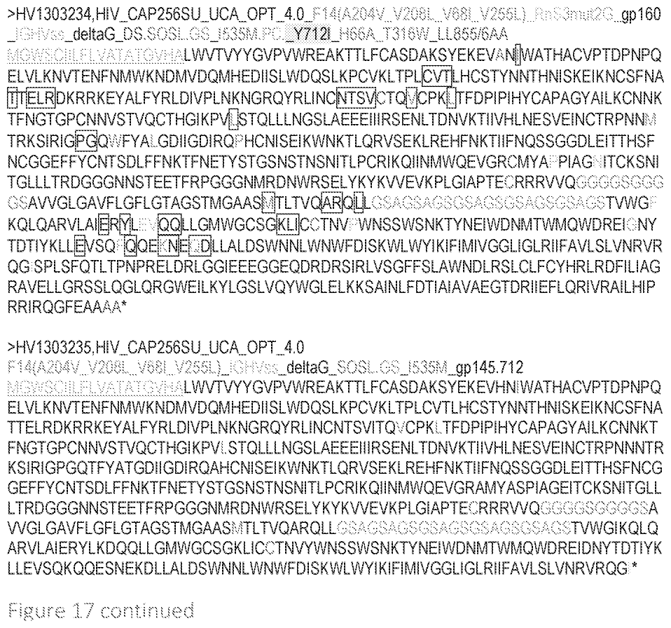

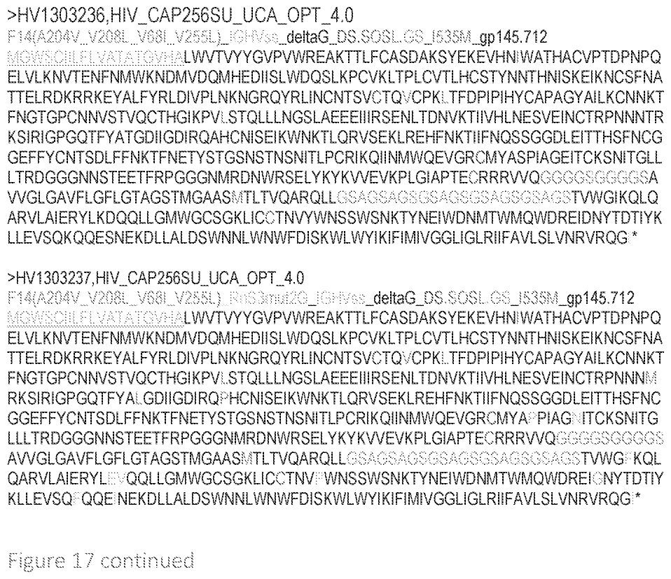

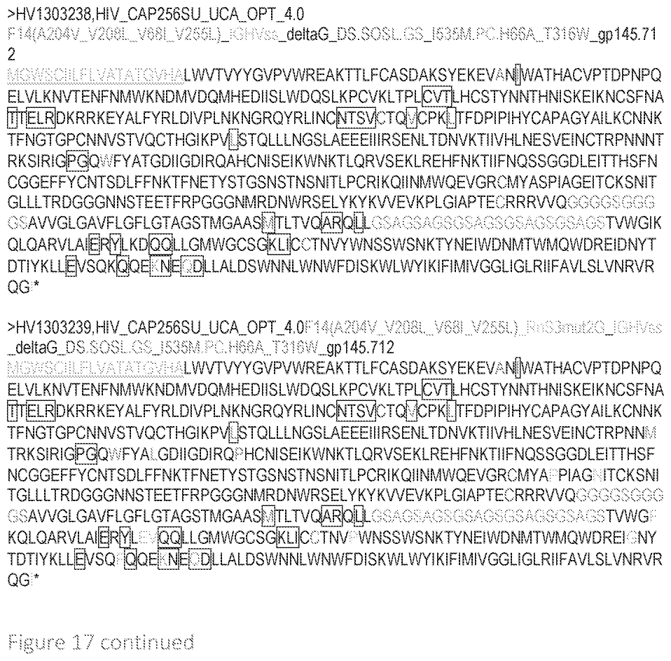

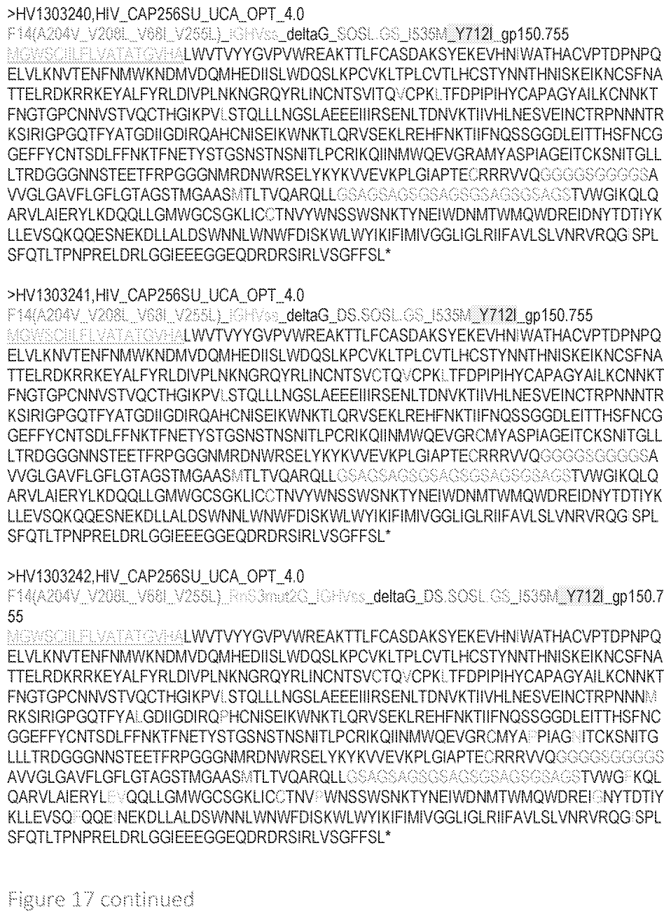

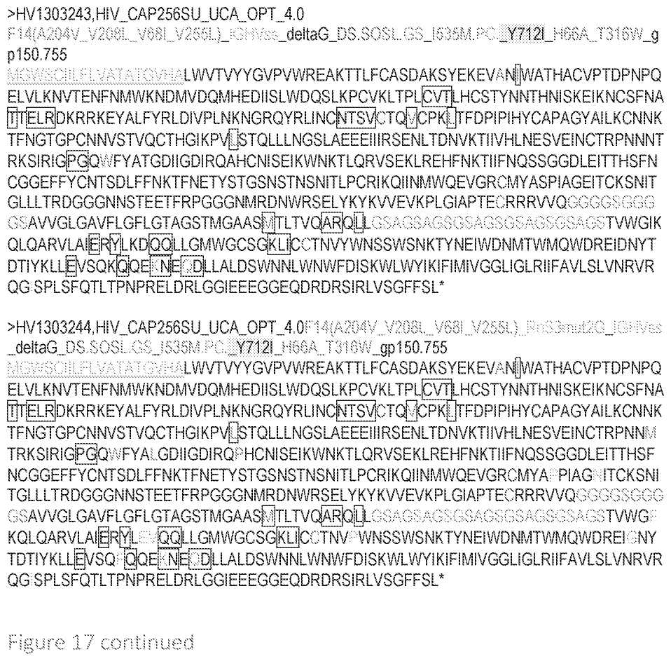

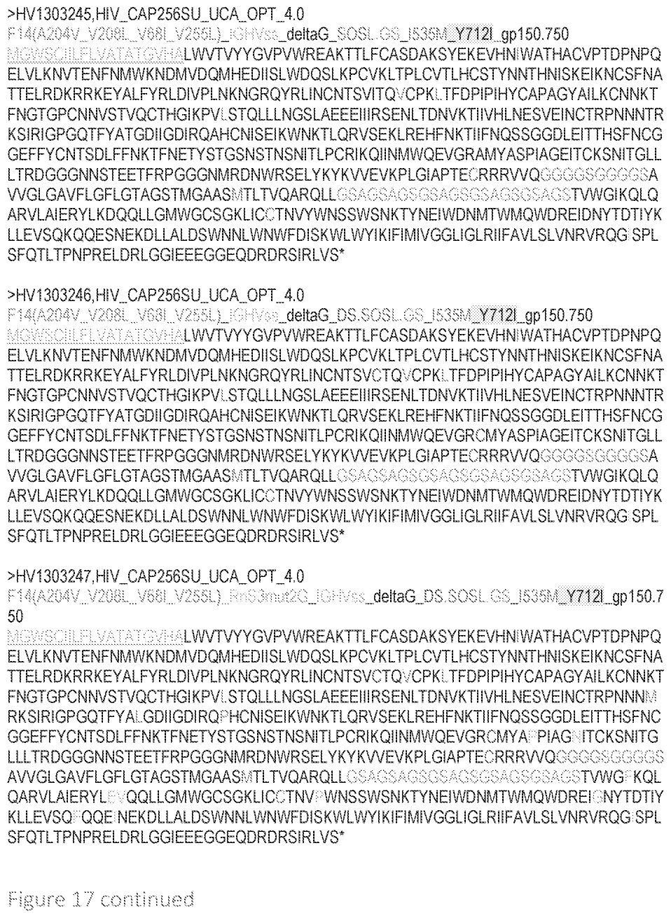

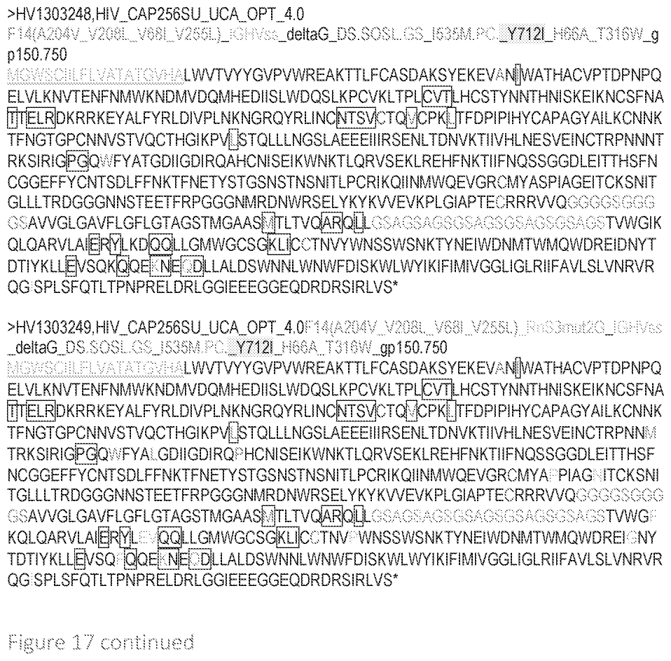

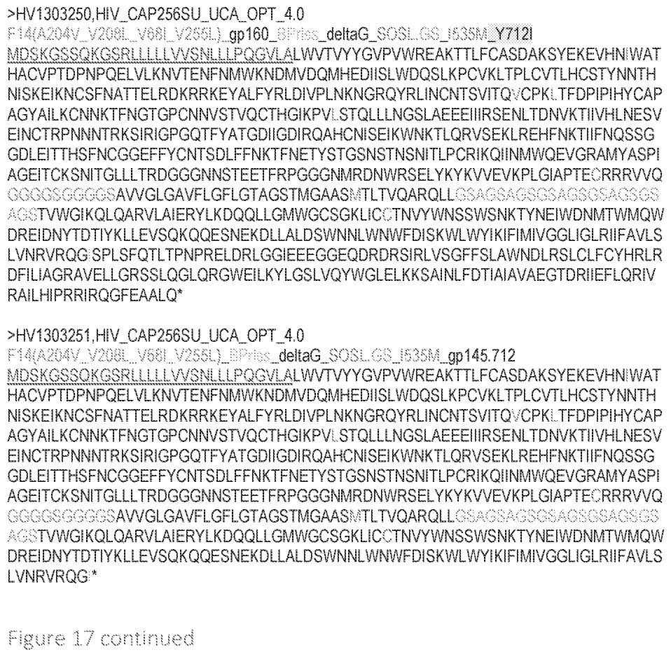

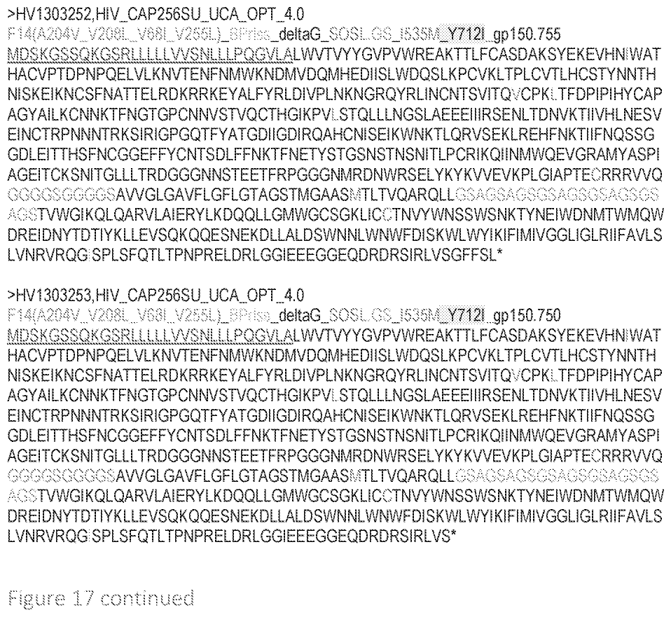

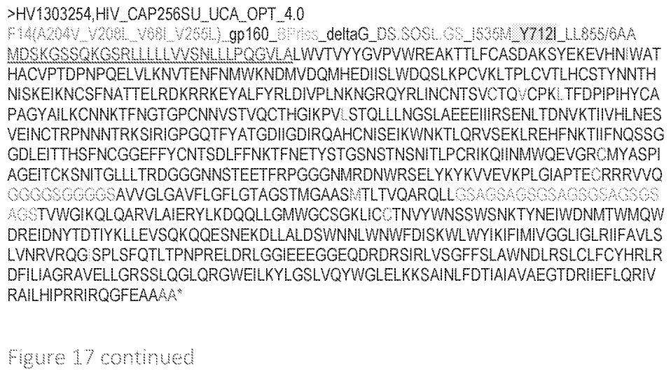

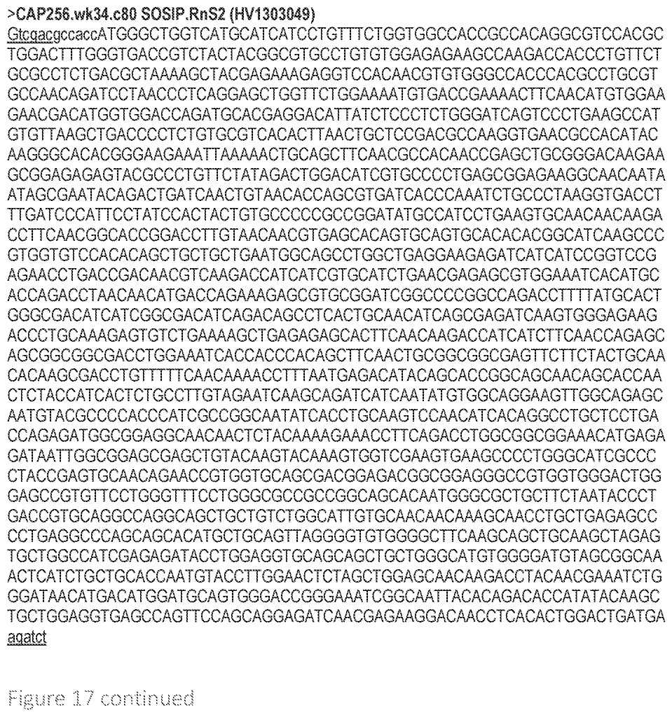

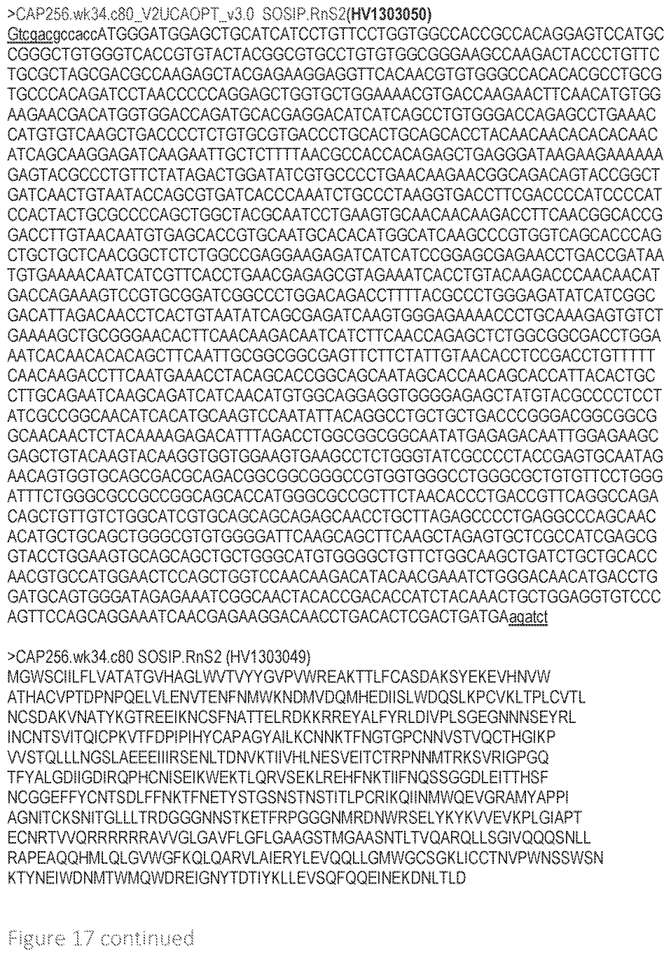

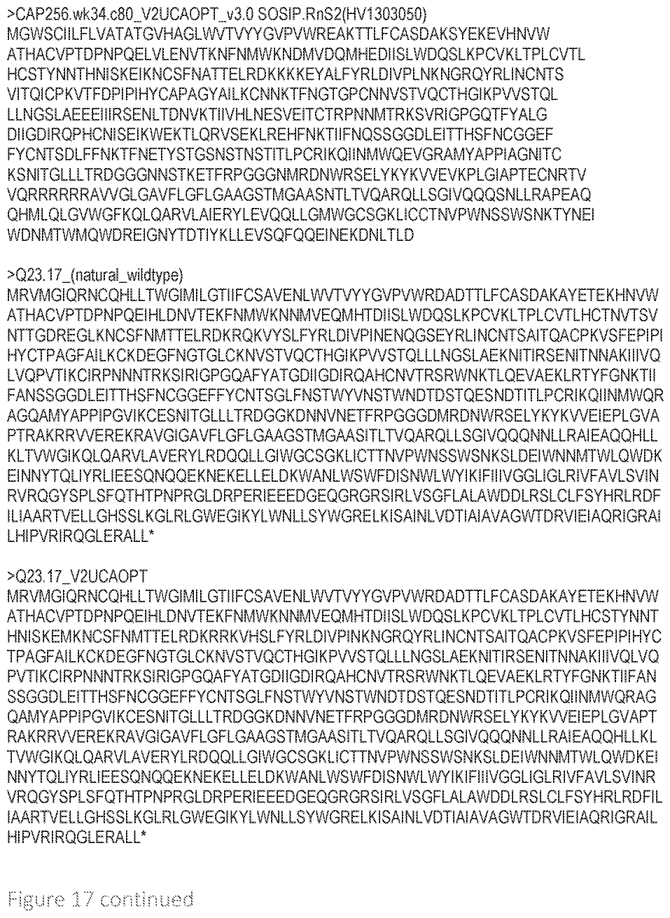

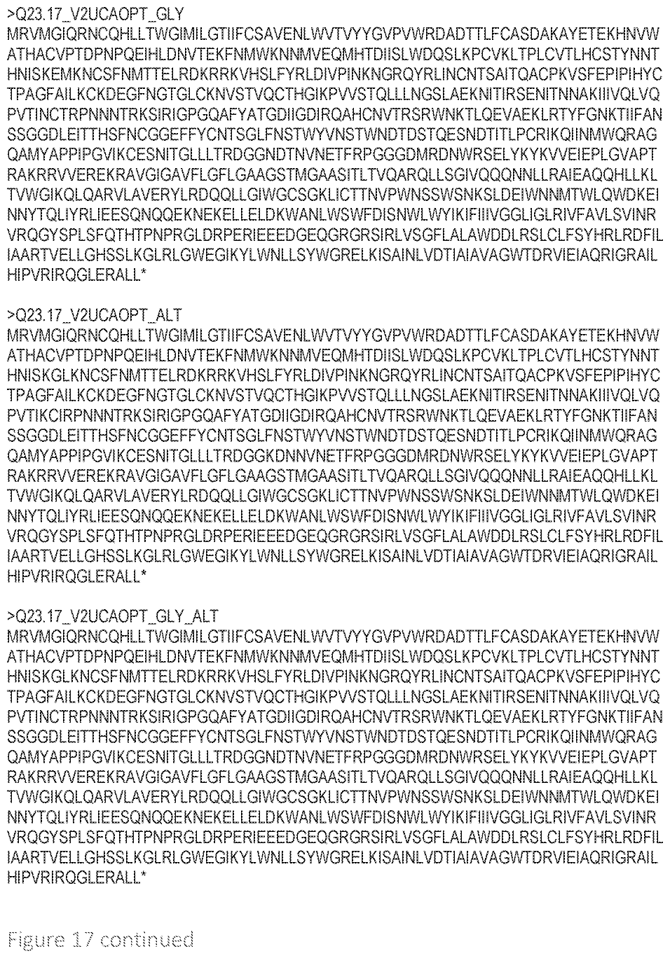

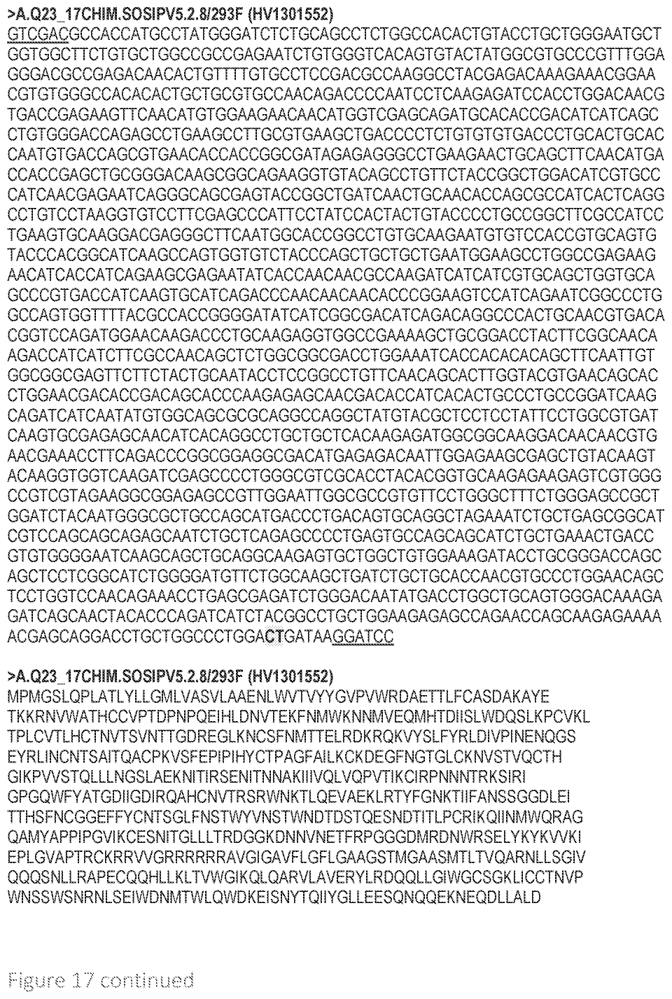

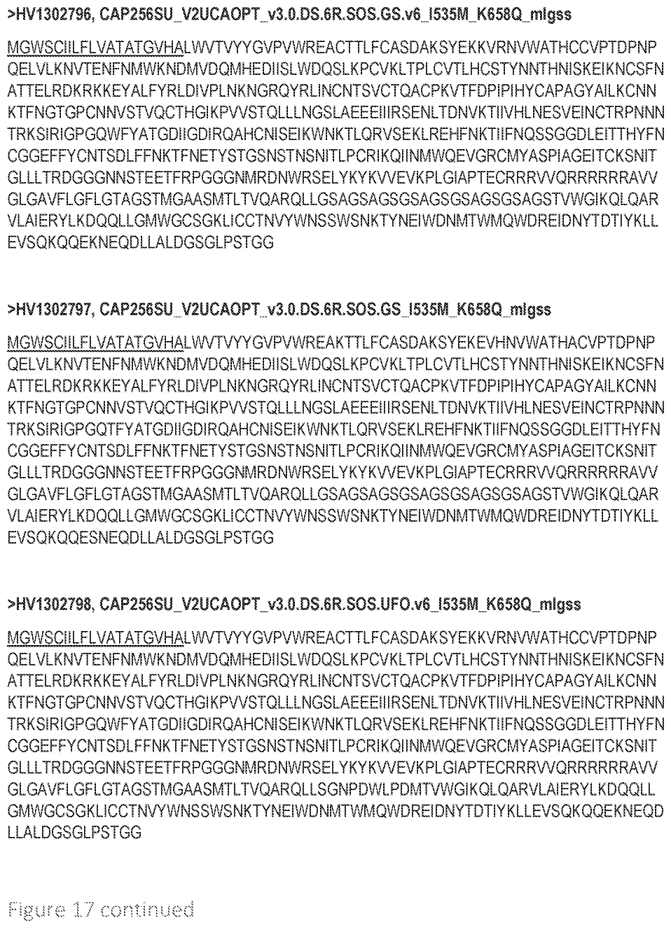

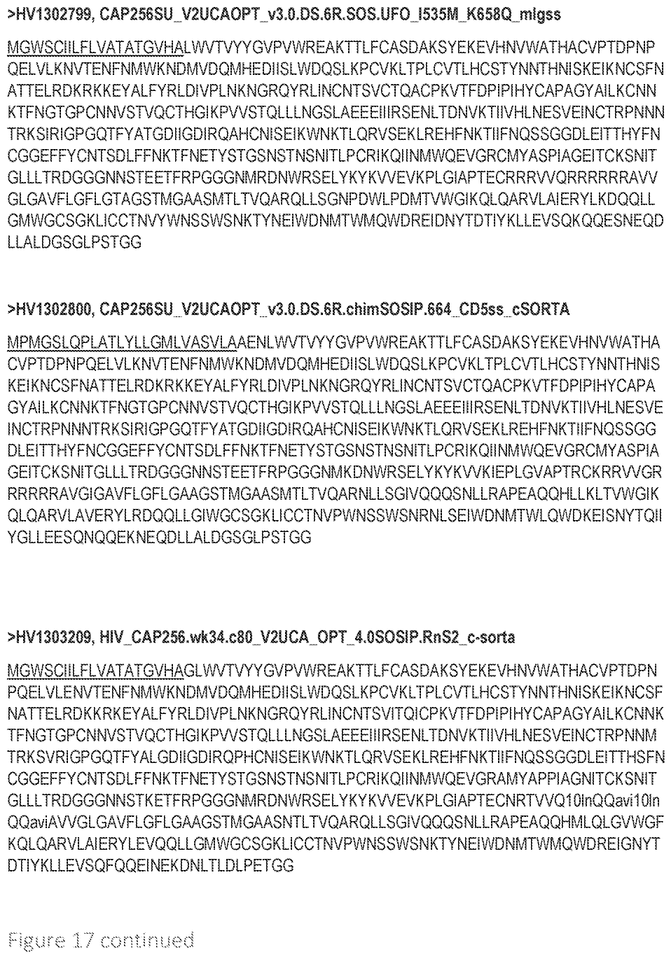

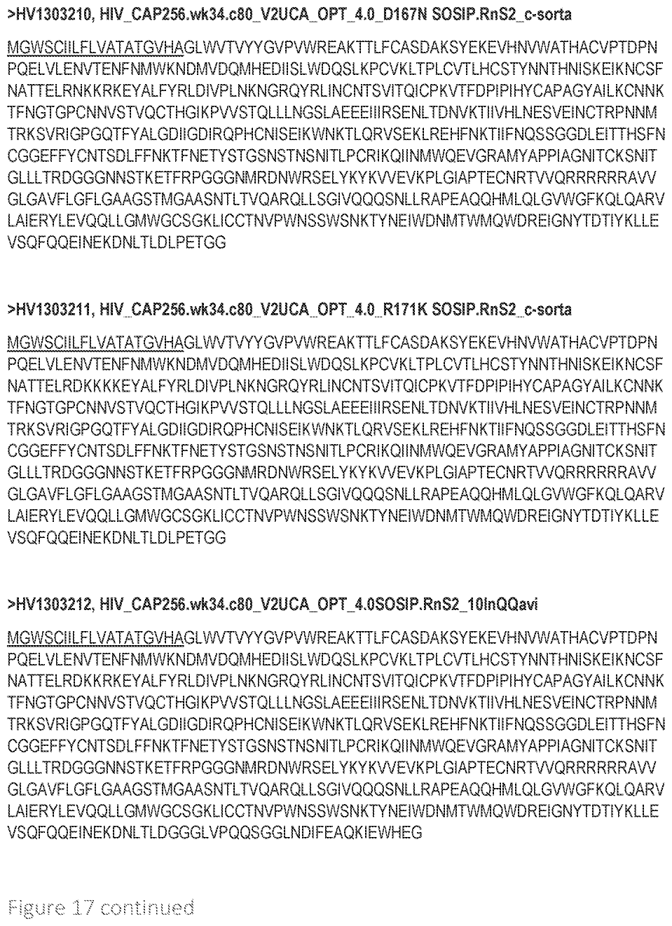

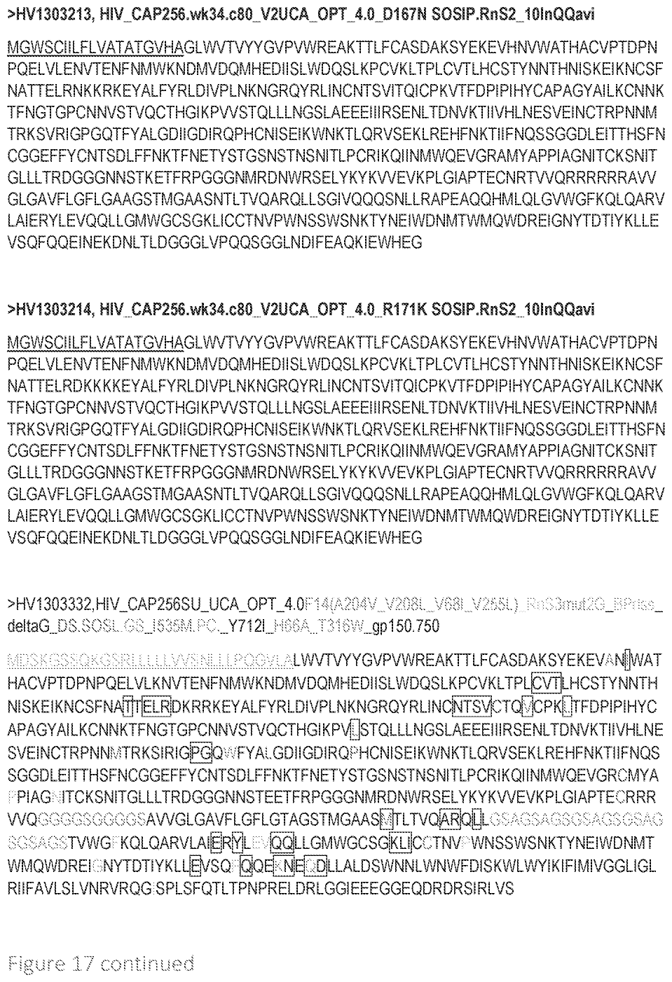

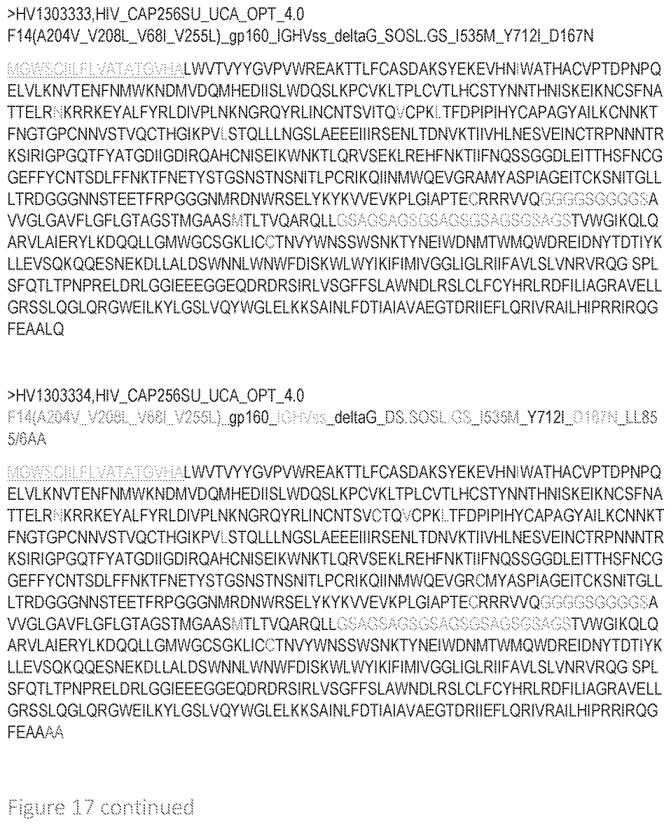

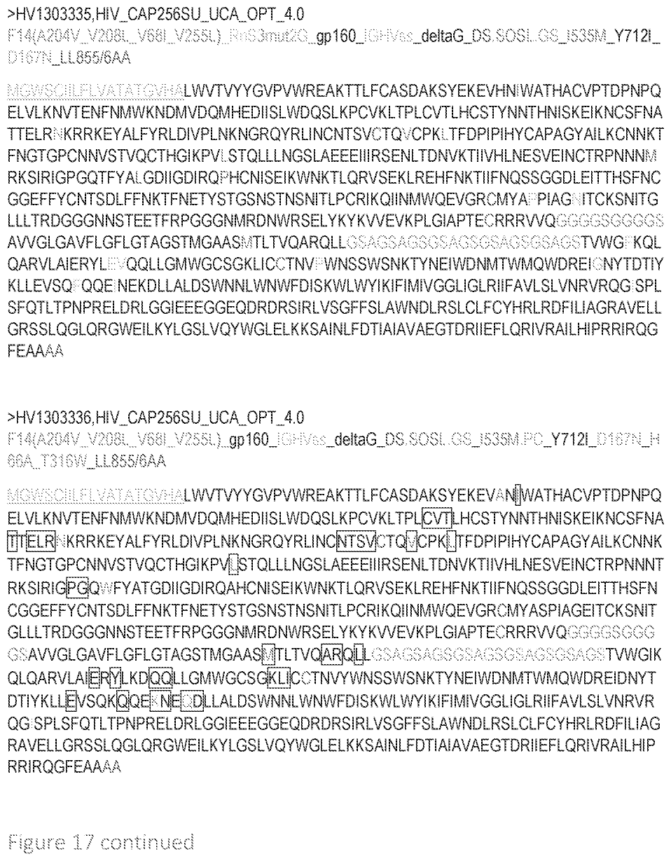

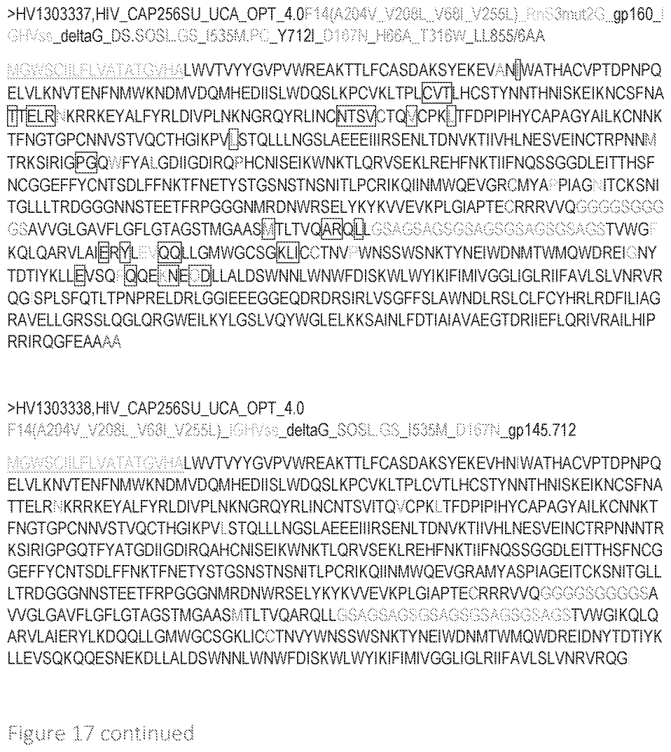

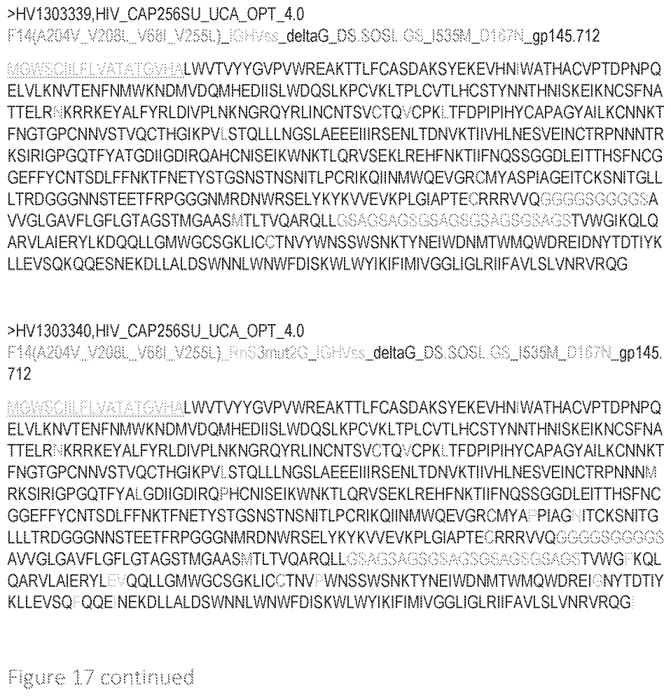

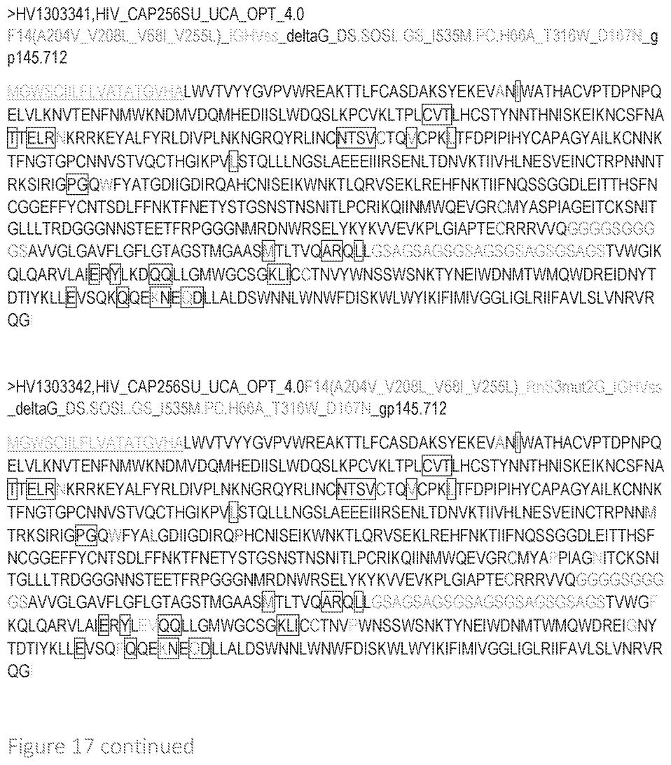

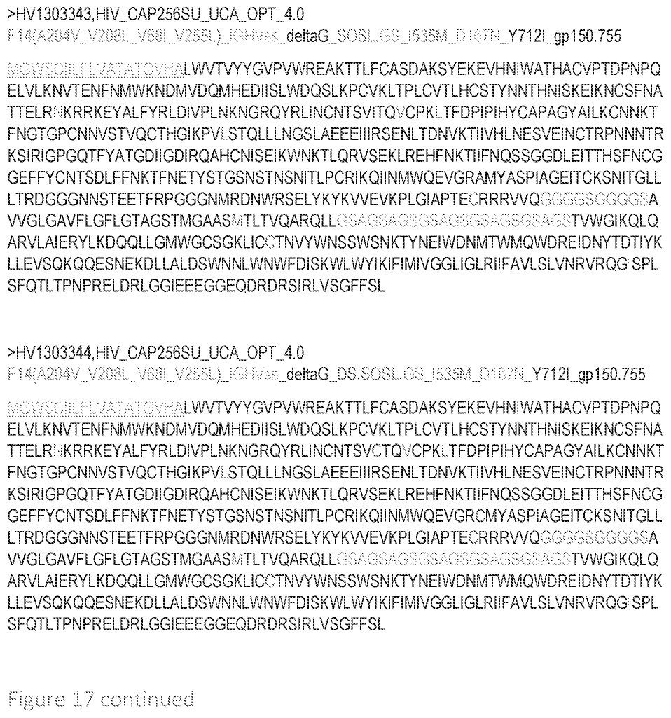

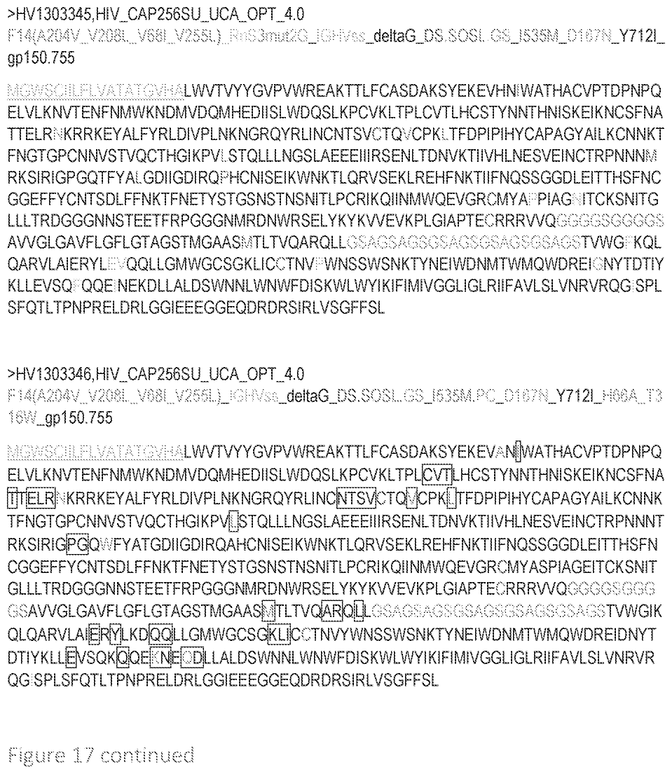

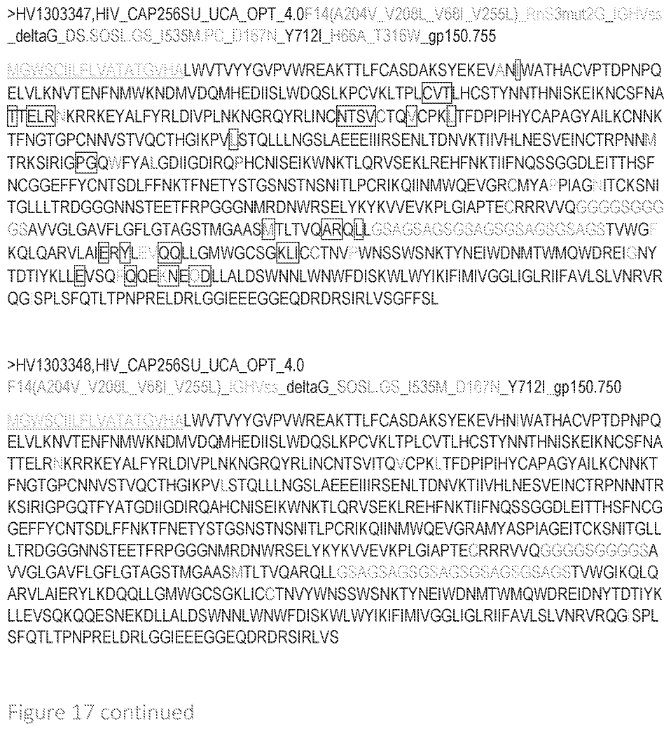

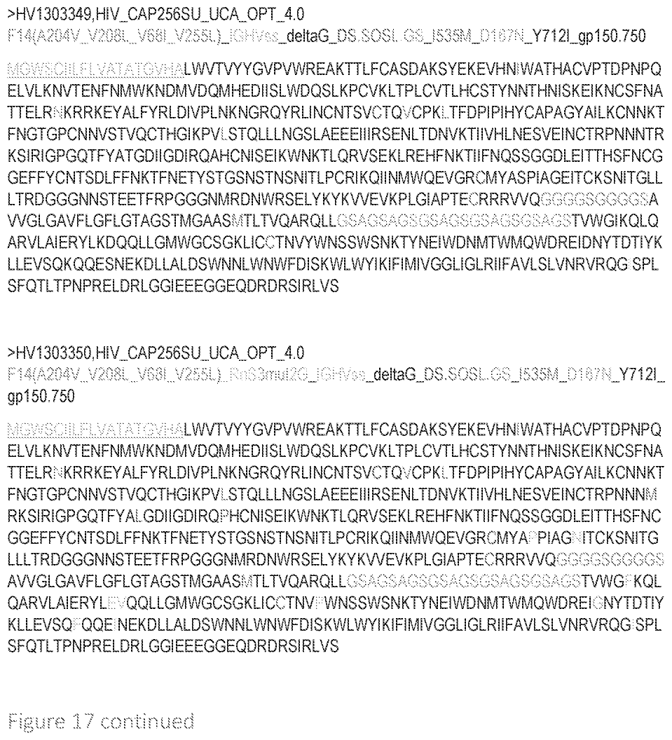

Background: V2 apex bNAbs are an attractive target for immunogen design. FIG. 9A. V2 apex bNAbs arise frequently in HIV-1 infected humans (12-15%) and in SHIV infected RMs (11%). Low levels of somatic hypermutation are required (Wiehe et al Cell Host Microbe 23 (6): 759 (2018)). Low levels of poly- and autoreactivity are also preferred (Liu et al J Virol 89:784 (2015)). Long anionic CDRH3s (>24aa) encoded by germline. Precursors are rare, so germline targeting immunogens are critical. No natural Envs that can target multiple V2 apex bNAb lineages, therefor requiring immunogen design.

CH505 Envs can induce V2 apex (b) NAbs. CH505 TF can trigger germline a V2 apex UCA carrying B-cell line (CH01 UCA Ramos cells). One rhesus macaque (out of 4) immunized with CH505 Envs (gp140) developed tier-2 heterologous NAbs directed at the V2 apex. (Saunders et al Cell Rep 2017 21 (13) 3681-90). RM5695 infected with SHIV CH505 based quasispecies post vaccination developed V2 apex bNAbs. (Roark et al. Science 2020 371 (6525): eabd2638). FIG. 9B. Therefore, it is desired to design CH505 TF immunogens with improved antigenicity to mature and UCAs of V2 apex bNAbs.

Initial Design: A schematic of the signature based approach of immunogen design used is depicted in FIG. 9C. See also Bricault et al. Cell Host Microbe 2019 25 (1) 59-72. Signatures are amino acids or glycan motifs statistically associated with one group of viruses vs others. Previously, sequence patterns associated with sensitivity to mature V2 bNAbs had been identified (Bricault et al. Cell Host Microbe 2019 25 (1) 59-72). FIG. 1. These displayed phylogenetic and/or contact sites, robustness across bNAbs and datasets, and were used for designing CH505 SET OPT. FIG. 9D. Three classes of sites were considered for mutation to increase sensitivity: wildtype resistant (replace with sensitive amino acid or non-significant); wildtype non-significant (replace with sensitive amino acid if available); and wildtype sensitive, but multiple sensitivity signatures at site (replace with more sensitive amino acid if available). FIG. 9P. Additional design considerations included (i) frequency of mutant (M-group & clade C); (ii) number of V2 bNAbs include the signature; and (iii) strength of each signature. For example, NxST 130 may be mutated to H-13. NxST 130 displays strong resistance signature for several bNAbs. H is robust across V2 bNAbs and datasets (vs D) and is not infrequent. On the other hand, T-297 may be retained as there is no sensitivity signature or alternatives identified and T is the most common form. 11 mutations total were present in the final, initial design. FIG. 9Q. 8 resistant or non-significant with sensitive signatures were replaced. Additional mutations were one sensitive to more sensitive (R169K), one neutral to neutral (E170Q, remove-ve charge), one resistance signature for completing glycan shield (NxST332; no impact on sensitivity).

Neutralization data for 208 global viruses against CH04 & CAP256 UCAs, and heavy and/or light chain germline reverted PG9 was generated. (Gorman et al. NSMB 23 81-90 (2016)) FIG. 9E. Unlike other bNAb classes, V2 apex precursors can neutralize heterologous strains. CH04 UCA shows 4% breadth. PG9 with both heavy and light chain reverted provides 2% breadth. CAP256 UCA only neutralizes 1 autologous virus. Partial germline reverted PG9 (heavy or light) display a higher breadth. These data were used to calculate signatures.

Robust signature sites met at least 2 criteria: (a) contact site; (b) phylogenetically corrected signature; and/or (c) strong association (q<0.1). FIGS. 2, 7D. Few signatures for CH04 UCA, and PCT64 early bNAbs were identified. Several signatures for PG9 either heavy or light chain reverted were identified, due to their relatively higher breadth. UCA OPT_1 (CH505 TF UCA_OPT1) includes mature V2 apex signatures, with 5 additional for UCAs. FIG. 9R.

For CAP256 IA4, weak signatures were found due to low statistical power (3 out of 208 viruses neutralized). Only resistant signatures outside the epitope were identified. Change to neutral residues at most sites would involve mutation to rare amino acid and/or removing glycans that could introduce vulnerable gaps in the glycan shield. Only two mutations were introduced at positions 736 and 842. Designed UCA optimized constructs without (UCA_OPT1) and with (UCA_OPT2) these weak signatures. FIG. 9F.

Hypervariable loops cannot be aligned due to extreme length and sequence variation. Rather, tests are performed to identify associations with net charge, length and number of glycans. Two significant hypervariable loop associations with sensitivity to V2 apex bNAbs were identified: (a) positively charged V2 loops (V2 apex bNAbs have long anionic CDRH3); and (b) smaller hypervariable V1 & V2 combined (possible steric hindrance due to the dynamic loops). FIG. 9G-9I.

Mature signature introduction displays an increased sensitivity to neutralization by mature V2 bNAbs. Germline signatures displayed further increased sensitivity to neturalization by mature V2 bNAbs. FIG. 9J-9N. UCA signatures increased the sensitivity of CH505 to neutralization by both CH01 and the PCT64 V2 bNAb UCAs. FIG. 9K. V2 SET OPT also gains CH01 UCA sensitivity, likely due to H-130. UCA_OPT2 that had CAP256 VRC26 UCA signatures also did not confer sensitivity to this UCA. Because UCA_OPT_2 displays low infectivity, it could not be tested.

Introduction of V2 apex mature signatures in CH505 TF improved sensitivity to mature bNAbs, and gained sensitivity to CH01 UCA. Introduction of UCA signatures further improved sensitivity to mature bNAbs, to CHOI UCA and gained sensitivity to PCT64 LMCA. FIGS. 9N, 9S.

SET OPT & UCA_OPT constructs were expressed as chimeric CH505-BG505 SOSIPs (Saunders). Different constructs tested with varying quality & expression. The best was UCA_OPT1 with NxST 332 and gp41 mutations. Binding data was consistent with neutralization results. FIG. 9O.

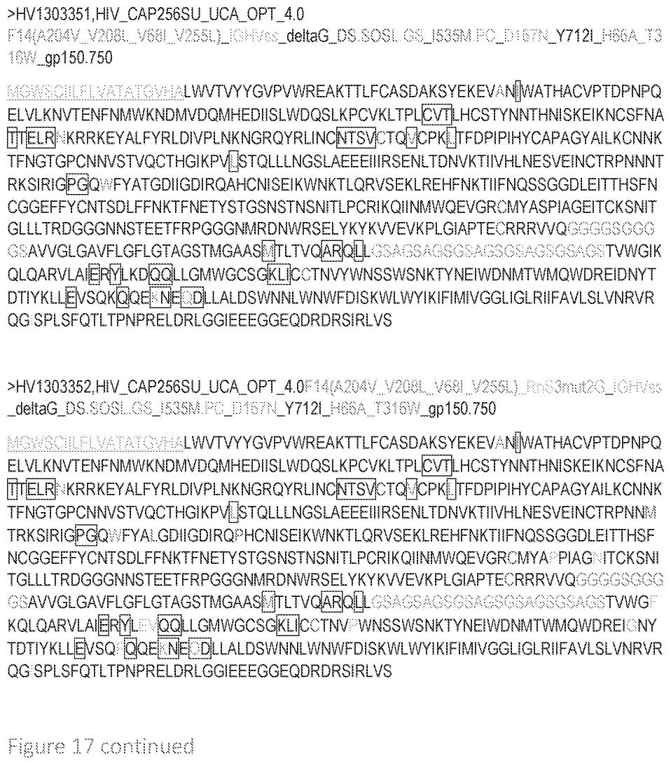

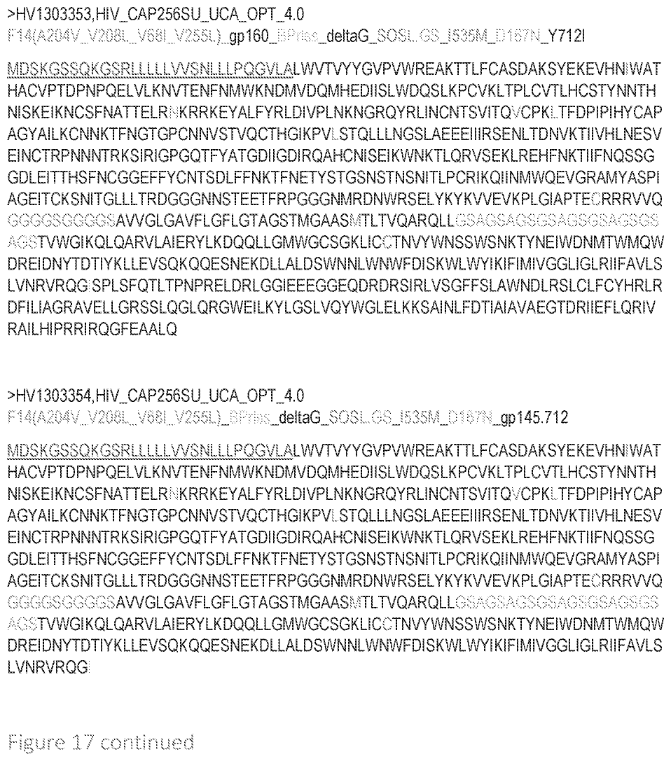

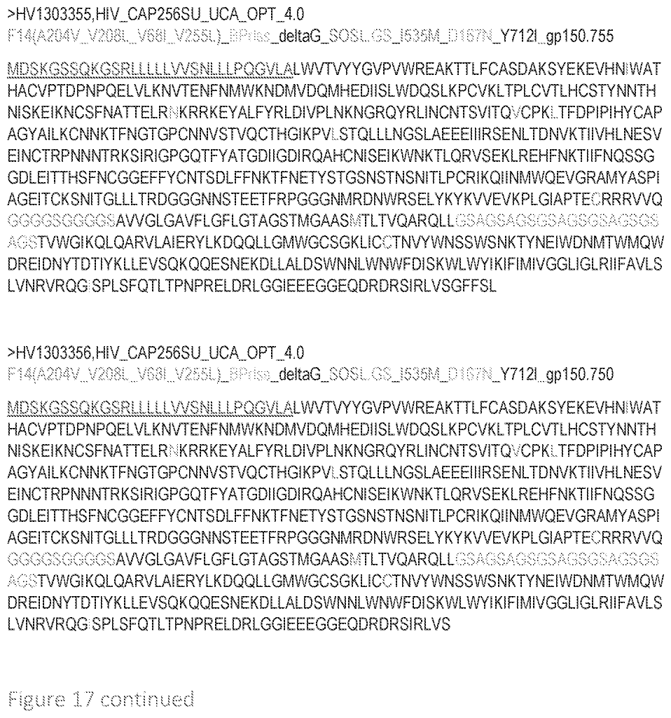

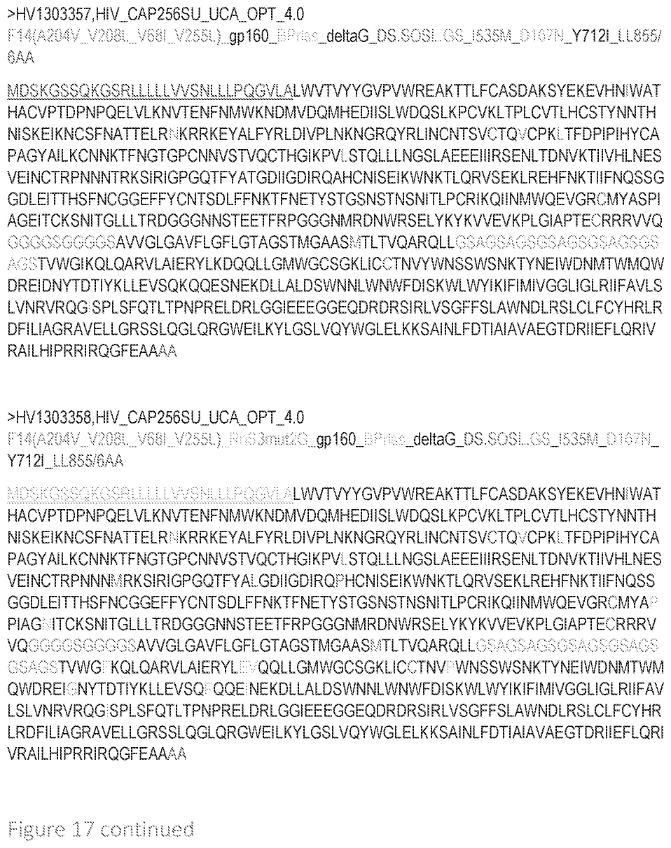

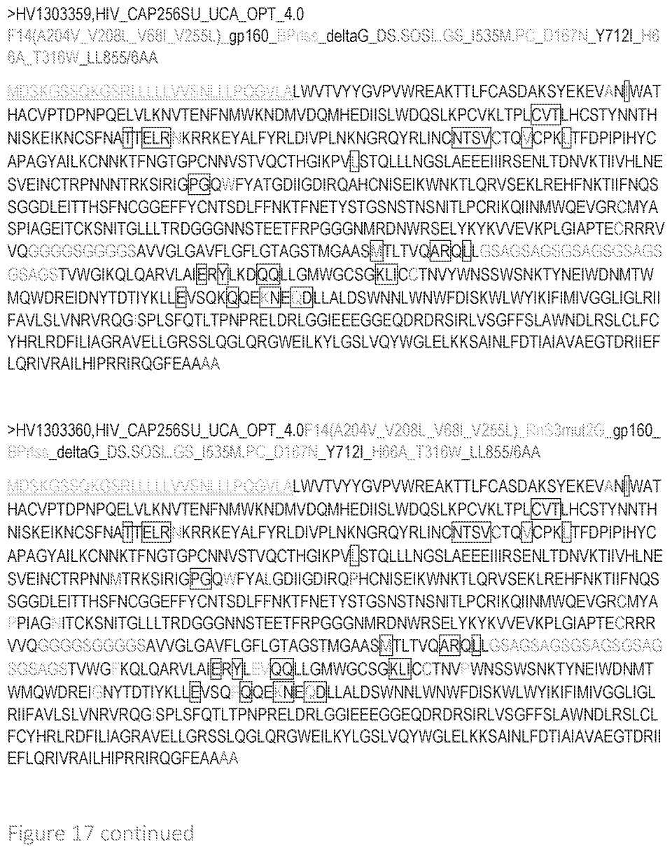

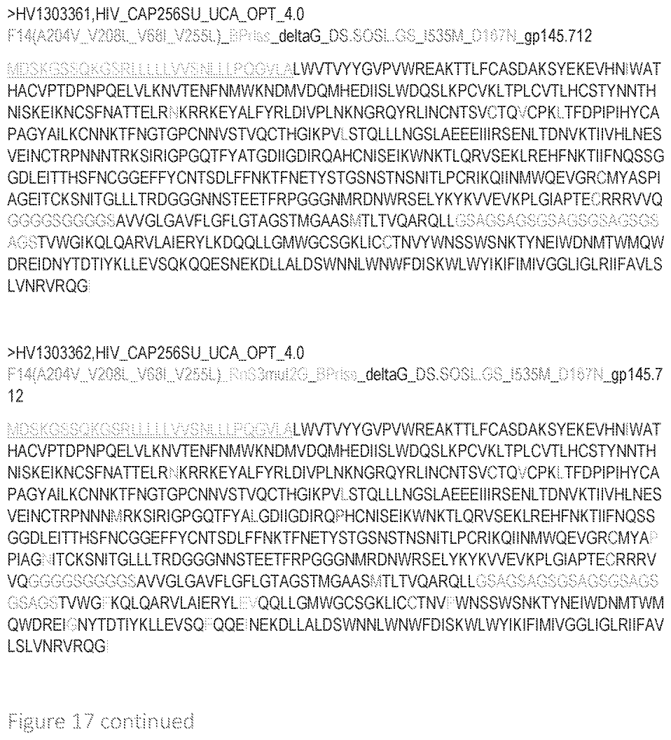

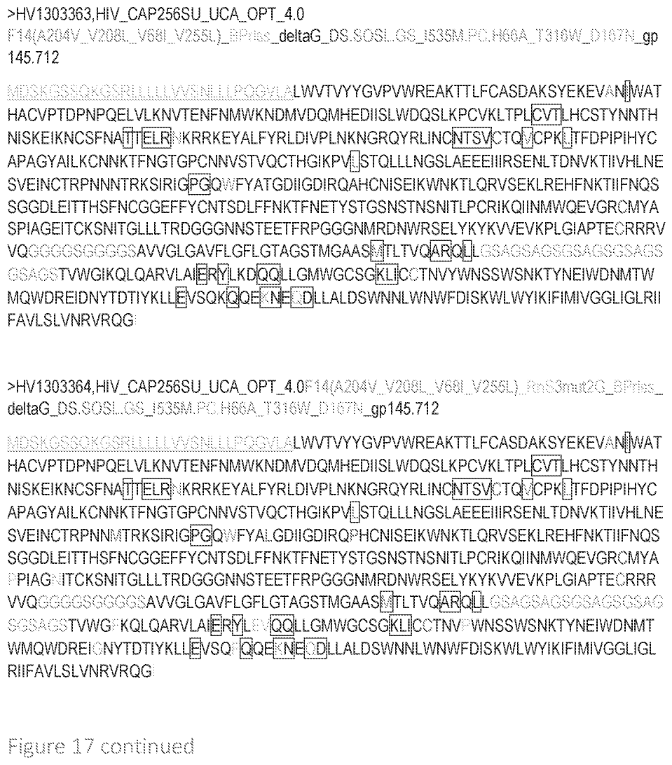

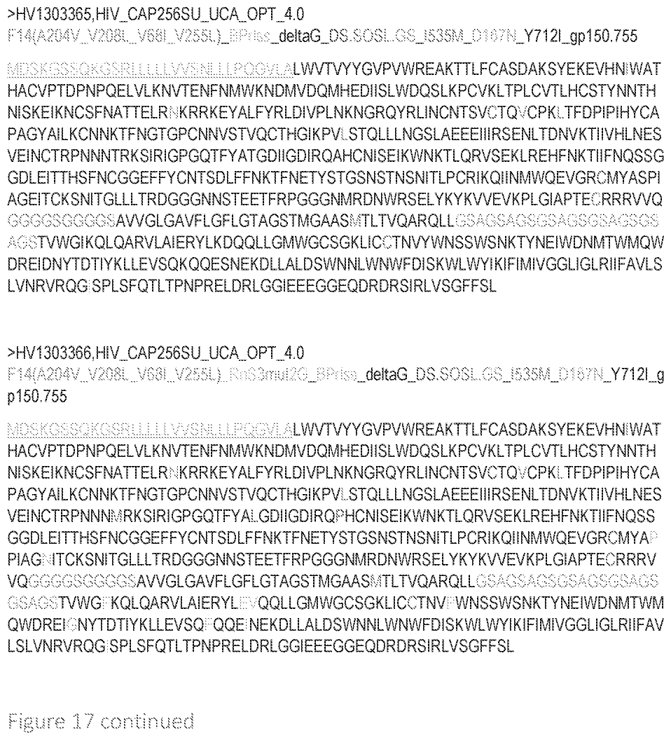

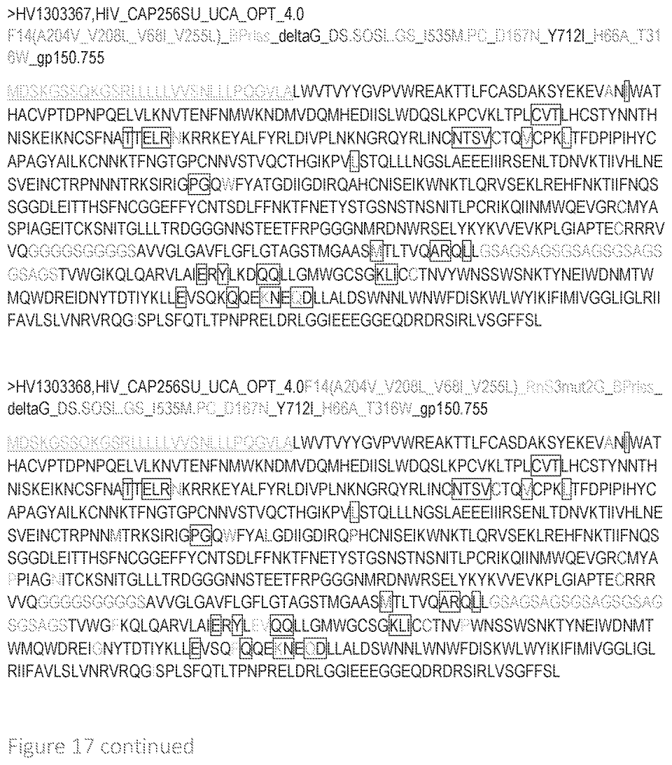

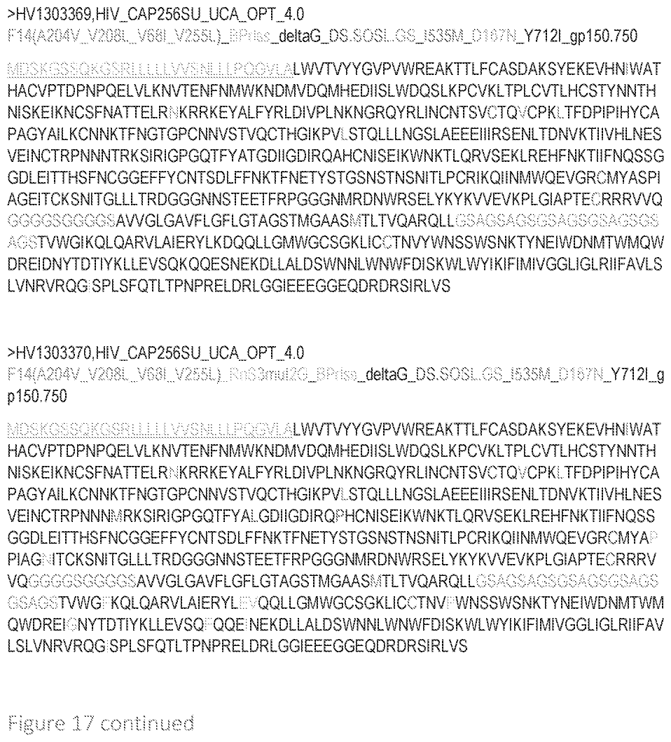

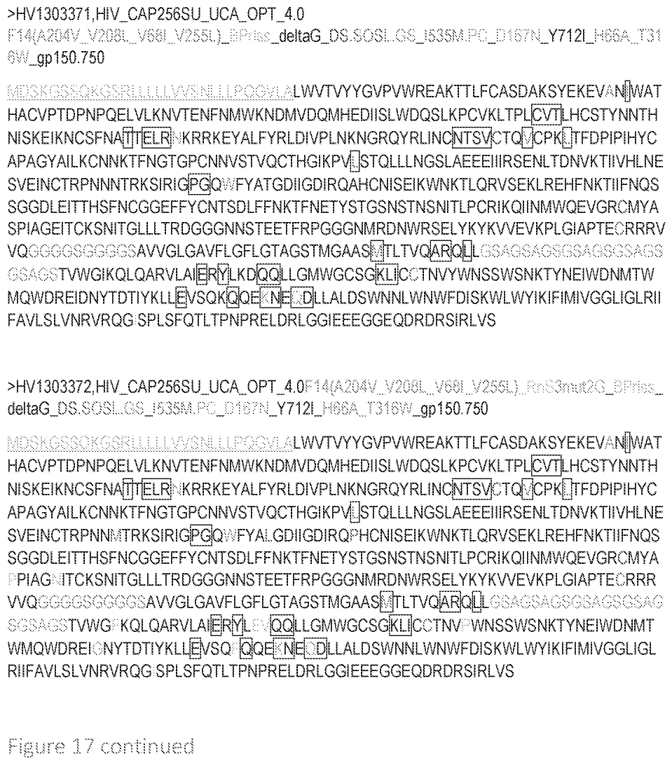

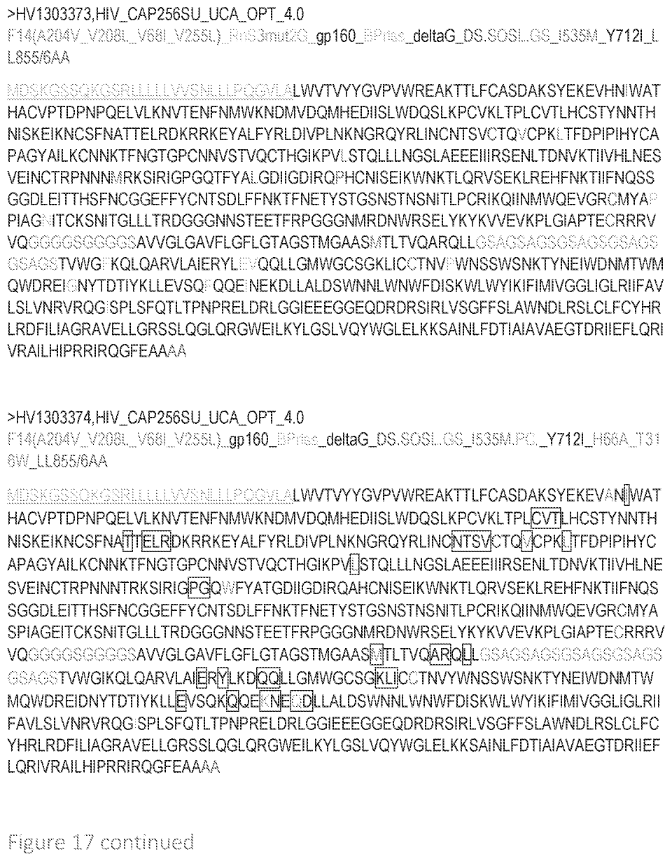

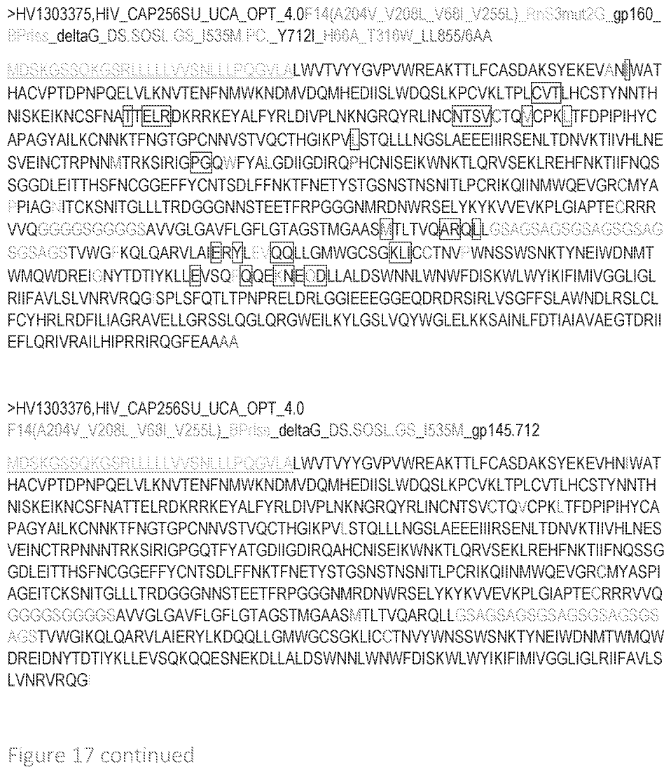

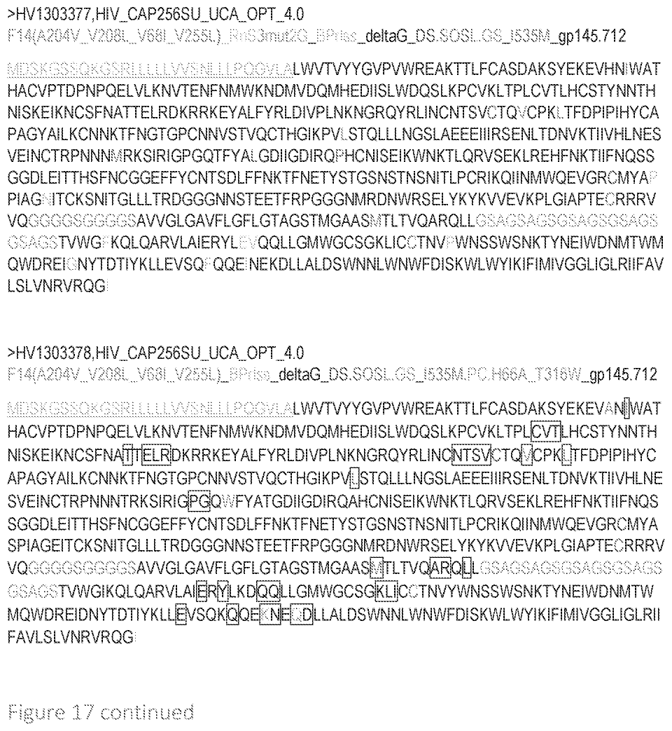

Longitudinal Env evolution shows escape predominantly at sites 166, 167, 168 and 169 (Landais et al. Immunity 2017). FIG. 10A. Therefore, TF amino acids at these sites may be associated with sensitivity to PCT64 UCA. All constructs so far have possessed R-166, K˜ 168 and R-169. However, they all have D-167, which is associated with escape from early PCT64 lineage Abs. Therefore, it may be beneficial to introduce mutation D167N.

D167N was shown to be sensitive for PCT64 LMCA. D167N is associated with escape from early (13 month) PCT64 lineage Abs. FIG. 10B. Intriguingly, later PCT64 Abs (month 18 onwards) become more reliant on N-167. Month 18 Ab is agnostic and Month 24 Ab onwards become more sensitive with D167N. M4C054 is an autologous Env from 4 months that is sensitive to PCT64-LMCA with glycan deletions at 130 and 133. FIG. 10C. This Env has N-167. M18C043 is not neutralized by PCT64-LMCA even with 130 and/or 133 glycan deletion. This Env has D-167. CH505.V2UCAOPT.v3.D167N design and neutralization testing is depicted in FIG. 10D.

### CAM13RRK V2 UCA Optimization: K130H for Improving CH01 UCA Sensitivity and Swapping the Very Long Hypervariable V1 and Negative V2

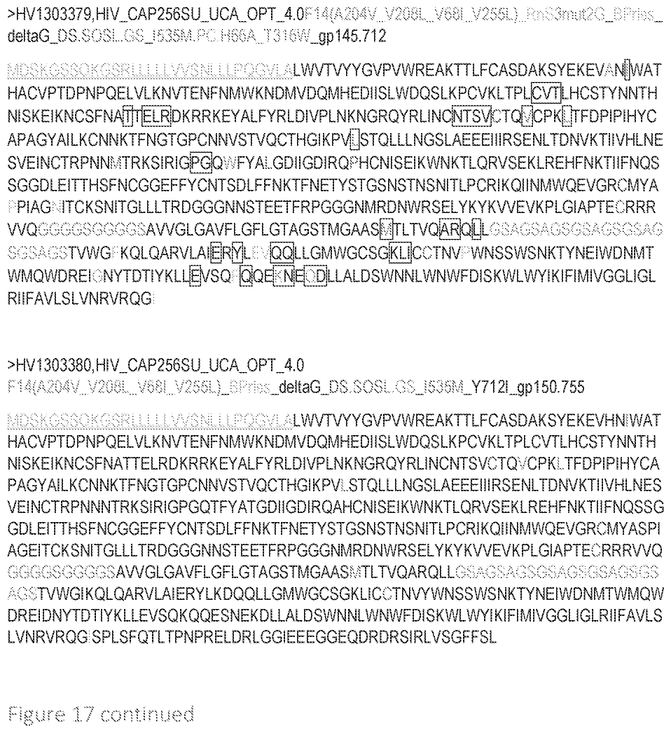

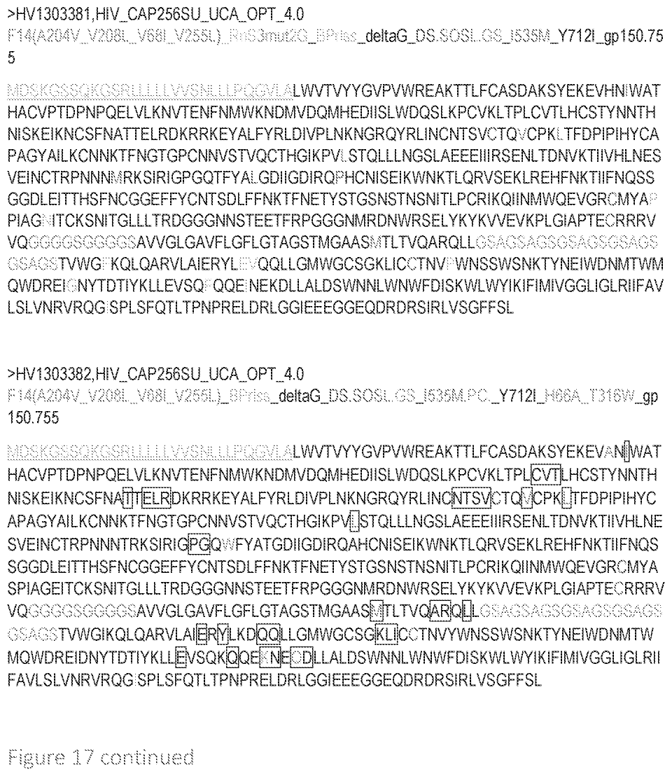

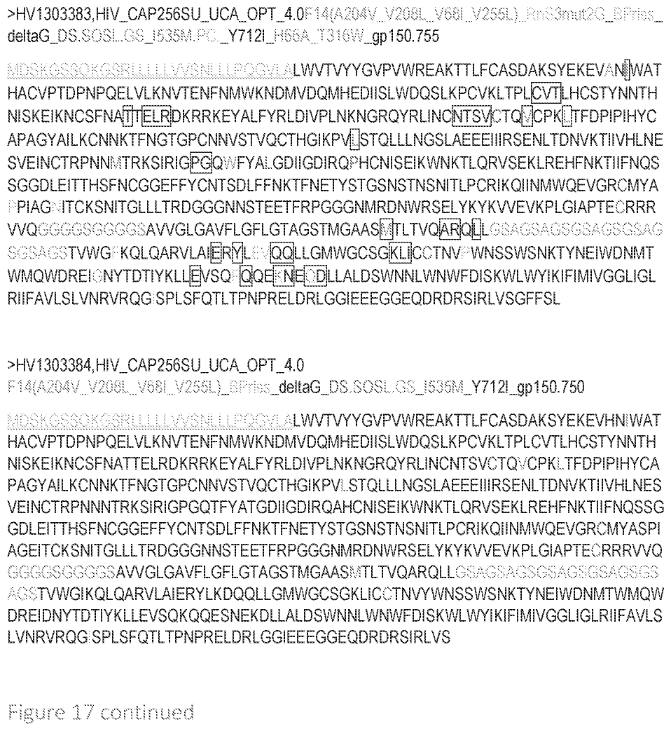

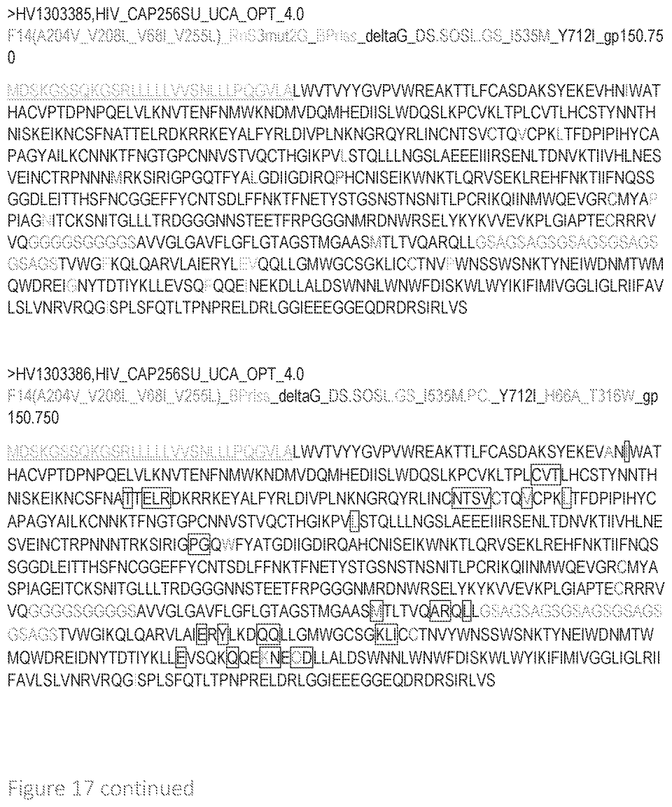

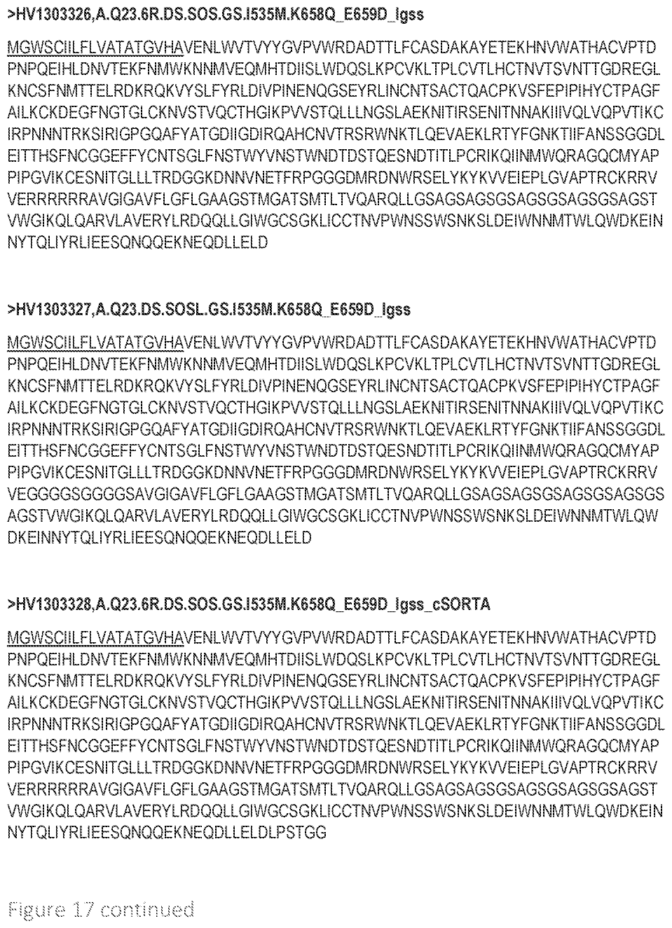

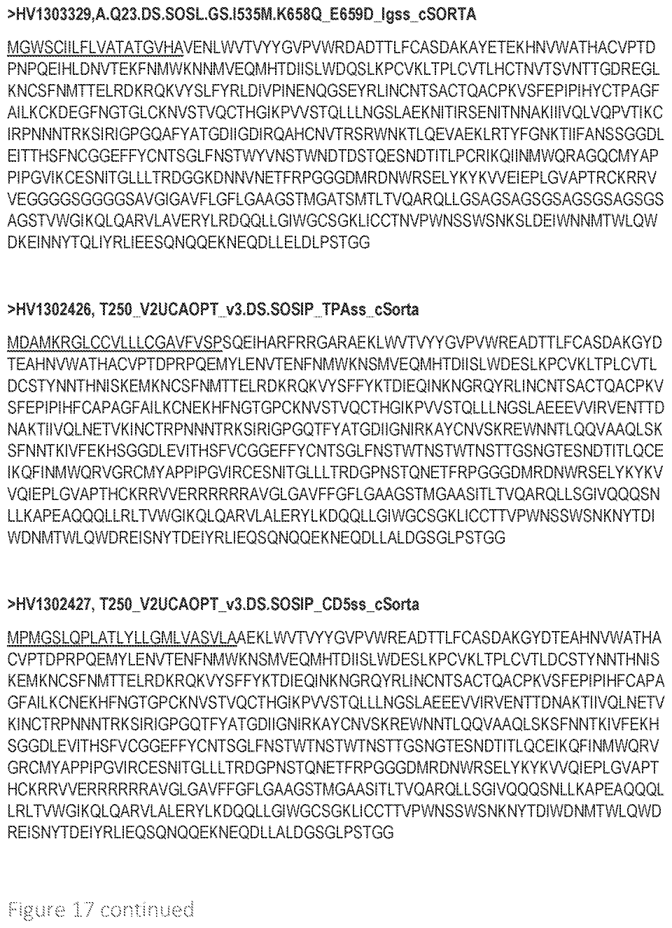

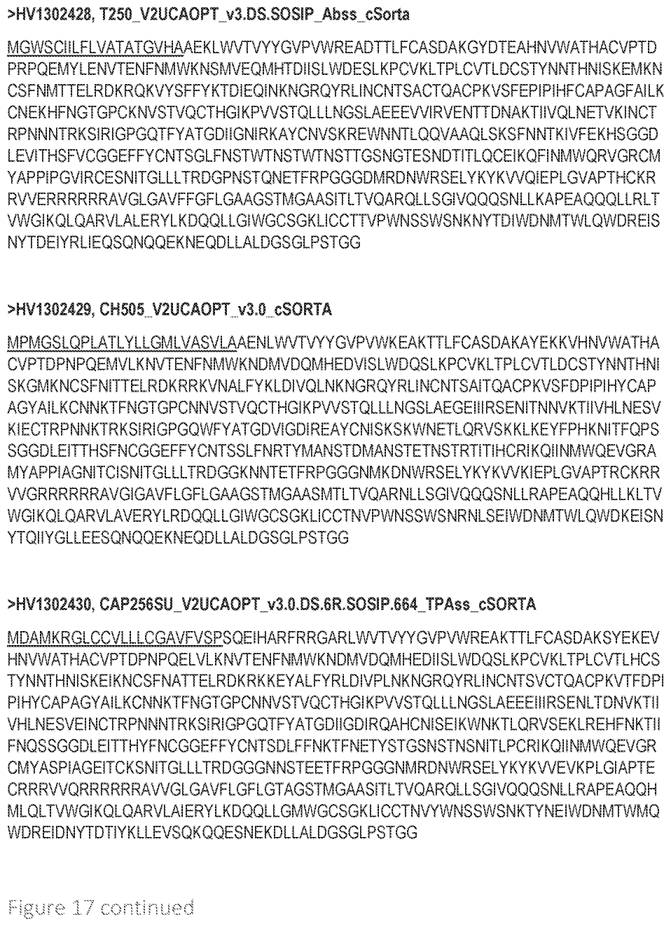

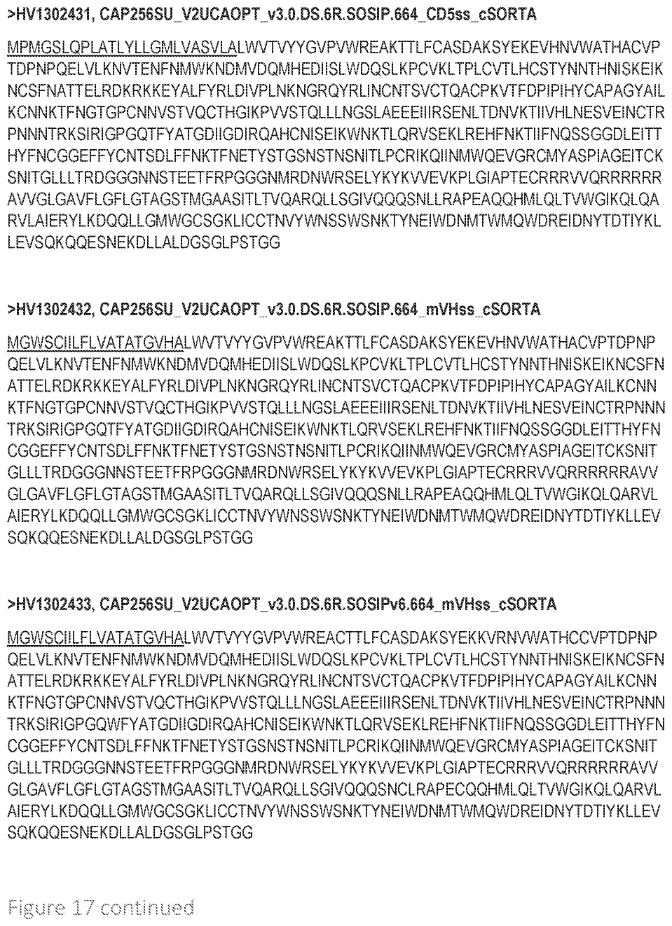

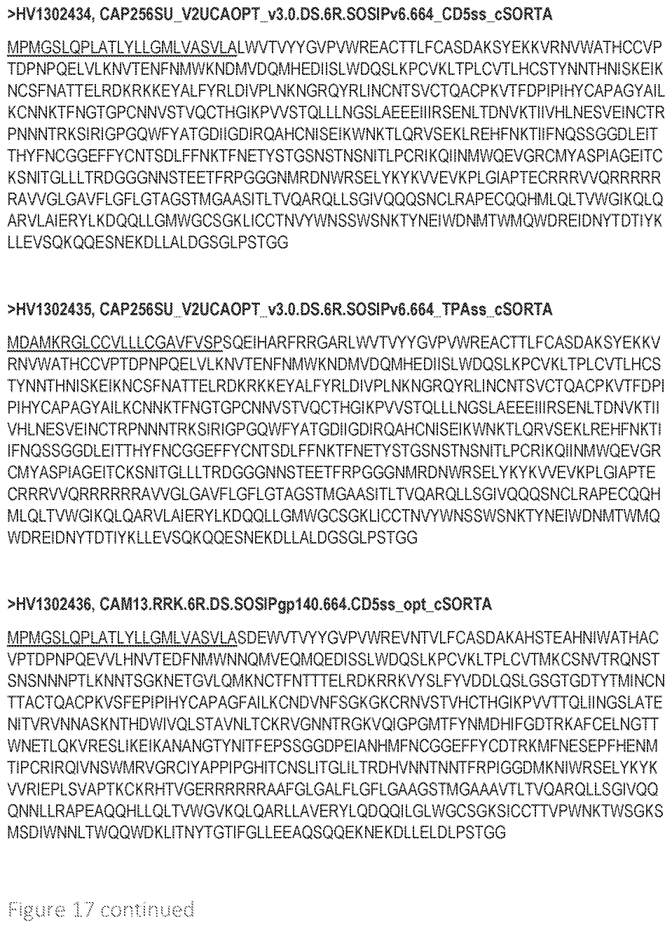

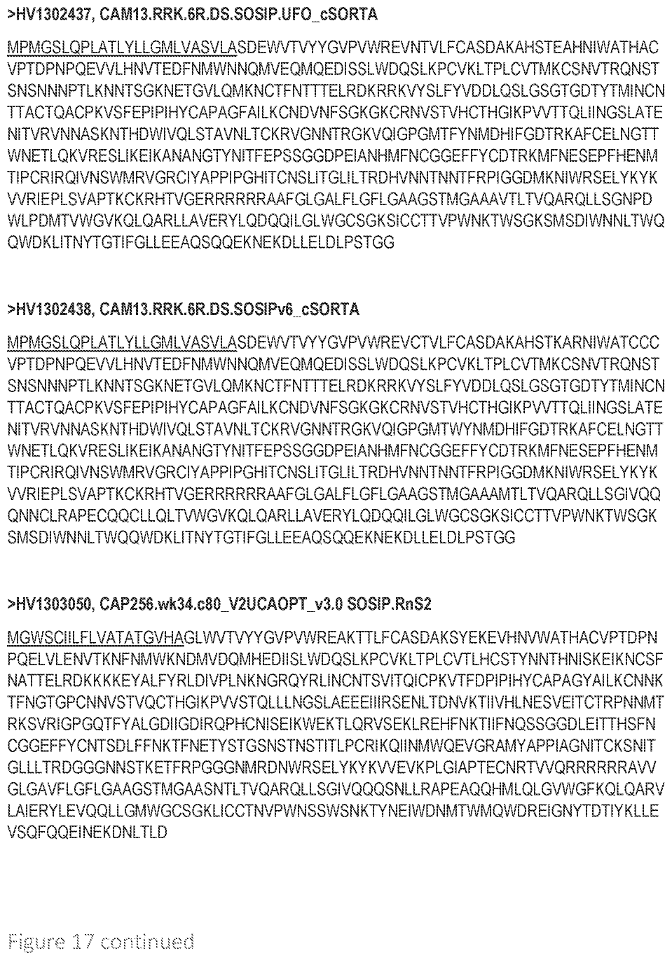

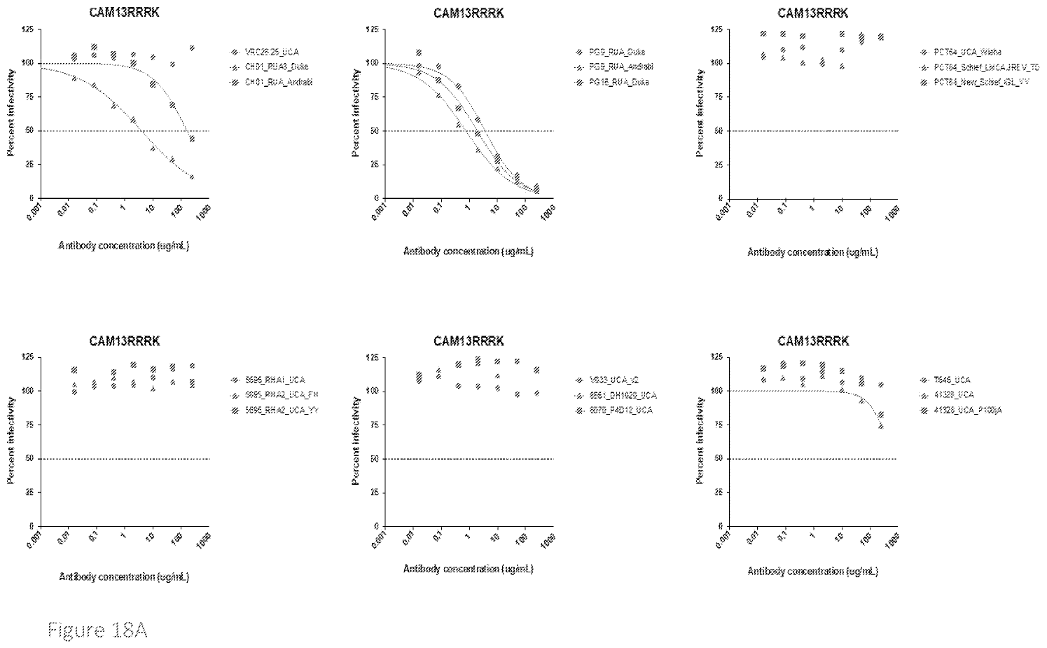

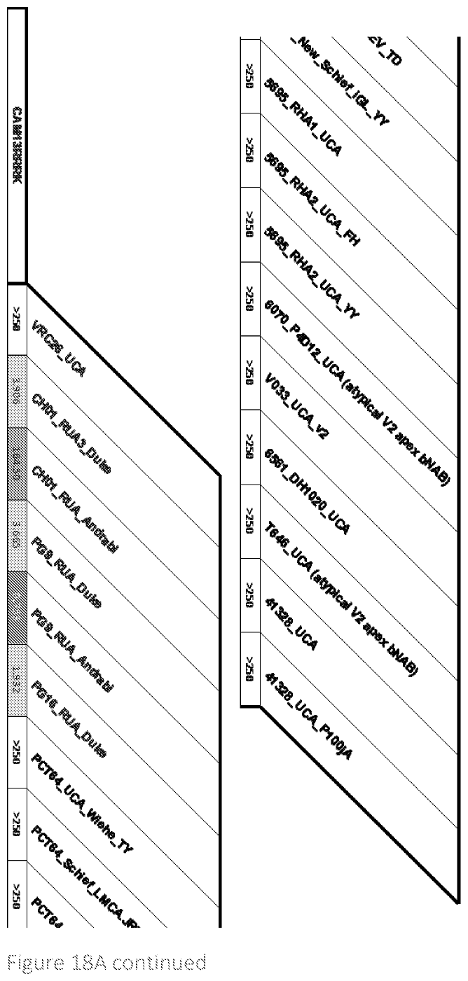

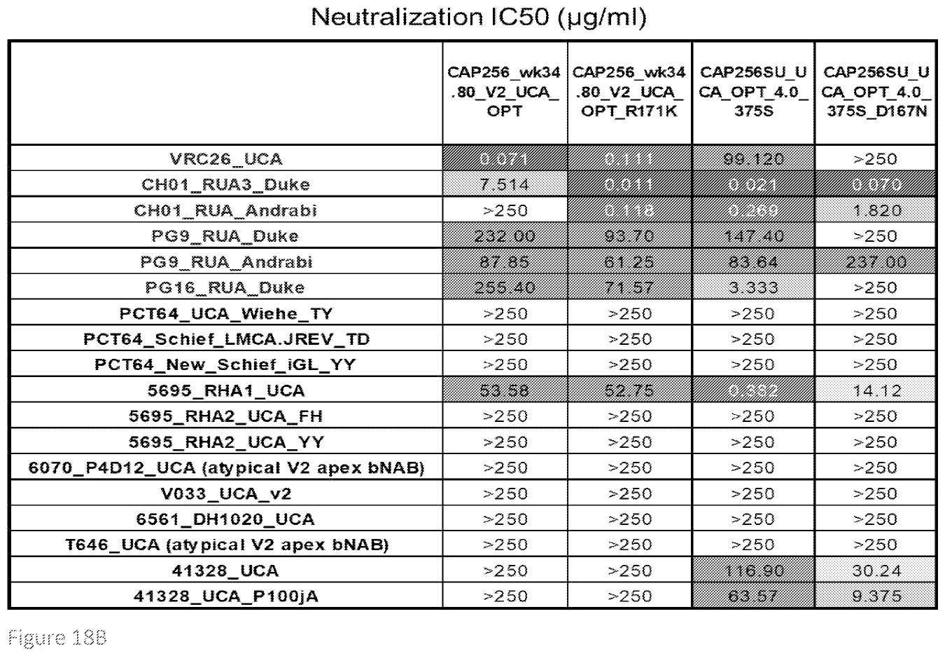

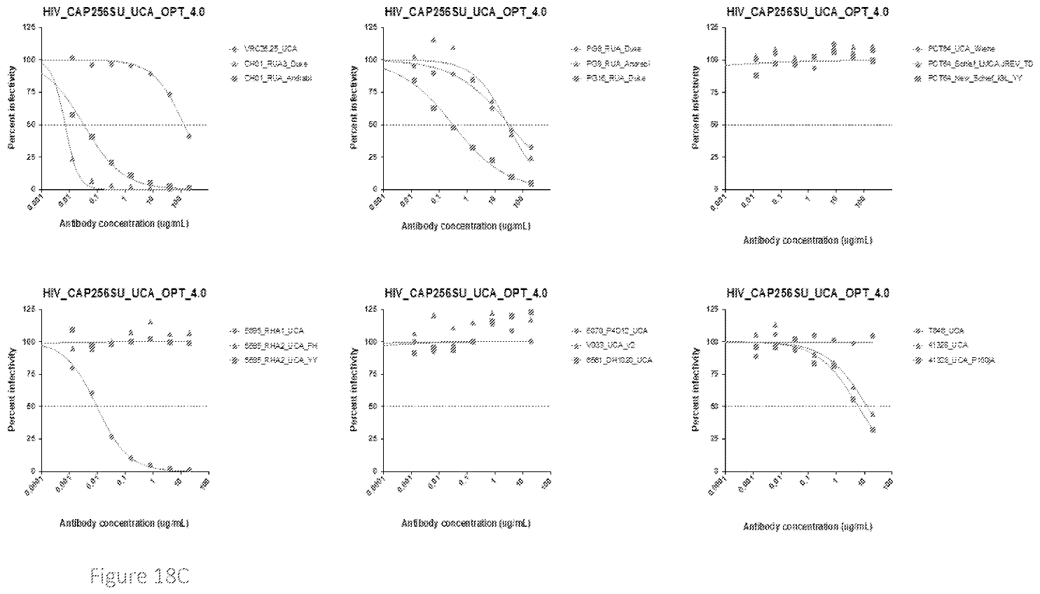

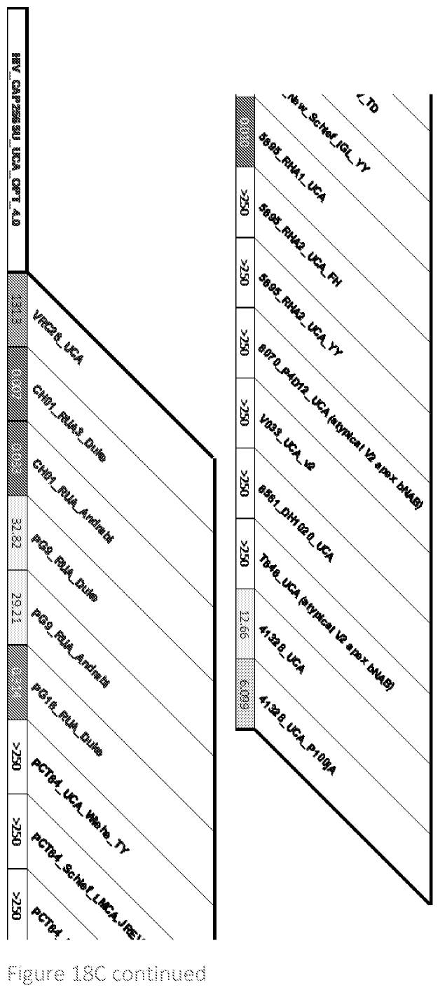

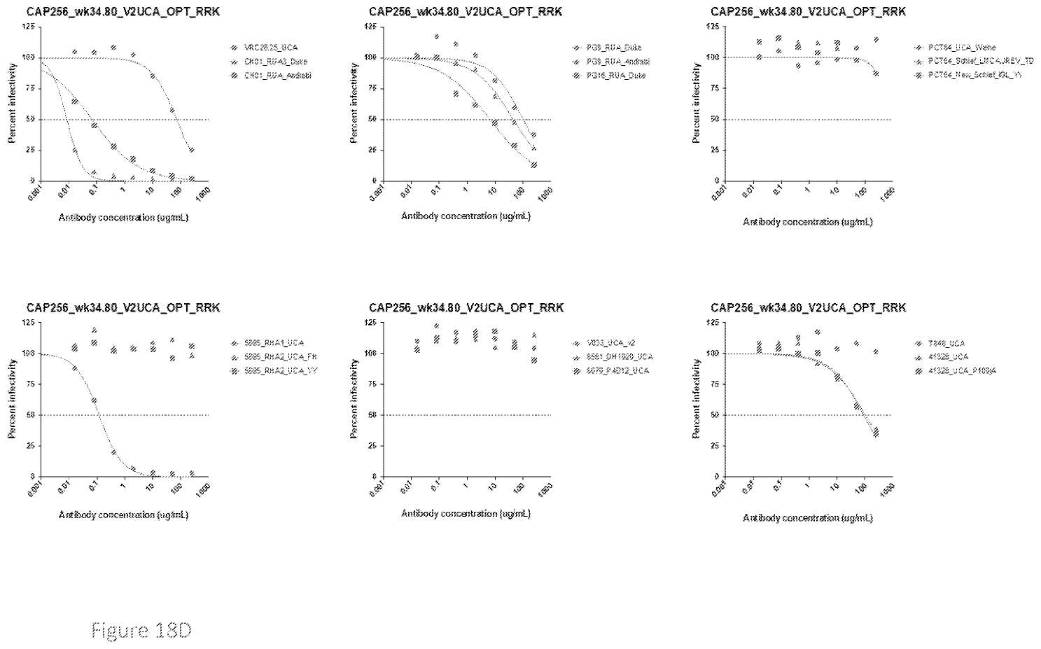

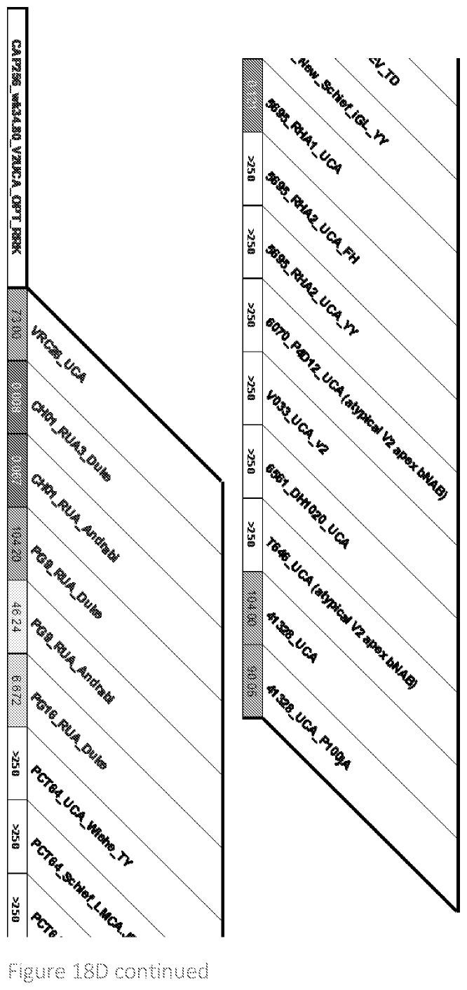

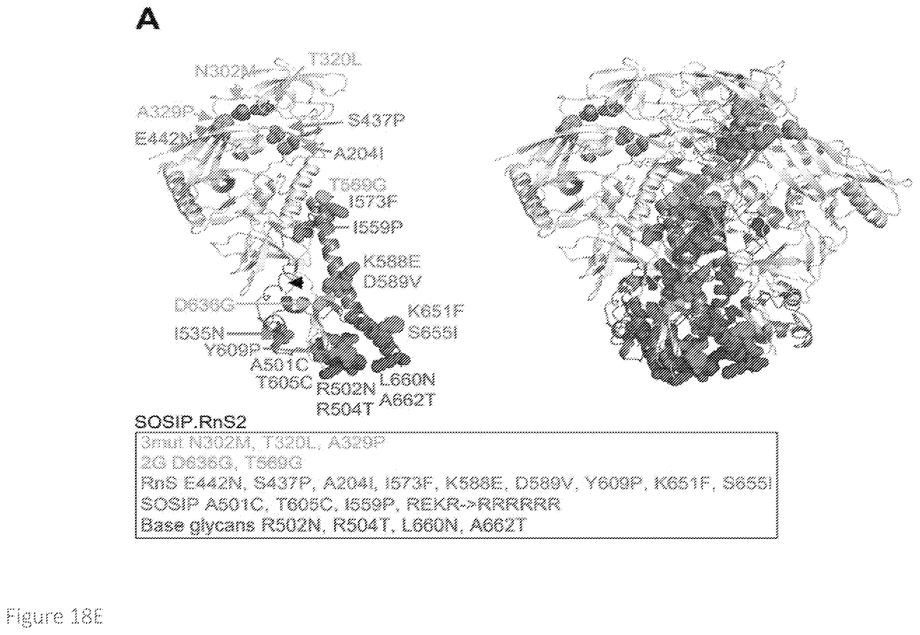

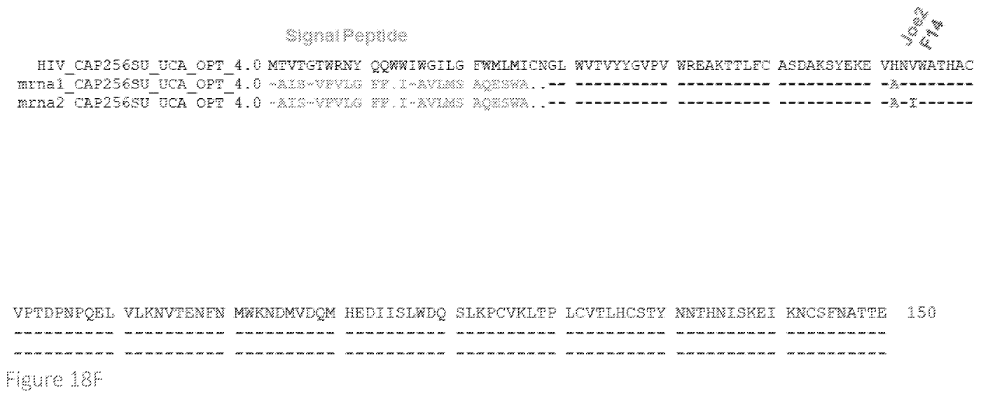

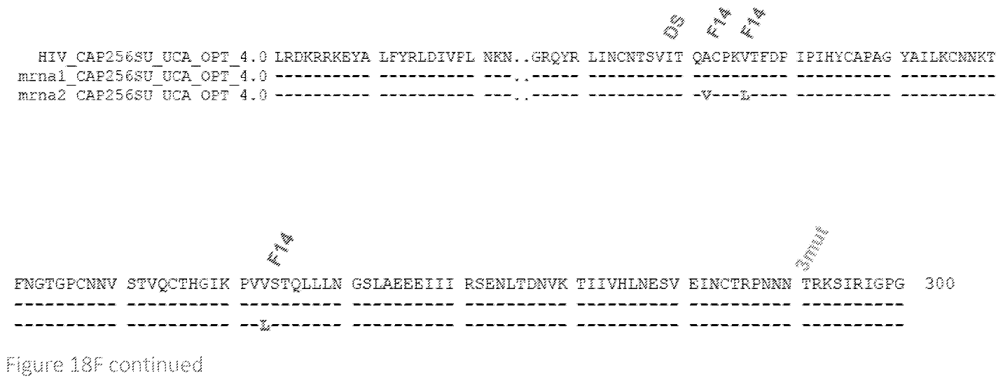

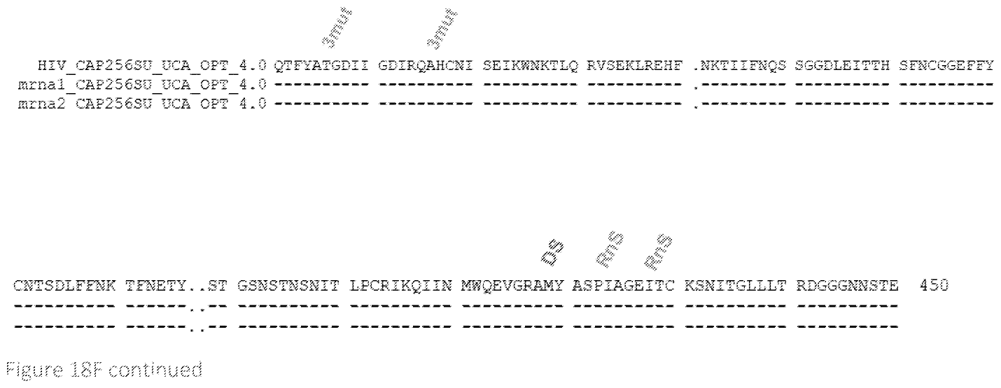

CAM13 is natural SIVcpz Env (Nerrienet et al. J Virol. 2005 January; 79 (2): 1312-1319. doi: 10.1128/JVI.79.2.1312-1319.2005). It has been shown that CAM13 mutated to R-169, R-170 and K-171 (called ‘CAM13RRK’) becomes sensitive to CH01, PG9 and PG16 UCAs. FIG. 11A.

CAM13RRK bas poor reactivity with CHOI UCA. Several experiments have shown that H-130 is the strongest signature for CH01 UCA sensitivity. So the K130H mutation was introduced. For PCT64, position 315 could be improved. However, M-315 in CAM13RRK is very uncommon in HIV, so it was not possible to determine its impact. The 315 signature is only for month 24. Therefore, no change was recommended. CAM13RRK has uncommon HIV amino acids for several outside epitope signatures for PG9 heavy/light reverted. In the epitope, T161M and Y173H can be considered. However, since there is good reactivity with PG9/PG16 UCAs, no change is needed. The signatures for CAM13RRK are shown in FIG. 11B.

CAM13RRK has very long hypervariable V1 loop. Design construct CAM13RRK delV1 changes the hypervariable V1 loop length from 31 to 23 amino acids. FIG. 11C. The natural loops were modified to introduce deletions and positive charges. FIG. 11D. No gain was identified in hypervariable V1 changes, but gains of +3 net charge (−1 for wildtype to +2 for the construct) was identified. Substantial change in hypervariable V1 length was provided from 31 amino acids for the wildtype region to 12-16 for constructs.

CAM13RRK bas 5 glycan holes: N130+hyp V2 hole (this should be retained as filling it may reduce V2 apex UCA reactivity); N295+N332 hole (interestingly, this is filled by N442 in one RM (T927)); N386 hole (filled by 2 RMs T927 and T925); and N234 and N616 holes (filling them will likely not impact V2 UCA sensitivity and does not create bNAb sensitivity). FIG. 11E. Natural best hypervariable region has N442 and N386 holes filled. Opt has N234 and N616 filled on top of these two.

### Constructs for Testing:

CAM13RRK+K130H+Natural Vlh V2h swap+natural gly. Expected to have improved CH01 UCA reactivity. Hyp V1 & V2 loops from best SCIV infected RMs. Based on glycan shielding from RMs, added N442 and N386.

CAM13RRK+K130H+Opt Vlh V2h swap+opt gly. Expected to have further improved V1 & V2 hyp loops based on best loops from CAM13K/RRK infected RMs. May improve reactivity. Better glycan shielding as N234 and N616 are added.

Neutralization testing was performed. FIG. 11F. Given that both K130H mutation and V1 hypervariable loop deletion improve sensitivity, a variant that includes both these changes was designed (CAM13RRK_K130H_delV1). Testing is ongoing,

### CAP256SU Based Designed Envs

Strategy: CAP256SU is quite sensitive to V2 apex mature bNAbs (IC50=0.0004-2.2 μg/ml for CAP256 bNAbs, CH01, PG9, PGDM1400 & PGT145). It is also neutralized by CAP256 UCA (IC50 ˜35 μg/ml). Thus, a variant that is optimized to carry sensitivity signatures for PG9 germline reverted Abs, CH04 UCA, and PCT64 intermediate Abs was designed.

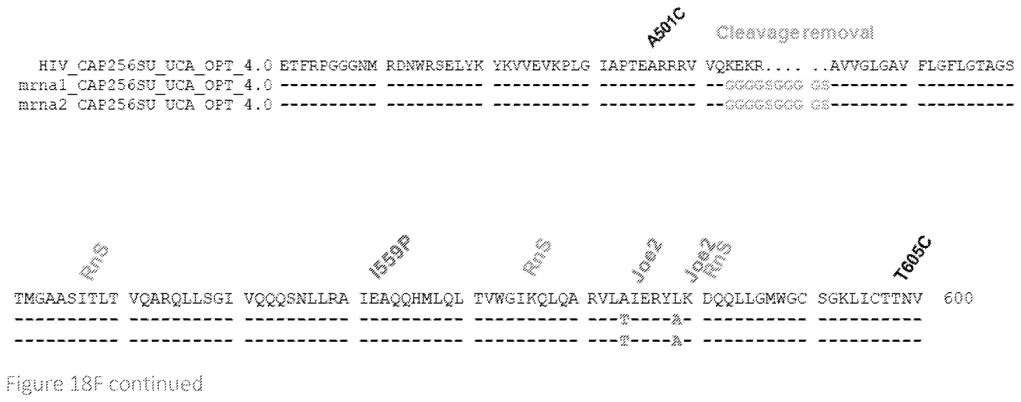

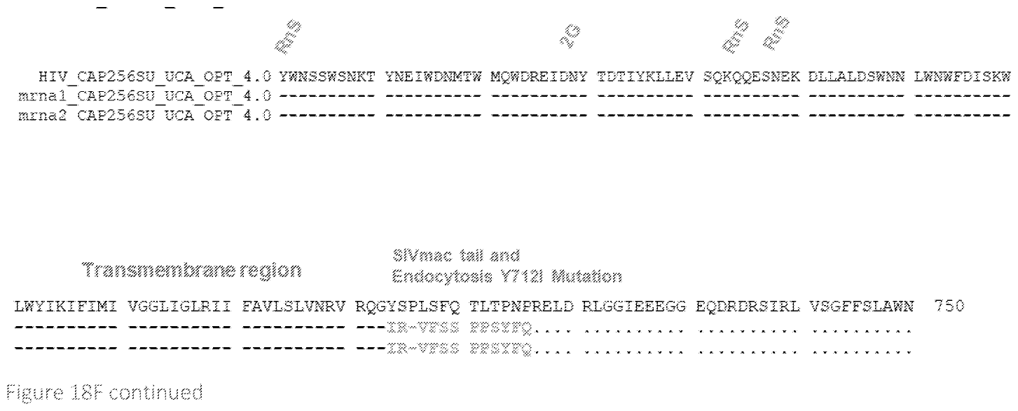

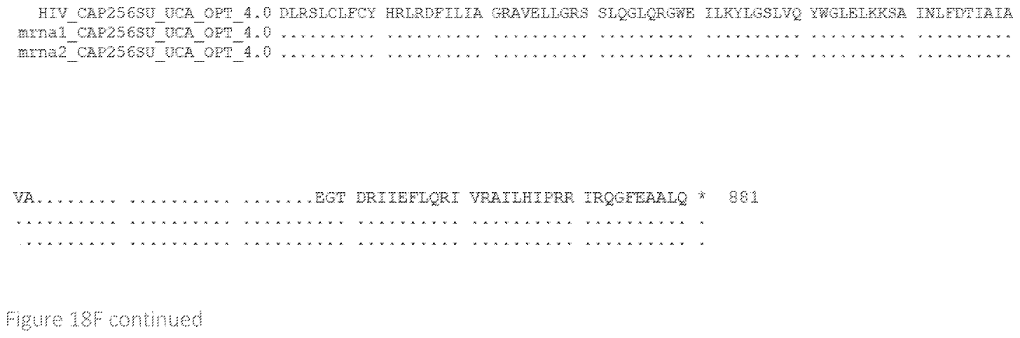

As before, signatures were calculated for binary phenotypes and sites of interest were found to have at least 2 of the 3: (a) contact site, (b) phylogenetic signature, and/or (c) strong q-value <0.1. For month 35 Abs (35B, 35D, 35G, 350 and 35S; no 35M since on a different branch), only signature sites of interest were 130 and 166. FIG. 12A. These were the same for Month 18 Ab, 18D. 166 already carries sensitive R. H130 was chosen because it is the only signature for CH04 UCA. H-130 is slightly sensitive for month 18, 24 and 35 Abs (odd's ratio=2.6-3.5, p=0.19-0.25 for simple Fisher's). For Month 24 (24F; no 24E since on a different branch), additional sites found are 164, 165 and 315 (all contact sites). Each has the sensitive aa in WT.

Several other signatures were identified. FIG. 12B.

Use M-84: Two sensitive signatures M-174 (odds ratio (OR)=2.8-3.4, p=0.007-0.017) and I-174 (OR=2.2-2.3, p=0.015-0.028). Choose M because higher OR and more frequent in C (36.02% vs 35.66% for I), even though it is less frequent in M-group (15.3% vs 44.5% for I).

Use H-130: H130 is the only sensitive signature for CH04UCA (OR=40-42, p=3.1E-6-8.3E-5. It is rare (6.1% in M, 4.6% in C), similar to D, which is favorable for PG9 germline Abs. D is modestly sensitive for PG9 germline Abs (OR=3.4-4.5, p=0.019-0.024).

Use M-161: M is most favorable (OR=2.8-3.4, p=0.0007-0.02). It is at 8.8% in C and 18.9% in M-group. A is borderline sensitive signature for PG9 germline (OR=3.3, p=0.03). It has higher frequency in subtype C (21.8% vs 8.8% for M-161). Since M is not that rare and is stronger signature, use M

Retain D-167: No sensitive signature. Since D is most common in M-group and is in wildtype, we retain it.

Use Q-170: Q is the only sensitive signature (OR=2.1, p=0.017). Fairly frequent in C and M-group (35% and 47%, respectively). Experimentally validated for CAM13 vs PG16 UCA.

Use V-172: V is the only sensitive signature (OR=3.2-4.2, p=5.SE-6-0.004). Fairly frequent (35% in M, 33% in C) Also beneficial to remove the negative E.

Use N-173: N is the strongest sensitive association (OR=13-inf, p=0.0005-0.06). It is rare (2.8% in M, 3.7% in C), but the only other sensitive signature S is also rare (3.8%, 5.6%) (OR=4.1, p=0.065). H is more frequent (16.6% in M, 13.1% in C), but only borderline sensitive signature (OR=2.2-3.1, p=0.08-0.09). Choose N-173 since it is the strongest signature, and while it is rare, it is still found in 51 of 1377 subtype C Envs.

Retain A-174: Only sensitive signature is S (OR=2.4-2.6, p=0.02-0.08). However it is very rare in subtype C (1.8%), in spite of 10.5% in M. A is only weakly resistant (OR=0.37-0.39, p=0.011-0.057). So, change from A is not warranted. The proposed sequence 172-174 VNA though rare is found multiple times (1.9% in C, 26 out of 1377 and 1.1% in M-group, 49 out of 4399).

Use A-200: A is the only sensitive signature (OR=2.8-3, p=0.0005-0.0096). It is moderately frequent (25% in M, 34% in C). Site 200 is a contact site (<8.5 Å from V2 apex bNAbs).

Retain E-269: No sensitive signature, so no need to mutate.

Use S-336: S is the only sensitive signature (OR=3.5-5.8, p=0.0005-0.0098). It is at 13.9% in C, and less frequent in M-group (8.2%).

Use N-636: N is the strongest sensitive signature (OR=2.2-16.4, p=0.0002-0.076). Other sensitive signature is S (OR=1.9, p=0.035). N is somewhat common in C (31.3%), slightly rarer in M-group (18%). S is more frequent (53.7% in C, 40.9% in M-group), but it is not chosen since it is a weaker signature than N.

Use R-732: Only sensitive signature is R (OR=5.4-8.9, p=1.33E-8-0.0084). It is moderately common (35% in M, 61% in C).

For mature V2 apex bNAbs, positively charged V2 and V2 bypervariable, and shorter V1+V2 hypervariable loops are preferred. For UCA/germline Abs, positively charged V2 and V1+V2 loops are preferred. (V3 charge association is likely due to charged aa signatures in V3, which are accounted for later). Thus, preferred short and positively charged V1 and V2 hypervariable loops were identified. These variants include-SET OPT, UCA_OPT_1 and UCA_OPT_2-which will use the same hypervariable loops. The 208 global virus panel based on most charge per unit hypervariable V1 or hypervariable V2 length were sorted, and ZM233.6 and T250-4 were found to be the most preferred, respectively. FIG. 12C.

ZM233.6 hyp V1 loop and T250's hyp V2 loop were used. The M-group distributions of V1, V2 and V1+V2 length and charge with CAP256SU WT are shown in FIG. 12D (each in blue, medians in red and constructs in purple).

Final design includes 10 mutations. FIG. 12E. H-130 accounts for both CH04 UCA and PCT64 intermediate signatures, and the rest are for PG9 germline reverted Abs. Hyp V1 was used from ZM233.6 and hyp V2 was used from T250. The last mutation, G732R, is not in the SHIV construct.

When CAP256 UCA_OPT was tested, it lost neutralization by CAP256_UCA and gained neutralization only by CH01 UCA (IC50=1.92 μg/ml). To see if neutralization could be regained by CAP256_UCA, all of the changes, except H-130 and hypervariable V1 and V2, were reverted. This is CAP256SU_UCA_OPT_2.0. FIG. 12F.

CAP256SU constructs were tested without glycan shield filling. (T250 and CH505 TF were glycan shield optimized). FIG. 12H. Background from SHIV_CAP256SU RMs: N339 was predicted to fill the TF glycan hole never comes up in SHIV_CAP256SU RMs; N396 partially fills TF glycan hole and arises in all 3 RMs before breadth detected. Sporadic gain in RM43037 without breadth; N411->N413 shift also in 3 RMs with breadth and not in RM43037 without breadth. This does not impact glycan shield, as we calculate it, but it could improve glycosylation efficiency of the 408 and 413 glycans, or could impact breadth development by some unknown reasons. Based on these data fully glycan shielded CAP256SU_UCA_OPT_2.0 construct with the following glycans added (N396+N413+N339) was tested. K169R and K170R were also added. With glycans added and K169R is CAP256SU_UCA_OPT_3.0 and with K170R added to this is CAP256 UCA_OPT_3.0_K170R. FIG. 12F.

Neutralize of VR26UCA or VRC26.25, CH01 or CH01 RUA, PG9 or PG9999 RUA, PG16 or PG16 RUA, PCT64 LMCA or PCT64, or Rh-1A or RhA-I neutralization by CAP256SU_V2UCAOPTv3.0K170R_UCA or CAP256SU_V2UCAOPTv3.0K170R_maturebNAb was determined. FIG. 12G.

Any one of these immunogens could be tested in any suitable animal study to determine immunogenicity of the envelopes.

### Example 6

This example shows information and sequences of a CAP256_wk34.80 V2 UCA Optimization. In this Example 6 and FIG. 15, CAP256SU_OPT_4.0 is the same as CAP256SU_UCA_OPT_3.0_K170R in FIGS. 8-12 and FIG. 13.

Previously, 3 design mutations have been successful: N130H; R-169+R-170; and Hyp V1 & V2 loop swaps. It was desired to introduce N130H as it is needed for CH01 UCA reactivity, does not impact CAP256 UCA reactivity and could improve PCT64UCA reactivity. CAP256wk34.80 has 168-KKRR-171 motif. K169R reduces CAP256UCA reactivity by 3-4 fold (CAP256UCAOPT_v2 vs v3). So this motif could be used. A predicted structure is depicted in figure. 15A.

Hypervariable V1 loop may be improved in charge by +2 units and in length by 2 amino acids (although one more V1 glycan will be added and 130 glycan will be removed). FIG. 15B. Hypervariable V2 loop may be improved in charge by +4 units and in length by 3 amino acids. Further the one V2 loop glycan can be removed to avoid potential steric hindrance. FIG. 15C.

For CAP256wk34.80, 2 missing glycans (295 and 339) create glycan holes. FIG. 15D. For CAP256SU, glycans were introduced at positions 339, 396 (already present in wk34.80) and 413. 396 and 413 holes are based on longitudinal SHIV_CAP256 evolution. Adding these glycans did not impact CAP256 UCA neutralization. Thus, N413 was also added to the CAP256wk34.80 constructs.

PCT64UCA escape mutations were investigated. FIG. 15E. N167D was chosen because there are clear signs of escape and structural rationale. Structurally, R-169 and K-169 make sense for investigation. Escape mutations are typically uncharged or negative. However, K-169 is sampled rarely and is not a dominant escape. FIG. 10A. For positions 170 and 171, no or very little escape has been seen in PCT64. Structurally no close interactions appear between these residues and PCT64UCA.

PCT64UCA could prefer a negative V2 loop. Typically it has been observed that that positive and shorter V2 loops are preferred by V2 apex bNAbs, but for PCT64 UCA predicted structure a positively charged region (light chain) interacts with the hypervariable V2 loop. FIG. 15F. Therefore, designs were optimized for negatively charged loops, using the PCT64 early Env diversity.

Very little variation in PCT64 Envs was observed up to month 7. FIG. 15G. The PCT64OPT construct has both a shorter loop that still preserves the interaction between the ends of V2 loop of PCT64 Envs with PCT64UCA. Predicted electrostatic energy is improved by 60KJ/mol when this V2 loop used. Also, previously it was identified found that PCT64 mo18-35 Abs are negatively impacted by V2 length and number of glycans.

The designed V2 loop removes that. A summary of the designs is depicted in FIG. 15H. Neutralization testing experimental data for V2 apex UCA neutralization is depicted in FIG. 15I.

Two additional designs are proposed. FIG. 15J. CAP256SU_UCA_OPT_4.0 performs the best, and has K-171, while CAP256wk34.80_V2UCA_OPT has R-171. It is hypothesized that the R171K mutation will improve V2 UCA reactivity of

CAP256wk34.80_V2UCA_OPT. CAP256SU_UCA_OPT_4.0 has the best presentation of V2 UCA sensitive features. However, it has D-167, and it has been shown that PCT64 UCA requires N-167. It is therefore proposed that D167N mutation will improve the chance of CAP256SU_UCA_OPT_4.0 to be sensitive to PCT64 UCA.

Any one of these immunogens and/or any combination thereof could be tested in any suitable animal study to determine immunogenicity of the envelopes.

### Example 7

This example shows information and sequences of development of improved constructs and mRNAs.

Using cleavage site predictions and SignalP (https://services.healthtech.dtu.dk/service.php?SignalP-6.0), it was found that the motif ¹⁶⁸KRRK¹⁷¹ could introduce an aberrant cleavage site into CAM13RRK. To alleviate this, the mutation K168R is predicted to reduce aberrant cleavage site creation, while not significantly impacting V2 apex bNAb sensitivity. Based on this CAM13RRK+K168R (CAM13RRRK) was constructed and tested. FIG. 18A.

Since CAP256SU_UCA_OPT_4.0 is based on the SHIV_CAP256SU, it has SIVmac239 cytoplasmic tail and Y-375. The reversion Y375S to HIV-1 Ser-375 was tested as CAP256SU_UCA_OPT_4.0 375S and CAP256SU_UCA_OPT_4.0 375S_D167N. Given the advantage of K-171 in other constructs, the R171K mutation was introduced in CAP256wk34.80_V2_UCA_OPT construct. This CAP256wk34.80_V2_UCA_OPT_R17IK construct improved reactivity to several UCAs. FIG. 18B.

The best CAP256SU construct, CAP256SU_UCA_OPT_4.0, was based on SHIV.CAP256SU (i.e. SIVmac239 cytoplasmic tail). Since HIV-1 constructs will be favorable vaccines, all CAP256SU_UCA_OPT_4.0 design mutations were introduced in the backbone of HIV-1 CAP256SU. Testing as a pseudovirus showed that neutralization profile was comparable if not slightly better than the SHIV-based construct. FIG. 18C.

CAP256SU_UCAOPT_4.0 is the best CAP256SU based construct. However, this Env has been difficult to stabilize as SOSIP trimers. CAP256wk34.80 is closely related Env to CAP256SU that can make well-folded SOSIPs (Gorman et al. Cell Rep 31 (1): 107448 2020). Therefore the K169R was transformed to CAP256_wk34.80_V2UCA_OPT_R17IK construct to match all the mutations introduced in CAP256SU_UCA_OPT4.0, and tested this CAP256 wk34.80 V2UCA_OPT_RRK Env. FIG. 18D. Based on the bending of neutralization curve for PCT64 UCA, CAP256_wk34.80_V2UCA_OPT_RRK_D167N will also be tested, which has been shown to be a necessary requirement for PCT64 UCA reactivity.

Strategy 1 for HIV_CAP256SU_UCA_OPT_4.0 mRNA designs. FIG. 18E. Four mRNA constructs that introduce stabilization mutations gradually:

$$mRNA 1 : Joe 2$$

$$mRNA 2 : Joe 2 + F 14$$

$$* SOSIP = A 501 C + T 605 C + I 559 P$$

$$\# Kwong\_muts added are 3 mut + 2 G + RnS ( FIG . 18 E )$$

1535N may also be included. Added the RnS mutations because CAP256SU and CAP256wk34.80 are quite similar to each other.

All mRNA constructs have the signal peptide & cytoplasmic tail from CH848 mRNA constructs. From PDB 6VTT (Gorman et al.) it appears to be the bolded following: MTVTGTWRNYQQWWIWGILGFWMLMICNGLWV. Alignment of sequences for HIV_CAP256SU_UCA_OPT_4.0; mRNAI_CAP256SU_UCA_OPT_4.0; and mRNA2 CAP256SU_UCA_OPT_4.0 is depicted in FIG. 18 F. Dots indicate deletions and dashes indicate identities.

Strategy 2 for CAP256SU_UCA_OPT_4.0 mRNA designs. Using stabilization and expression strategies from Mu et al. Cell Rep 38 (11): 110514 (2022), gp150 and gp160 mRNA constructs were designed for HIV_CAP256SU_UCA_OPT_v4.0. These sequences are denoted HV1303230 to HV1303254. FIG. 17.

Any one of these immunogens and/or any combination thereof could be tested in any suitable animal study to determine immunogenicity of the envelopes.

1. A recombinant HIV-1 envelope polypeptide from Table 2, FIG. 4C, FIG. 12F, FIG. 13, or Table 3, FIG. 14, FIG. 15, FIG. 16, FIG. 17, or FIG. 18F, or Table 4 or encoded by a nucleic acid according to FIG. 19.

2. The recombinant HIV-1 envelope of claim 1, wherein the polypeptide is a non-naturally occurring protomer designed to form an envelope trimer.

3. A nucleic acid encoding the recombinant HIV-1 envelope polypeptide of claim 1.

4. A recombinant trimer comprising three identical protomers of an envelope from Table 2, FIG. 4C, FIG. 12F, FIG. 13, Table 3, FIG. 14, FIG. 15, FIG. 16, FIG. 17, or FIG. 18F, or Table 4 or encoded by a nucleic acid according to FIG. 19.

5. An immunogenic composition comprising the recombinant trimer of claim 4 and a carrier.

6. An immunogenic composition comprising the nucleic acid of claim 3 and a carrier.

7. The immunogenic composition of claim 5 er 6 further comprising an adjuvant.

8. The nucleic acid of claim 3, wherein the nucleic acid is operably linked to a promoter, and optionally wherein the nucleic acid is inserted in an expression vector.

9. A method of inducing an immune response in a subject comprising administering a composition comprising any suitable form of the polypeptide of claim 1 or a nucleic acid encoding the polypeptide in an amount sufficient to induce an immune response.

10. The method of claim 9 wherein the nucleic acid encodes a gp120 envelope, gp120D8 envelope, a gp140 envelope (gp140C, gp140CF, gp140CFI) as soluble or stabilized protomer of a SOSIP trimer, a gp145 envelope, a gp150 envelope, a transmembrane bound envelope, a gp160 envelope or an envelope designed to multimerize.

11. The method of claim 9 wherein the polypeptide is gp120 envelope, gp120D8 envelope, a gp140 envelope (gp140C, gp140CF, gp140CFI) as soluble or stabilized protomer of a SOSIP trimer, a gp145 envelope, a gp150 envelope, a transmembrane bound envelope, or an envelope designed to multimerize.

12. The method of claim 9, wherein the composition further comprises an adjuvant.

13. The method of claim 9, further comprising administering an agent which modulates host immune tolerance.

14. The method of claim 11, wherein the polypeptide administered is multimerized in a liposome or nanoparticle.

15. The method of claim 10, wherein the nucleic acid administered is a mRNA.

16. The method of claim 10, wherein the nucleic acid is encapsulated in a lipid nanoparticle.

17. (canceled)

18. A composition comprising a nanoparticle and a carrier, wherein the nanoparticle comprises any one of the envelopes of claim 1.

19. (canceled)

20. A composition comprising a nanoparticle and a carrier, wherein the nanoparticle comprises any one of the trimers of claim 2.

21-23. (canceled)

24. A composition comprising a nanoparticle and carrier, wherein the nanoparticle comprises any one of the nucleic acids of claim 3.

25. The composition of claim 24, wherein the nucleic acid is a mRNA.

26-52. (canceled)
